SEQUENCE LISTING (98 sequences)

SEQ ID NO: 1 - protein, 133 aa, Homo sapiens [sequence omitted]

SEQ ID NO: 2 - protein, 153 aa, Homo sapiens
  REGION 1..153: note = misc_feature - IL-2 (homo sapiens) (precursor) NCBI Accession No.: AAB46883.1
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 3 - protein, 132 aa, synthetic construct
  REGION 1..132: note = Synthetic: aldesleukin
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 4 - protein, 133 aa, synthetic construct
  REGION 1..133: note = Synthetic: IL-2_C125S
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 5 - protein, 133 aa, synthetic construct
  REGION 1..133: note = Synthetic: IL-2_P65X
  VARIANT 65: note = Xaa is an unnatural amino acid
APTSSSTKKTQLQLEHLLLDLQMILNGINNYKNPKLTRMLTFKFYMPKKATELKHLQCLEEELKXLEEVLNLAQSKNFHLRPRDLISNINVIVLELKGSETTFMCEYADETATIVEFLNRWITFSQSIISTLT

SEQ ID NO: 6 - protein, 133 aa, synthetic construct
  REGION 1..133: note = Synthetic: IL-2_E62X
  VARIANT 62: note = Xaa is an unnatural amino acid
APTSSSTKKTQLQLEHLLLDLQMILNGINNYKNPKLTRMLTFKFYMPKKATELKHLQCLEEXLKPLEEVLNLAQSKNFHLRPRDLISNINVIVLELKGSETTFMCEYADETATIVEFLNRWITFSQSIISTLT

SEQ ID NO: 7 - protein, 133 aa, synthetic construct
  REGION 1..133: note = Synthetic: IL-2_F42X
  VARIANT 42: note = Xaa is an unnatural amino acid
APTSSSTKKTQLQLEHLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 8 - protein, 133 aa, synthetic construct
  REGION 1..133: note = Synthetic: IL-2_K43X
  VARIANT 43: note = Xaa is an unnatural amino acid
APTSSSTKKTQLQLEHLLLDLQMILNGINNYKNPKLTRMLTFXFYMPKKATELKHLQCLEEELKPLEEVLNLAQSKNFHLRPRDLISNINVIVLELKGSETTFMCEYADETATIVEFLNRWITFSQSIISTLT

SEQ ID NO: 9 - protein, 133 aa, synthetic construct
  REGION 1..133: note = Synthetic: IL-2_K35X
  VARIANT 35: note = Xaa is an unnatural amino acid
APTSSSTKKTQLQLEHLLLDLQMILNGINNYKNPXLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 10 - protein, 133 aa, synthetic construct; identical to SEQ ID NO: 5
  REGION 1..133: note = Synthetic: IL-2_P65[AzK]
  SITE 65: note = Xaa is [AzK]: N6-((2-azidoethoxy)-carbonyl)-L-lysine

SEQ ID NO: 11 - protein, 133 aa, synthetic construct; identical to SEQ ID NO: 6
  REGION 1..133: note = Synthetic: IL-2_E62[AzK]
  SITE 62: note = Xaa is [AzK]: N6-((2-azidoethoxy)-carbonyl)-L-lysine

SEQ ID NO: 12 - protein, 133 aa, synthetic construct; identical to SEQ ID NO: 7
  REGION 1..133: note = Synthetic: IL-2_F42[AzK]
  SITE 42: note = Xaa is [AzK]: N6-((2-azidoethoxy)-carbonyl)-L-lysine

SEQ ID NO: 13 - protein, 133 aa, synthetic construct; identical to SEQ ID NO: 8
  REGION 1..133: note = Synthetic: IL-2_K43[AzK]
  SITE 43: note = Xaa is [AzK]: N6-((2-azidoethoxy)-carbonyl)-L-lysine

SEQ ID NO: 14 - protein, 133 aa, synthetic construct; identical to SEQ ID NO: 9
  REGION 1..133: note = Synthetic: IL-2_K35[AzK]
  SITE 35: note = Xaa is [AzK]: N6-((2-azidoethoxy)-carbonyl)-L-lysine

SEQ ID NO: 15 - protein, 133 aa, synthetic construct; identical to SEQ ID NO: 5
  REGION 1..133: note = Synthetic: IL-2_P65[AzK_PEG]
  SITE 65: note = Xaa is [AzK_PEG]: N6-((2-azidoethoxy)-carbonyl)-L-lysine stably-conjugated to PEG

SEQ ID NO: 16 - protein, 133 aa, synthetic construct; identical to SEQ ID NO: 6
  REGION 1..133: note = Synthetic: IL-2_E62[AzK_PEG]
  SITE 62: note = Xaa is [AzK_PEG]: N6-((2-azidoethoxy)-carbonyl)-L-lysine stably-conjugated to PEG

SEQ ID NO: 17 - protein, 133 aa, synthetic construct; identical to SEQ ID NO: 7
  REGION 1..133: note = Synthetic: IL-2_F42[AzK_PEG]
  SITE 42: note = Xaa is [AzK_PEG]: N6-((2-azidoethoxy)-carbonyl)-L-lysine stably-conjugated to PEG

SEQ ID NO: 18 - protein, 133 aa, synthetic construct; identical to SEQ ID NO: 8
  REGION 1..133: note = Synthetic: IL-2_K43[AzK_PEG]
  SITE 43: note = Xaa is [AzK_PEG]: N6-((2-azidoethoxy)-carbonyl)-L-lysine stably-conjugated to PEG

SEQ ID NO: 19 - protein, 133 aa, synthetic construct; identical to SEQ ID NO: 9
  REGION 1..133: note = Synthetic: IL-2_K35[AzK_PEG]
  SITE 35: note = Xaa is [AzK_PEG]: N6-((2-azidoethoxy)-carbonyl)-L-lysine stably-conjugated to PEG

SEQ ID NO: 20 - protein, 133 aa, synthetic construct; identical to SEQ ID NO: 5
  REGION 1..133: note = Synthetic: IL-2_P65[AzK_PEG5kD]
  SITE 65: note = Xaa is [AzK_PEG5kD]: N6-((2-azidoethoxy)-carbonyl)-L-lysine stably-conjugated to PEG

SEQ ID NO: 21 - protein, 133 aa, synthetic construct; identical to SEQ ID NO: 6
  REGION 1..133: note = Synthetic: IL-2_E62[AzK_PEG5kD]
  SITE 62: note = Xaa is [AzK_PEG5kD]: N6-((2-azidoethoxy)-carbonyl)-L-lysine stably-conjugated to PEG

SEQ ID NO: 22 - protein, 133 aa, synthetic construct; identical to SEQ ID NO: 7
  REGION 1..133: note = Synthetic: IL-2_F42[AzK_PEG5kD]
  SITE 42: note = Xaa is [AzK_PEG5kD]: N6-((2-azidoethoxy)-carbonyl)-L-lysine stably-conjugated to PEG

SEQ ID NO: 23 - protein, 133 aa, synthetic construct; identical to SEQ ID NO: 8
  REGION 1..133: note = Synthetic: IL-2_K43[AzK_PEG5kD]
  SITE 43: note = Xaa is [AzK_PEG5kD]: N6-((2-azidoethoxy)-carbonyl)-L-lysine stably-conjugated to PEG

SEQ ID NO: 24 - protein, 133 aa, synthetic construct; identical to SEQ ID NO: 9
  REGION 1..133: note = Synthetic: IL-2_K35[AzK_PEG5kD]
  SITE 35: note = Xaa is [AzK_PEG5kD]: N6-((2-azidoethoxy)-carbonyl)-L-lysine stably-conjugated to PEG

SEQ ID NO: 25 - protein, 133 aa, synthetic construct; identical to SEQ ID NO: 5
  REGION 1..133: note = Synthetic: IL-2_P65[AzK_PEG30kD]
  SITE 65: note = Xaa is [AzK_PEG30kD]: N6-((2-azidoethoxy)-carbonyl)-L-lysine stably-conjugated to PEG

SEQ ID NO: 26 - protein, 133 aa, synthetic construct; identical to SEQ ID NO: 6
  REGION 1..133: note = Synthetic: IL-2_E62[AzK_PEG30kD]
  SITE 62: note = Xaa is [AzK_PEG30kD]: N6-((2-azidoethoxy)-carbonyl)-L-lysine stably-conjugated to PEG

SEQ ID NO: 27 - protein, 133 aa, synthetic construct; identical to SEQ ID NO: 7
  REGION 1..133: note = Synthetic: IL-2_F42[AzK_PEG30kD]
  SITE 42: note = Xaa is [AzK_PEG30kD]: N6-((2-azidoethoxy)-carbonyl)-L-lysine stably-conjugated to PEG

SEQ ID NO: 28 - protein, 133 aa, synthetic construct; identical to SEQ ID NO: 8
  REGION 1..133: note = Synthetic: IL-2_K43[AzK_PEG30kD]
  SITE 43: note = Xaa is [AzK_PEG30kD]: N6-((2-azidoethoxy)-carbonyl)-L-lysine stably-conjugated to PEG

SEQ ID NO: 29 - protein, 133 aa, synthetic construct; identical to SEQ ID NO: 9
  REGION 1..133: note = Synthetic: IL-2_K35[AzK_PEG30kD]
  SITE 35: note = Xaa is [AzK_PEG30kD]: N6-((2-azidoethoxy)-carbonyl)-L-lysine stably-conjugated to PEG

SEQ ID NO: 30 - protein, 132 aa, synthetic construct
  REGION 1..132: note = Synthetic: IL-2_P65X-1
  VARIANT 64: note = Xaa is an unnatural amino acid
PTSSSTKKTQLQLEHLLLDLQMILNGINNYKNPKLTRMLTFKFYMPKKATELKHLQCLEEELKXLEEVLNLAQSKNFHLRPRDLISNINVIVLELKGSETTFMCEYADETATIVEFLNRWITFSQSIISTLT

SEQ ID NO: 31 - protein, 132 aa, synthetic construct
  REGION 1..132: note = Synthetic: IL-2_E62X-1
  VARIANT 61: note = Xaa is an unnatural amino acid
PTSSSTKKTQLQLEHLLLDLQMILNGINNYKNPKLTRMLTFKFYMPKKATELKHLQCLEEXLKPLEEVLNLAQSKNFHLRPRDLISNINVIVLELKGSETTFMCEYADETATIVEFLNRWITFSQSIISTLT

SEQ ID NO: 32 - protein, 132 aa, synthetic construct
  REGION 1..132: note = Synthetic: IL-2_F42X-1
  VARIANT 41: note = Xaa is an unnatural amino acid
PTSSSTKKTQLQLEHLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 33 - protein, 132 aa, synthetic construct
  REGION 1..132: note = Synthetic: IL-2_K43X-1
  VARIANT 42: note = Xaa is an unnatural amino acid
PTSSSTKKTQLQLEHLLLDLQMILNGINNYKNPKLTRMLTFXFYMPKKATELKHLQCLEEELKPLEEVLNLAQSKNFHLRPRDLISNINVIVLELKGSETTFMCEYADETATIVEFLNRWITFSQSIISTLT

SEQ ID NO: 34 - protein, 132 aa, synthetic construct
  REGION 1..132: note = Synthetic: IL-2_K35X-1
  VARIANT 34: note = Xaa is an unnatural amino acid
PTSSSTKKTQLQLEHLLLDLQMILNGINNYKNPXLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 35 - protein, 132 aa, synthetic construct; identical to SEQ ID NO: 30
  REGION 1..132: note = Synthetic: IL-2_P65[AzK]-1
  SITE 64: note = Xaa is [AzK]: N6-((2-azidoethoxy)-carbonyl)-L-lysine

SEQ ID NO: 36 - protein, 132 aa, synthetic construct; identical to SEQ ID NO: 31
  REGION 1..132: note = Synthetic: IL-2_E62[AzK]-1
  SITE 61: note = Xaa is [AzK]: N6-((2-azidoethoxy)-carbonyl)-L-lysine

SEQ ID NO: 37 - protein, 132 aa, synthetic construct; identical to SEQ ID NO: 32
  REGION 1..132: note = Synthetic: IL-2_F42[AzK]-1
  SITE 41: note = Xaa is [AzK]: N6-((2-azidoethoxy)-carbonyl)-L-lysine

SEQ ID NO: 38 - protein, 132 aa, synthetic construct; identical to SEQ ID NO: 33
  REGION 1..132: note = Synthetic: IL-2_K43[AzK]-1
  SITE 42: note = Xaa is [AzK]: N6-((2-azidoethoxy)-carbonyl)-L-lysine

SEQ ID NO: 39 - protein, 132 aa, synthetic construct; identical to SEQ ID NO: 34
  REGION 1..132: note = Synthetic: IL-2_K35[AzK]-1
  SITE 34: note = Xaa is [AzK]: N6-((2-azidoethoxy)-carbonyl)-L-lysine

SEQ ID NO: 40 - protein, 132 aa, synthetic construct; identical to SEQ ID NO: 30
  REGION 1..132: note = Synthetic: IL-2_P65[AzK_L1_PEG]-1
  SITE 64: note = Xaa is [AzK_L1_PEG]: N6-((2-azidoethoxy)-carbonyl)-L-lysine stably-conjugated to PEG

SEQ ID NO: 41 - protein, 132 aa, synthetic construct; identical to SEQ ID NO: 31
  REGION 1..132: note = Synthetic: IL-2_E62[AzK_ L1_PEG]-1
  SITE 61: note = Xaa is [AzK_ L1_PEG]: N6-((2-azidoethoxy)-carbonyl)-L-lysine stably-conjugated to PEG

SEQ ID NO: 42 - protein, 132 aa, synthetic construct; identical to SEQ ID NO: 32
  REGION 1..132: note = Synthetic: IL-2_F42[AzK_ L1_PEG]-1
  SITE 41: note = Xaa is [AzK_ L1_PEG]: N6-((2-azidoethoxy)-carbonyl)-L-lysine stably-conjugated to PEG

SEQ ID NO: 43 - protein, 132 aa, synthetic construct; identical to SEQ ID NO: 33
  REGION 1..132: note = Synthetic: IL-2_K43[AzK_ L1_PEG]-1
  SITE 42: note = Xaa is [AzK_ L1_PEG]: N6-((2-azidoethoxy)-carbonyl)-L-lysine stably-conjugated to PEG

SEQ ID NO: 44 - protein, 132 aa, synthetic construct; identical to SEQ ID NO: 34
  REGION 1..132: note = Synthetic: IL-2_K35[AzK_ L1_PEG]-1
  SITE 34: note = Xaa is [AzK_ L1_PEG]: N6-((2-azidoethoxy)-carbonyl)-L-lysine stably-conjugated to PEG

SEQ ID NO: 45 - protein, 132 aa, synthetic construct; identical to SEQ ID NO: 30
  REGION 1..132: note = Synthetic: IL-2_P65[AzK_ L1_PEG5kD]-1
  SITE 64: note = Xaa is [AzK_ L1_PEG5kD]: N6-((2-azidoethoxy)-carbonyl)-L-lysine stably-conjugated to PEG

SEQ ID NO: 46 - protein, 132 aa, synthetic construct; identical to SEQ ID NO: 31
  REGION 1..132: note = Synthetic: IL-2_E62[AzK_ L1_PEG5kD]-1
  SITE 61: note = Xaa is [AzK_ L1_PEG5kD]: N6-((2-azidoethoxy)-carbonyl)-L-lysine stably-conjugated to PEG

SEQ ID NO: 47 - protein, 132 aa, synthetic construct; identical to SEQ ID NO: 32
  REGION 1..132: note = Synthetic: IL-2_F42[AzK_ L1_PEG5kD]-1
  SITE 41: note = Xaa is [AzK_ L1_PEG5kD]: N6-((2-azidoethoxy)-carbonyl)-L-lysine stably-conjugated to PEG

SEQ ID NO: 48 - protein, 132 aa, synthetic construct; identical to SEQ ID NO: 33
  REGION 1..132: note = Synthetic: IL-2_K43[AzK_ L1_PEG5kD]-1
  SITE 42: note = Xaa is [AzK_ L1_PEG5kD]: N6-((2-azidoethoxy)-carbonyl)-L-lysine stably-conjugated to PEG

SEQ ID NO: 49 - protein, 132 aa, synthetic construct; identical to SEQ ID NO: 34
  REGION 1..132: note = Synthetic: IL-2_K35[AzK_ L1_PEG5kD]-1
  SITE 34: note = Xaa is [AzK_ L1_PEG5kD]: N6-((2-azidoethoxy)-carbonyl)-L-lysine stably-conjugated to PEG

SEQ ID NO: 50 - protein, 132 aa, synthetic construct; identical to SEQ ID NO: 30
  REGION 1..132: note = Synthetic: IL-2_P65[AzK_ L1_PEG30kD]-1
  SITE 64: note = Xaa is [AzK_ L1_PEG30kD]: N6-((2-azidoethoxy)-carbonyl)-L-lysine stably-conjugated to PEG

SEQ ID NO: 51 - protein, 132 aa, synthetic construct; identical to SEQ ID NO: 31
  REGION 1..132: note = Synthetic: IL-2_E62[AzK_ L1_PEG30kD]-1
  SITE 61: note = Xaa is [AzK_ L1_PEG30kD]: N6-((2-azidoethoxy)-carbonyl)-L-lysine stably-conjugated to PEG

SEQ ID NO: 52 - protein, 132 aa, synthetic construct; identical to SEQ ID NO: 32
  REGION 1..132: note = Synthetic: IL-2_F42[AzK_ L1_PEG30kD]-1
  SITE 41: note = Xaa is [AzK_ L1_PEG30kD]: N6-((2-azidoethoxy)-carbonyl)-L-lysine stably-conjugated to PEG

SEQ ID NO: 53 - protein, 132 aa, synthetic construct; identical to SEQ ID NO: 33
  REGION 1..132: note = Synthetic: IL-2_K43[AzK_ L1_PEG30kD]-1
  SITE 42: note = Xaa is [AzK_ L1_PEG30kD]: N6-((2-azidoethoxy)-carbonyl)-L-lysine stably-conjugated to PEG

SEQ ID NO: 54 - protein, 132 aa, synthetic construct; identical to SEQ ID NO: 34
  REGION 1..132: note = Synthetic: IL-2_K35[AzK_ L1_PEG30kD]-1
  SITE 34: note = Xaa is [AzK_ L1_PEG30kD]: N6-((2-azidoethoxy)-carbonyl)-L-lysine stably-conjugated to PEG

SEQ ID NO: 55 - protein, 133 aa, synthetic construct; identical to SEQ ID NO: 5
  REGION 1..133: note = Synthetic: IL-2_P65[AzK_L1_PEG]-2
  SITE 65: note = Xaa is [AzK_L1_PEG]: N6-((2-azidoethoxy)-carbonyl)-L-lysine stably-conjugated to PEG

SEQ ID NO: 56 - protein, 133 aa, synthetic construct; identical to SEQ ID NO: 6
  REGION 1..133: note = Synthetic: IL-2_E62[AzK_ L1_PEG]-2
  SITE 62: note = Xaa is [AzK_ L1_PEG]: N6-((2-azidoethoxy)-carbonyl)-L-lysine stably-conjugated to PEG

SEQ ID NO: 57 - protein, 133 aa, synthetic construct; identical to SEQ ID NO: 7
  REGION 1..133: note = Synthetic: IL-2_F42[AzK_ L1_PEG]-2
  SITE 42: note = Xaa is [AzK_ L1_PEG]: N6-((2-azidoethoxy)-carbonyl)-L-lysine stably-conjugated to PEG

SEQ ID NO: 58 - protein, 133 aa, synthetic construct; identical to SEQ ID NO: 8
  REGION 1..133: note = Synthetic: IL-2_K43[AzK_ L1_PEG]-2
  SITE 43: note = Xaa is [AzK_ L1_PEG]: N6-((2-azidoethoxy)-carbonyl)-L-lysine stably-conjugated to PEG

SEQ ID NO: 59 - protein, 133 aa, synthetic construct; identical to SEQ ID NO: 9
  REGION 1..133: note = Synthetic: IL-2_K35[AzK_ L1_PEG]-2
  SITE 35: note = Xaa is [AzK_ L1_PEG]: N6-((2-azidoethoxy)-carbonyl)-L-lysine stably-conjugated to PEG

SEQ ID NO: 60 - protein, 133 aa, synthetic construct; identical to SEQ ID NO: 5
  REGION 1..133: note = Synthetic: IL-2_P65[AzK_ L1_PEG5kD]-2
  SITE 65: note = Xaa is [AzK_ L1_PEG5kD]: N6-((2-azidoethoxy)-carbonyl)-L-lysine stably-conjugated to PEG

SEQ ID NO: 61 - protein, 133 aa, synthetic construct; identical to SEQ ID NO: 6
  REGION 1..133: note = Synthetic: IL-2_E62[AzK_ L1_PEG5kD]-2
  SITE 62: note = Xaa is [AzK_ L1_PEG5kD]: N6-((2-azidoethoxy)-carbonyl)-L-lysine stably-conjugated to PEG

SEQ ID NO: 62 - protein, 133 aa, synthetic construct; identical to SEQ ID NO: 7
  REGION 1..133: note = Synthetic: IL-2_F42[AzK_ L1_PEG5kD]-2
  SITE 42: note = Xaa is [AzK_ L1_PEG5kD]: N6-((2-azidoethoxy)-carbonyl)-L-lysine stably-conjugated to PEG

SEQ ID NO: 63 - protein, 133 aa, synthetic construct; identical to SEQ ID NO: 8
  REGION 1..133: note = Synthetic: IL-2_K43[AzK_ L1_PEG5kD]-2
  SITE 43: note = Xaa is [AzK_ L1_PEG5kD]: N6-((2-azidoethoxy)-carbonyl)-L-lysine stably-conjugated to PEG

SEQ ID NO: 64 - protein, 133 aa, synthetic construct; identical to SEQ ID NO: 9
  REGION 1..133: note = Synthetic: IL-2_K35[AzK_ L1_PEG5kD]-2
  SITE 35: note = Xaa is [AzK_ L1_PEG5kD]: N6-((2-azidoethoxy)-carbonyl)-L-lysine stably-conjugated to PEG

SEQ ID NO: 65 - protein, 133 aa, synthetic construct; identical to SEQ ID NO: 5
  REGION 1..133: note = Synthetic: IL-2_P65[AzK_ L1_PEG30kD]-2
  SITE 65: note = Xaa is [AzK_ L1_PEG30kD]: N6-((2-azidoethoxy)-carbonyl)-L-lysine stably-conjugated to PEG

SEQ ID NO: 66 - protein, 133 aa, synthetic construct; identical to SEQ ID NO: 6
  REGION 1..133: note = Synthetic: IL-2_E62[AzK_ L1_PEG30kD]-2
  SITE 62: note = Xaa is [AzK_ L1_PEG30kD]: N6-((2-azidoethoxy)-carbonyl)-L-lysine stably-conjugated to PEG

SEQ ID NO: 67 - protein, 133 aa, synthetic construct; identical to SEQ ID NO: 7
  REGION 1..133: note = Synthetic: IL-2_F42[AzK_ L1_PEG30kD]-2
  SITE 42: note = Xaa is [AzK_ L1_PEG30kD]: N6-((2-azidoethoxy)-carbonyl)-L-lysine stably-conjugated to PEG

SEQ ID NO: 68 - protein, 133 aa, synthetic construct; identical to SEQ ID NO: 8
  REGION 1..133: note = Synthetic: IL-2_K43[AzK_ L1_PEG30kD]-2
  SITE 43: note = Xaa is [AzK_ L1_PEG30kD]: N6-((2-azidoethoxy)-carbonyl)-L-lysine stably-conjugated to PEG

SEQ ID NO: 69 - protein, 133 aa, synthetic construct; identical to SEQ ID NO: 9
  REGION 1..133: note = Synthetic: IL-2_K35[AzK_ L1_PEG30kD]-2
  SITE 35: note = Xaa is [AzK_ L1_PEG30kD]: N6-((2-azidoethoxy)-carbonyl)-L-lysine stably-conjugated to PEG

SEQ ID NO: 70 - protein, 132 aa, synthetic construct; identical to SEQ ID NO: 30
  REGION 1..132: note = Synthetic: IL-2_P65[AzK_PEG]-1
  SITE 64: note = Xaa is [AzK_PEG]: N6-((2-azidoethoxy)-carbonyl)-L-lysine stably-conjugated to PEG

SEQ ID NO: 71 - protein, 132 aa, synthetic construct; identical to SEQ ID NO: 31
  REGION 1..132: note = Synthetic: IL-2_E62[AzK_ PEG]-1
  SITE 61: note = Xaa is [AzK_ PEG]: N6-((2-azidoethoxy)-carbonyl)-L-lysine stably-conjugated to PEG

SEQ ID NO: 72 - protein, 132 aa, synthetic construct; identical to SEQ ID NO: 32
  REGION 1..132: note = Synthetic: IL-2_F42[AzK_ PEG]-1
  SITE 41: note = Xaa is [AzK_ PEG]: N6-((2-azidoethoxy)-carbonyl)-L-lysine stably-conjugated to PEG

SEQ ID NO: 73 - protein, 132 aa, synthetic construct; identical to SEQ ID NO: 33
  REGION 1..132: note = Synthetic: IL-2_K43[AzK_ PEG]-1
  SITE 42: note = Xaa is [AzK_ PEG]: N6-((2-azidoethoxy)-carbonyl)-L-lysine stably-conjugated to PEG

SEQ ID NO: 74 - protein, 132 aa, synthetic construct; identical to SEQ ID NO: 34
  REGION 1..132: note = Synthetic: IL-2_K35[AzK_ PEG]-1
  SITE 34: note = Xaa is [AzK_ PEG]: N6-((2-azidoethoxy)-carbonyl)-L-lysine stably-conjugated to PEG

SEQ ID NO: 75 - protein, 132 aa, synthetic construct; identical to SEQ ID NO: 30
  REGION 1..132: note = Synthetic: IL-2_P65[AzK_ PEG5kD]-1
  SITE 64: note = Xaa is [AzK_ PEG5kD]: N6-((2-azidoethoxy)-carbonyl)-L-lysine stably-conjugated to PEG

SEQ ID NO: 76 - protein, 132 aa, synthetic construct; identical to SEQ ID NO: 31
  REGION 1..132: note = Synthetic: IL-2_E62[AzK_PEG5kD]-1
  SITE 61: note = Xaa is [AzK_PEG5kD]: N6-((2-azidoethoxy)-carbonyl)-L-lysine stably-conjugated to PEG

SEQ ID NO: 77 - protein, 132 aa, synthetic construct; identical to SEQ ID NO: 32
  REGION 1..132: note = Synthetic: IL-2_F42[AzK_PEG5kD]-1
  SITE 41: note = Xaa is [AzK_PEG5kD]: N6-((2-azidoethoxy)-carbonyl)-L-lysine stably-conjugated to PEG

SEQ ID NO: 78 - protein, 132 aa, synthetic construct; identical to SEQ ID NO: 33
  REGION 1..132: note = Synthetic: IL-2_K43[AzK_PEG5kD]-1
  SITE 42: note = Xaa is [AzK_PEG5kD]: N6-((2-azidoethoxy)-carbonyl)-L-lysine stably-conjugated to PEG

SEQ ID NO: 79 - protein, 132 aa, synthetic construct; identical to SEQ ID NO: 34
  REGION 1..132: note = Synthetic: IL-2_K35[AzK_PEG5kD]-1
  SITE 34: note = Xaa is [AzK_PEG5kD]: N6-((2-azidoethoxy)-carbonyl)-L-lysine stably-conjugated to PEG

SEQ ID NO: 80 - protein, 132 aa, synthetic construct; identical to SEQ ID NO: 30
  REGION 1..132: note = Synthetic: IL-2_P65[AzK_PEG30kD]-1
  SITE 64: note = Xaa is [AzK_PEG30kD]: N6-((2-azidoethoxy)-carbonyl)-L-lysine stably-conjugated to PEG

SEQ ID NO: 81 - protein, 132 aa, synthetic construct; identical to SEQ ID NO: 31
  REGION 1..132: note = Synthetic: IL-2_E62[AzK_PEG30kD]-1
  SITE 61: note = Xaa is [AzK_PEG30kD]: N6-((2-azidoethoxy)-carbonyl)-L-lysine stably-conjugated to PEG

SEQ ID NO: 82 - protein, 132 aa, synthetic construct; identical to SEQ ID NO: 32
  REGION 1..132: note = Synthetic: IL-2_F42[AzK_PEG30kD]-1
  SITE 41: note = Xaa is [AzK_PEG30kD]: N6-((2-azidoethoxy)-carbonyl)-L-lysine stably-conjugated to PEG

SEQ ID NO: 83 - protein, 132 aa, synthetic construct; identical to SEQ ID NO: 33
  REGION 1..132: note = Synthetic: IL-2_K43[AzK_PEG30kD]-1
  SITE 42: note = Xaa is [AzK_PEG30kD]: N6-((2-azidoethoxy)-carbonyl)-L-lysine stably-conjugated to PEG

SEQ ID NO: 84 - protein, 132 aa, synthetic construct; identical to SEQ ID NO: 34
  REGION 1..132: note = Synthetic: IL-2_K35[AzK_PEG30kD]-1
  SITE 34: note = Xaa is [AzK_PEG30kD]: N6-((2-azidoethoxy)-carbonyl)-L-lysine stably-conjugated to PEG

SEQ ID NO: 85 - protein, 133 aa, synthetic construct
  REGION 1..133: note = Synthetic: IL-2_F44X
  VARIANT 44: note = Xaa is an unnatural amino acid
APTSSSTKKTQLQLEHLLLDLQMILNGINNYKNPKLTRMLTFKXYMPKKATELKHLQCLEEELKPLEEVLNLAQSKNFHLRPRDLISNINVIVLELKGSETTFMCEYADETATIVEFLNRWITFCQSIISTLT

SEQ ID NO: 86 - protein, 132 aa, synthetic construct
  REGION 1..132: note = Synthetic: IL-2_F44X-1
  VARIANT 43: note = Xaa is an unnatural amino acid
PTSSSTKKTQLQLEHLLLDLQMILNGINNYKNPKLTRMLTFKXYMPKKATELKHLQCLEEELKPLEEVLNLAQSKNFHLRPRDLISNINVIVLELKGSETTFMCEYADETATIVEFLNRWITFCQSIISTLT

SEQ ID NO: 87 - protein, 133 aa, synthetic construct
  REGION 1..133: note = Synthetic: IL-2_R38X
  VARIANT 38: note = Xaa is an unnatural amino acid
APTSSSTKKTQLQLEHLLLDLQMILNGINNYKNPKLTXMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 88 - protein, 132 aa, synthetic construct
  REGION 1..132: note = Synthetic: IL-2_R38X-1
  VARIANT 37: note = Xaa is an unnatural amino acid
PTSSSTKKTQLQLEHLLLDLQMILNGINNYKNPKLTXMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 89 - protein, 133 aa, synthetic construct
  REGION 1..133: note = Synthetic: IL-2_T41X
  VARIANT 41: note = Xaa is an unnatural amino acid
APTSSSTKKTQLQLEHLLLDLQMILNGINNYKNPKLTRMLXFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 90 - protein, 132 aa, synthetic construct
  REGION 1..132: note = Synthetic: IL-2_T41X-1
  VARIANT 40: note = Xaa is an unnatural amino acid
PTSSSTKKTQLQLEHLLLDLQMILNGINNYKNPKLTRMLXFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 91 - protein, 133 aa, synthetic construct
  REGION 1..133: note = Synthetic: IL-2_E68X
  VARIANT 68: note = Xaa is an unnatural amino acid
APTSSSTKKTQLQLEHLLLDLQMILNGINNYKNPKLTRMLTFKFYMPKKATELKHLQCLEEELKPLEXVLNLAQSKNFHLRPRDLISNINVIVLELKGSETTFMCEYADETATIVEFLNRWITFCQSIISTLT

SEQ ID NO: 92 - protein, 132 aa, synthetic construct
  REGION 1..132: note = Synthetic: IL-2_E68X-1
  VARIANT 67: note = Xaa is an unnatural amino acid
PTSSSTKKTQLQLEHLLLDLQMILNGINNYKNPKLTRMLTFKFYMPKKATELKHLQCLEEELKPLEXVLNLAQSKNFHLRPRDLISNINVIVLELKGSETTFMCEYADETATIVEFLNRWITFCQSIISTLT

SEQ ID NO: 93 - protein, 133 aa, synthetic construct
  REGION 1..133: note = Synthetic: IL-2_Y45X
  VARIANT 45: note = Xaa is an unnatural amino acid
APTSSSTKKTQLQLEHLLLDLQMILNGINNYKNPKLTRMLTFKFXMPKKATELKHLQCLEEELKPLEEVLNLAQSKNFHLRPRDLISNINVIVLELKGSETTFMCEYADETATIVEFLNRWITFCQSIISTLT

SEQ ID NO: 94 - protein, 132 aa, synthetic construct
  REGION 1..132: note = Synthetic: IL-2_Y45X-1
  VARIANT 44: note = Xaa is an unnatural amino acid
PTSSSTKKTQLQLEHLLLDLQMILNGINNYKNPKLTRMLTFKFXMPKKATELKHLQCLEEELKPLEEVLNLAQSKNFHLRPRDLISNINVIVLELKGSETTFMCEYADETATIVEFLNRWITFCQSIISTLT

SEQ ID NO: 95 - protein, 133 aa, synthetic construct
  REGION 1..133: note = Synthetic: IL-2_V69X
  VARIANT 69: note = Xaa is an unnatural amino acid
APTSSSTKKTQLQLEHLLLDLQMILNGINNYKNPKLTRMLTFKFYMPKKATELKHLQCLEEELKPLEEXLNLAQSKNFHLRPRDLISNINVIVLELKGSETTFMCEYADETATIVEFLNRWITFCQSIISTLT

SEQ ID NO: 96 - protein, 132 aa, synthetic construct
  REGION 1..132: note = Synthetic: IL-2_V69X-1
  VARIANT 68: note = Xaa is an unnatural amino acid
PTSSSTKKTQLQLEHLLLDLQMILNGINNYKNPKLTRMLTFKFYMPKKATELKHLQCLEEELKPLEEXLNLAQSKNFHLRPRDLISNINVIVLELKGSETTFMCEYADETATIVEFLNRWITFCQSIISTLT

SEQ ID NO: 97 - protein, 133 aa, synthetic construct
  REGION 1..133: note = Synthetic: IL-2_L72X
  VARIANT 72: note = Xaa is an unnatural amino acid
APTSSSTKKTQLQLEHLLLDLQMILNGINNYKNPKLTRMLTFKFYMPKKATELKHLQCLEEELKPLEEVLNXAQSKNFHLRPRDLISNINVIVLELKGSETTFMCEYADETATIVEFLNRWITFCQSIISTLT

SEQ ID NO: 98 - protein, 132 aa, synthetic construct
  REGION 1..132: note = Synthetic: IL-2_L72X-1
  VARIANT 71: note = Xaa is an unnatural amino acid
PTSSSTKKTQLQLEHLLLDLQMILNGINNYKNPKLTRMLTFKFYMPKKATELKHLQCLEEELKPLEEVLNXAQSKNFHLRPRDLISNINVIVLELKGSETTFMCEYADETATIVEFLNRWITFCQSIISTLT

(19) United States
(12) Patent Application Publication
(10) Pub. No.: US 2026/0115295 A1
(43) Pub. Date: Apr. 30, 2026

(54) IMMUNO ONCOLOGY COMBINATION THERAPY WITH IL-2 CONJUGATES AND ANTI-EGFR ANTIBODIES

(71) Applicant: Synthorx, Inc., Bridgewater, NJ (US)

(72) Inventors: Giovanni ABBADESSA, Winchester, MA (US); Carolina E. CAFFARO, San Diego, CA (US); Joseph LEVEQUE, San Diego, CA (US); Marcos MILLA, Carlsbad, CA (US); Jerod PTACIN, San Diego, CA (US); Timothy R. WAGENAAR, Sudbury, MA (US)

(73) Assignee: Synthorx, Inc., Bridgewater, NJ (US)

(21) Appl. No.: 19/305,326

(22) Filed: Aug. 20, 2025

Related U.S. Application Data
(63) Continuation of application No. 17/357,615, filed on Jun. 24, 2021, now abandoned.
(60) Provisional application No. 63/044,199, filed on Jun. 25, 2020.

(51) Int. Cl. A61K 47/54; A61K 39/00; A61K 39/395; A61K 47/60; A61P 35/00; C07K 16/28

(52) U.S. Cl. CPC A61K 47/545; A61K 39/3955; A61K 47/60; A61P 35/00; C07K 16/2863; A61K 2039/505; C07K 2317/24; C07K 2317/732

(57) ABSTRACT

Disclosed herein are methods for treating a cancer in a subject in need thereof, comprising administering IL-2 conjugates in combination with an anti-EGFR antibody.

### CROSS-REFERENCE TO RELATED APPLICATIONS

This application is a continuation of U.S. application Ser. No. 17/357,615, filed on Jun. 24, 2021, which claims priority to U.S. Provisional Application No. 63/044,199, filed on Jun. 25, 2020, the disclosure of each application which is hereby incorporated by reference in its entirety.

### SEQUENCE LISTING

The instant application contains a Sequence Listing which has been submitted electronically in XML format and is hereby incorporated by reference in its entirety. Said XML copy, created on Nov. 6, 2025, is named “01183-0090-01US-SYN-Revised.xml” and is 176,407 bytes in size.

### BACKGROUND OF THE DISCLOSURE

Distinct populations of T cells modulate the immune system to maintain immune homeostasis and tolerance. For example, regulatory T (Treg) cells prevent inappropriate responses by the immune system by preventing pathological self-reactivity, while cytotoxic T cells target and destroy infected cells and/or cancerous cells. In some instances, modulation of the different populations of T cells provides an option for treatment of a disease or indication. Modulation of the different populations of T cells may be enhanced by the presence of additional agents or methods in combination therapy.

Cytokines comprise a family of cell signaling proteins such as chemokines, interferons, interleukins, lymphokines, tumor necrosis factors, and other growth factors playing roles in innate and adaptive immune cell homeostasis. Cytokines are produced by immune cells such as macrophages, B lymphocytes, T lymphocytes and mast cells, endothelial cells, fibroblasts, and different stromal cells. In some instances, cytokines modulate the balance between humoral and cell-based immune responses.

Interleukins are signaling proteins that modulate the development and differentiation of T and B lymphocytes, cells of the monocytic lineage, neutrophils, basophils, eosinophils, megakaryocytes, and hematopoietic cells. Interleukins are produced by helper CD4+T and B lymphocytes, monocytes, macrophages, endothelial cells, and other tissue residents.

In some instances, interleukin 2 (IL-2) signaling is used to modulate T cell responses and subsequently for treatment of a cancer. Accordingly, in one aspect, provided herein are methods of treating cancer in a subject comprising administering an IL-2 conjugate in combination with an anti-EGFR antibody.

### SUMMARY OF THE DISCLOSURE

Described herein are methods of treating a cancer in a subject in need thereof, comprising administering to the subject a therapeutically effective amount of (a) an IL-2 conjugate, and (b) an anti-EGFR antibody.

Exemplary embodiments include the following.

Embodiment 1. A method of treating a cancer in a subject in need thereof, comprising administering to the subject a therapeutically effective amount of (a) an IL-2 conjugate, and (b) an anti-EGFR antibody, wherein the IL-2 conjugate comprises the amino acid sequence of SEQ ID NO: 3 in which at least one amino acid residue in the IL-2 conjugate is replaced by the structure of Formula (I):

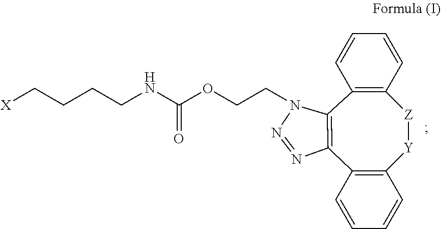

wherein:
Z is CH₂ and Y is

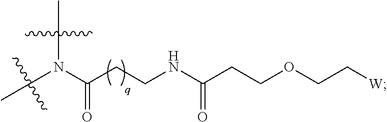

Y is CH₂ and Z is

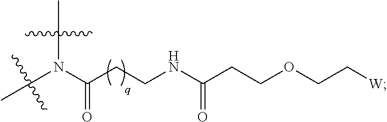

Z is CH₂ and Y is

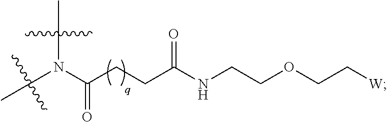

or
Y is CH₂ and Z is

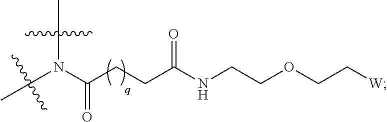

W is a PEG group having an average molecular weight of about 5 kDa, 10 kDa, 15 kDa, 20 kDa, 25 kDa, 30 kDa, 35 kDa, 40 kDa, 45 kDa, 50 kDa, or 60 kDa; and
X is an L-amino acid having the structure:

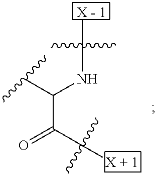

X−1 indicates the point of attachment to the preceding amino acid residue; and
X+1 indicates the point of attachment to the following amino acid residue;
wherein the position of the structure of Formula (I) in the amino acid sequence of the IL-2 conjugate is selected from K34, F41, F43, K42, E61, P64, R37, T40, E67, Y44, V68, and L71.

Embodiment 2. The method of embodiment 1, wherein in the IL-2 conjugate Z is CH₂ and Y is

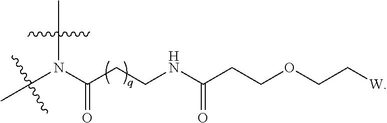

Embodiment 3. The method of embodiment 1, wherein in the IL-2 conjugate Y is CH₂ and Z is

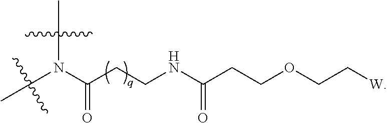

Embodiment 4. The method of embodiment 1, wherein in the IL-2 conjugate Z is CH₂ and Y is

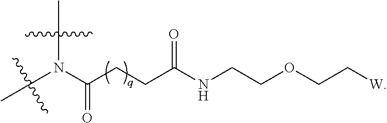

Embodiment 5. The method of embodiment 1, wherein in the IL-2 conjugate Y is CH₂ and Z is

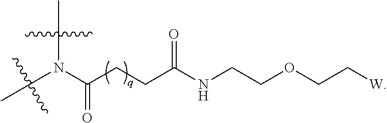

Embodiment 6. The method of any one of embodiments 1-5, wherein in the IL-2 conjugate the PEG group has an average molecular weight of about 25 kDa, 30 kDa, or 35 kDa.

Embodiment 7. The method of embodiment 6, wherein in the IL-2 conjugate the PEG group has an average molecular weight of about 30 kDa.

Embodiment 8. The method of any one of embodiments 1-7, wherein the position of the structure of Formula (I) in the amino acid sequence of the IL-2 conjugate is P64.

Embodiment 9. The method of embodiment 1, wherein the structure of Formula (I) has the structure of Formula (IV) or Formula (V), or is a mixture of the structures of Formula (IV) and Formula (V):

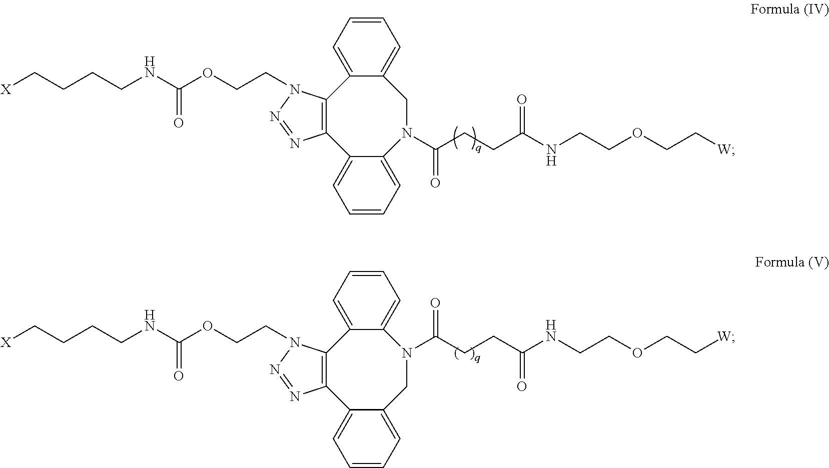

wherein:
W is a PEG group having an average molecular weight of about 25 kDa, 30 kDa, or 30 kDa;
q is 1, 2, or 3;
X is an L-amino acid having the structure:

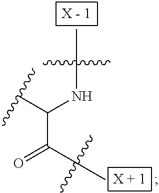 [SMILES: *NC(*)C(*)=O]

X−1 indicates the point of attachment to the preceding amino acid residue; and
X+1 indicates the point of attachment to the following amino acid residue.

Embodiment 10. The method of embodiment 9, wherein the position of the structure of Formula (IV) or Formula (V) in the amino acid sequence of the IL-2 conjugate is P64.

Embodiment 11. The method according to any one of embodiments 1-10, wherein the anti-EGFR antibody is cetuximab.

Embodiment 12. A method of treating a cancer in a subject in need thereof, comprising administering to the subject a therapeutically effective amount of (a) an IL-2 conjugate, and (b) cetuximab, wherein the IL-2 conjugate comprises the amino acid sequence of SEQ ID NO: 50, wherein [AzK_L1_PEG30 kD] has the structure of Formula (XII) or Formula (XIII), or is a mixture of the structures of Formula (XII) and Formula (XIII):

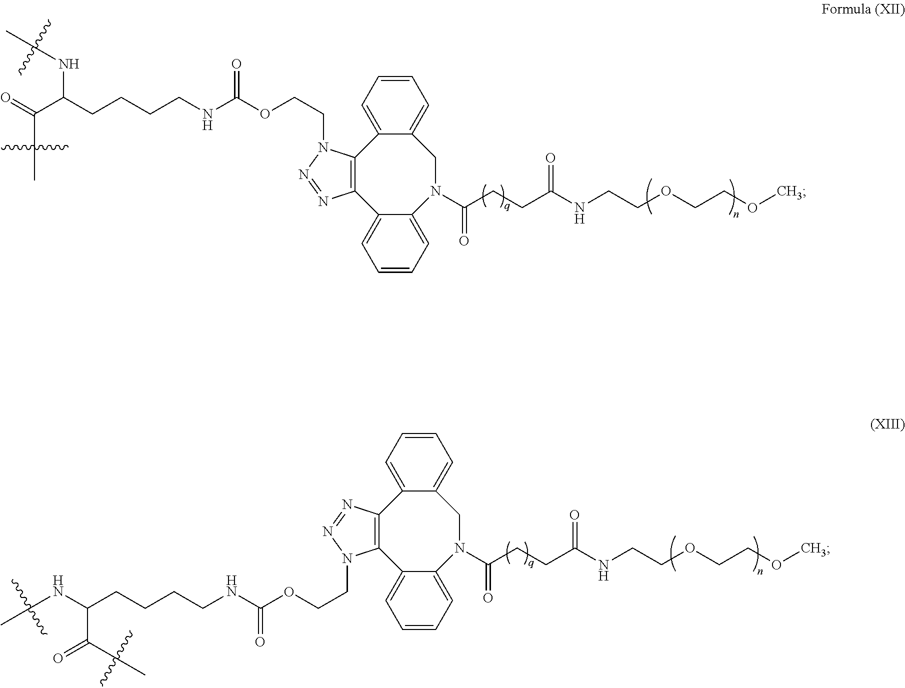

wherein:
n is an integer n is an integer such that —(OCH₂CH₂)ₙ—OCH₃ has a molecular weight of about 30 kDa;
q is 1, 2, or 3; and
the wavy lines indicate covalent bonds to amino acid residues within SEQ ID NO: 50 that are not replaced.

Embodiment 13. The method of any one of embodiments 1-12, wherein q is 1.

Embodiment 14. The method of any one of embodiments 1-12, wherein q is 2.

Embodiment 15. The method of any one of embodiments 1-12, wherein q is 3.

Embodiment 16. The method of any one of embodiments 1-15, wherein the average molecular weight is a number average molecular weight.

Embodiment 17. The method of any one of embodiments 1-15, wherein the average molecular weight is a weight average molecular weight.

Embodiment 18. The method of any one of embodiments 1-17, wherein the IL-2 conjugate is a pharmaceutically acceptable salt, solvate, or hydrate.

Embodiment 19. The method according to any one of embodiments 1-18, wherein the IL-2 conjugate is administered to the subject about once every two weeks, about once every three weeks, or about once every 4 weeks.

Embodiment 20. The method according to any one of embodiments 1-19, wherein the anti-EGFR antibody is administered to the subject about once every week, about once every two weeks, about once every three weeks, or about once every 4 weeks.

Embodiment 21. The method according to any one of embodiments 1-20, wherein the IL-2 conjugate is administered to a subject by intravenous administration.

Embodiment 22. The method according to any one of embodiments 1-21, wherein the IL-2 conjugate and the anti-EGFR antibody are administered separately.

Embodiment 23. The method of embodiment 23, wherein the IL-2 conjugate and the anti-EGFR antibody are administered sequentially.

Embodiment 24. The method according to any one of embodiments 1-23, wherein the cancer is selected from renal cell carcinoma (RCC), non-small cell lung cancer (NSCLC), head and neck squamous cell cancer (HNSCC), classical Hodgkin lymphoma (cHL), primary mediastinal large B-cell lymphoma (PMBCL), urothelial carcinoma, microsatellite unstable cancer, microsatellite stable cancer, gastric cancer, colon cancer, colorectal cancer (CRC), cervical cancer, hepatocellular carcinoma (HCC), Merkel cell carcinoma (MCC), melanoma, small cell lung cancer (SCLC), esophageal, esophageal squamous cell carcinoma (ESCC), glioblastoma, mesothelioma, breast cancer, triple-negative breast cancer, prostate cancer, castrate-resistant prostate cancer, metastatic castrate-resistant prostate cancer, or metastatic castrate-resistant prostate cancer having DNA damage response (DDR) defects, bladder cancer, ovarian cancer, tumors of moderate to low mutational burden, cutaneous squamous cell carcinoma (CSCC), squamous cell skin cancer (SCSC), tumors of low-to non-expressing PD-L1, tumors disseminated systemically to the liver and CNS beyond their primary anatomic originating site, and diffuse large B-cell lymphoma.

Embodiment 25. The method of any one of embodiments 1-24, comprising administering to the subject about 16 μg/kg of the IL-2 conjugate.

Embodiment 26. The method of any one of embodiments 1-24, comprising administering to the subject about 24 μg/kg of the IL-2 conjugate.

Embodiment 27. The method of any one of embodiments 1-10 or 12-26, wherein the anti-EGFR antibody is selected from panitumumab (Vectibix), necitumumab (Portrazza), JNJ-61186372 (Amivantamab), IMC-C225, ABX-EGF, ICR62, and EMD 55900.

Embodiment 28. An IL-2 conjugate for use in the method of any one of embodiments 1-27.

Embodiment 29. Use of an IL-2 conjugate for the manufacture of a medicament for the method of any one of embodiments 1-27.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-C show the % cytotoxicity in CAL27 cells co-cultured with 3 separate donor human PBMCs and varying amounts of an IL-2 conjugate and cetuximab.

FIG. 2A shows the % cytotoxicity in CAL27 cells co-cultured with human PBMCs and varying amounts of an IL-2 conjugate and cetuximab.

FIG. 2B shows the % cytotoxicity in A431 cells co-cultured with human PBMCs and varying amounts of an IL-2 conjugate and cetuximab.

FIG. 3A shows the cytotoxic effect on A431 cells co-cultured with NK92 cells and treated varying amounts of an IL-2 conjugate and cetuximab.

FIG. 3B shows the % cytotoxicity on DLD-1 cells co-cultured with NK92 cells and treated varying amounts of an IL-2 conjugate and cetuximab.

FIG. 3C shows the % cytotoxicity on FaDu cells co-cultured with NK92 cells and treated varying amounts of an IL-2 conjugate and cetuximab.

FIG. 3D shows the % cytotoxicity on CAL27 cells co-cultured with NK92 cells and treated varying amounts of an IL-2 conjugate and cetuximab.

FIG. 4A shows the peripheral CD8+T_{eff} cell counts in the indicated subjects treated with 16 μg/kg of the IL-2 conjugate in combination with cetuximab at specified times following administration of IL-2 conjugate.

FIG. 4B shows the change in peripheral CD8+T_{eff} cell counts in the indicated subjects treated with 16 μg/kg of the IL-2 conjugate in combination with cetuximab at specified times following administration of IL-2 conjugate. Data is normalized to pre-treatment (C1D1) CD8+T_{eff} cell count.

FIG. 5A shows the peripheral NK cell counts in the indicated subjects treated with 16 μg/kg of the IL-2 conjugate in combination with cetuximab at specified times following administration of IL-2 conjugate.

FIG. 5B shows the change in peripheral NK cell counts in the indicated subjects treated with 16 μg/kg of the IL-2 conjugate in combination with cetuximab at specified times following administration of IL-2 conjugate. Data is normalized to pre-treatment (C1D1) NK cell count.

FIG. 6A shows the peripheral CD4+T_{reg} cell counts in the indicated subjects treated with 16 μg/kg of the IL-2 conjugate in combination with cetuximab at specified times following administration of IL-2 conjugate.

FIG. 6B shows the change in peripheral CD4+T_{reg} cell counts in the indicated subjects treated with 16 μg/kg of the IL-2 conjugate in combination with cetuximab at specified times following administration of IL-2 conjugate. Data is normalized to pre-treatment (C1D1) CD4+T_{reg} cell count.

FIG. 7A shows the eosinophil cell counts in the indicated subjects treated with 16 μg/kg of the IL-2 conjugate in combination with cetuximab at specified times following administration of IL-2 conjugate.

FIG. 7B shows the change in eosinophil cell counts in the indicated subjects treated with 16 μg/kg of the IL-2 conjugate in combination with cetuximab at specified times following administration of IL-2 conjugate. Data is normalized to pre-treatment (C1D1) eosinophil cell count.

FIG. 8A shows the lymphocyte cell counts in the indicated subjects treated with 16 μg/kg of the IL-2 conjugate in combination with cetuximab at specified times following administration of IL-2 conjugate.

FIG. 8B shows the change in lymphocyte cell counts in the indicated subjects treated with 16 μg/kg of the IL-2 conjugate in combination with cetuximab at specified times following administration of IL-2 conjugate. Data is normalized to pre-treatment (C1D1) lymphocyte cell count.

### DETAILED DESCRIPTION OF THE DISCLOSURE

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. To the extent any material incorporated herein by reference is inconsistent with the express content of this disclosure, the express content controls. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error, such as for example, within 15%, 10%, or 5%.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

As used herein, the term “significant” or “significantly” in reference to binding affinity means a change in the binding affinity of the cytokine (e.g., IL-2 polypeptide) sufficient to impact binding of the cytokine (e.g., IL-2 polypeptide) to a target receptor. In some instances, the term refers to a change of at least 10%, 20%, 30%, 40%, 50%, 60%, 70%, 80%, 90%, 95%, or more. In some instances, the term means a change of at least 2-fold, 3-fold, 4-fold, 5-fold, 6-fold, 7-fold, 8-fold, 9-fold, 10-fold, 50-fold, 100-fold, 500-fold, 1000-fold, or more.

In some instances, the term “significant” or “significantly” in reference to activation of one or more cell populations via a cytokine signaling complex means a change sufficient to activate the cell population. In some cases, the change to activate the cell population is measured as a receptor signaling potency. In such cases, an EC50 value may be provided. In other cases, an ED50 value may be provided. In additional cases, a concentration or dosage of the cytokine may be provided.

As used herein, the term “potency” refers to the amount of a cytokine (e.g., IL-2 polypeptide) required to produce a target effect. In some instances, the term “potency” refers to the amount of cytokine (e.g., IL-2 polypeptide) required to activate a target cytokine receptor (e.g., IL-2 receptor). In other instances, the term “potency” refers to the amount of cytokine (e.g., IL-2 polypeptide) required to activate a target cell population. In some cases, potency is measured as ED50 (Effective Dose 50), or the dose required to produce 50% of a maximal effect. In other cases, potency is measured as EC50 (Effective Concentration 50), or the dose required to produce the target effect in 50% of the population.

As used herein, the term “unnatural amino acid” refers to an amino acid other than one of the 20 naturally occurring amino acids. Exemplary unnatural amino acids are described in Young et al., “Beyond the canonical 20 amino acids: expanding the genetic lexicon,” J. of Biological Chemistry 285 (15): 11039-11044 (2010), the disclosure of which is herein incorporated by reference.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. In some embodiments, the antigen is EGFR.

The term “monoclonal antibody(ies)” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

As used herein, “nucleotide” refers to a compound comprising a nucleoside moiety and a phosphate moiety. Exemplary natural nucleotides include, without limitation, adenosine triphosphate (ATP), uridine triphosphate (UTP), cytidine triphosphate (CTP), guanosine triphosphate (GTP), adenosine diphosphate (ADP), uridine diphosphate (UDP), cytidine diphosphate (CDP), guanosine diphosphate (GDP), adenosine monophosphate (AMP), uridine monophosphate (UMP), cytidine monophosphate (CMP), and guanosine monophosphate (GMP), deoxyadenosine triphosphate (dATP), deoxythymidine triphosphate (dTTP), deoxycytidine triphosphate (dCTP), deoxyguanosine triphosphate (dGTP), deoxyadenosine diphosphate (dADP), thymidine diphosphate (dTDP), deoxycytidine diphosphate (dCDP), deoxyguanosine diphosphate (dGDP), deoxyadenosine monophosphate (dAMP), deoxythymidine monophosphate (dTMP), deoxycytidine monophosphate (dCMP), and deoxyguanosine monophosphate (dGMP). Exemplary natural deoxyribonucleotides, which comprise a deoxyribose as the sugar moiety, include dATP, dTTP, dCTP, dGTP, dADP, dTDP, dCDP, dGDP, dAMP, dTMP, dCMP, and dGMP. Exemplary natural ribonucleotides, which comprise a ribose as the sugar moiety, include ATP, UTP, CTP, GTP, ADP, UDP, CDP, GDP, AMP, UMP, CMP, and GMP.

As used herein, “base” or “nucleobase” refers to at least the nucleobase portion of a nucleoside or nucleotide (nucleoside and nucleotide encompass the ribo or deoxyribo variants), which may in some cases contain further modifications to the sugar portion of the nucleoside or nucleotide. In some cases, “base” is also used to represent the entire nucleoside or nucleotide (for example, a “base” may be incorporated by a DNA polymerase into DNA, or by an RNA polymerase into RNA). However, the term “base” should not be interpreted as necessarily representing the entire nucleoside or nucleotide unless required by the context. In the chemical structures provided herein of a base or nucleobase, only the base of the nucleoside or nucleotide is shown, with the sugar moiety and, optionally, any phosphate residues omitted for clarity. As used in the chemical structures provided herein of a base or nucleobase, the wavy line represents connection to a nucleoside or nucleotide, in which the sugar portion of the nucleoside or nucleotide may be further modified. In some embodiments, the wavy line represents attachment of the base or nucleobase to the sugar portion, such as a pentose, of the nucleoside or nucleotide. In some embodiments, the pentose is a ribose or a deoxyribose.

In some embodiments, a nucleobase is generally the heterocyclic base portion of a nucleoside. Nucleobases may be naturally occurring, may be modified, may bear no similarity to natural bases, and/or may be synthesized, e.g., by organic synthesis. In certain embodiments, a nucleobase comprises any atom or group of atoms in a nucleoside or nucleotide, where the atom or group of atoms is capable of interacting with a base of another nucleic acid with or without the use of hydrogen bonds. In certain embodiments, an unnatural nucleobase is not derived from a natural nucleobase. It should be noted that unnatural nucleobases do not necessarily possess basic properties, however, they are referred to as nucleobases for simplicity. In some embodiments, when referring to a nucleobase, a “(d)” indicates that the nucleobase can be attached to a deoxyribose or a ribose, while “d” without parentheses indicates that the nucleobase is attached to deoxyribose.

As used herein, a “nucleoside” is a compound comprising a nucleobase moiety and a sugar moiety. Nucleosides include, but are not limited to, naturally occurring nucleosides (as found in DNA and RNA), abasic nucleosides, modified nucleosides, and nucleosides having mimetic bases and/or sugar groups. Nucleosides include nucleosides comprising any variety of substituents. A nucleoside can be a glycoside compound formed through glycosidic linking between a nucleic acid base and a reducing group of a sugar.

An “analog” of a chemical structure, as the term is used herein, refers to a chemical structure that preserves substantial similarity with the parent structure, although it may not be readily derived synthetically from the parent structure. In some embodiments, a nucleotide analog is an unnatural nucleotide. In some embodiments, a nucleoside analog is an unnatural nucleoside. A related chemical structure that is readily derived synthetically from a parent chemical structure is referred to as a “derivative.”

As used herein, “dose-limiting toxicity” (DLT) is defined as an adverse event occurring within Day 1 through Day 29 (inclusive)±1 day of a treatment cycle that was not clearly or incontrovertibly solely related to an extraneous cause and that meets the criteria set forth in Example 5 for DLT.

As used herein, “cetuximab” refers to the chimeric (mouse/human) anti-EGFR antibody marketed under the brand name “Erbitux” by Eli Lilly and Co.

Although various features of the invention may be described in the context of a single embodiment, the features may also be provided separately or in any suitable combination. Conversely, although the invention may be described herein in the context of separate embodiments for clarity, the invention may also be implemented in a single embodiment.

### IL-2 Conjugates

Interleukin 2 (IL-2) is a pleiotropic type-1 cytokine whose structure comprises a 15.5 kDa four α-helix bundle. The precursor form of IL-2 is 153 amino acid residues in length, with the first 20 amino acids forming a signal peptide and residues 21-153 forming the mature form. IL-2 is produced primarily by CD4+ T cells post antigen stimulation and to a lesser extent, by CD8+ cells, Natural Killer (NK) cells, and Natural killer T (NKT) cells, activated dendritic cells (DCs), and mast cells. IL-2 signaling occurs through interaction with specific combinations of IL-2 receptor (IL-2R) subunits, IL-2Rα (also known as CD25), IL-2Rβ (also known as CD122), and IL-2Rγ (also known as CD132). Interaction of IL-2 with the IL-2Rα forms the “low-affinity” IL-2 receptor complex with a K_{d} of about 10⁻⁸ M. Interaction of IL-2 with IL-2Rβ and IL-2Rγ forms the “intermediate-affinity” IL-2 receptor complex with a K_{d} of about 10⁻⁹ M. Interaction of IL-2 with all three subunits, IL-2Rα, IL-2Rβ, and IL-2Rγ, forms the “high-affinity” IL-2 receptor complex with a K_{d} of about >10⁻¹¹ M.

In some instances, IL-2 signaling via the “high-affinity” IL-2Rαβγ complex modulates the activation and proliferation of regulatory T cells. Regulatory T cells, or CD4⁺CD25⁺Foxp3⁺ regulatory T (T_{reg}) cells, mediate maintenance of immune homeostasis by suppression of effector cells such as CD4⁺ T cells, CD8⁺ T cells, B cells, NK cells, and NKT cells. In some instances, T_{reg} cells are generated from the thymus (tTreg cells) or are induced from naïve T cells in the periphery (pTreg cells). In some cases, Treg cells are considered as the mediator of peripheral tolerance. Indeed, in one study, transfer of CD25-depleted peripheral CD4⁺ T cells produced a variety of autoimmune diseases in nude mice, whereas cotransfer of CD4⁺CD25⁺ T cells suppressed the development of autoimmunity (Sakaguchi, et al., “Immunologic self-tolerance maintained by activated T cells expressing IL-2 receptor alpha-chains (CD25),” J. Immunol. 155 (3): 1151-1164 (1995), the disclosure of each of which is herein incorporated by reference). Augmentation of the Treg cell population down-regulates effector T cell proliferation and suppresses autoimmunity and T cell anti-tumor responses.

IL-2 signaling via the “intermediate-affinity” IL-2Rβγ complex modulates the activation and proliferation of CD8⁺ effector T (Teff) cells, NK cells, and NKT cells. CD8⁺ Teff cells (also known as cytotoxic T cells, Tc cells, cytotoxic T lymphocytes, CTLs, T-killer cells, cytolytic T cells, Tcon, or killer T cells) are T lymphocytes that recognize and kill damaged cells, cancerous cells, and pathogen-infected cells. NK and NKT cells are types of lymphocytes that, similar to CD8⁺ Teff cells, target cancerous cells and pathogen-infected cells.

In some instances, IL-2 signaling is utilized to modulate T cell responses and subsequently for treatment of a cancer. For example, IL-2 is administered in a high-dose form to induce expansion of Teff cell populations for treatment of a cancer. However, high-dose IL-2 further leads to concomitant stimulation of Treg cells that dampen anti-tumor immune responses. High-dose IL-2 also induces toxic adverse events mediated by the engagement of IL-2R alpha chain-expressing cells in the vasculature, including type 2 innate immune cells (ILC-2), eosinophils and endothelial cells. This leads to eosinophilia, capillary leak and vascular leak syndrome VLS).

Adoptive cell therapy enables physicians to effectively harness a patient's own immune cells to fight diseases such as proliferative disease (e.g., cancer) as well as infectious disease. The effect of IL-2 signaling may be further enhanced by the presence of additional agents or methods in combination therapy. For example, epidermal growth factor receptor (EGFR) is a cell surface receptor overexpressed in many types of cancer. Activation of EGFR promotes cell proliferation and survival, as well as angiogenesis, leading to tumor growth and metastasis. Cell growth and angiogenesis may be regulated by blocking the binding of EGFR to epidermal growth factor (EGF). Anti-EGFR antibodies bind to the extracellular domain of EGFR and prevent EGF from binding to EGFR, thereby inhibiting downstream signal transduction cascade and leading to decreased cell growth. Anti-EGFR antibodies can cause the same effect by also competitively inhibiting transforming growth factor alpha (TGF-α) from binding to EGFR.

Provided herein are methods of treating a cancer in a subject in need thereof, comprising administering to the subject a therapeutically effective amount of (a) an IL-2 conjugate, and (b) an anti-EGFR antibody. Certain exemplary IL-2 polypeptides and IL-2 conjugates are provided in Table 1.

TABLE 1

| | | SEQ ID |
|---|---|---|
| Name | Sequence | NO: |
| IL-2 | APTSSSTKKTQLQLEHLLLDLQMILNGINNYKNPKLTRM | 1 |
| (homo sapiens) | LTFKFYMPKKATELKHLQCLEEELKPLEEVLNLAQSKNF | |
| (mature form) | HLRPRDLISNINVIVLELKGSETTFMCEYADETATIVEF | |
| | LNRWITFCQSIISTLT | |
| IL-2 | MYRMQLLSCIALSLALVTNSAPTSSSTKKTQLQLEHLLL | 2 |
| (homo sapiens) | DLQMILNGINNYKNPKLTRMLTFKFYMPKKATELKHLQC | |
| (precursor) | LEEELKPLEEVLNLAQSKNFHLRPRDLISNINVIVLELK | |
| NCBI Accession | GSETTFMCEYADETATIVEFLNRWITFCQSIISTLT | |
| No.: AAB46883.1 | | |
| aldesleukin | PTSSSTKKTQLQLEHLLLDLQMILNGINNYKNPKLTRML | 3 |
| | TFKFYMPKKATELKHLQCLEEELKPLEEVLNLAQSKNFH | |
| | LRPRDLISNINVIVLELKGSETTFMCEYADETATIVEFL | |
| | NRWITFSQSIISTLT | |
| IL-2_C125S | APTSSSTKKTQLQLEHLLLDLQMILNGINNYKNPKLTRM | 4 |
| | LTFKFYMPKKATELKHLQCLEEELKPLEEVLNLAQSKNF | |
| | HLRPRDLISNINVIVLELKGSETTFMCEYADETATIVEF | |
| | LNRWITFSQSIISTLT | |
| IL-2_P65X | APTSSSTKKTQLQLEHLLLDLQMILNGINNYKNPKLTRM | 5 |
| | LTFKFYMPKKATELKHLQCLEEELKXLEEVLNLAQSKNF | |
| | HLRPRDLISNINVIVLELKGSETTFMCEYADETATIVEF | |
| | LNRWITFSQSIISTLT | |
| IL-2_E62X | APTSSSTKKTQLQLEHLLLDLQMILNGINNYKNPKLTRM | 6 |
| | LTFKFYMPKKATELKHLQCLEEXLKPLEEVLNLAQSKNF | |
| | HLRPRDLISNINVIVLELKGSETTFMCEYADETATIVEF | |
| | LNRWITFSQSIISTLT | |
| IL-2_F42X | APTSSSTKKTQLQLEHLLLDLQMILNGINNYKNPKLTRM | 7 |
| | LTXKFYMPKKATELKHLQCLEEELKPLEEVLNLAQSKNF | |
| | HLRPRDLISNINVIVLELKGSETTFMCEYADETATIVEF | |
| | LNRWITFSQSIISTLT | |
| IL-2_K43X | APTSSSTKKTQLQLEHLLLDLQMILNGINNYKNPKLTRM | 8 |
| | LTFXFYMPKKATELKHLQCLEEELKPLEEVLNLAQSKNF | |
| | HLRPRDLISNINVIVLELKGSETTFMCEYADETATIVEF | |
| | LNRWITFSQSIISTLT | |
| IL-2_K35X | APTSSSTKKTQLQLEHLLLDLQMILNGINNYKNPXLTRM | 9 |
| | LTFKFYMPKKATELKHLQCLEEELKPLEEVLNLAQSKNF | |
| | HLRPRDLISNINVIVLELKGSETTFMCEYADETATIVEF | |
| | LNRWITFSQSIISTLT | |
| IL-2_P65[AzK] | APTSSSTKKTQLQLEHLLLDLQMILNGINNYKNPKLTRM | 10 |
| | LTFKFYMPKKATELKHLQCLEEELK[AzK]LEEVLNLAQ | |
| | SKNFHLRPRDLISNINVIVLELKGSETTFMCEYADETAT | |
| | IVEFLNRWITFSQSIISTLT | |
| IL-2_E62[AzK] | APTSSSTKKTQLQLEHLLLDLQMILNGINNYKNPKLTRM | 11 |
| | LTFKFYMPKKATELKHLQCLEE[AzK]LKPLEEVLNLAQ | |
| | SKNFHLRPRDLISNINVIVLELKGSETTFMCEYADETAT | |
| | IVEFLNRWITFSQSIISTLT | |
| IL-2_F42[AzK] | APTSSSTKKTQLQLEHLLLDLQMILNGINNYKNPKLTRM | 12 |
| | LT[AzK]KFYMPKKATELKHLQCLEEELKPLEEVLNLAQ | |
| | SKNFHLRPRDLISNINVIVLELKGSETTFMCEYADETAT | |
| | IVEFLNRWITFSQSIISTLT | |
| IL-2_K43[AzK] | APTSSSTKKTQLQLEHLLLDLQMILNGINNYKNPKLTRM | 13 |
| | LTF[AzK]FYMPKKATELKHLQCLEEELKPLEEVLNLAQ | |
| | SKNFHLRPRDLISNINVIVLELKGSETTFMCEYADETAT | |
| | IVEFLNRWITFSQSIISTLT | |
| IL-2_K35[AzK] | APTSSSTKKTQLQLEHLLLDLQMILNGINNYKNP[AzK] | 14 |
| | LTRMLTFKFYMPKKATELKHLQCLEEELKPLEEVLNLAQ | |
| | SKNFHLRPRDLISNINVIVLELKGSETTFMCEYADETAT | |
| | IVEFLNRWITFSQSIISTLT | |
| IL-2P65[AzK_PEG] | APTSSSTKKTQLQLEHLLLDLQMILNGINNYKNPKLTRM | 15 |
| | LTFKFYMPKKATELKHLQCLEEELK[AzK_PEG]LEEVL | |
| | NLAQSKNFHLRPRDLISNINVIVLELKGSETTFMCEYAD | |
| | ETATIVEFLNRWITFSQSIISTLT | |
| IL-2_E62[AzK_PEG] | APTSSSTKKTQLQLEHLLLDLQMILNGINNYKNPKLTRM | 16 |
| | LTFKFYMPKKATELKHLQCLEE[AzK_PEG]LKPLEEVL | |
| | NLAQSKNFHLRPRDLISNINVIVLELKGSETTFMCEYAD | |
| | ETATIVEFLNRWITFSQSIISTLT | |
| IL-2F42[AzK_PEG] | APTSSSTKKTQLQLEHLLLDLQMILNGINNYKNPKLTRM | 17 |
| | LT[AzK_PEG]KFYMPKKATELKHLQCLEEELKPLEEVL | |
| | NLAQSKNFHLRPRDLISNINVIVLELKGSETTFMCEYAD | |
| | ETATIVEFLNRWITFSQSIISTLT | |
| IL-2_K43[AzK_PEG] | APTSSSTKKTQLQLEHLLLDLQMILNGINNYKNPKLTRM | 18 |
| | LTF[AzK_PEG]FYMPKKATELKHLQCLEEELKPLEEVL | |
| | NLAQSKNFHLRPRDLISNINVIVLELKGSETTFMCEYAD | |
| | ETATIVEFLNRWITFSQSIISTLT | |
| IL-2_K35[AzK_PEG] | APTSSSTKKTQLQLEHLLLDLQMILNGINNYKNP | 19 |
| | [AzK_PEG]LTRMLTFKFYMPKKATELKHLQCLEEELKP | |
| | LEEVLNLAQSKNFHLRPRDLISNINVIVLELKGSETTFM | |
| | CEYADETATIVEFLNRWITFSQSIISTLT | |
| IL-2_P65[AzK_PEG5kD] | APTSSSTKKTQLQLEHLLLDLQMILNGINNYKNPKLTRM | 20 |
| | LTFKFYMPKKATELKHLQCLEEELK[AzK_PEG5kD]LE | |
| | EVLNLAQSKNFHLRPRDLISNINVIVLELKGSETTFMCE | |
| | YADETATIVEFLNRWITFSQSIISTLT | |
| IL-2_E62[AzK_PEG5kD] | APTSSSTKKTQLQLEHLLLDLQMILNGINNYKNPKLTRM | 21 |
| | LTFKFYMPKKATELKHLQCLEE[AzK_PEG5kD]LKPLE | |
| | EVLNLAQSKNFHLRPRDLISNINVIVLELKGSETTFMCE | |
| | YADETATIVEFLNRWITFSQSIISTLT | |
| IL-2F42[AzK_PEG5kD] | APTSSSTKKTQLQLEHLLLDLQMILNGINNYKNPKLTRM | 22 |
| | LT[AzK_PEG5kD]KFYMPKKATELKHLQCLEEELKPLE | |
| | EVLNLAQSKNFHLRPRDLISNINVIVLELKGSETTFMCE | |
| | YADETATIVEFLNRWITFSQSIISTLT | |
| IL-2_K43[AzK_PEG5kD] | APTSSSTKKTQLQLEHLLLDLQMILNGINNYKNPKLTRM | 23 |
| | LTF[AzK_PEG5kD]FYMPKKATELKHLQCLEEELKPLE | |
| | EVLNLAQSKNFHLRPRDLISNINVIVLELKGSETTFMCE | |
| | YADETATIVEFLNRWITFSQSIISTLT | |
| IL-2_K35[AzK_PEG5kD] | APTSSSTKKTQLQLEHLLLDLQMILNGINNYKNP | 24 |
| | [AzK_PEG5kD]LTRMLTFKFYMPKKATELKHLQCLEEE | |
| | LKPLEEVLNLAQSKNFHLRPRDLISNINVIVLELKGSET | |
| | TFMCEYADETATIVEFLNRWITFSQSIISTLT | |
| IL-2_P65 | APTSSSTKKTQLQLEHLLLDLQMILNGINNYKNPKLTRM | 25 |
| [AzK_PEG30kD] | LTFKFYMPKKATELKHLQCLEEELK[AzK_PEG30kD]L | |
| | EEVLNLAQSKNFHLRPRDLISNINVIVLELKGSETTFMC | |
| | EYADETATIVEFLNRWITFSQSIISTLT | |
| IL-2_E62 | APTSSSTKKTQLQLEHLLLDLQMILNGINNYKNPKLTRM | 26 |
| [AzK_PEG30kD] | LTFKFYMPKKATELKHLQCLEE[AzK_PEG30kD]LKPL | |
| | EEVLNLAQSKNFHLRPRDLISNINVIVLELKGSETTFMC | |
| | EYADETATIVEFLNRWITFSQSIISTLT | |
| IL-2_F42 | APTSSSTKKTQLQLEHLLLDLQMILNGINNYKNPKLTRM | 27 |
| [AzK_PEG30kD] | LT[AzK_PEG30kD]KFYMPKKATELKHLQCLEEELKPL | |
| | EEVLNLAQSKNFHLRPRDLISNINVIVLELKGSETTFMC | |
| | EYADETATIVEFLNRWITFSQSIISTLT | |
| IL-2_K43 | APTSSSTKKTQLQLEHLLLDLQMILNGINNYKNPKLTRM | 28 |
| [AzK_PEG30kD] | LTF[AzK_PEG30kD]FYMPKKATELKHLQCLEEELKPL | |
| | EEVLNLAQSKNFHLRPRDLISNINVIVLELKGSETTFMC | |
| | EYADETATIVEFLNRWITFSQSIISTLT | |
| IL-2_K35 | APTSSSTKKTQLQLEHLLLDLQMILNGINNYKNP | 29 |
| [AzK_PEG30kD] | [AzK_PEG30kD]LTRIVILTFKFYMPKKATELKHLQCL | |
| | EEELKPLEEVLNLAQSKNFHLRPRDLISNINVIVLELKG | |
| | SETTFMCEYADETATIVEFLNRWITFSQSIISTLT | |
| IL-2_P65X-1 | PTSSSTKKTQLQLEHLLLDLQMILNGINNYKNPKLTRML | 30 |
| | TFKFYMPKKATELKHLQCLEEELKXLEEVLNLAQSKNFH | |
| | LRPRDLISNINVIVLELKGSETTFMCEYADETATIVEFL | |
| | NRWITFSQSIISTLT | |
| IL-2_E62X-1 | PTSSSTKKTQLQLEHLLLDLQMILNGINNYKNPKLTRML | 31 |
| | TFKFYMPKKATELKHLQCLEEXLKPLEEVLNLAQSKNFH | |
| | LRPRDLISNINVIVLELKGSETTFMCEYADETATIVEFL | |
| | NRWITFSQSIISTLT | |
| IL-2_F42X-1 | PTSSSTKKTQLQLEHLLLDLQMILNGINNYKNPKLTRML | 32 |
| | TXKFYMPKKATELKHLQCLEEELKPLEEVLNLAQSKNFH | |
| | LRPRDLISNINVIVLELKGSETTFMCEYADETATIVEFL | |
| | NRWITFSQSIISTLT | |
| IL-2_K43X-1 | PTSSSTKKTQLQLEHLLLDLQMILNGINNYKNPKLTRML | 33 |
| | TFXFYMPKKATELKHLQCLEEELKPLEEVLNLAQSKNFH | |
| | LRPRDLISNINVIVLELKGSETTFMCEYADETATIVEFL | |
| | NRWITFSQSIISTLT | |
| IL-2_K35X-1 | PTSSSTKKTQLQLEHLLLDLQMILNGINNYKNPXLTRML | 34 |
| | TFKFYMPKKATELKHLQCLEEELKPLEEVLNLAQSKNFH | |
| | LRPRDLISNINVIVLELKGSETTFMCEYADETATIVEFL | |
| | NRWITFSQSIISTLT | |
| IL-2_P65[AzK]-1 | PTSSSTKKTQLQLEHLLLDLQMILNGINNYKNPKLTRML | 35 |
| | TFKFYMPKKATELKHLQCLEEELK[AzK]LEEVLNLAQS | |
| | KNFHLRPRDLISNINVIVLELKGSETTFMCEYADETATI | |
| | VEFLNRWITFSQSIISTLT | |
| IL-2_E62[AzK]-1 | PTSSSTKKTQLQLEHLLLDLQMILNGINNYKNPKLTRML | 36 |
| | TFKFYMPKKATELKHLQCLEE[AzK]LKPLEEVLNLAQS | |
| | KNFHLRPRDLISNINVIVLELKGSETTFMCEYADETATI | |
| | VEFLNRWITFSQSIISTLT | |
| IL-2_F42[AzK]-1 | PTSSSTKKTQLQLEHLLLDLQMILNGINNYKNPKLTRML | 37 |
| | T[AzK]KFYMPKKATELKHLQCLEEELKPLEEVLNLAQS | |
| | KNFHLRPRDLISNINVIVLELKGSETTFMCEYADETATI | |
| | VEFLNRWITFSQSIISTLT | |
| IL-2_K43[AzK]-1 | PTSSSTKKTQLQLEHLLLDLQMILNGINNYKNPKLTRML | 38 |
| | TF[AzK]FYMPKKATELKHLQCLEEELKPLEEVLNLAQS | |
| | KNFHLRPRDLISNINVIVLELKGSETTFMCEYADETATI | |
| | VEFLNRWITFSQSIISTLT | |
| IL-2_K35[AzK]-1 | PTSSSTKKTQLQLEHLLLDLQMILNGINNYKNP[AzK]L | 39 |
| | TRMLTFKFYMPKKATELKHLQCLEEELKPLEEVLNLAQS | |
| | KNFHLRPRDLISNINVIVLELKGSETTFMCEYADETATI | |
| | VEFLNRWITFSQSIISTLT | |
| IL-2_P65 | PTSSSTKKTQLQLEHLLLDLQMILNGINNYKNPKLTRML | 40 |
| [AzK_L1_PEG]-1 | TFKFYMPKKATELKHLQCLEEELK[AzK_L1_PEG]LEE | |
| | VLNLAQSKNFHLRPRDLISNINVIVLELKGSETTFMCEY | |
| | ADETATIVEFLNRWITFSQSIISTLT | |
| IL-2_E62 | PTSSSTKKTQLQLEHLLLDLQMILNGINNYKNPKLTRML | 41 |
| [AzK_L1_PEG]-1 | TFKFYMPKKATELKHLQCLEE[AzK_L1_PEG]LKPLEE | |
| | VLNLAQSKNFHLRPRDLISNINVIVLELKGSETTFMCEY | |
| | ADETATIVEFLNRWITFSQSIISTLT | |
| IL-2_F42 | PTSSSTKKTQLQLEHLLLDLQMILNGINNYKNPKLTRML | 42 |
| [AzK_L1_PEG]-1 | T[AzK_L1_PEG]KFYMPKKATELKHLQCLEEELKPLEE | |
| | VLNLAQSKNFHLRPRDLISNINVIVLELKGSETTFMCEY | |
| | ADETATIVEFLNRWITFSQSIISTLT | |
| IL-2_K43 | PTSSSTKKTQLQLEHLLLDLQMILNGINNYKNPKLTRML | 43 |
| [AzK_L1_PEG]-1 | TF[AzK_L1_PEG]FYMPKKATELKHLQCLEEELKPLEE | |
| | VLNLAQSKNFHLRPRDLISNINVIVLELKGSETTFMCEY | |
| | ADETATIVEFLNRWITFSQSIISTLT | |
| IL-2_K35 | PTSSSTKKTQLQLEHLLLDLQMILNGINNYKNP | 44 |
| [AzK_L1_PEG]-1 | [AzK_L1_PEG]LTRMLTFKFYMPKKATELKHLQCLEEE | |
| | LKPLEEVLNLAQSKNFHLRPRDLISNINVIVLELKGSET | |
| | TFMCEYADETATIVEFLNRWITFSQSIISTLT | |
| IL-2_P65 | PTSSSTKKTQLQLEHLLLDLQMILNGINNYKNPKLTRML | 45 |
| [AzK_L1_PEG5kD]-1 | TFKFYMPKKATELKHLQCLEEELK[AzK_L1_PEG5kD] | |
| | LEEVLNLAQSKNFHLRPRDLISNINVIVLELKGSETTFM | |
| | CEYADETATIVEFLNRWITFSQSIISTLT | |
| IL-2_E62 | PTSSSTKKTQLQLEHLLLDLQMILNGINNYKNPKLTRML | 46 |
| [AzK_L1_PEG5kD]-1 | TFKFYMPKKATELKHLQCLEE[AzK_L1_PEG5kD]LKP | |
| | LEEVLNLAQSKNFHLRPRDLISNINVIVLELKGSETTFM | |
| | CEYADETATIVEFLNRWITFSQSIISTLT | |
| IL-2_F42 | PTSSSTKKTQLQLEHLLLDLQMILNGINNYKNPKLTRML | 47 |
| [AzK_L1_PEG5kD]-1 | T[AzK_L1_PEG5kD]KFYMPKKATELKHLQCLEEELKP | |
| | LEEVLNLAQSKNFHLRPRDLISNINVIVLELKGSETTFM | |
| | CEYADETATIVEFLNRWITFSQSIISTLT | |
| IL-2_K43 | PTSSSTKKTQLQLEHLLLDLQMILNGINNYKNPKLTRML | 48 |
| [AzK_L1_PEG5kD]-1 | TF[AzK_L1_PEG5kD]FYMPKKATELKHLQCLEEELKP | |
| | LEEVLNLAQSKNFHLRPRDLISNINVIVLELKGSETTFM | |
| | CEYADETATIVEFLNRWITFSQSIISTLT | |
| IL-2_K35 | PTSSSTKKTQLQLEHLLLDLQMILNGINNYKNP | 49 |
| [AzK_L1_PEG5kD]-1 | [AzK_L1_PEG5kD]LTRMLTFKFYMPKKATELKHLQCL | |
| | EEELKPLEEVLNLAQSKNFHLRPRDLISNINVIVLELKG | |
| | SETTFMCEYADETATIVEFLNRWITFSQSIISTLT | |
| IL-2_P65 | PTSSSTKKTQLQLEHLLLDLQMILNGINNYKNPKLTRML | 50 |
| [AzK_L1_PEG30kD]-1 | TFKFYMPKKATELKHLQCLEEELK[AzK_L1_PEG30kD] | |
| | LEEVLNLAQSKNFHLRPRDLISNINVIVLELKGSETTFM | |
| | CEYADETATIVEFLNRWITFSQSIISTLT | |
| IL-2_E62 | PTSSSTKKTQLQLEHLLLDLQMILNGINNYKNPKLTRML | 51 |
| [AzK_L1_PEG30kD]-1 | TFKFYMPKKATELKHLQCLEE[AzK_L1_PEG30kD]LK | |
| | PLEEVLNLAQSKNFHLRPRDLISNINVIVLELKGSETTF | |
| | MCEYADETATIVEFLNRWITFSQSIISTLT | |
| IL-2_F42 | PTSSSTKKTQLQLEHLLLDLQMILNGINNYKNPKLTRML | 52 |
| [AzK_L1_PEG30kD]-1 | T[AzK_L1_PEG30kD]KFYMPKKATELKHLQCLEEELK | |
| | PLEEVLNLAQSKNFHLRPRDLISNINVIVLELKGSETTF | |
| | MCEYADETATIVEFLNRWITFSQSIISTLT | |
| IL-2_K43 | PTSSSTKKTQLQLEHLLLDLQMILNGINNYKNPKLTRML | 53 |
| [AzK_L1_PEG30kD]-1 | TF[AzK_L1_PEG30kD]FYMPKKATELKHLQCLEEELK | |
| | PLEEVLNLAQSKNFHLRPRDLISNINVIVLELKGSETTF | |
| | MCEYADETATIVEFLNRWITFSQSIISTLT | |
| IL-2_K35 | PTSSSTKKTQLQLEHLLLDLQMILNGINNYKNP | 54 |
| [AzK_L1_PEG30kD]-1 | [AzK_L1_PEG30kD]LTRMLTFKFYMPKKATELKHLQC | |
| | LEEELKPLEEVLNLAQSKNFHLRPRDLISNINVIVLELK | |
| | GSETTFMCEYADETATIVEFLNRWITFSQSIISTLT | |
| IL-2_P65 | APTSSSTKKTQLQLEHLLLDLQMILNGINNYKNPKLTRM | 55 |
| [AzK_L1_PEG]-2 | LTFKFYMPKKATELKHLQCLEEELK[AzK_L1_PEG]LE | |
| | EVLNLAQSKNFHLRPRDLISNINVIVLELKGSETTFMCE | |
| | YADETATIVEFLNRWITFSQSIISTLT | |
| IL-2_E62 | APTSSSTKKTQLQLEHLLLDLQMILNGINNYKNPKLTRM | 56 |
| [AzK_L1_PEG]-2 | LTFKFYMPKKATELKHLQCLEE[AzK_L1_PEG]LKPLE | |
| | EVLNLAQSKNFHLRPRDLISNINVIVLELKGSETTFMCE | |
| | YADETATIVEFLNRWITFSQSIISTLT | |
| IL-2_F42 | APTSSSTKKTQLQLEHLLLDLQMILNGINNYKNPKLTRM | 57 |
| [AzK_L1_PEG]-2 | LT[AzK_L1_PEG]KFYMPKKATELKHLQCLEEELKPLE | |
| | EVLNLAQSKNFHLRPRDLISNINVIVLELKGSETTFMCE | |
| | YADETATIVEFLNRWITFSQSIISTLT | |
| IL-2_K43 | APTSSSTKKTQLQLEHLLLDLQMILNGINNYKNPKLTRM | 58 |
| [AzK_L1_PEG]-2 | LTF[AzK_L1_PEG]FYMPKKATELKHLQCLEEELKPLE | |
| | EVLNLAQSKNFHLRPRDLISNINVIVLELKGSETTFMCE | |
| | YADETATIVEFLNRWITFSQSIISTLT | |
| IL-2_K35 | APTSSSTKKTQLQLEHLLLDLQMILNGINNYKNP | 59 |
| [AzK_L1_PEG]-2 | [AzK_L1_PEG]LTRMLTFKFYMPKKATELKHLQCLEEE | |
| | LKPLEEVLNLAQSKNFHLRPRDLISNINVIVLELKGSET | |
| | TFMCEYADETATIVEFLNRWITFSQSIISTLT | |
| IL-2_P65 | APTSSSTKKTQLQLEHLLLDLQMILNGINNYKNPKLTRM | 60 |
| [AzK_L1_PEG5kD]-2 | LTFKFYMPKKATELKHLQCLEEELK[AzK_L1_PEG5kD] | |
| | LEEVLNLAQSKNFHLRPRDLISNINVIVLELKGSETTFM | |
| | CEYADETATIVEFLNRWITFSQSIISTLT | |
| IL-2_E62 | APTSSSTKKTQLQLEHLLLDLQMILNGINNYKNPKLTRM | 61 |
| [AzK_L1_PEG5kD]-2 | LTFKFYMPKKATELKHLQCLEE[AzK_L1_PEG5kD]LK | |
| | PLEEVLNLAQSKNFHLRPRDLISNINVIVLELKGSETTF | |
| | MCEYADETATIVEFLNRWITFSQSIISTLT | |
| IL-2_F42 | APTSSSTKKTQLQLEHLLLDLQMILNGINNYKNPKLTRM | 62 |
| [AzK_L1_PEG5kD]-2 | LT[AzK_L1_PEG5kD]KFYMPKKATELKHLQCLEEELK | |
| | PLEEVLNLAQSKNFHLRPRDLISNINVIVLELKGSETTF | |
| | MCEYADETATIVEFLNRWITFSQSIISTLT | |
| IL-2_K43 | APTSSSTKKTQLQLEHLLLDLQMILNGINNYKNPKLTRM | 63 |
| [AzK_L1_PEG5kD]-2 | LTF[AzK_L1_PEG5kD]FYMPKKATELKHLQCLEEELK | |
| | PLEEVLNLAQSKNFHLRPRDLISNINVIVLELKGSETTF | |
| | MCEYADETATIVEFLNRWITFSQSIISTLT | |
| IL-2_K35 | APTSSSTKKTQLQLEHLLLDLQMILNGINNYKNP | 64 |
| [AzK_L1_PEG5kD]-2 | [AzK_L1_PEG5kD]LTRMLTFKFYMPKKATELKHLQCL | |
| | EEELKPLEEVLNLAQSKNFHLRPRDLISNINVIVLELKG | |
| | SETTFMCEYADETATIVEFLNRWITFSQSIISTLT | |
| IL-2_P65 | APTSSSTKKTQLQLEHLLLDLQMILNGINNYKNPKLTRM | 65 |
| [AzK_L1_PEG30kD]-2 | LTFKFYMPKKATELKHLQCLEEELK | |
| | [AzK_L1_PEG30kD]LEEVLNLAQSKNFHLRPRDLISN | |
| | INVIVLELKGSETTFMCEYADETATIVEFLNRWITFSQS | |
| | IISTLT | |
| IL-2_E62 | APTSSSTKKTQLQLEHLLLDLQMILNGINNYKNPKLTRM | 66 |
| [AzK_L1_PEG30kD]-2 | LTFKFYMPKKATELKHLQCLEE[AzK_L1_PEG30kD]L | |
| | KPLEEVLNLAQSKNFHLRPRDLISNINVIVLELKGSETT | |
| | FMCEYADETATIVEFLNRWITFSQSIISTLT | |
| IL-2_F42 | APTSSSTKKTQLQLEHLLLDLQMILNGINNYKNPKLTRM | 67 |
| [AzK_L1_PEG30kD]-2 | LT[AzK_L1_PEG30kD]KFYMPKKATELKHLQCLEEEL | |
| | KPLEEVLNLAQSKNFHLRPRDLISNINVIVLELKGSETT | |
| | FMCEYADETATIVEFLNRWITFSQSIISTLT | |
| IL-2_K43 | APTSSSTKKTQLQLEHLLLDLQMILNGINNYKNPKLTRM | 68 |
| [AzK_L1_PEG30kD]-2 | LTF[AzK_L1_PEG30kD]FYMPKKATELKHLQCLEEEL | |
| | KPLEEVLNLAQSKNFHLRPRDLISNINVIVLELKGSETT | |
| | FMCEYADETATIVEFLNRWITFSQSIISTLT | |
| IL-2_K35 | APTSSSTKKTQLQLEHLLLDLQMILNGINNYKNP | 69 |
| [AzK_L1_PEG30kD]-2 | [AzK_L1_PEG30kD]LTRMLTFKFYMPKKATELKHLQC | |
| | LEEELKPLEEVLNLAQSKNFHLRPRDLISNINVIVLELK | |
| | GSETTFMCEYADETATIVEFLNRWITFSQSIISTLT | |
| IL-2_P65 | PTSSSTKKTQLQLEHLLLDLQMILNGINNYKNPKLTRML | 70 |
| [AzK_PEG]-1 | TFKFYMPKKATELKHLQCLEEELK[AzK_PEG]LEEVLN | |
| | LAQSKNFHLRPRDLISNINVIVLELKGSETTFMCEYADE | |
| | TATIVEFLNRWITFSQSIISTLT | |
| IL-2_E62 | PTSSSTKKTQLQLEHLLLDLQMILNGINNYKNPKLTRML | 71 |
| [AzK_PEG]-1 | TFKFYMPKKATELKHLQCLEE[AzK_PEG]LKPLEEVLN | |
| | LAQSKNFHLRPRDLISNINVIVLELKGSETTFMCEYADE | |
| | TATIVEFLNRWITFSQSIISTLT | |
| IL-2_F42 | PTSSSTKKTQLQLEHLLLDLQMILNGINNYKNPKLTRML | 72 |
| [AzK_PEG]-1 | T[AzK_PEG]KFYMPKKATELKHLQCLEEELKPLEEVLN | |
| | LAQSKNFHLRPRDLISNINVIVLELKGSETTFMCEYADE | |
| | TATIVEFLNRWITFSQSIISTLT | |
| IL-2_K43 | PTSSSTKKTQLQLEHLLLDLQMILNGINNYKNPKLTRML | 73 |
| [AzK_PEG]-1 | TF[AzK_PEG]FYMPKKATELKHLQCLEEELKPLEEVLN | |
| | LAQSKNFHLRPRDLISNINVIVLELKGSETTFMCEYADE | |
| | TATIVEFLNRWITFSQSIISTLT | |
| IL-2_K35 | PTSSSTKKTQLQLEHLLLDLQMILNGINNYKNP | 74 |
| [AzK_PEG]-1 | [AzK_PEG]LTRMLTFKFYMPKKATELKHLQCLEEELKP | |
| | LEEVLNLAQSKNFHLRPRDLISNINVIVLELKGSETTFM | |
| | CEYADETATIVEFLNRWITFSQSIISTLT | |
| IL-2_P65 | PTSSSTKKTQLQLEHLLLDLQMILNGINNYKNPKLTRML | |
| [AzK_PEG5kD]-1 | TFKFYMPKKATELKHLQCLEEELK[AzK_PEG5kD]LEE | 75 |
| | VLNLAQSKNFHLRPRDLISNINVIVLELKGSETTFMCEY | |
| | ADETATIVEFLNRWITFSQSIISTLT | |
| IL-2_E62 | PTSSSTKKTQLQLEHLLLDLQMILNGINNYKNPKLTRML | 76 |
| [AzK_PEG5kD]-1 | TFKFYMPKKATELKHLQCLEE[AzK_PEG5kD]LKPLEE | |
| | VLNLAQSKNFHLRPRDLISNINVIVLELKGSETTFMCEY | |
| | ADETATIVEFLNRWITFSQSIISTLT | |
| IL-2_F42 | PTSSSTKKTQLQLEHLLLDLQMILNGINNYKNPKLTRML | 77 |
| [AzK_PEG5kD]-1 | T[AzK_PEG5kD]KFYMPKKATELKHLQCLEEELKPLEE | |
| | VLNLAQSKNFHLRPRDLISNINVIVLELKGSETTFMCEY | |
| | ADETATIVEFLNRWITFSQSIISTLT | |
| IL-2_K43 | PTSSSTKKTQLQLEHLLLDLQMILNGINNYKNPKLTRML | 78 |
| [AzK_PEG5kD]-1 | TF[AzK_PEG5kD]FYMPKKATELKHLQCLEEELKPLEE | |
| | VLNLAQSKNFHLRPRDLISNINVIVLELKGSETTFMCEY | |
| | ADETATIVEFLNRWITFSQSIISTLT | |
| IL-2_K35 | PTSSSTKKTQLQLEHLLLDLQMILNGINNYKNP | 79 |
| [AzK_PEG5kD]-1 | [AzK_PEG5kD]LTRMLTFKFYMPKKATELKHLQCLEEE | |
| | LKPLEEVLNLAQSKNFHLRPRDLISNINVIVLELKGSET | |
| | TFMCEYADETATIVEFLNRWITFSQSIISTLT | |
| IL-2_P65 | PTSSSTKKTQLQLEHLLLDLQMILNGINNYKNPKLTRML | 80 |
| [AzK_PEG30kD]-1 | TFKFYMPKKATELKHLQCLEEELK[AzK_PEG30kD]LE | |
| | EVLNLAQSKNFHLRPRDLISNINVIVLELKGSETTFMCE | |
| | YADETATIVEFLNRWITFSQSIISTLT | |
| IL-2_E62 | PTSSSTKKTQLQLEHLLLDLQMILNGINNYKNPKLTRML | 81 |
| [AzK_PEG30kD]-1 | TFKFYMPKKATELKHLQCLEE[AzK_PEG30kD]LKPLE | |
| | EVLNLAQSKNFHLRPRDLISNINVIVLELKGSETTFMCE | |
| | YADETATIVEFLNRWITFSQSIISTLT | |
| IL-2_F42 | PTSSSTKKTQLQLEHLLLDLQMILNGINNYKNPKLTRML | 82 |
| [AzK_PEG30kD]-1 | T[AzK_PEG30kD]KFYMPKKATELKHLQCLEEELKPLE | |
| | EVLNLAQSKNFHLRPRDLISNINVIVLELKGSETTFMCE | |
| | YADETATIVEFLNRWITFSQSIISTLT | |
| IL-2_K43 | PTSSSTKKTQLQLEHLLLDLQMILNGINNYKNPKLTRML | 83 |
| [AzK_PEG30kD]-1 | TF[AzK_PEG30kD]FYMPKKATELKHLQCLEEELKPLE | |
| | EVLNLAQSKNFHLRPRDLISNINVIVLELKGSETTFMCE | |
| | YADETATIVEFLNRWITFSQSIISTLT | |
| IL-2_K35 | PTSSSTKKTQLQLEHLLLDLQMILNGINNYKNP | 84 |
| [AzK_PEG30kD]-1 | [AzK_PEG30kD]LTRMLTFKFYMPKKATELKHLQCLEE | |
| | ELKPLEEVLNLAQSKNFHLRPRDLISNINVIVLELKGSE | |
| | TTFMCEYADETATIVEFLNRWITFSQSIISTLT | |
| IL-2_F44X | APTSSSTKKTQLQLEHLLLDLQMILNGINNYKNPKLTRM | 85 |
| | LTFKXYMPKKATELKHLQCLEEELKPLEEVLNLAQSKNF | |
| | HLRPRDLISNINVIVLELKGSETTFMCEYADETATIVEF | |
| | LNRWITFCQSIISTLT | |
| IL-2_F44X-1 | PTSSSTKKTQLQLEHLLLDLQMILNGINNYKNPKLTRML | 86 |
| | TFKXYMPKKATELKHLQCLEEELKPLEEVLNLAQSKNFH | |
| | LRPRDLISNINVIVLELKGSETTFMCEYADETATIVEFL | |
| | NRWITFCQSIISTLT | |
| IL-2_R38X | APTSSSTKKTQLQLEHLLLDLQMILNGINNYKNPKLTXM | 87 |
| | LTFKFYMPKKATELKHLQCLEEELKPLEEVLNLAQSKNF | |
| | HLRPRDLISNINVIVLELKGSETTFMCEYADETATIVEF | |
| | LNRWITFCQSIISTLT | |
| IL-2_R38X-1 | PTSSSTKKTQLQLEHLLLDLQMILNGINNYKNPKLTXML | 88 |
| | TFKFYMPKKATELKHLQCLEEELKPLEEVLNLAQSKNFH | |
| | LRPRDLISNINVIVLELKGSETTFMCEYADETATIVEFL | |
| | NRWITFCQSIISTLT | |
| IL-2_T41X | APTSSSTKKTQLQLEHLLLDLQMILNGINNYKNPKLTRM | 89 |
| | LXFKFYMPKKATELKHLQCLEEELKPLEEVLNLAQSKNF | |
| | HLRPRDLISNINVIVLELKGSETTFMCEYADETATIVEF | |
| | LNRWITFCQSIISTLT | |
| IL-2_T41X-1 | PTSSSTKKTQLQLEHLLLDLQMILNGINNYKNPKLTRML | 90 |
| | XFKFYMPKKATELKHLQCLEEELKPLEEVLNLAQSKNFH | |
| | LRPRDLISNINVIVLELKGSETTFMCEYADETATIVEFL | |
| | NRWITFCQSIISTLT | |
| IL-2_E68X | APTSSSTKKTQLQLEHLLLDLQMILNGINNYKNPKLTRM | 91 |
| | LTFKFYMPKKATELKHLQCLEEELKPLEXVLNLAQSKNF | |
| | HLRPRDLISNINVIVLELKGSETTFMCEYADETATIVEF | |
| | LNRWITFCQSIISTLT | |
| IL-2_E68X-1 | PTSSSTKKTQLQLEHLLLDLQMILNGINNYKNPKLTRML | 92 |
| | TFKFYMPKKATELKHLQCLEEELKPLEXVLNLAQSKNFH | |
| | LRPRDLISNINVIVLELKGSETTFMCEYADETATIVEFL | |
| | NRWITFCQSIISTLT | |
| IL-2_Y45X | APTSSSTKKTQLQLEHLLLDLQMILNGINNYKNPKLTRM | 93 |
| | LTFKFXMPKKATELKHLQCLEEELKPLEEVLNLAQSKNF | |
| | HLRPRDLISNINVIVLELKGSETTFMCEYADETATIVEF | |
| | LNRWITFCQSIISTLT | |
| IL-2_Y45X-1 | PTSSSTKKTQLQLEHLLLDLQMILNGINNYKNPKLTRML | 94 |
| | TFKFXMPKKATELKHLQCLEEELKPLEEVLNLAQSKNFH | |
| | LRPRDLISNINVIVLELKGSETTFMCEYADETATIVEFL | |
| | NRWITFCQSIISTLT | |
| IL-2_V69X | APTSSSTKKTQLQLEHLLLDLQMILNGINNYKNPKLTRM | 95 |
| | LTFKFYMPKKATELKHLQCLEEELKPLEEXLNLAQSKNF | |
| | HLRPRDLISNINVIVLELKGSETTFMCEYADETATIVEF | |
| | LNRWITFCQSIISTLT | |
| IL-2_V69X-1 | PTSSSTKKTQLQLEHLLLDLQMILNGINNYKNPKLTRML | 96 |
| | TFKFYMPKKATELKHLQCLEEELKPLEEXLNLAQSKNFH | |
| | LRPRDLISNINVIVLELKGSETTFMCEYADETATIVEFL | |
| | NRWITFCQSIISTLT | |
| IL-2_L72X | APTSSSTKKTQLQLEHLLLDLQMILNGINNYKNPKLTRM | 97 |
| | LTFKFYMPKKATELKHLQCLEEELKPLEEVLNXAQSKNF | |
| | HLRPRDLISNINVIVLELKGSETTFMCEYADETATIVEF | |
| | LNRWITFCQSIISTLT | |
| IL-2_L72X-1 | PTSSSTKKTQLQLEHLLLDLQMILNGINNYKNPKLTRML | 98 |
| | TFKFYMPKKATELKHLQCLEEELKPLEEVLNXAQSKNFH | |
| | LRPRDLISNINVIVLELKGSETTFMCEYADETATIVEFL | |
| | NRWITFCQSIISTLT | |

X = site comprising an unnatural amino acid.
[AzK]= N6-((2-azidoethoxy)-carbonyl)-L-lysine, having Chemical Abstracts Registry No. 1167421-25-1.
[AzK_PEG] = N6-((2-azidoethoxy)-carbonyl)-L-lysine stably-conjugated to PEG via DBCO-mediated click chemistry, to form a compound comprising a structure of Formula (II) or Formula (III). For example, if specified, PEG5kD indicates a linear polyethylene glycol chain with an average molecular weight of 5 kiloDaltons, capped with a methoxy group. The ratio of regioisomers generated from the click reaction is about 1:1 or greater than 1:1. The term “DBCO” means a chemical moiety comprising a dibenzocyclooctyne group.
[AzK_L1_PEG] = N6-((2-azidoethoxy)-carbonyl)-L-lysine stably-conjugated to PEG via DBCO-mediated click chemistry to form a compound comprising a structure of Formula (IV) or Formula (V). For example, if specified, PEG5kD indicates a linear polyethylene glycol chain with an average molecular weight of 5 kiloDaltons, capped with a methoxy group. The ratio of regioisomers generated from the click reaction is about 1:1 or greater than 1:1. The term “DBCO” means a chemical moiety comprising a dibenzocyclooctyne group.

In some embodiments, the IL-2 conjugate comprises the amino acid sequence of SEQ ID NO: 3 in which at least one amino acid residue in the IL-2 conjugate is replaced by the structure of Formula (I):

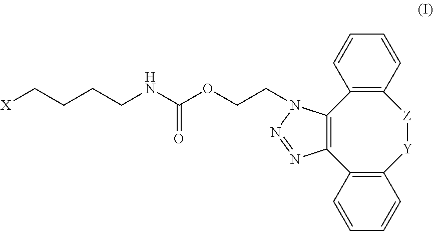

wherein:
Z is CH₂ and Y is

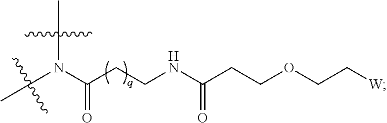

Y is CH₂ and Z is

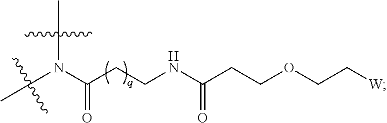

Z is CH₂ and Y is

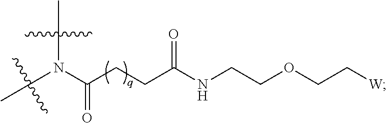

or
Y is CH₂ and Z is

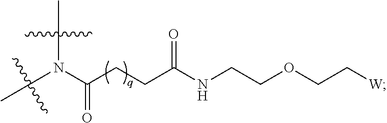

W is a PEG group having an average molecular weight selected from about 5 kDa, 10 kDa, 15 kDa, 20 kDa, 25 kDa, 30 kDa, 35 kDa, 40 kDa, 45 kDa, 50 kDa, and 60 kDa;
q is 1, 2, or 3;
X has the structure:

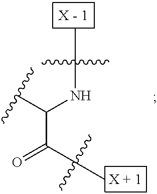

X−1 indicates the point of attachment to the preceding amino acid residue; and
X+1 indicates the point of attachment to the following amino acid residue;
wherein the position of the structure of Formula (I) in the amino acid sequence of the IL-2 conjugate is selected from K34, F41, F43, K42, E61, P64, R37, T40, E67, Y44, V68, and L71.

In any of the embodiments or variations of Formula (I) described herein, the IL-2 conjugate is a pharmaceutically acceptable salt, solvate, or hydrate. In some embodiments, the IL-2 conjugate is a pharmaceutically acceptable salt. In some embodiments, the IL-2 conjugate is a solvate. In some embodiments, the IL-2 conjugate is a hydrate.

In some embodiments or variations of Formula (I) described herein, X is an L-amino acid.

In some embodiments of Formula (I), Z is CH₂ and Y is

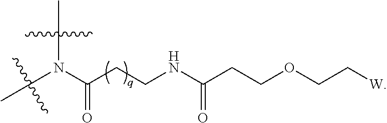

In some embodiments of Formula (I), Y is CH₂ and Z is

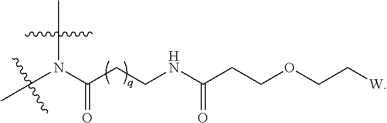

In some embodiments of Formula (I), Z is CH₂ and Y is

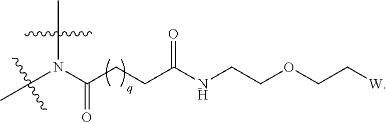

In some embodiments of Formula (I), Y is CH₂ and Z is

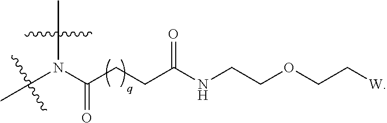

In some embodiments of Formula (I), q is 1. In some embodiments of Formula (I), q is 2. In some embodiments of Formula (I), q is 3.

In some embodiments of Formula (I), q is 1 and the structure of Formula (I) is the structure of Formula (Ia):

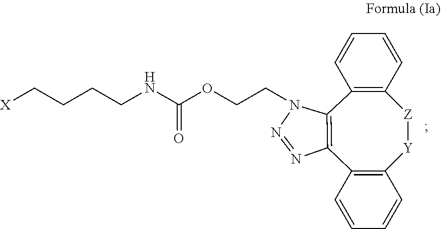

wherein:
Z is CH₂ and Y is

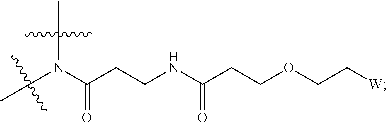

Y is CH₂ and Z is

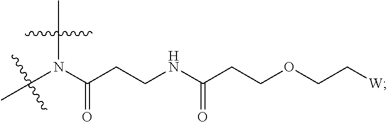

Z is CH₂ and Y is

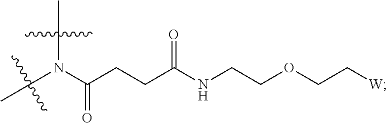

or
Y is CH₂ and Z is

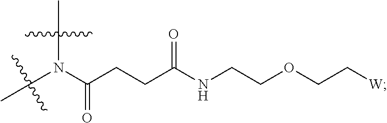

W is a PEG group having an average molecular weight selected from about 5 kDa, 10 kDa, 15 kDa, 20 kDa, 25 kDa, 30 kDa, 35 kDa, 40 kDa, 45 kDa, 50 kDa, and 60 kDa; and
X has the structure:

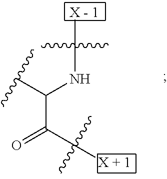

X−1 indicates the point of attachment to the preceding amino acid residue; and
X+1 indicates the point of attachment to the following amino acid residue;
wherein the position of the structure of Formula (Ia) in the amino acid sequence of the IL-2 conjugate is selected from K34, F41, F43, K42, E61, P64, R37, T40, E67, Y44, V68, and L71.

In some embodiments of Formula (Ia), Z is CH₂ and Y is

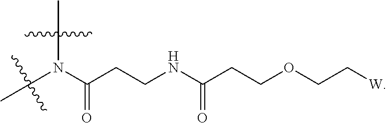

In some embodiments of Formula (Ia), Y is CH₂ and Z is

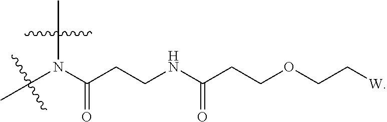

In some embodiments of Formula (Ia), Z is CH₂ and Y is

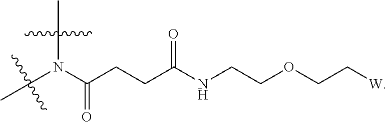

In some embodiments of Formula (Ia), Z is CH₂ and Y is

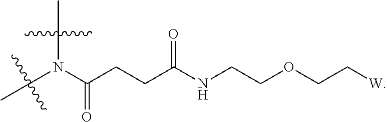

In some embodiments of Formula (Ia), Y is CH₂ and Z is

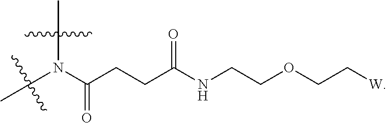

In some embodiments, the PEG group has an average molecular weight selected from about 5 kDa, 10 kDa, 20 kDa and 30 kDa. In some embodiments, the PEG group has an average molecular weight of about 5 kDa. In some embodiments, the PEG group has an average molecular weight of about 10 kDa. In some embodiments, in the PEG group has an average molecular weight of about 15 kDa. In some embodiments, the PEG group has an average molecular weight of about 20 kDa. In some embodiments, the PEG group has an average molecular weight of about 25 kDa. In some embodiments, the PEG group has an average molecular weight of about 30 kDa. In some embodiments, the PEG group has an average molecular weight of about 35 kDa. In some embodiments, the PEG group has an average molecular weight of about 40 kDa. In some embodiments, the PEG group has an average molecular weight of about 45 kDa. In some embodiments, the PEG group has an average molecular weight of about 50 kDa. In some embodiments, the PEG group has an average molecular weight of about 60 kDa.

In some embodiments, the structure of Formula (I) in the amino acid sequence of the IL-2 conjugate is selected from K34, F41, F43, K42, E61, P64, R37, T40, E67, Y44, V68, and L71, wherein the position of the structure of Formula (I) in the amino acid sequence of the IL-2 conjugate is in reference to the positions in SEQ ID NO: 3. In some embodiments, the position of the structure of Formula (I) in the amino acid sequence of the IL-2 conjugate is selected from F41, E61, and P64, wherein the position of the structure of Formula (I) in the amino acid sequence of the IL-2 conjugate is in reference to the positions in SEQ ID NO: 3. In some embodiments, the structure of Formula (I) in the amino acid sequence of the IL-2 conjugate is K34, wherein the position of the structure of Formula (I) in the amino acid sequence of the IL-2 conjugate is in reference to the positions in SEQ ID NO: 3. In some embodiments, the structure of Formula (I) in the amino acid sequence of the IL-2 conjugate is F41, wherein the position of the structure of Formula (I) in the amino acid sequence of the IL-2 conjugate is in reference to the positions in SEQ ID NO: 3. In some embodiments, the structure of Formula (I) in the amino acid sequence of the IL-2 conjugate is selected from F43, wherein the position of the structure of Formula (I) in the amino acid sequence of the IL-2 conjugate is in reference to the positions in SEQ ID NO: 3. In some embodiments, the structure of Formula (I) in the amino acid sequence of the IL-2 conjugate is K42, wherein the position of the structure of Formula (I) in the amino acid sequence of the IL-2 conjugate is in reference to the positions in SEQ ID NO: 3. In some embodiments, the structure of Formula (I) in the amino acid sequence of the IL-2 conjugate is E61, wherein the position of the structure of Formula (I) in the amino acid sequence of the IL-2 conjugate is in reference to the positions in SEQ ID NO: 3. In some embodiments, the structure of Formula (I) in the amino acid sequence of the IL-2 conjugate is P64, wherein the position of the structure of Formula (I) in the amino acid sequence of the IL-2 conjugate is in reference to the positions in SEQ ID NO: 3. In some embodiments, the structure of Formula (I) in the amino acid sequence of the IL-2 conjugate is R37, wherein the position of the structure of Formula (I) in the amino acid sequence of the IL-2 conjugate is in reference to the positions in SEQ ID NO: 3. In some embodiments, the structure of Formula (I) in the amino acid sequence of the IL-2 conjugate is T40, wherein the position of the structure of Formula (I) in the amino acid sequence of the IL-2 conjugate is in reference to the positions in SEQ ID NO: 3. In some embodiments, the structure of Formula (I) in the amino acid sequence of the IL-2 conjugate is selected from E67, wherein the position of the structure of Formula (I) in the amino acid sequence of the IL-2 conjugate is in reference to the positions in SEQ ID NO: 3. In some embodiments, the structure of Formula (I) in the amino acid sequence of the IL-2 conjugate is Y44, wherein the position of the structure of Formula (I) in the amino acid sequence of the IL-2 conjugate is in reference to the positions in SEQ ID NO: 3. In some embodiments, the structure of Formula (I) in the amino acid sequence of the IL-2 conjugate is V68, wherein the position of the structure of Formula (I) in the amino acid sequence of the IL-2 conjugate is in reference to the positions in SEQ ID NO: 3. In some embodiments, the structure of Formula (I) in the amino acid sequence of the IL-2 conjugate is L71, wherein the position of the structure of Formula (I) in the amino acid sequence of the IL-2 conjugate is in reference to the positions in SEQ ID NO: 3.

In some embodiments, the IL-2 conjugate comprises the amino acid sequence of any one of SEQ ID NOS: 15-19, wherein [AzK_PEG] has the structure of Formula (II) or Formula (III), or a mixture of Formula (II) and Formula (III):

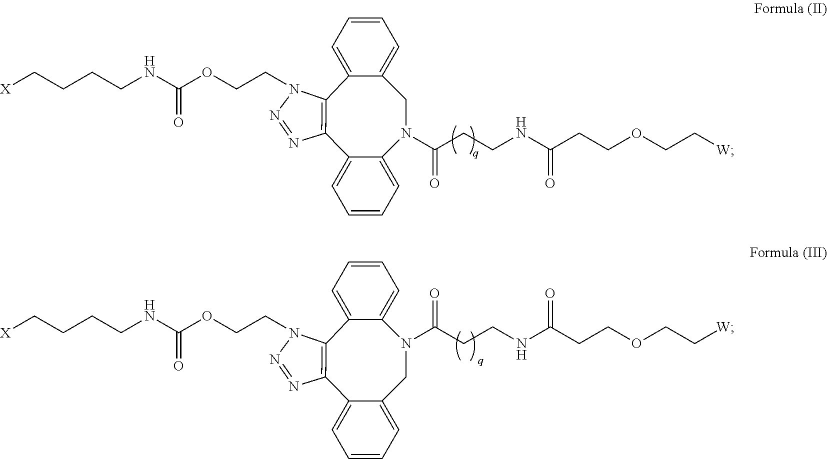

W is a PEG group having an average molecular weight selected from about 5 kDa, 10 kDa, 15 kDa, 20 kDa, 25 kDa, 30 kDa, 35 kDa, 40 kDa, 45 kDa, 50 kDa, and 60 kDa;
q is 1, 2, or 3; and
X has the structure:

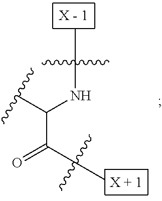

X−1 indicates the point of attachment to the preceding amino acid residue; and
X+1 indicates the point of attachment to the following amino acid residue.

Here and throughout, the structure of Formula (II) encompasses pharmaceutically acceptable salts, solvates, or hydrates thereof. Here and throughout, the structure of Formula (III) encompasses pharmaceutically acceptable salts, solvates, or hydrates thereof. In some embodiments, the IL-2 conjugate is a pharmaceutically acceptable salt, solvate, or hydrate.

In some embodiments, q is 1 and the structures of Formula (II) and Formula (III) are the structures of Formula (IIa) and Formula (IIIa):

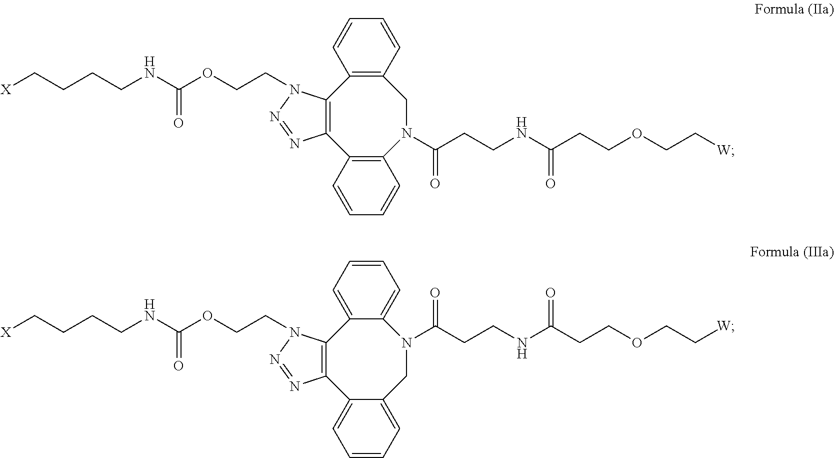

wherein:
W is a PEG group having an average molecular weight selected from about 5 kDa, 10 kDa, 15 kDa, 20 kDa, 25 kDa, 30 kDa, 35 kDa, 40 kDa, 45 kDa, 50 kDa, and 60 kDa; and
X has the structure:

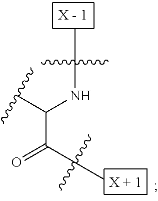

X−1 indicates the point of attachment to the preceding amino acid residue; and
X+1 indicates the point of attachment to the following amino acid residue.

In some embodiments, the [AzK_PEG] is a mixture of Formula (II) and Formula (III). In some embodiments, the [AzK_PEG] is a mixture of Formula (IIa) and Formula (IIIa).

In some embodiments, the [AzK_PEG] has the structure of Formula (II). In some embodiments, the [AzK_PEG] has the structure of Formula (IIa).

In some embodiments, the [AzK_PEG] has the structure of Formula (III). In some embodiments, the [AzK_PEG] has the structure of Formula (IIIa).

In some embodiments, the IL-2 conjugate has the amino acid sequence of SEQ ID NO: 15. In some embodiments, W in the structure of Formula (II) is a PEG group having an average molecular weight selected from about 5 kDa, 10 kDa, 15 kDa, 20 kDa, 25 kDa, and 30 kDa. In some embodiments, W in the structure of Formula (II) is a PEG group having an average molecular weight selected from about 5 kDa and 30 kDa. In some embodiments, W in the structure of Formula (II) is a PEG group having an average molecular weight of about 5 kDa. In some embodiments, W in the structure of Formula (II) is a PEG group having an average molecular weight of about 30 kDa.

In some embodiments, the IL-2 conjugate has the amino acid sequence of SEQ ID NO: 16. In some embodiments, W in the structure of Formula (II) is a PEG group having an average molecular weight selected from about 5 kDa, 10 kDa, 15 kDa, 20 kDa, 25 kDa, and 30 kDa. In some embodiments, W in the structure of Formula (II) is a PEG group having an average molecular weight selected from 5 about kDa and 30 kDa. In some embodiments, W in the structure of Formula (II) is a PEG group having an average molecular weight of about 5 kDa. In some embodiments, W in the structure of Formula (II) is a PEG group having an average molecular weight of about 30 kDa.

In some embodiments, the IL-2 conjugate has the amino acid sequence of SEQ ID NO: 17. In some embodiments, W in the structure of Formula (II) is a PEG group having an average molecular weight selected from about 5 kDa, 10 kDa, 15 kDa, 20 kDa, 25 kDa, and 30 kDa. In some embodiments, W in the structure of Formula (II) is a PEG group having an average molecular weight selected from about 5 kDa and 30 kDa. In some embodiments, W in the structure of Formula (II) is a PEG group having an average molecular weight of about 5 kDa. In some embodiments, W in the structure of Formula (II) is a PEG group having an average molecular weight of about 30 kDa.

In some embodiments, the IL-2 conjugate has the amino acid sequence of SEQ ID NO: 18. In some embodiments, W in the structure of Formula (II) is a PEG group having an average molecular weight selected from about 5 kDa, 10 kDa, 15 kDa, 20 kDa, 25 kDa, and 30 kDa. In some embodiments, W in the structure of Formula (II) is a PEG group having an average molecular weight selected from about 5 kDa and 30 kDa. In some embodiments, W in the structure of Formula (II) is a PEG group having an average molecular weight of about 5 kDa. In some embodiments, W in the structure of Formula (II) is a PEG group having an average molecular weight of about 30 kDa.

In some embodiments, the IL-2 conjugate has the amino acid sequence of SEQ ID NO: 19. In some embodiments, W in the structure of Formula (II) is a PEG group having an average molecular weight selected from about 5 kDa, 10 kDa, 15 kDa, 20 kDa, 25 kDa, and 30 kDa. In some embodiments, W in the structure of Formula (II) is a PEG group having an average molecular weight selected from about 5 kDa and 30 kDa. In some embodiments, W in the structure of Formula (II) is a PEG group having an average molecular weight of about 5 kDa. In some embodiments, W in the structure of Formula (II) is a PEG group having an average molecular weight of about 30 kDa.

In some embodiments, the [AzK_PEG] has the structure of Formula (III). In some embodiments, the [AzK_PEG] has the structure of Formula (IIIa).

In some embodiments, the IL-2 conjugate has the amino acid sequence of SEQ ID NO: 15. In some embodiments, W in the structure of Formula (III) is a PEG group having an average molecular weight selected from about 5 kDa, 10 kDa, 15 kDa, 20 kDa, 25 kDa, and 30 kDa. In some embodiments, W in the structure of Formula (III) is a PEG group having an average molecular weight selected from about 5 kDa and 30 kDa. In some embodiments, W in the structure of Formula (III) is a PEG group having an average molecular weight of about 5 kDa. In some embodiments, W in the structure of Formula (III) is a PEG group having an average molecular weight of about 30 kDa.

In some embodiments, the IL-2 conjugate has the amino acid sequence of SEQ ID NO: 16. In some embodiments, W in the structure of Formula (III) is a PEG group having an average molecular weight selected from about 5 kDa, 10 kDa, 15 kDa, 20 kDa, 25 kDa, and 30 kDa. In some embodiments, W in the structure of Formula (III) is a PEG group having an average molecular weight selected from about 5 kDa and 30 kDa. In some embodiments, W in the structure of Formula (III) is a PEG group having an average molecular weight of about 5 kDa. In some embodiments, W in the structure of Formula (III) is a PEG group having an average molecular weight of about 30 kDa.

In some embodiments, the IL-2 conjugate has the amino acid sequence of SEQ ID NO: 17. In some embodiments, W in the structure of Formula (III) is a PEG group having an average molecular weight selected from about 5 kDa, 10 kDa, 15 kDa, 20 kDa, 25 kDa, and 30 kDa. In some embodiments, W in the structure of Formula (III) is a PEG group having an average molecular weight selected from about 5 kDa and 30 kDa. In some embodiments, W in the structure of Formula (III) is a PEG group having an average molecular weight of about 5 kDa. In some embodiments, W in the structure of Formula (III) is a PEG group having an average molecular weight of about 30 kDa.

In some embodiments, the IL-2 conjugate has the amino acid sequence of SEQ ID NO: 18. In some embodiments, W in the structure of Formula (III) is a PEG group having an average molecular weight selected from about 5 kDa, 10 kDa, 15 kDa, 20 kDa, 25 kDa, and 30 kDa. In some embodiments, W in the structure of Formula (III) is a PEG group having an average molecular weight selected from about 5 kDa and 30 kDa. In some embodiments, W in the structure of Formula (III) is a PEG group having an average molecular weight of about 5 kDa. In some embodiments, W in the structure of Formula (III) is a PEG group having an average molecular weight of about 30 kDa.

In some embodiments, the IL-2 conjugate has the amino acid sequence of SEQ ID NO: 19. In some embodiments, W in the structure of Formula (III) is a PEG group having an average molecular weight selected from about 5 kDa, 10 kDa, 15 kDa, 20 kDa, 25 kDa, and 30 kDa. In some embodiments, W in the structure of Formula (III) is a PEG group having an average molecular weight selected from about 5 kDa and 30 kDa. In some embodiments, W in the structure of Formula (III) is a PEG group having an average molecular weight of about 5 kDa. In some embodiments, W in the structure of Formula (III) is a PEG group having an average molecular weight of about 30 kDa.

In some embodiments, the IL-2 conjugate has the amino acid sequence selected from any one of SEQ ID NO: 15, 16, 17, 18, and 19, wherein [AzK_PEG] contains a PEG group having an average molecular weight selected from about 5 kDa, 10 kDa, 15 kDa, 20 kDa, 25 kDa, 30 kDa, 35 kDa, 40 kDa, 45 kDa, 50 kDa, and 60 kDa. In some embodiments, the IL-2 conjugate has the amino acid sequence selected from any one of SEQ ID NO: 15, 16, 17, 18, and 19, wherein [AzK_PEG] contains a PEG group having an average molecular weight of about 5 kDa. In some embodiments, the IL-2 conjugate has the amino acid sequence selected from any one of SEQ ID NO: 15, 16, 17, 18, and 19, wherein [AzK_PEG] contains a PEG group having an average molecular weight of about 10 kDa. In some embodiments, the IL-2 conjugate has the amino acid sequence selected from any one of SEQ ID NO: 15, 16, 17, 18, and 19, wherein [AzK_PEG] contains a PEG group having an average molecular weight of about 15 kDa. In some embodiments, the IL-2 conjugate has the amino acid sequence selected from any one of SEQ ID NO: 15, 16, 17, 18, and 19, wherein [AzK_PEG] contains a PEG group having an average molecular weight of about 20 kDa. In some embodiments, the IL-2 conjugate has the amino acid sequence selected from any one of SEQ ID NO: 15, 16, 17, 18, and 19, wherein [AzK_PEG] contains a PEG group having an average molecular weight of about 25 kDa. In some embodiments, the IL-2 conjugate has the amino acid sequence selected from any one of SEQ ID NO: 15, 16, 17, 18, and 19, wherein [AzK_PEG] contains a PEG group having an average molecular weight of about 30 kDa. In some embodiments, the IL-2 conjugate has the amino acid sequence selected from any one of SEQ ID NO: 15, 16, 17, 18, and 19, wherein [AzK_PEG] contains a PEG group having an average molecular weight of about 35 kDa. In some embodiments, the IL-2 conjugate has the amino acid sequence selected from any one of SEQ ID NO: 15, 16, 17, 18, and 19, wherein [AzK_PEG] contains a PEG group having an average molecular weight of about 40 kDa. In some embodiments, the IL-2 conjugate has the amino acid sequence selected from any one of SEQ ID NO: 15, 16, 17, 18, and 19, wherein [AzK_PEG] contains a PEG group having an average molecular weight of about 45 kDa. In some embodiments, the IL-2 conjugate has the amino acid sequence selected from any one of SEQ ID NO: 15, 16, 17, 18, and 19, [AzK_PEG] contains a PEG group having an average molecular weight of about 50 kDa. In some embodiments, the IL-2 conjugate has the amino acid sequence selected from any one of SEQ ID NO: 15, 16, 17, 18, and 19, wherein [AzK_PEG] contains a PEG group having an average molecular weight of about 60 kDa. In some embodiments, the IL-2 conjugate has the amino acid sequence selected from any one of SEQ ID NO: 15, 16, 17, 18, and 19, wherein [AzK_PEG] contains a PEG group having an average molecular weight selected from about 5 kDa, 10 kDa, 15 kDa, 20 kDa, 25 kDa, 30 kDa, 35 kDa, 40 kDa, 45 kDa, 50 kDa, and 60 kDa, and the PEG group is a methoxy PEG group, a linear methoxy PEG group, or a branched methoxy PEG group.

In some embodiments, the IL-2 conjugate comprises the amino acid sequence of any one of SEQ ID NOS: 20-24, wherein [AzK_PEG5 kD] has the structure of Formula (II) or Formula (III), or a mixture of Formula (II) and Formula (III).

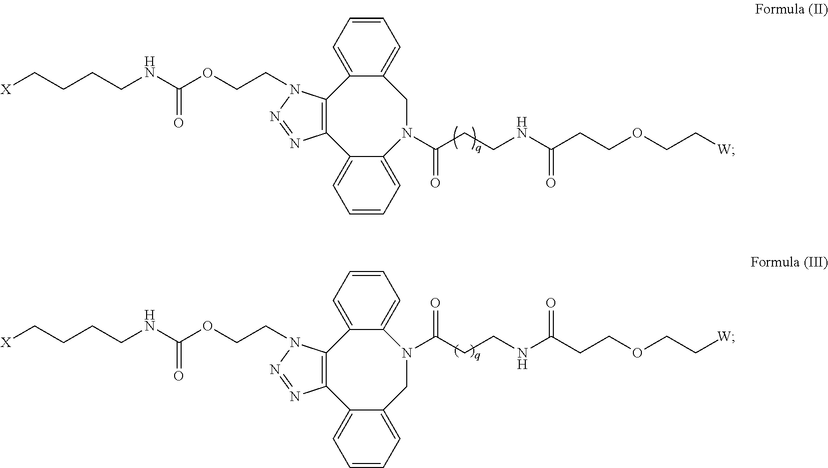

wherein:
W is a PEG group having an average molecular weight of about 5 kDa;
q is 1, 2, or 3; and
X has the structure:

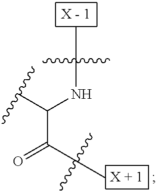 [SMILES: *NC(*)C(*)=O]

X−1 indicates the point of attachment to the preceding amino acid residue; and
X+1 indicates the point of attachment to the following amino acid residue.

In some embodiments, q is 1 and the [AzK_PEG5 kD] has the structure of Formula (IIa) or Formula (IIIa), or a mixture of Formula (IIa) and Formula (IIIa).

In some embodiments, the IL-2 conjugate has the amino acid sequence of SEQ ID NO: 20. In some embodiments, the IL-2 conjugate has the amino acid sequence of SEQ ID NO: 21. In some embodiments, the IL-2 conjugate has the amino acid sequence of SEQ ID NO: 22. In some embodiments, the IL-2 conjugate has the amino acid sequence of SEQ ID NO: 23. In some embodiments, the IL-2 conjugate has the amino acid sequence of SEQ ID NO: 24.

In some embodiments, the [AzK_PEG5 kD] has the structure of Formula (II)

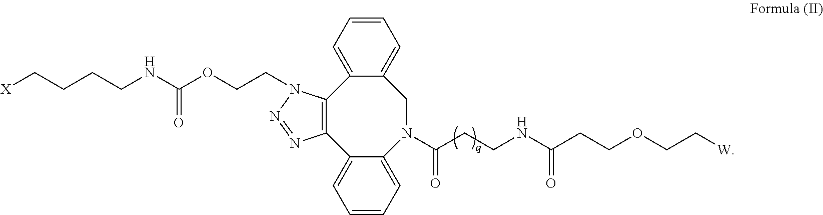

In some embodiments, q is 1 and the [AzK_PEG5 kD] has the structure of Formula (IIa).

In some embodiments, the [AzK_PEG5 kD] has the structure of Formula (III):

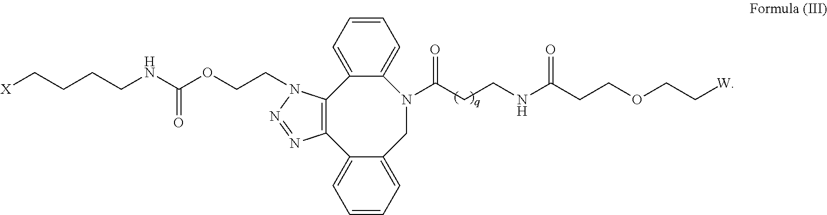

In some embodiments, q is 1 and the [AzK_PEG5 kD] has the structure of Formula (IIIa).

In some embodiments, the IL-2 conjugate comprises the amino acid sequence of any one of SEQ ID NOS: 25-29, wherein [AzK_PEG30 kD] has the structure of Formula (II) or Formula (III), or is a mixture of the structures of Formula (II) and Formula (III).

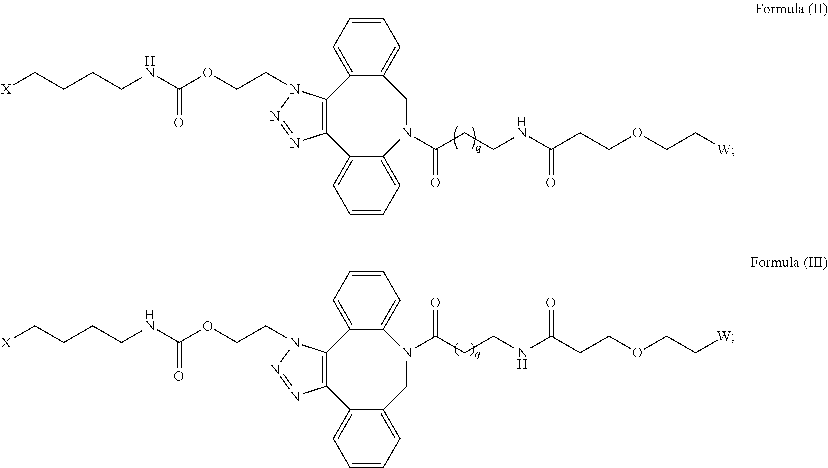

wherein:
W is a PEG group having an average molecular weight of about 30 kDa;
q is 1, 2, or 3; and
X has the structure:

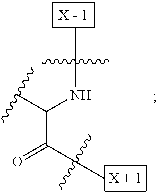

X−1 indicates the point of attachment to the preceding amino acid residue; and
X+1 indicates the point of attachment to the following amino acid residue.

In some embodiments, q is 1 and the [AzK_PEG30 kD] has the structure of Formula (IIa) or Formula (IIIa), or is a mixture of the structures of Formula (IIa) and Formula (IIIa).

In some embodiments, the IL-2 conjugate has the amino acid sequence of SEQ ID NO: 25. In some embodiments, the IL-2 conjugate has the amino acid sequence of SEQ ID NO: 26. In some embodiments, the IL-2 conjugate has the amino acid sequence of SEQ ID NO: 27. In some embodiments, the IL-2 conjugate has the amino acid sequence of SEQ ID NO: 28. In some embodiments, the IL-2 conjugate has the amino acid sequence of SEQ ID NO: 29.

In some embodiments, the [AzK_PEG30 kD] has the structure of Formula (II):

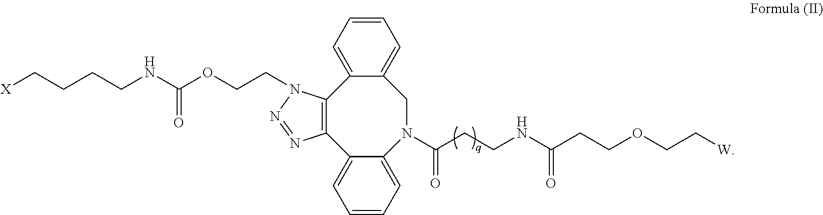

In some embodiments, q is 1 and the [AzK_PEG30 kD] has the structure of Formula (IIa).

In some embodiments, the methods use an IL-2 conjugate in which the [AzK_PEG30 kD] has the structure of Formula (III):

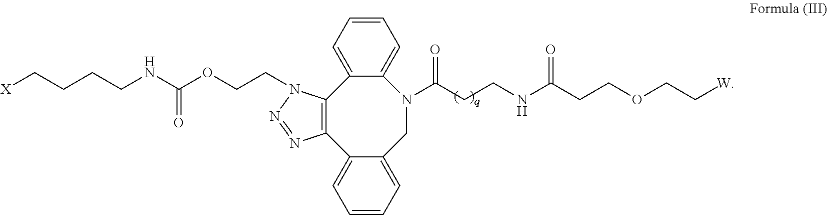

In some embodiments, q is 1 and the [AzK_PEG30 kD] has the structure of Formula (IIIa).

In some embodiments, the [AzK_PEG] is a mixture of the structures of Formula (II) and Formula (III):

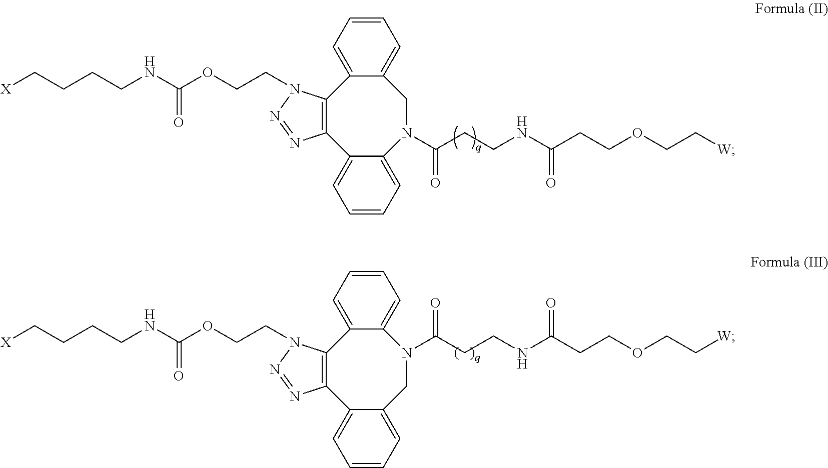

wherein:
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa;
q is 1, 2, or 3; and
X has the structure:

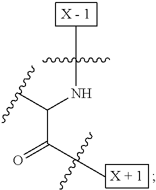 [SMILES: *NC(*)C(*)=O]

X−1 indicates the point of attachment to the preceding amino acid residue; and
X+1 indicates the point of attachment to the following amino acid residue.

In some embodiments, q is 1 and the [AzK_PEG] is a mixture of the structures of Formula (IIa) and Formula (IIIa).

In some embodiments, the ratio of the amount of the structure of Formula (II) to the amount of the structure of Formula (III) comprising the total amount of [AzK_PEG] in the IL-2 conjugate is about 1:1. In some embodiments, the ratio of the amount of the structure of Formula (II) to the amount of the structure of Formula (III) comprising the total amount of [AzK_PEG] in the IL-2 conjugate is greater than 1:1. In some embodiments, the ratio of the amount of the structure of Formula (II) to the amount of the structure of Formula (III) comprising the total amount of [AzK_PEG] in the IL-2 conjugate is less than 1:1.

In some embodiments, W is a linear or branched PEG group. In some embodiments, W is a linear PEG group. In some embodiments W is a branched PEG group. In some embodiments, W is a methoxy PEG group. In some embodiments, the methoxy PEG group is linear or branched. In some embodiments, the methoxy PEG group is linear. In some embodiments, the methoxy PEG group is branched.

In some embodiments, the IL-2 conjugate comprises the amino acid sequence of any one of SEQ ID NOS: 20 to 24, wherein [AzK_PEG5 kD] is a mixture of the structures of Formula (II) and Formula (III):

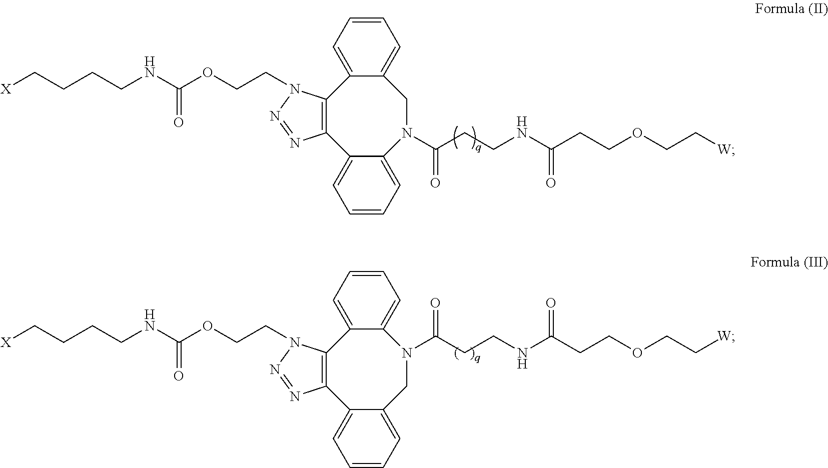

wherein:
W is a PEG group having an average molecular weight of 5 kDa;
q is 1, 2, or 3; and
X has the structure:

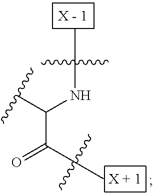

X−1 indicates the point of attachment to the preceding amino acid residue; and
X+1 indicates the point of attachment to the following amino acid residue.

In some embodiments, q is 1 and the [AzK_PEG5 kD] is a mixture of the structures of Formula (IIa) and Formula (IIIa).

In some embodiments, the ratio of the amount of the structure of Formula (II) to the amount of the structure of Formula (III) comprising the total amount of [AzK_PEG5 kD] in the IL-2 conjugate is about 1:1. In some embodiments, the ratio of the amount of the structure of Formula (II) to the amount of the structure of Formula (III) comprising the total amount of [AzK_PEG5 kD] in the IL-2 conjugate is greater than 1:1. In some embodiments, the ratio of the amount of the structure of Formula (II) to the amount of the structure of Formula (III) comprising the total amount of [AzK_PEG5 kD] in the IL-2 conjugate is less than 1:1.

In some embodiments, the IL-2 conjugate comprises the amino acid sequence of any one of SEQ ID NOS: 25-29, wherein [AzK_PEG30 kD] is a mixture of the structures of Formula (II) and Formula (III):

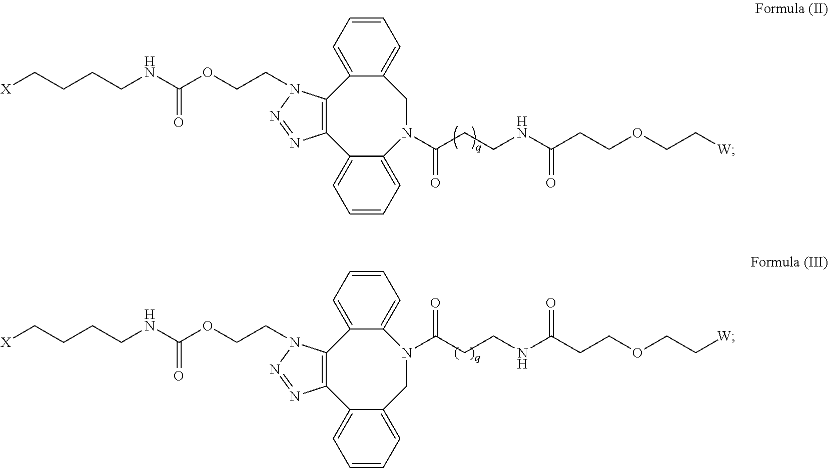

wherein:
W is a PEG group having an average molecular weight of 30 kDa;
q is 1, 2, or 3; and
X has the structure:

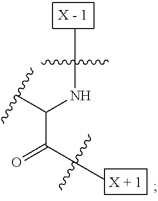

X−1 indicates the point of attachment to the preceding amino acid residue; and
X+1 indicates the point of attachment to the following amino acid residue.

In some embodiments, q is 1 and the [AzK_PEG30 kD] is a mixture of the structures of Formula (IIa) and Formula (IIIa).

In some embodiments, the ratio of the amount of the structure of Formula (II) to the amount of the structure of Formula (III) comprising the total amount of [AzK_PEG30 kD] in the IL-2 conjugate is about 1:1. In some embodiments, the ratio of the amount of the structure of Formula (II) to the amount of the structure of Formula (III) comprising the total amount of [AzK_PEG30 kD] in the IL-2 conjugate is greater than 1:1. In some embodiments, the ratio of the amount of the structure of Formula (II) to the amount of the structure of Formula (III) comprising the total amount of [AzK_PEG30 kD] in the IL-2 conjugate is less than 1:1.

In some embodiments, the IL-2 conjugate comprises the structure of Formula (II) or Formula (III), or a mixture of Formula (II) and Formula (III), wherein W is a linear or branched PEG group, or a pharmaceutically acceptable salt, solvate, or hydrate thereof. In some embodiments, W in the structure of Formula (II) or Formula (III) is a linear PEG group. In some embodiments, W in the structure of Formula (II) or Formula (III) is a branched PEG group. In some embodiments, W in the structure of Formula (II) or Formula (III) is a methoxy PEG group. In some embodiments, W in the structure of Formula (II) or Formula (III) is a methoxy PEG group that is linear or branched. In some embodiments, the methoxy PEG group in the structure of Formula (II) or Formula (III) is linear. In some embodiments, the methoxy PEG group in the structure of Formula (II) or Formula (III) is branched.

In some embodiments, the IL-2 conjugate comprises the amino acid sequence of any one of SEQ ID NOS: 40-44, wherein [AzK_L1_PEG] has the structure of Formula (IV) or Formula (V), or a mixture of Formula (IV) and Formula (V):

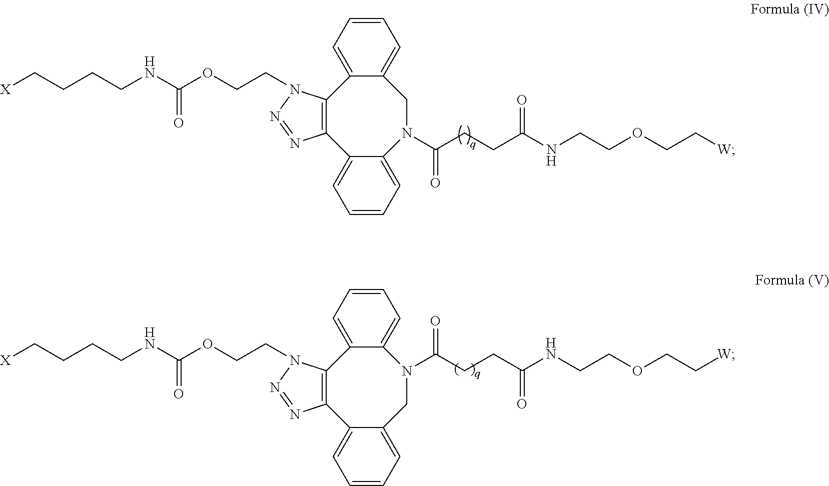

wherein:
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa;
q is 1, 2, or 3; and
X has the structure:

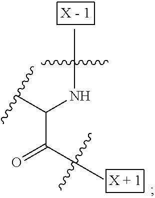 [SMILES: *NC(*)C(*)=O]

X−1 indicates the point of attachment to the preceding amino acid residue; and
X+1 indicates the point of attachment to the following amino acid residue.

In some embodiments of Formula (IV) or Formula (V), or a mixture of Formula (IV) or Formula (V), q is 1. In some embodiments of Formula (IV) or Formula (V), or a mixture of Formula (IV) or Formula (V), q is 2. In some embodiments of Formula (IV) or Formula (V), or a mixture of Formula (IV) or Formula (V), q is 3.

Here and throughout, the structure of Formula (IV) encompasses pharmaceutically acceptable salts, solvates, or hydrates thereof. Here and throughout, the structure of Formula (V) encompasses pharmaceutically acceptable salts, solvates, or hydrates thereof. In some embodiments, the IL-2 conjugate is a pharmaceutically acceptable salt, solvate, or hydrate.

In some embodiments, q is 1 and the structures of Formula (IV) and Formula (V) are Formula (IVa) and Formula (Va):

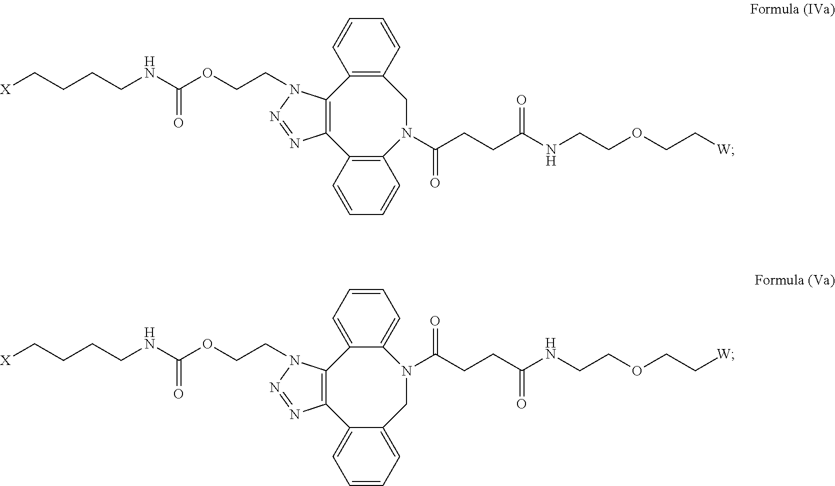

wherein:
W is a PEG group having an average molecular weight selected from about 5 kDa, 10 kDa, 15 kDa, 20 kDa, 25 kDa, 30 kDa, 35 kDa, 40 kDa, 45 kDa, 50 kDa, and 60 kDa; and
X has the structure:

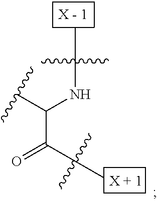

X−1 indicates the point of attachment to the preceding amino acid residue; and
X+1 indicates the point of attachment to the following amino acid residue.

In some embodiments, the [AzK_L1_PEG] is a mixture of Formula (IV) and Formula (V). In some embodiments, the [AzK_L1_PEG] is a mixture of Formula (IVa) and Formula (Va).

In some embodiments, the [AzK_L1_PEG] has the structure of Formula (IV):

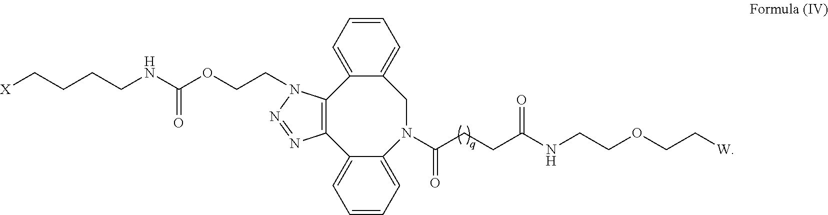

In some embodiments, q is 1 and the [AzK_L1_PEG] has the structure of Formula (IVa).

In some embodiments, the IL-2 conjugate has the amino acid sequence of SEQ ID NO: 40. In some embodiments, W in the structure of Formula (IV) is a PEG group having an average molecular weight selected from about 5 kDa, 10 kDa, 15 kDa, 20 kDa, 25 kDa, and 30 kDa. In some embodiments, W in the structure of Formula (IV) is a PEG group having an average molecular weight selected from about 5 kDa and 30 kDa. In some embodiments, W in the structure of Formula (IV) is a PEG group having an average molecular weight of about 5 kDa. In some embodiments, W in the structure of Formula (IV) is a PEG group having an average molecular weight of about 30 kDa.

In some embodiments, the IL-2 conjugate has the amino acid sequence of SEQ ID NO: 41. In some embodiments, W in the structure of Formula (IV) is a PEG group having an average molecular weight selected from about 5 kDa, 10 kDa, 15 kDa, 20 kDa, 25 kDa, and 30 kDa. In some embodiments, W in the structure of Formula (IV) is a PEG group having an average molecular weight selected from about 5 kDa and 30 kDa. In some embodiments, W in the structure of Formula (IV) is a PEG group having an average molecular weight of about 5 kDa. In some embodiments, W in the structure of Formula (IV) is a PEG group having an average molecular weight of about 30 kDa.

In some embodiments, the IL-2 conjugate has the amino acid sequence of SEQ ID NO: 42. In some embodiments, W in the structure of Formula (IV) is a PEG group having an average molecular weight selected from about 5 kDa, 10 kDa, 15 kDa, 20 kDa, 25 kDa, and 30 kDa. In some embodiments, W in the structure of Formula (IV) is a PEG group having an average molecular weight selected from about 5 kDa and 30 kDa. In some embodiments, W is a PEG group having an average molecular weight of about 5 kDa. In some embodiments, W in the structure of Formula (IV) is a PEG group having an average molecular weight of about 30 kDa.

In some embodiments, the IL-2 conjugate has the amino acid sequence of SEQ ID NO: 43. In some embodiments, W in the structure of Formula (IV) is a PEG group having an average molecular weight selected from about 5 kDa, 10 kDa, 15 kDa, 20 kDa, 25 kDa, and 30 kDa. In some embodiments, W in the structure of Formula (IV) is a PEG group having an average molecular weight selected from about 5 kDa and 30 kDa. In some embodiments, W in the structure of Formula (IV) is a PEG group having an average molecular weight of about 5 kDa. In some embodiments, W in the structure of Formula (IV) is a PEG group having an average molecular weight of about 30 kDa.

In some embodiments, the IL-2 conjugate has the amino acid sequence of SEQ ID NO: 44. In some embodiments, W in the structure of Formula (IV) is a PEG group having an average molecular weight selected from about 5 kDa, 10 kDa, 15 kDa, 20 kDa, 25 kDa, and 30 kDa. In some embodiments, W in the structure of Formula (IV) is a PEG group having an average molecular weight selected from about 5 kDa and 30 kDa. In some embodiments, W in the structure of Formula (IV) is a PEG group having an average molecular weight of about 5 kDa. In some embodiments, W in the structure of Formula (IV) is a PEG group having an average molecular weight of about 30 kDa.

In some embodiments, the [AzK_L1_PEG] has the structure of Formula (V):

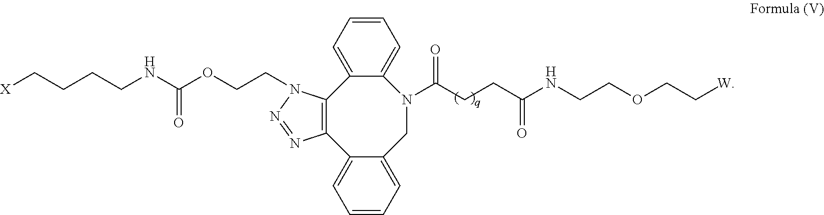

In some embodiments, q is 1 and the [AzK_L1_PEG] has the structure of Formula (V).

In some embodiments, the IL-2 conjugate has the amino acid sequence of SEQ ID NO: 40. In some embodiments, W in the structure of Formula (V) is a PEG group having an average molecular weight selected from about 5 kDa, 10 kDa, 15 kDa, 20 kDa, 25 kDa, and 30 kDa. In some embodiments, W in the structure of Formula (V) is a PEG group having an average molecular weight selected from about 5 kDa and 30 kDa. In some embodiments, W in the structure of Formula (V) is a PEG group having an average molecular weight of about 5 kDa. In some embodiments, W in the structure of Formula (V) is a PEG group having an average molecular weight of about 30 kDa.

In some embodiments, the IL-2 conjugate has the amino acid sequence of SEQ ID NO: 41. In some embodiments, W in the structure of Formula (V) is a PEG group having an average molecular weight selected from about 5 kDa, 10 kDa, 15 kDa, 20 kDa, 25 kDa, and 30 kDa. In some embodiments, W in the structure of Formula (V) is a PEG group having an average molecular weight selected from about 5 kDa and 30 kDa. In some embodiments, W in the structure of Formula (V) is a PEG group having an average molecular weight of about 5 kDa. In some embodiments, W in the structure of Formula (V) is a PEG group having an average molecular weight of about 30 kDa.

In some embodiments, the IL-2 conjugate has the amino acid sequence of SEQ ID NO: 42. In some embodiments, W in the structure of Formula (V) is a PEG group having an average molecular weight selected from about 5 kDa, 10 kDa, 15 kDa, 20 kDa, 25 kDa, and 30 kDa. In some embodiments, W in the structure of Formula (V) is a PEG group having an average molecular weight selected from about 5 kDa and 30 kDa. In some embodiments, W in the structure of Formula (V) is a PEG group having an average molecular weight of about 5 kDa. In some embodiments W in the structure of Formula (V) is a PEG group having an average molecular weight of about 30 kDa.

In some embodiments, the IL-2 conjugate has the amino acid sequence of SEQ ID NO: 43. In some embodiments, W in the structure of Formula (V) is a PEG group having an average molecular weight selected from about 5 kDa, 10 kDa, 15 kDa, 20 kDa, 25 kDa, and 30 kDa. In some embodiments, W in the structure of Formula (V) is a PEG group having an average molecular weight selected from about 5 kDa and 30 kDa. In some embodiments, W in the structure of Formula (V) is a PEG group having an average molecular weight of about 5 kDa. In some embodiments W in the structure of Formula (V) is a PEG group having an average molecular weight of about 30 kDa.

In some embodiments, the IL-2 conjugate has the amino acid sequence selected from any one of SEQ ID NO: 40, 41, 42, 43, and 44, wherein [AzK_L1_PEG] contains a PEG group having an average molecular weight selected from about 5 kDa, 10 kDa, 15 kDa, 20 kDa, 25 kDa, 30 kDa, 35 kDa, 40 kDa, 45 kDa, 50 kDa, and 60 kDa. In some embodiments, the IL-2 conjugate has the amino acid sequence selected from any one of SEQ ID NO: 40, 41, 42, 43, and 44, wherein [AzK_L1_PEG] contains a PEG group having an average molecular weight of about 5 kDa. In some embodiments, the IL-2 conjugate has the amino acid sequence selected from any one of SEQ ID NO: 40, 41, 42, 43, and 44, wherein [AzK_L1_PEG] contains a PEG group having an average molecular weight of about 10 kDa. In some embodiments, the IL-2 conjugate has the amino acid sequence selected from any one of SEQ ID NO: 40, 41, 42, 43, and 44, wherein [AzK_L1_PEG] contains a PEG group having an average molecular weight of about 15 kDa. In some embodiments, the IL-2 conjugate has the amino acid sequence selected from any one of SEQ ID NO: 40, 41, 42, 43, and 44, wherein [AzK_L1_PEG] contains a PEG group having an average molecular weight of about 20 kDa. In some embodiments, the IL-2 conjugate has the amino acid sequence selected from any one of SEQ ID NO: 40, 41, 42, 43, and 44, wherein [AzK_L1_PEG] contains a PEG group having an average molecular weight of about 25 kDa. In some embodiments, the IL-2 conjugate has the amino acid sequence selected from any one of SEQ ID NO: 40, 41, 42, 43, and 44, wherein [AzK_L1_PEG] contains a PEG group having an average molecular weight of about 30 kDa. In some embodiments, the IL-2 conjugate has the amino acid sequence selected from any one of SEQ ID NO: 40, 41, 42, 43, and 44, wherein [AzK_L1_PEG] contains a PEG group having an average molecular weight of about 35 kDa. In some embodiments, the IL-2 conjugate has the amino acid sequence selected from any one of SEQ ID NO: 40, 41, 42, 43, and 44, wherein [AzK_L1_PEG] contains a PEG group having an average molecular weight of about 40 kDa. In some embodiments, the IL-2 conjugate has the amino acid sequence selected from any one of SEQ ID NO: 40, 41, 42, 43, and 44, wherein [AzK_L1_PEG] contains a PEG group having an average molecular weight of about 45 kDa In some embodiments, the IL-2 conjugate has the amino acid sequence selected from any one of SEQ ID NO: 40, 41, 42, 43, and 44, wherein [AzK_L1_PEG] contains a PEG group having an average molecular weight of about 50 kDa. In some embodiments, the IL-2 conjugate has the amino acid sequence selected from any one of SEQ ID NO: 40, 41, 42, 43, and 44, wherein [AzK_L1_PEG] contains a PEG group having an average molecular weight of about 60 kDa. In some embodiments, the IL-2 conjugate has the amino acid sequence selected from any one of SEQ ID NO: 40, 41, 42, 43, and 44, wherein [AzK_L1_PEG] contains a PEG group having an average molecular weight selected from about 5 kDa, 10 kDa, 15 kDa, 20 kDa, 25 kDa, 30 kDa, 35 kDa, 40 kDa, 45 kDa, 50 kDa, and 60 kDa, and wherein the PEG group is a methoxy PEG group, a linear methoxy PEG group, or a branched methoxy PEG group.

In some embodiments, the IL-2 conjugate comprises the amino acid sequence of any one of SEQ ID NOS: 45-49, wherein [AzK_L1_PEG5 kD] has the structure of Formula (IV) or Formula (V), or a mixture of Formula (IV) and Formula (V):

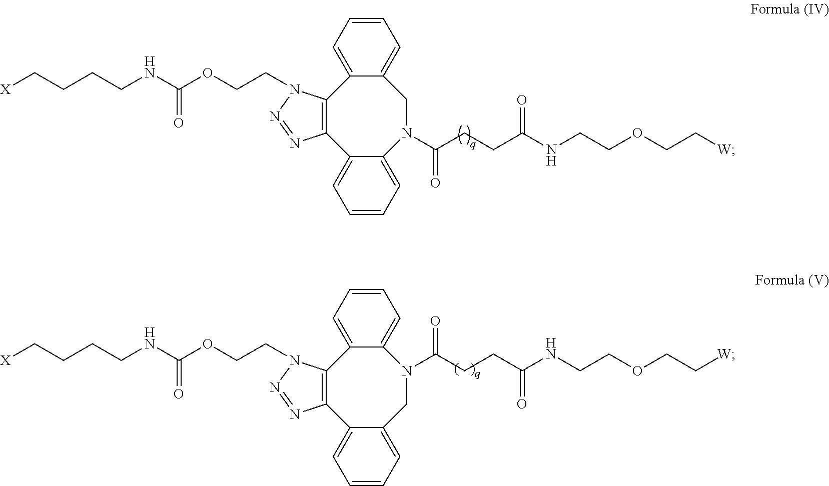

wherein:
W is a PEG group having an average molecular weight of about 5 kDa; and
X has the structure:

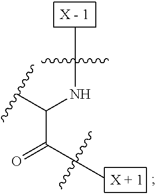 [SMILES: *NC(*)C(*)=O]

X−1 indicates the point of attachment to the preceding amino acid residue; and
X+1 indicates the point of attachment to the following amino acid residue.

In some embodiments, q is 1 and the [AzK_L1_PEG5 kD] has the structure of Formula (IVa) or Formula (Va), or a mixture of Formula (IVa) and Formula (Va).

In some embodiments, the IL-2 conjugate has the amino acid sequence of SEQ ID NO: 45. In some embodiments, the IL-2 conjugate has the amino acid sequence of SEQ ID NO: 46. In some embodiments, the IL-2 conjugate has the amino acid sequence of SEQ ID NO: 47. In some embodiments, the IL-2 conjugate has the amino acid sequence of SEQ ID NO: 48. In some embodiments, the IL-2 conjugate has the amino acid sequence of SEQ ID NO: 49.

In some embodiments, the [AzK_L1_PEG5 kD] has the structure of Formula (IV)

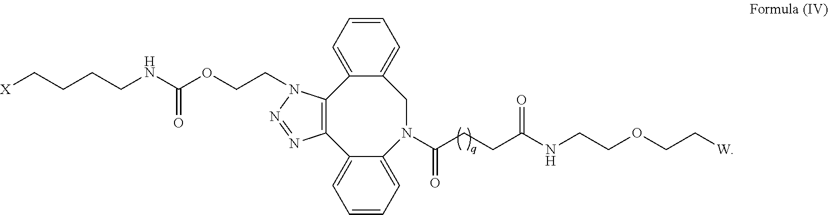

In some embodiments, q is 1 and the [AzK_L1_PEG5 kD] has the structure of Formula (IVa).

In some embodiments, the [AzK_L1_PEG5 kD] has the structure of Formula (V)

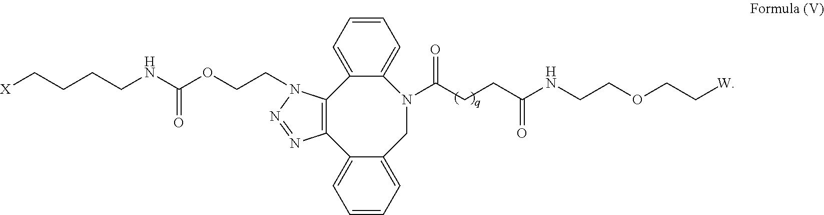

In some embodiments, q is 1 and the [AzK_L1_PEG5 kD] has the structure of Formula (Va).

In some embodiments, the IL-2 conjugate comprises the amino acid sequence of any one of SEQ ID NOS: 50-54, wherein [AzK_L1_PEG30 kD] has the structure of Formula (IV) or Formula (V), or is a mixture of the structures of Formula (IV) and Formula (V):

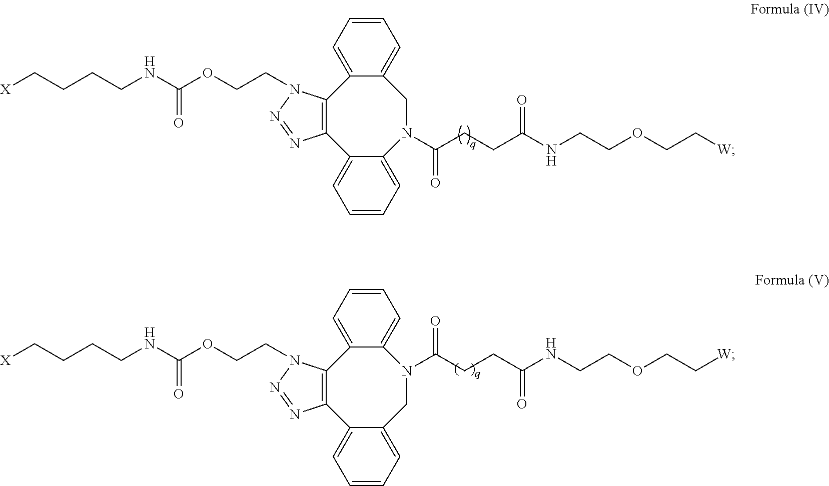

wherein:
W is a PEG group having an average molecular weight of about 30 kDa;
q is 1, 2, or 3; and
X has the structure:

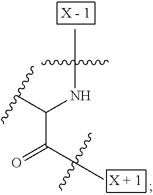 [SMILES: *NC(*)C(*)=O]

X−1 indicates the point of attachment to the preceding amino acid residue; and
X+1 indicates the point of attachment to the following amino acid residue.

In some embodiments, q is 1 and the [AzK_L1_PEG30 kD] has the structure of Formula (IVa) or Formula (Va), or is a mixture of the structures of Formula (IVa) and Formula (Va).

In some embodiments, the IL-2 conjugate has the amino acid sequence of SEQ ID NO: 50. In some embodiments, the IL-2 conjugate has the amino acid sequence of SEQ ID NO: 51. In some embodiments, the IL-2 conjugate has the amino acid sequence of SEQ ID NO: 52. In some embodiments, the IL-2 conjugate has the amino acid sequence of SEQ ID NO: 53. In some embodiments, the IL-2 conjugate has the amino acid sequence of SEQ ID NO: 54.

In some embodiments, the [AzK_L1_PEG30 kD] has the structure of Formula (IV):

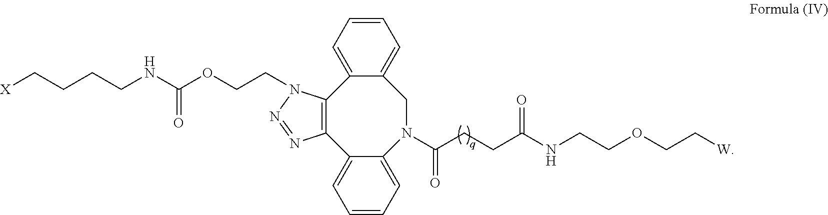

In some embodiments, q is 1 and the [AzK_L1_PEG30 kD] has the structure of Formula (IVa).

In some embodiments, the [AzK_L1_PEG30 kD] has the structure of Formula (V):

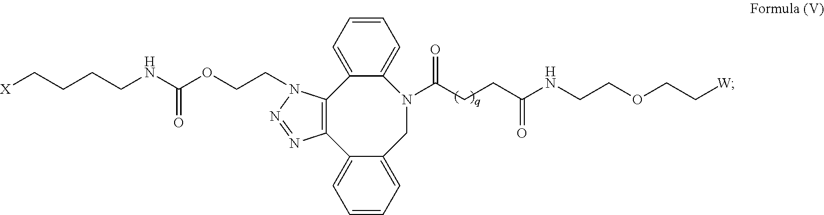

In some embodiments, q is 1 and the [AzK_L1_PEG30 kD] has the structure of Formula (Va).

In some embodiments, the IL-2 conjugate comprises the amino acid sequence of any one of SEQ ID NOS: 40-44, wherein [Azk_L1_PEG] is a mixture of the structures of Formula (IV) and Formula (V):

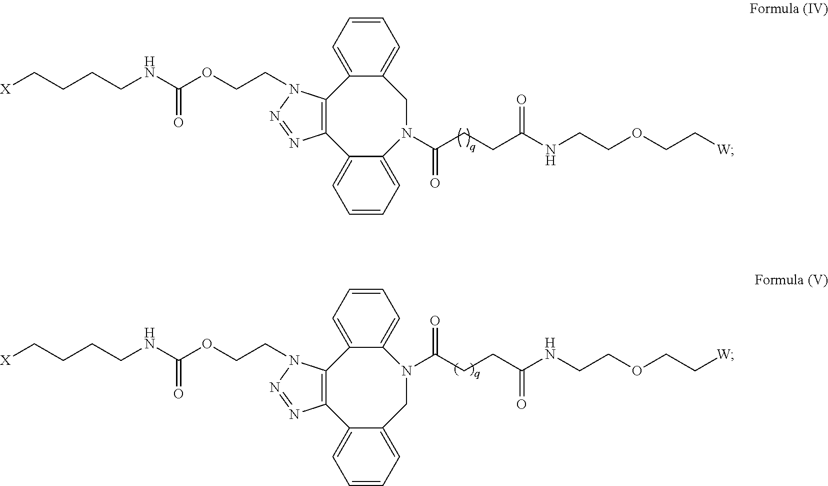

wherein:
W is a PEG group having an average molecular weight selected from about 5 kDa, 10 kDa, 15 kDa, 20 kDa, 25 kDa, 30 kDa, 35 kDa, 40 kDa, 45 kDa, 50 kDa, and 60 kDa;
q is 1, 2, or 3; and
X has the structure:

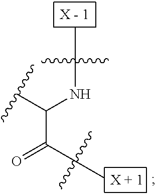 [SMILES: *NC(*)C(*)=O]

X−1 indicates the point of attachment to the preceding amino acid residue; and
X+1 indicates the point of attachment to the following amino acid residue.

In some embodiments, the ratio of the amount of the structure of Formula (IV) to the amount of the structure of Formula (V) comprising the total amount of [AzK_L1_PEG] in the IL-2 conjugate is about 1:1. In some embodiments, the ratio of the amount of the structure of Formula (IV) to the amount of the structure of Formula (V) comprising the total amount of [AzK_L1_PEG] in the IL-2 conjugate is greater than 1:1. In some embodiments, the ratio of the amount of the structure of Formula (IV) to the amount of the structure of Formula (V) comprising the total amount of [AzK_L1_PEG] in the IL-2 conjugate is less than 1:1.

In some embodiments, the IL-2 conjugate comprises the amino acid sequence of any one of SEQ ID NOS: 45 to 49, wherein [AzK_L1_PEG5 kD] is a mixture of the structures of Formula (IV) and Formula (V):

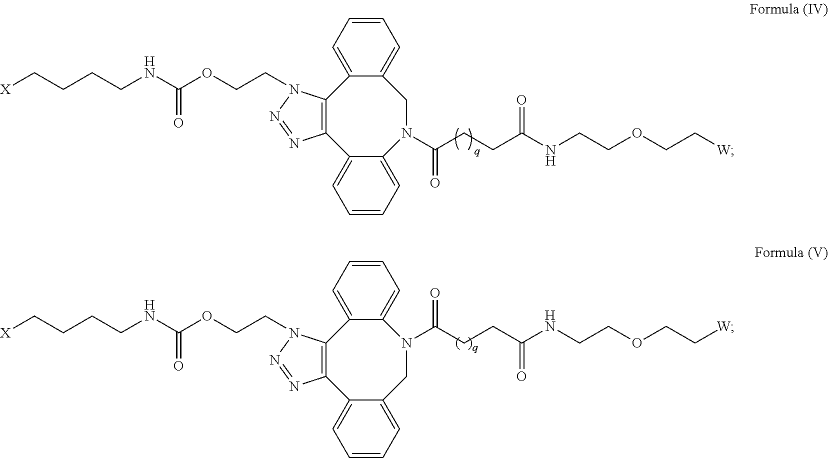

wherein:
W is a PEG group having an average molecular weight of about 5 kDa;
q is 1, 2, or 3; and
X has the structure:

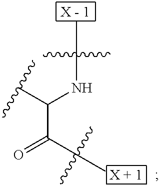 [SMILES: *NC(*)C(*)=O]

X−1 indicates the point of attachment to the preceding amino acid residue; and
X+1 indicates the point of attachment to the following amino acid residue.

In some embodiments, q is 1 and the [AzK_L1_PEG5 kD] is a mixture of the structures of Formula (IVa) and Formula (Va).

In some embodiments, the ratio of the amount of the structure of Formula (IV) to the amount of the structure of Formula (V) comprising the total amount of [AzK_L1_PEG5 kD] in the IL-2 conjugate is about 1:1. In some embodiments, the ratio of the amount of the structure of Formula (IV) to the amount of the structure of Formula (V) comprising the total amount of [AzK_L1_PEG5 kD] in the IL-2 conjugate is greater than 1:1. In some embodiments, the ratio of the amount of the structure of Formula (IV) to the amount of the structure of Formula (V) comprising the total amount of [AzK_L1_PEG5 kD] in the IL-2 conjugate is less than 1:1.

In some embodiments, the IL-2 conjugate comprises the amino acid sequence of any one of SEQ ID NOS: 50-54, wherein [AzK_L1 PEG30 kD] is a mixture of the structures of Formula (IV) and Formula (V):

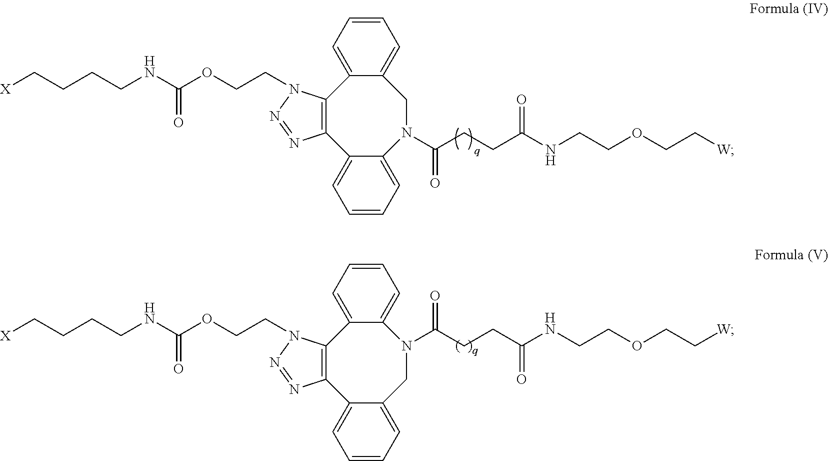

wherein:
W is a PEG group having an average molecular weight of about 30 kDa;
q is 1, 2, or 3; and
X has the structure:

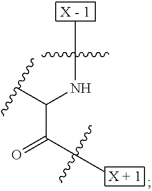 [SMILES: *NC(*)C(*)=O]

X−1 indicates the point of attachment to the preceding amino acid residue; and
X+1 indicates the point of attachment to the following amino acid residue.

In some embodiments, q is 1 and the [AzK_L1 PEG30 kD] is a mixture of the structures of Formula (IVa) and Formula (Va).

In some embodiments, the ratio of the amount of the structure of Formula (IV) to the amount of the structure of Formula (V) comprising the total amount of [AzK_L1_PEG30 kD] in the IL-2 conjugate is about 1:1. In some embodiments, the ratio of the amount of the structure of Formula (IV) to the amount of the structure of Formula (V) comprising the total amount of [AzK_L1_PEG30 kD] in the IL-2 conjugate is greater than 1:1. In some embodiments, the ratio of the amount of the structure of Formula (IV) to the amount of the structure of Formula (V) comprising the total amount of [AzK_L1_PEG30 kD] in the IL-2 conjugate is less than 1:1.

In some embodiments, W in the structure of Formula (IV) or (V) is a PEG group having an average molecular weight selected from about 5 kDa, 10 kDa, 15 kDa, 20 kDa, 25 kDa, and 30 kDa. In some embodiments, W in the structure of Formula (IV) or (V) is a PEG group having an average molecular weight of about 5 kDa. In some embodiments, W in the structure of Formula (IV) or (V) is a PEG group having an average molecular weight of about 30 kDa. In some embodiments, W in the structure of Formula (IV) or (V) is a PEG group having an average molecular weight of about 10 kDa. In some embodiments, W in the structure of Formula (IV) or (V) is a PEG group having an average molecular weight of about 15 kDa. In some embodiments, W in the structure of Formula (IV) or (V) is a PEG group having an average molecular weight of about 20 kDa. In some embodiments, W in the structure of Formula (IV) or (V) is a PEG group having an average molecular weight of about 25 kDa. In some embodiments, W in the structure of Formula (IV) or (V) is a PEG group having an average molecular weight of about 30 kDa. In some embodiments, W in the structure of Formula (IV) or (V) is a PEG group having an average molecular weight of about 35 kDa. In some embodiments, W in the structure of Formula (IV) or (V) is a PEG group having an average molecular weight of about 40 kDa. In some embodiments, W in the structure of Formula (IV) or (V) is a PEG group having an average molecular weight of about 45 kDa. In some embodiments, W in the structure of Formula (IV) or (V) is a PEG group having an average molecular weight of about 50 kDa. In some embodiments, W in the structure of Formula (IV) or (V) is a PEG group having an average molecular weight of about 55 kDa. In some embodiments, W in the structure of Formula (IV) or (V) is a PEG group having an average molecular weight of about 60 kDa.

In some embodiments, the IL-2 conjugate described herein comprises the structure of Formula (IV) or Formula (V), or a mixture of Formula (II) and Formula (III), wherein W is a linear or branched PEG group. In some embodiments, W in the structure of Formula (IV) or Formula (V) is a linear PEG group. In some embodiments, W in the structure of Formula (IV) or Formula (V) is a branched PEG group. In some embodiments, W in the structure of Formula (IV) or Formula (V) is a methoxy PEG group. In some embodiments, W in the structure of Formula (IV) or Formula (V) is a methoxy PEG group that is linear or branched. In some embodiments, the methoxy PEG group in the structure of Formula (IV) or Formula (V) is linear. In some embodiments, the methoxy PEG group in the structure of Formula (IV) or Formula (V) is branched.

With respect to the IL-2 conjugates used in the methods described herein, an exemplary structure of a methoxy PEG group is illustrated in the mPEG-DBCO structures below.

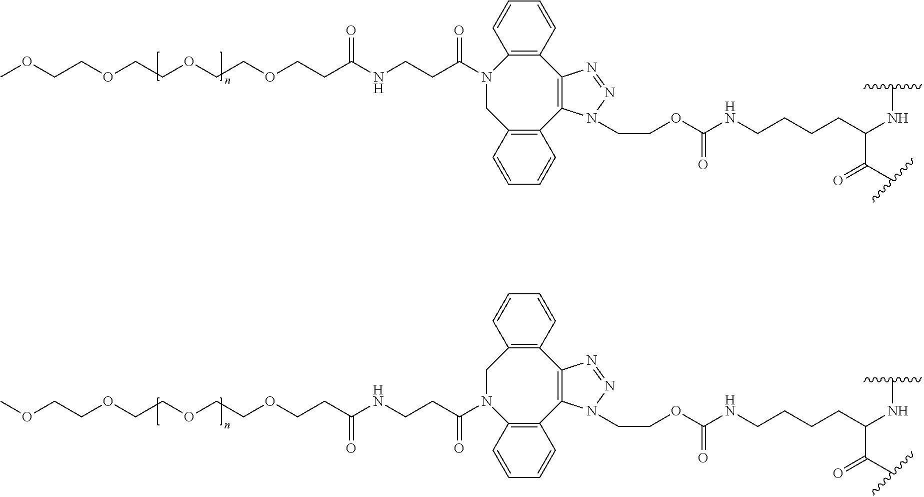

In some embodiments, the IL-2 conjugate comprises the amino acid sequence of SEQ ID NO: 3 in which at least one amino acid residue in the IL-2 conjugate is replaced by the structure of Formula (VI) or (VII), or a mixture of (VI) and (VII):

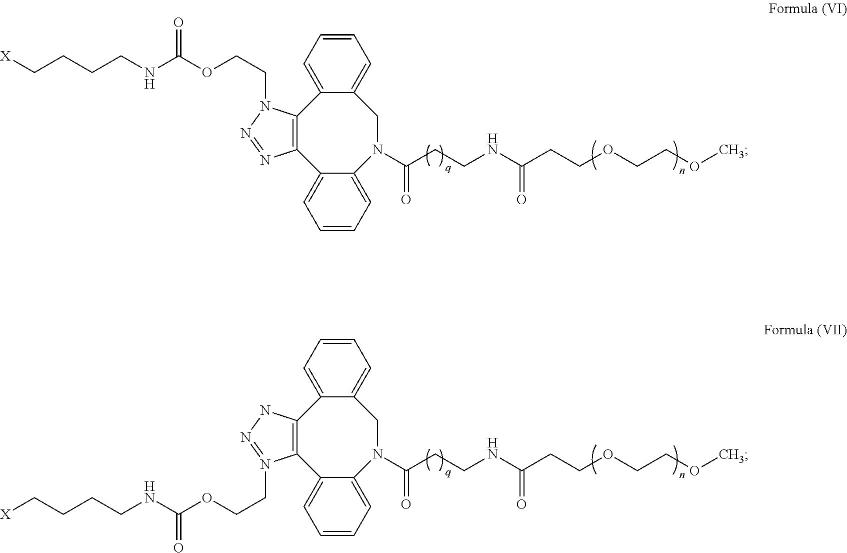

wherein:
n is an integer in the range from about 2 to about 5000;
q is 1, 2, or 3; and
X has the structure:

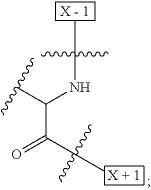 [SMILES: *NC(*)C(*)=O]

X−1 indicates the point of attachment to the preceding amino acid residue; and
X+1 indicates the point of attachment to the following amino acid residue.

In some embodiments of Formula (VI) or Formula (VII), or a mixture of Formula (VI) or Formula (VII), q is 1. In some embodiments of Formula (VI) or Formula (VII), or a mixture of Formula (VI) or Formula (VII), q is 2. In some embodiments of Formula (VI) or Formula (VII), or a mixture of Formula (VI) or Formula (VII), q is 3.

Here and throughout, the structure of Formula (VI) encompasses pharmaceutically acceptable salts, solvates, or hydrates thereof. Here and throughout, the structure of Formula (VII) encompasses pharmaceutically acceptable salts, solvates, or hydrates thereof. In some embodiments, the IL-2 conjugate is a pharmaceutically acceptable salt, solvate, or hydrate.

In some embodiments, q is 1 and the structures of Formula (VI) and Formula (VII) are the structures of Formula (VIa) and Formula (VIIa):

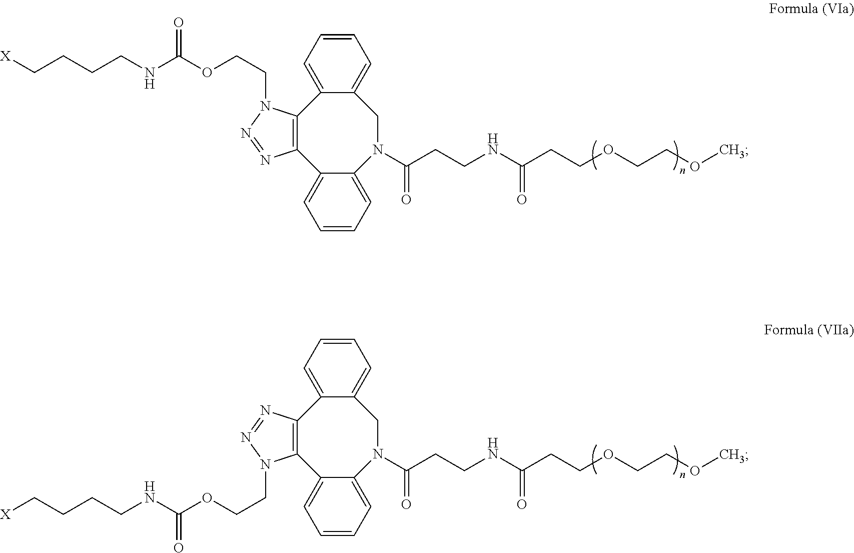

wherein:
n is an integer in the range from about 2 to about 5000; and
X has the structure:

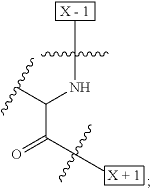 [SMILES: *NC(*)C(*)=O]

X−1 indicates the point of attachment to the preceding amino acid residue; and
X+1 indicates the point of attachment to the following amino acid residue.

In some embodiments, n in the compounds of Formula (VI) and (VII) is in the range from about 5 to about 4600, or from about 10 to about 4000, or from about 20 to about 3000, or from about 100 to about 3000, or from about 100 to about 2900, or from about 150 to about 2900, or from about 125 to about 2900, or from about 100 to about 2500, or from about 100 to about 2000, or from about 100 to about 1900, or from about 100 to about 1850, or from about 100 to about 1750, or from about 100 to about 1650, or from about 100 to about 1500, or from about 100 to about 1400, or from about 100 to about 1300, or from about 100 to about 1250, or from about 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575. In some embodiments, n in the compounds of Formula (VI) and (VII) is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546.

In some embodiments, the position of the structure of Formula (VI), Formula (VII), or a mixture of Formula (VI) and (VII) in the amino acid sequence of the IL-2 conjugate is selected from K34, F41, F43, K42, E61, P64, R37, T40, E67, Y44, V68, and L71, wherein the position of the structure of Formula (I) in the amino acid sequence of the IL-2 conjugate is in reference to the positions in SEQ ID NO: 3. In some embodiments, the position of the structure of Formula (VI), Formula (VII), or a mixture of Formula (VI) and (VII) in the amino acid sequence of the IL-2 conjugate of SEQ ID NO: 3 is selected from K34, F41, F43, K42, E61, P64, R37, T40, E67, Y44, V68, and L71. In some embodiments, the position of the structure of Formula (VI), Formula (VII), or a mixture of Formula (VI) and (VII) in the amino acid sequence of the IL-2 conjugate of SEQ ID NO: 3 is at position K34. In some embodiments, the position of the structure of Formula (VI), Formula (VII), or a mixture of Formula (VI) and (VII) in the amino acid sequence of the IL-2 conjugate of SEQ ID NO: 3 is at position F41. In some embodiments, the position of the structure of Formula (VI), Formula (VII), or a mixture of Formula (VI) and (VII) in the amino acid sequence of the IL-2 conjugate of SEQ ID NO: 3 is at position F43. In some embodiments, the position of the structure of Formula (VI), Formula (VII), or a mixture of Formula (VI) and (VII) in the amino acid sequence of the IL-2 conjugate of SEQ ID NO: 3 is at position K42. In some embodiments, the position of the structure of Formula (VI), Formula (VII), or a mixture of Formula (VI) and (VII) in the amino acid sequence of the IL-2 conjugate of SEQ ID NO: 3 is at position E61. In some embodiments, the position of the structure of Formula (VI), Formula (VII), or a mixture of Formula (VI) and (VII) in the amino acid sequence of the IL-2 conjugate of SEQ ID NO: 3 is at position P64. In some embodiments, the position of the structure of Formula (VI), Formula (VII), or a mixture of Formula (VI) and (VII) in the amino acid sequence of the IL-2 conjugate of SEQ ID NO: 3 is at position R37. In some embodiments, the position of the structure of Formula (VI), Formula (VII), or a mixture of Formula (VI) and (VII) in the amino acid sequence of the IL-2 conjugate of SEQ ID NO: 3 is at position T40. In some embodiments, the position of the structure of Formula (VI), Formula (VII), or a mixture of Formula (VI) and (VII) in the amino acid sequence of the IL-2 conjugate of SEQ ID NO: 3 is at position E67. In some embodiments, the position of the structure of Formula (VI), Formula (VII), or a mixture of Formula (VI) and (VII) in the amino acid sequence of the IL-2 conjugate of SEQ ID NO: 3 is at position Y44. In some embodiments, the position of the structure of Formula (VI), Formula (VII), or a mixture of Formula (VI) and (VII) in the amino acid sequence of the IL-2 conjugate of SEQ ID NO: 3 is at position V68. In some embodiments, the position of the structure of Formula (VI), Formula (VII), or a mixture of Formula (VI) and (VII) in the amino acid sequence of the IL-2 conjugate of SEQ ID NO: 3 is at position L71.

In some embodiments, the ratio of the amount of the structure of Formula (VI) to the amount of the structure of Formula (VII) comprising the total amount of the IL-2 conjugate is about 1:1. In some embodiments, the ratio of the amount of the structure of Formula (VI) to the amount of the structure of Formula (VII) comprising the total amount of the IL-2 conjugate is greater than 1:1. In some embodiments, the ratio of the amount of the structure of Formula (VI) to the amount of the structure of Formula (VII) comprising the total amount of the IL-2 conjugate is less than 1:1.

In some embodiments, the IL-2 conjugate comprises the amino acid sequence of SEQ ID NO: 3 in which at least one amino acid residue in the IL-2 conjugate is replaced by the structure of Formula (VI) or (VII), or a mixture of (VI) and (VII), wherein the amino acid residue in SEQ ID NO: 3 that is replaced is selected from K34, F41, F43, K42, E61, P64, R37, T40, E67, Y44, V68, and L71, and wherein n is an integer from 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575. In some embodiments, n in the compounds of Formula (VI) and (VII) is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546.

In some embodiments, the IL-2 conjugate comprises the amino acid sequence of SEQ ID NO: 3 in which at least one amino acid residue in the IL-2 conjugate is replaced by the structure of Formula (VI) or (VII), or a mixture of (VI) and (VII), wherein the amino acid residue in SEQ ID NO: 3 that is replaced is selected from F41, F43, K42, E61, and P64, and wherein n is an integer from about 450 to about 800, or from about 454 to about 796, or from about 454 to about 682, or from about 568 to about 909. In some embodiments, n in the compounds of Formula (VI) and (VII) is an integer selected from 454, 455, 568, 569, 680, 681, 682, 794, 795, 796, 908, 909, 910, 1021, 1022, 1023, 1135, 1136, 1137, and 1249.

In some embodiments, the IL-2 conjugate comprises the amino acid sequence of SEQ ID NO: 3 in which at least one amino acid residue in the IL-2 conjugate is replaced by the structure of Formula (VI) or (VII), or a mixture of (VI) and (VII), wherein the amino acid residue in SEQ ID NO: 3 that is replaced is selected from E61 and P64, and wherein n is an integer from about 450 to about 800, or from about 454 to about 796, or from about 454 to about 682, or from about 568 to about 909. In some embodiments, n in the compounds of Formula (VI) and (VII) is an integer selected from 454, 455, 568, 569, 680, 681, 682, 794, 795, 796, 908, 909, and 910.

In some embodiments, the IL-2 conjugate comprises the amino acid sequence of SEQ ID NO: 3 in which at least one amino acid residue in the IL-2 conjugate is replaced by the structure of Formula (VI) or (VII), or a mixture of (VI) and (VII), wherein the amino acid residue in SEQ ID NO: 3 that is replaced is E61, and wherein n is an integer from about 450 to about 800, or from about 454 to about 796, or from about 454 to about 682, or from about 568 to about 909. In some embodiments, n in the compounds of Formula (VI) and (VII) is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

In some embodiments, the IL-2 conjugate comprises the amino acid sequence of SEQ ID NO: 3 in which at least one amino acid residue in the IL-2 conjugate is replaced by the structure of Formula (VI) or (VII), or a mixture of (VI) and (VII), wherein the amino acid residue in in SEQ ID NO: 3 that is replaced is P64, and wherein n is an integer from about 450 to about 800, or from about 454 to about 796, or from about 454 to about 682, or from about 568 to about 909. In some embodiments, n in the compounds of Formula (VI) and (VII) is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

In some embodiments n in the structures of Formula (VI) and (VII) is an integer such that the molecular weight of the PEG moiety is in the range from about 1,000 Daltons to about 200,000 Daltons, or from about 2,000 Daltons to about 150,000 Daltons, or from about 3,000 Daltons to about 125,000 Daltons, or from about 4,000 Daltons to about 100,000 Daltons, or from about 5,000 Daltons to about 100,000 Daltons, or from about 6,000 Daltons to about 90,000 Daltons, or from about 7,000 Daltons to about 80,000 Daltons, or from about 8,000 Daltons to about 70,000 Daltons, or from about 5,000 Daltons to about 70,000 Daltons, or from about 5,000 Daltons to about 65,000 Daltons, or from about 5,000 Daltons to about 60,000 Daltons, or from about 5,000 Daltons to about 50,000 Daltons, or from about 6,000 Daltons to about 50,000 Daltons, or from about 7,000 Daltons to about 50,000 Daltons, or from about 7,000 Daltons to about 45,000 Daltons, or from about 7,000 Daltons to about 40,000 Daltons, or from about 8,000 Daltons to about 40,000 Daltons, or from about 8,500 Daltons to about 40,000 Daltons, or from about 8,500 Daltons to about 35,000 Daltons, or from about 9,000 Daltons to about 50,000 Daltons, or from about 9,000 Daltons to about 45,000 Daltons, or from about 9,000 Daltons to about 40,000 Daltons, or from about 9,000 Daltons to about 35,000 Daltons, or from about 9,000 Daltons to about 30,000 Daltons, or from about 9,500 Daltons to about 35,000 Daltons, or from about 9,500 Daltons to about 30,000 Daltons, or from about 10,000 Daltons to about 50,000 Daltons, or from about 10,000 Daltons to about 45,000 Daltons, or from about 10,000 Daltons to about 40,000 Daltons, or from about 10,000 Daltons to about 35,000 Daltons, or from about 10,000 Daltons to about 30,000 Daltons, or from about 15,000 Daltons to about 50,000 Daltons, or from about 15,000 Daltons to about 45,000 Daltons, or from about 15,000 Daltons to about 40,000 Daltons, or from about 15,000 Daltons to about 35,000 Daltons, or from about 15,000 Daltons to about 30,000 Daltons, or from about 20,000 Daltons to about 50,000 Daltons, or from about 20,000 Daltons to about 45,000 Daltons, or from about 20,000 Daltons to about 40,000 Daltons, or from about 20,000 Daltons to about 35,000 Daltons, or from about 20,000 Daltons to about 30,000 Daltons.

In some embodiments, the IL-2 conjugate comprises the amino acid sequence of SEQ ID NO: 3 in which at least one amino acid residue in the IL-2 conjugate is replaced by the structure of Formula (VI) or (VII), or a mixture of (VI) and (VII), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, about 50,000 Daltons, about 60,000 Daltons, about 70,000 Daltons, about 80,000 Daltons, about 90,000 Daltons, about 100,000 Daltons, about 125,000 Daltons, about 150,000 Daltons, about 175,000 Daltons or about 200,000 Daltons.

In some embodiments, the IL-2 conjugate comprises the amino acid sequence of SEQ ID NO: 3 in which at least one amino acid residue in the IL-2 conjugate is replaced by the structure of Formula (VI) or (VII), or a mixture of (VI) and (VII), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, or about 50,000 Daltons.

In some embodiments, the IL-2 conjugate comprises the amino acid sequence of SEQ ID NO: 3 in which at least one amino acid residue in the IL-2 conjugate is replaced by the structure of Formula (VIII) or (IX), or a mixture of (VIII) and (IX):

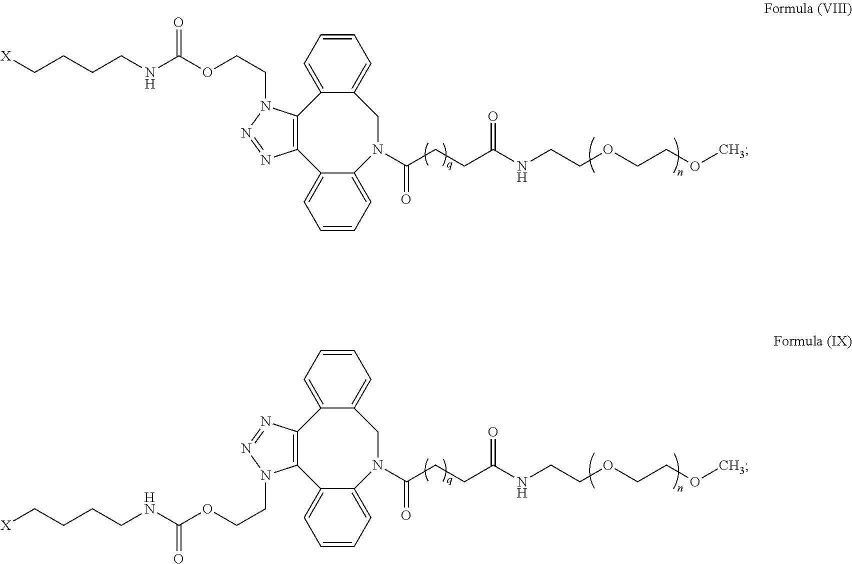

wherein:
n is an integer in the range from about 2 to about 5000;
q is 1, 2, or 3; and
X has the structure:

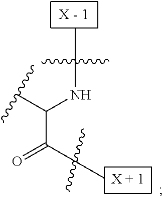 [SMILES: *NC(*)C(*)=O]

X−1 indicates the point of attachment to the preceding amino acid residue; and
X+1 indicates the point of attachment to the following amino acid residue.

In some embodiments of Formula (VIII) or Formula (IX), or a mixture of Formula (VIII) or Formula (IX), q is 1. In some embodiments of Formula (VIII) or Formula (IX), or a mixture of Formula (VIII) or Formula (IX), q is 2. In some embodiments of Formula (VIII) or Formula (IX), or a mixture of Formula (VIII) or Formula (IX), q is 3.

Here and throughout, the structure of Formula (VIII) encompasses pharmaceutically acceptable salts, solvates, or hydrates thereof. Here and throughout, the structure of Formula (IX) encompasses pharmaceutically acceptable salts, solvates, or hydrates thereof. In some embodiments, the IL-2 conjugate is a pharmaceutically acceptable salt, solvate, or hydrate.

In some embodiments, q is 1 and the structures of Formula (VIII) and Formula (IX) are the structures of Formula (VIIIa) or (IXa):

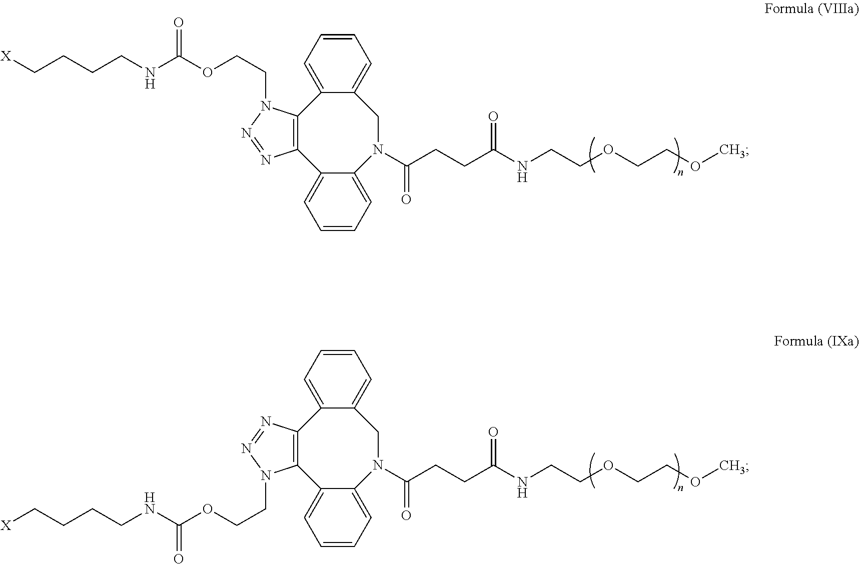

wherein:
n is an integer in the range from about 2 to about 5000; and
X has the structure:

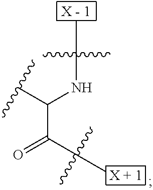 [SMILES: *NC(*)C(*)=O]

X−1 indicates the point of attachment to the preceding amino acid residue; and
X+1 indicates the point of attachment to the following amino acid residue.

In some embodiments, n in the compounds of Formula (VIII) and (IX) is in the range from about 5 to about 4600, or from about 10 to about 4000, or from about 20 to about 3000, or from about 100 to about 3000, or from about 100 to about 2900, or from about 150 to about 2900, or from about 125 to about 2900, or from about 100 to about 2500, or from about 100 to about 2000, or from about 100 to about 1900, or from about 100 to about 1850, or from about 100 to about 1750, or from about 100 to about 1650, or from about 100 to about 1500, or from about 100 to about 1400, or from about 100 to about 1300, or from about 100 to about 1250, or from about 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575. In some embodiments, n in the compounds of Formula (VIII) and (IX) is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546.

In some embodiments, the position of the structure of Formula (VIII), Formula (IX), or a mixture of Formula (VIII) and (IX) in the amino acid sequence of the IL-2 conjugate is selected from K34, F41, F43, K42, E61, P64, R37, T40, E67, Y44, V68, and L71, wherein the position of the structures of Formula (VIII), Formula (IX), or mixture thereof in the amino acid sequence of the IL-2 conjugate is in reference to the positions in SEQ ID NO: 3. In some embodiments, the position of the structure of Formula (VIII), Formula (IX), or a mixture of Formula (VIII) and Formula (IX) in the amino acid sequence of the IL-2 conjugate of SEQ ID NO: 3 is selected from K34, F41, F43, K42, E61, P64, R37, T40, E67, Y44, V68, and L71. In some embodiments, the position of the structure of Formula (VIII), Formula (IX), or a mixture of Formula (VIII) and Formula (IX) in the amino acid sequence of the IL-2 conjugate of SEQ ID NO: 3 is at position K34. In some embodiments, the position of the structure of Formula (VIII), Formula (IX), or a mixture of Formula (VIII) and Formula (IX) in the amino acid sequence of the IL-2 conjugate of SEQ ID NO: 3 is at position F41. In some embodiments, the position of the structure of Formula (VIII), Formula (IX), or a mixture of Formula (VIII) and Formula (IX) in the amino acid sequence of the IL-2 conjugate of SEQ ID NO: 3 is at position F43. In some embodiments, the position of the structure of Formula (VIII), Formula (IX), or a mixture of Formula (VIII) and Formula (IX) in the amino acid sequence of the IL-2 conjugate of SEQ ID NO: 3 is at position K42. In some embodiments, the position of the structure of Formula (VIII), Formula (IX), or a mixture of Formula (VIII) and Formula (IX) in the amino acid sequence of the IL-2 conjugate of SEQ ID NO: 3 is at position E61. In some embodiments, the position of the structure of Formula (VIII), Formula (IX), or a mixture of Formula (VIII) and Formula (IX) in the amino acid sequence of the IL-2 conjugate of SEQ ID NO: 3 is at position P64. In some embodiments, the position of the structure of Formula (VIII), Formula (IX), or a mixture of Formula (VIII) and Formula (IX) in the amino acid sequence of the IL-2 conjugate of SEQ ID NO: 3 is at position R37. In some embodiments, the position of the structure of Formula (VIII), Formula (IX), or a mixture of Formula (VIII) and Formula (IX) in the amino acid sequence of the IL-2 conjugate of SEQ ID NO: 3 is at position T40. In some embodiments, the position of the structure of Formula (VIII), Formula (IX), or a mixture of Formula (VIII) and Formula (IX) in the amino acid sequence of the IL-2 conjugate of SEQ ID NO: 3 is at position E67. In some embodiments, the position of the structure of Formula (VIII), Formula (IX), or a mixture of Formula (VIII) and Formula (IX) in the amino acid sequence of the IL-2 conjugate of SEQ ID NO: 3 is at position Y44. In some embodiments, the position of the structure of Formula (VIII), Formula (IX), or a mixture of Formula (VIII) and Formula (IX) in the amino acid sequence of the IL-2 conjugate of SEQ ID NO: 3 is at position V68. In some embodiments, the position of the structure of Formula (VIII), Formula (IX), or a mixture of Formula (VIII) and Formula (IX) in the amino acid sequence of the IL-2 conjugate of SEQ ID NO: 3 is at position L71.

In some embodiments, the ratio of the amount of the structure of Formula (VIII) to the amount of the structure of Formula (IX) comprising the total amount of the IL-2 conjugate is about 1:1. In some embodiments, the ratio of the amount of the structure of Formula (VIII) to the amount of the structure of Formula (IX) comprising the total amount of the IL-2 conjugate is greater than 1:1. In some embodiments, the ratio of the amount of the structure of Formula (VIII) to the amount of the structure of Formula (IX) comprising the total amount of the IL-2 conjugate is less than 1:1.

In some embodiments, the IL-2 conjugate comprises the amino acid sequence of SEQ ID NO: 3 in which at least one amino acid residue in the IL-2 conjugate is replaced by the structure of Formula (VIII) or (IX), or a mixture of (VIII) and (IX), wherein the amino acid residue in in SEQ ID NO: 3 that is replaced is selected from K34, F41, F43, K42, E61, P64, R37, T40, E67, Y44, V68, and L71, and wherein n is an integer from 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575. In some embodiments, n in the compounds of Formula (VIII) and (IX) is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546.

In some embodiments, the IL-2 conjugate comprises the amino acid sequence of SEQ ID NO: 3 in which at least one amino acid residue in the IL-2 conjugate is replaced by the structure of Formula (VIII) or (IX), or a mixture of Formula (VIII) and Formula (IX), wherein the amino acid residue in in SEQ ID NO: 3 that is replaced is selected from F41, F43, K42, E61, and P64, and wherein n is an integer from about 450 to about 800, or from about 454 to about 796, or from about 454 to about 682, or from about 568 to about 909. In some embodiments, n in the compounds of Formula (VIII) and Formula (IX) is an integer selected from 454, 455, 568, 569, 680, 681, 682, 794, 795, 796, 908, 909, 910, 1021, 1022, 1023, 1135, 1136, 1137, and 1249.

In some embodiments, the IL-2 conjugate comprises the amino acid sequence of SEQ ID NO: 3 in which at least one amino acid residue in the IL-2 conjugate is replaced by the structure of Formula (VIII) or (IX), or a mixture of Formula (VIII) and Formula (IX), wherein the amino acid residue in SEQ ID NO: 3 that is replaced is selected from E61 and P64, and wherein n is an integer from about 450 to about 800, or from about 454 to about 796, or from about 454 to about 682, or from about 568 to about 909. In some embodiments, n in the compounds of Formula (VIII) and Formula (IX) is an integer selected from 454, 455, 568, 569, 680, 681, 682, 794, 795, 796, 908, 909, and 910.

In some embodiments, the IL-2 conjugate comprises the amino acid sequence of SEQ ID NO: 3 in which at least one amino acid residue in the IL-2 conjugate is replaced by the structure of Formula (VIII) or (IX), or a mixture of Formula (VIII) and Formula (IX), wherein the amino acid residue in SEQ ID NO: 3 that is replaced is E61, and wherein n is an integer from about 450 to about 800, or from about 454 to about 796, or from about 454 to about 682, or from about 568 to about 909. In some embodiments, n in the compounds of Formula (VIII) and Formula (IX) is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

In some embodiments, the IL-2 conjugate comprises the amino acid sequence of SEQ ID NO: 3 in which at least one amino acid residue in the IL-2 conjugate is replaced by the structure of Formula (VIII) or Formula (IX), or a mixture of Formula (VIII) and Formula (IX), wherein the amino acid residue in in SEQ ID NO: 3 that is replaced is P64, and wherein n is an integer from about 450 to about 800, or from about 454 to about 796, or from about 454 to about 682, or from about 568 to about 909. In some embodiments, n in the compounds of Formula (VIII) and Formula (IX) is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

In some embodiments, the IL-2 conjugate comprises the amino acid sequence of SEQ ID NO: 3 in which at least one amino acid residue in the IL-2 conjugate is replaced by the structure of Formula (VIII) or Formula (IX), or a mixture of Formula (VIII) and Formula (IX), wherein n is an integer such that the molecular weight of the PEG moiety is in the range from about 1,000 Daltons about 200,000 Daltons, or from about 2,000 Daltons to about 150,000 Daltons, or from about 3,000 Daltons to about 125,000 Daltons, or from about 4,000 Daltons to about 100,000 Daltons, or from about 5,000 Daltons to about 100,000 Daltons, or from about 6,000 Daltons to about 90,000 Daltons, or from about 7,000 Daltons to about 80,000 Daltons, or from about 8,000 Daltons to about 70,000 Daltons, or from about 5,000 Daltons to about 70,000 Daltons, or from about 5,000 Daltons to about 65,000 Daltons, or from about 5,000 Daltons to about 60,000 Daltons, or from about 5,000 Daltons to about 50,000 Daltons, or from about 6,000 Daltons to about 50,000 Daltons, or from about 7,000 Daltons to about 50,000 Daltons, or from about 7,000 Daltons to about 45,000 Daltons, or from about 7,000 Daltons to about 40,000 Daltons, or from about 8,000 Daltons to about 40,000 Daltons, or from about 8,500 Daltons to about 40,000 Daltons, or from about 8,500 Daltons to about 35,000 Daltons, or from about 9,000 Daltons to about 50,000 Daltons, or from about 9,000 Daltons to about 45,000 Daltons, or from about 9,000 Daltons to about 40,000 Daltons, or from about 9,000 Daltons to about 35,000 Daltons, or from about 9,000 Daltons to about 30,000 Daltons, or from about 9,500 Daltons to about 35,000 Daltons, or from about 9,500 Daltons to about 30,000 Daltons, or from about 10,000 Daltons to about 50,000 Daltons, or from about 10,000 Daltons to about 45,000 Daltons, or from about 10,000 Daltons to about 40,000 Daltons, or from about 10,000 Daltons to about 35,000 Daltons, or from about 10,000 Daltons to about 30,000 Daltons, or from about 15,000 Daltons to about 50,000 Daltons, or from about 15,000 Daltons to about 45,000 Daltons, or from about 15,000 Daltons to about 40,000 Daltons, or from about 15,000 Daltons to about 35,000 Daltons, or from about 15,000 Daltons to about 30,000 Daltons, or from about 20,000 Daltons to about 50,000 Daltons, or from about 20,000 Daltons to about 45,000 Daltons, or from about 20,000 Daltons to about 40,000 Daltons, or from about 20,000 Daltons to about 35,000 Daltons, or from about 20,000 Daltons to about 30,000 Daltons. In some embodiments, the IL-2 conjugate comprises the amino acid sequence of SEQ ID NO: 3 in which at least one amino acid residue in the IL-2 conjugate is replaced by the structure of Formula (VIII) or Formula (IX), or a mixture of Formula (VIII) and Formula (IX), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, about 50,000 Daltons, about 60,000 Daltons, about 70,000 Daltons, about 80,000 Daltons, about 90,000 Daltons, about 100,000 Daltons, about 125,000 Daltons, about 150,000 Daltons, about 175,000 Daltons or about 200,000 Daltons. In some embodiments, the IL-2 conjugate comprises the amino acid sequence of SEQ ID NO: 3 in which at least one amino acid residue in the IL-2 conjugate is replaced by the structure of Formula (VIII) or Formula (IX), or a mixture of Formula (VIII) and Formula (IX), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, or about 50,000 Daltons.

In some embodiments, the IL-2 conjugate comprises the amino acid sequence of SEQ ID NO: 3 in which at least one amino acid residue in the IL-2 conjugate is replaced by the structure of Formula (X) or Formula (XI), or a mixture of Formula (X) and Formula (XI):

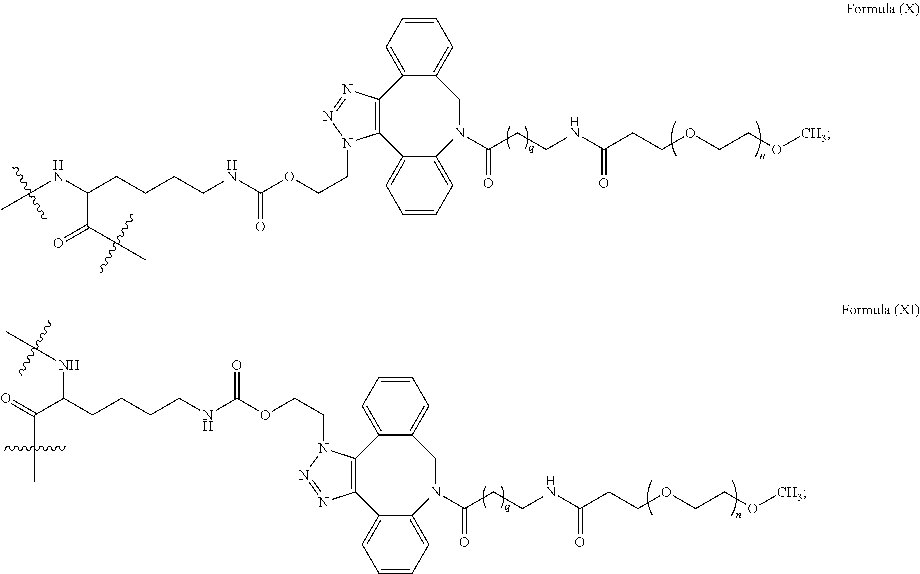

wherein:
n is an integer in the range from about 2 to about 5000;
q is 1, 2, or 3; and
the wavy lines indicate covalent bonds to amino acid residues within SEQ ID NO: 3 that are not replaced.

In some embodiments of Formula (X) or Formula (XI), or a mixture of Formula (X) or Formula (XI), q is 1. In some embodiments of Formula (X) or Formula (XI), or a mixture of Formula (X) or Formula (XI), q is 2. In some embodiments of Formula (X) or Formula (XI), or a mixture of Formula (X) or Formula (XI), q is 3.

Here and throughout, the structure of Formula (X) encompasses pharmaceutically acceptable salts, solvates, or hydrates thereof. Here and throughout, the structure of Formula (XI) encompasses pharmaceutically acceptable salts, solvates, or hydrates thereof. In some embodiments, the IL-2 conjugate is a pharmaceutically acceptable salt, solvate, or hydrate.

In some embodiments, q is 1 and the structures of Formula (X) and Formula (XI) are the structures of Formula (Xa) and Formula (XIa):

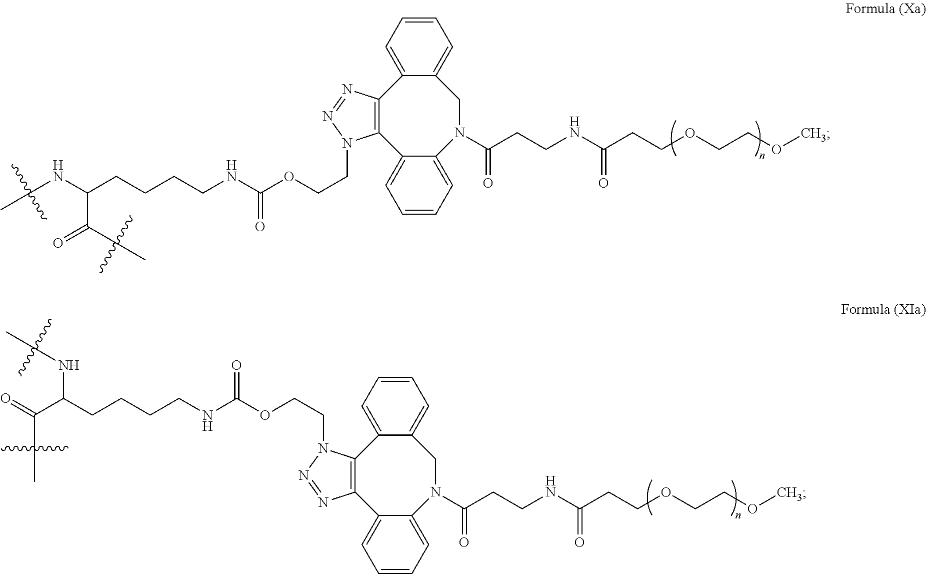

wherein:
n is an integer in the range from about 2 to about 5000; and
the wavy lines indicate covalent bonds to amino acid residues within SEQ ID NO: 3 that are not replaced.

In some embodiments, the stereochemistry of the chiral center within Formula (X) and Formula (XI) is racemic, is enriched in (R), is enriched in (S), is substantially (R), is substantially (S), is (R) or is (S). In some embodiments, the stereochemistry of the chiral center within Formula (X) and Formula (XI) is racemic. In some embodiments, the stereochemistry of the chiral center within Formula (X) and Formula (XI) is enriched in (R). In some embodiments, the stereochemistry of the chiral center within Formula (X) and Formula (XI) is enriched in (S). In some embodiments, the stereochemistry of the chiral center within Formula (X) and Formula (XI) is substantially (R). In some embodiments, the stereochemistry of the chiral center within Formula (X) and Formula (XI) is substantially (S). In some embodiments, the stereochemistry of the chiral center within Formula (X) and Formula (XI) is (R). In some embodiments, the stereochemistry of the chiral center within Formula (X) and Formula (XI) is (S).

In some embodiments, n in the compounds of Formula (X) and (XI) is in the range from about 5 to about 4600, or from about 10 to about 4000, or from about 20 to about 3000, or from about 100 to about 3000, or from about 100 to about 2900, or from about 150 to about 2900, or from about 125 to about 2900, or from about 100 to about 2500, or from about 100 to about 2000, or from about 100 to about 1900, or from about 100 to about 1850, or from about 100 to about 1750, or from about 100 to about 1650, or from about 100 to about 1500, or from about 100 to about 1400, or from about 100 to about 1300, or from about 100 to about 1250, or from about 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575. In some embodiments, n in the compounds of Formula (X) and (XI) is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546.

In some embodiments, the position of the structure of Formula (X), Formula (XI), or a mixture of Formula (X) and Formula (XI) in the amino acid sequence of the IL-2 conjugate is selected from K34, F41, F43, K42, E61, P64, R37, T40, E67, Y44, V68, and L71, wherein the position of the structure of Formula (X), Formula (XI), or a mixture thereof, in the amino acid sequence of the IL-2 conjugate is in reference to the positions in SEQ ID NO: 3. In some embodiments, the IL-2 conjugate in which the position of the structure of Formula (X), Formula (XI), or a mixture of Formula (X) and Formula (XI) in the amino acid sequence of the IL-2 conjugate of SEQ ID NO: 3 is selected from K34, F41, F43, K42, E61, P64, R37, T40, E67, Y44, V68, and L71. In some embodiments, the position of the structure of Formula (X), Formula (XI), or a mixture of Formula (X) and Formula (XI) in the amino acid sequence of the IL-2 conjugate of SEQ ID NO: 3 is at position K34. In some embodiments, the position of the structure of Formula (X), Formula (XI), or a mixture of Formula (X) and Formula (XI) in the amino acid sequence of the IL-2 conjugate of SEQ ID NO: 3 is at position F41. In some embodiments, the position of the structure of Formula (X), Formula (XI), or a mixture of Formula (X) and Formula (XI) in the amino acid sequence of the IL-2 conjugate of SEQ ID NO: 3 is at position F43. In some embodiments, the position of the structure of Formula (X), Formula (XI), or a mixture of Formula (X) and Formula (XI) in the amino acid sequence of the IL-2 conjugate of SEQ ID NO: 3 is at position K42. In some embodiments, the position of the structure of Formula (X), Formula (XI), or a mixture of Formula (X) and Formula (XI) in the amino acid sequence of the IL-2 conjugate of SEQ ID NO: 3 is at position E61. In some embodiments, the position of the structure of Formula (X), Formula (XI), or a mixture of Formula (X) and Formula (XI) in the amino acid sequence of the IL-2 conjugate of SEQ ID NO: 3 is at position P64. In some embodiments, the position of the structure of Formula (X), Formula (XI), or a mixture of Formula (X) and Formula (XI) in the amino acid sequence of the IL-2 conjugate of SEQ ID NO: 3 is at position R37. In some embodiments, the position of the structure of Formula (X), Formula (XI), or a mixture of Formula (X) and Formula (XI) in the amino acid sequence of the IL-2 conjugate of SEQ ID NO: 3 is at position T40. In some embodiments, the position of the structure of Formula (X), Formula (XI), or a mixture of Formula (X) and Formula (XI) in the amino acid sequence of the IL-2 conjugate of SEQ ID NO: 3 is at position E67. In some embodiments, the position of the structure of Formula (X), Formula (XI), or a mixture of Formula (X) and Formula (XI) in the amino acid sequence of the IL-2 conjugate of SEQ ID NO: 3 is at position Y44. In some embodiments, the position of the structure of Formula (X), Formula (XI), or a mixture of Formula (X) and Formula (XI) in the amino acid sequence of the IL-2 conjugate of SEQ ID NO: 3 is at position V68. In some embodiments, the position of the structure of Formula (X), Formula (XI), or a mixture of Formula (X) and Formula (XI) in the amino acid sequence of the IL-2 conjugate of SEQ ID NO: 3 is at position L71.

In some embodiments, the ratio of the amount of the structure of Formula (X) to the amount of the structure of Formula (XI) comprising the total amount of the IL-2 conjugate is about 1:1. In some embodiments, the ratio of the amount of the structure of Formula (X) to the amount of the structure of Formula (XI) comprising the total amount of the IL-2 conjugate is greater than 1:1. In some embodiments, the ratio of the amount of the structure of Formula (X) to the amount of the structure of Formula (XI) comprising the total amount of the IL-2 conjugate is less than 1:1.

In some embodiments, the IL-2 conjugate comprises the amino acid sequence of SEQ ID NO: 3 in which at least one amino acid residue in the IL-2 conjugate is replaced by the structure of Formula (X), Formula (XI), or a mixture of Formula (X) and Formula (XI), wherein the amino acid residue in SEQ ID NO: 3 that is replaced is selected from K34, F41, F43, K42, E61, P64, R37, T40, E67, Y44, V68, and L71, and wherein n is an integer from 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575. In some embodiments, n in the compounds of Formula (VI) and (VII) is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546.

In some embodiments, the IL-2 conjugate comprises the amino acid sequence of SEQ ID NO: 3 in which at least one amino acid residue in the IL-2 conjugate is replaced by the structure of Formula (X), Formula (XI), or a mixture of Formula (X) and Formula (XI), wherein the amino acid residue in in SEQ ID NO: 3 that is replaced is selected from F41, F43, K42, E61, and P64, and wherein n is an integer from about 450 to about 800, or from about 454 to about 796, or from about 454 to about 682, or from about 568 to about 909. In some embodiments, n in the compounds of Formula (X) and Formula (XI) is an integer selected from 454, 455, 568, 569, 680, 681, 682, 794, 795, 796, 908, 909, 910, 1021, 1022, 1023, 1135, 1136, 1137, and 1249.

In some embodiments, the IL-2 conjugate comprises the amino acid sequence of SEQ ID NO: 3 in which at least one amino acid residue in the IL-2 conjugate is replaced by the structure of Formula (X), Formula (XI), or a mixture of Formula (X) and Formula (XI), wherein the amino acid residue in SEQ ID NO: 3 that is replaced is selected from E61 and P64, and wherein n is an integer from about 450 to about 800, or from about 454 to about 796, or from about 454 to about 682, or from about 568 to about 909. In some embodiments, n in the compounds of Formula (X) and Formula (XI) is an integer selected from 454, 455, 568, 569, 680, 681, 682, 794, 795, 796, 908, 909, and 910.

In some embodiments, the IL-2 conjugate comprises the amino acid sequence of SEQ ID NO: 3 in which at least one amino acid residue in the IL-2 conjugate is replaced by the structure of Formula (X), Formula (XI), or a mixture of Formula (X) and Formula (XI), wherein the amino acid residue in SEQ ID NO: 3 that is replaced is E61, and wherein n is an integer from about 450 to about 800, or from about 454 to about 796, or from about 454 to about 682, or from about 568 to about 909. In some embodiments, n in the compounds of Formula (X) and Formula (XI) is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

In some embodiments, the IL-2 conjugate comprises the amino acid sequence of SEQ ID NO: 3 in which at least one amino acid residue in the IL-2 conjugate is replaced by the structure of Formula (X), Formula (XI), or a mixture of Formula (X) and Formula (XI), wherein the amino acid residue in SEQ ID NO: 3 that is replaced is P64, and wherein n is an integer from about 450 to about 800, or from about 454 to about 796, or from about 454 to about 682, or from about 568 to about 909. In some embodiments, n in the compounds of Formula (X) and Formula (XI) is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

In some embodiments, n in the structures of Formula (X) and Formula (XI) is an integer such that the molecular weight of the PEG moiety is in the range from about 1,000 Daltons about 200,000 Daltons, or from about 2,000 Daltons to about 150,000 Daltons, or from about 3,000 Daltons to about 125,000 Daltons, or from about 4,000 Daltons to about 100,000 Daltons, or from about 5,000 Daltons to about 100,000 Daltons, or from about 6,000 Daltons to about 90,000 Daltons, or from about 7,000 Daltons to about 80,000 Daltons, or from about 8,000 Daltons to about 70,000 Daltons, or from about 5,000 Daltons to about 70,000 Daltons, or from about 5,000 Daltons to about 65,000 Daltons, or from about 5,000 Daltons to about 60,000 Daltons, or from about 5,000 Daltons to about 50,000 Daltons, or from about 6,000 Daltons to about 50,000 Daltons, or from about 7,000 Daltons to about 50,000 Daltons, or from about 7,000 Daltons to about 45,000 Daltons, or from about 7,000 Daltons to about 40,000 Daltons, or from about 8,000 Daltons to about 40,000 Daltons, or from about 8,500 Daltons to about 40,000 Daltons, or from about 8,500 Daltons to about 35,000 Daltons, or from about 9,000 Daltons to about 50,000 Daltons, or from about 9,000 Daltons to about 45,000 Daltons, or from about 9,000 Daltons to about 40,000 Daltons, or from about 9,000 Daltons to about 35,000 Daltons, or from about 9,000 Daltons to about 30,000 Daltons, or from about 9,500 Daltons to about 35,000 Daltons, or from about 9,500 Daltons to about 30,000 Daltons, or from about 10,000 Daltons to about 50,000 Daltons, or from about 10,000 Daltons to about 45,000 Daltons, or from about 10,000 Daltons to about 40,000 Daltons, or from about 10,000 Daltons to about 35,000 Daltons, or from about 10,000 Daltons to about 30,000 Daltons, or from about 15,000 Daltons to about 50,000 Daltons, or from about 15,000 Daltons to about 45,000 Daltons, or from about 15,000 Daltons to about 40,000 Daltons, or from about 15,000 Daltons to about 35,000 Daltons, or from about 15,000 Daltons to about 30,000 Daltons, or from about 20,000 Daltons to about 50,000 Daltons, or from about 20,000 Daltons to about 45,000 Daltons, or from about 20,000 Daltons to about 40,000 Daltons, or from about 20,000 Daltons to about 35,000 Daltons, or from about 20,000 Daltons to about 30,000 Daltons.

In some embodiments, the IL-2 conjugate comprises the amino acid sequence of SEQ ID NO: 3 in which at least one amino acid residue in the IL-2 conjugate is replaced by the structure of Formula (X), Formula (XI), or a mixture of Formula (X) and Formula (XI), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, about 50,000 Daltons, about 60,000 Daltons, about 70,000 Daltons, about 80,000 Daltons, about 90,000 Daltons, about 100,000 Daltons, about 125,000 Daltons, about 150,000 Daltons, about 175,000 Daltons or about 200,000 Daltons.

In some embodiments, the IL-2 conjugate comprises the amino acid sequence of SEQ ID NO: 3 in which at least one amino acid residue in the IL-2 conjugate is replaced by the structure of Formula (X), Formula (XI), or a mixture of Formula (X) and Formula (XI), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, or about 50,000 Daltons.

In some embodiments, the IL-2 conjugate comprises the amino acid sequence of SEQ ID NO: 3 in which at least one amino acid residue in the IL-2 conjugate is replaced by the structure of Formula (XII) or Formula (XIII), or a mixture of Formula (XII) and Formula (XIII):

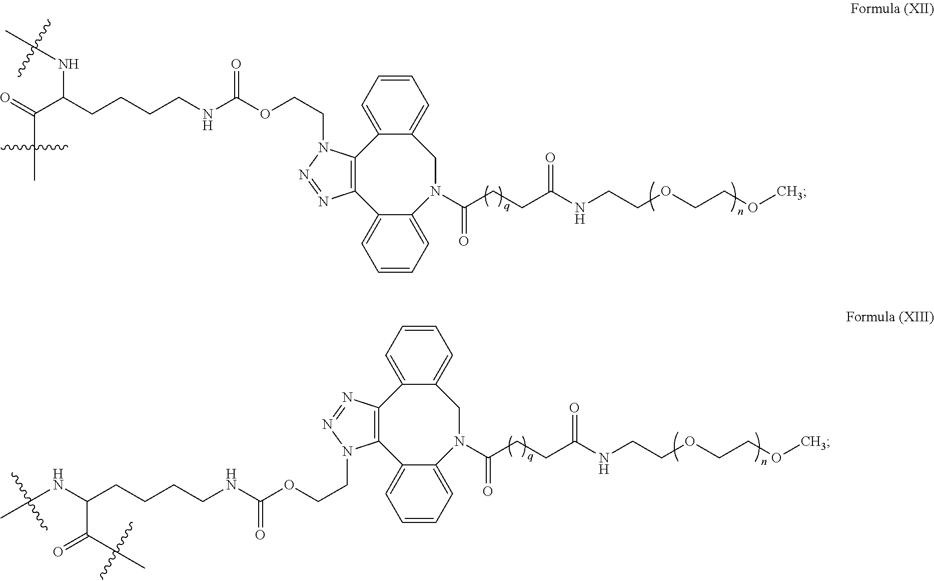

wherein:
n is an integer in the range from about 2 to about 5000;
q is 1, 2, or 3; and
the wavy lines indicate covalent bonds to amino acid residues within SEQ ID NO: 3 that are not replaced.

In some embodiments of Formula (XII) or Formula (XIII), or a mixture of Formula (XII) or Formula (XIII), q is 1. In some embodiments of Formula (XII) or Formula (XIII), or a mixture of Formula (XII) or Formula (XIII), q is 2. In some embodiments of Formula (XII) or Formula (XIII), or a mixture of Formula (XII) or Formula (XIII), q is 3.

Here and throughout, the structure of Formula (XII) encompasses pharmaceutically acceptable salts, solvates, or hydrates thereof. Here and throughout, the structure of Formula (XIII) encompasses pharmaceutically acceptable salts, solvates, or hydrates thereof. In some embodiments, the IL-2 conjugate is a pharmaceutically acceptable salt, solvate, or hydrate.

In some embodiments, q is 1 and the structures of Formula (XII) and Formula (XIII) are the structures of Formula (XIIa) and Formula (XIIIa):

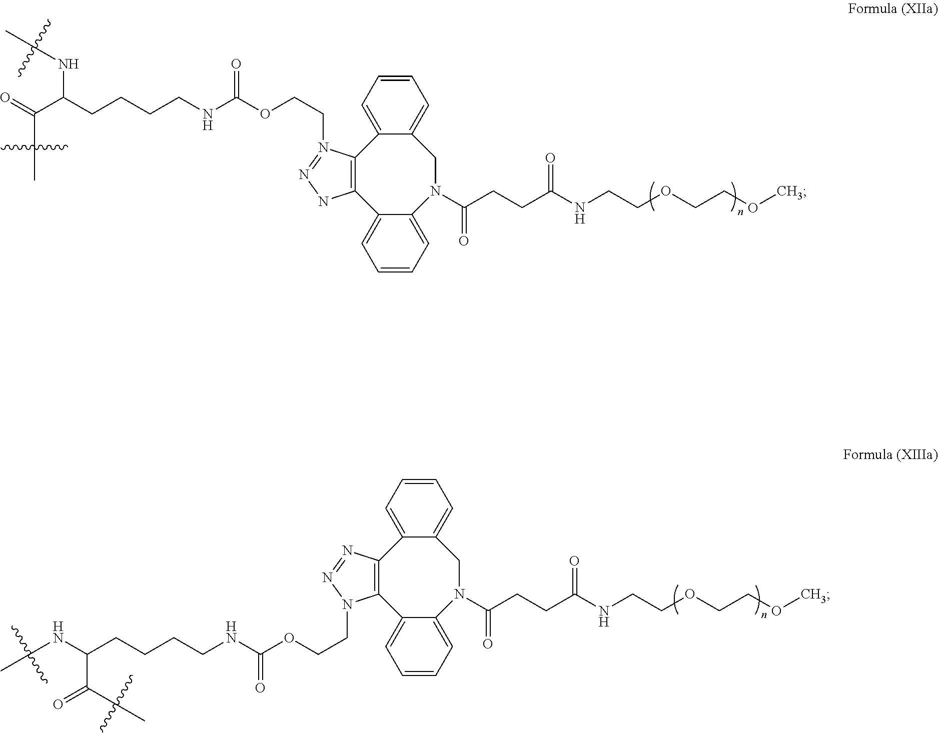

wherein:
n is an integer in the range from about 2 to about 5000; and
the wavy lines indicate covalent bonds to amino acid residues within SEQ ID NO: 3 that are not replaced.

In some embodiments, the stereochemistry of the chiral center within Formula (XII) and Formula (XIII) is racemic, is enriched in (R), is enriched in (S), is substantially (R), is substantially (S), is (R) or is (S). In some embodiments, the stereochemistry of the chiral center within Formula (XII) and Formula (XIII) is racemic. In some embodiments, the stereochemistry of the chiral center within Formula (XII) and Formula (XIII) is enriched in (R). In some embodiments, the stereochemistry of the chiral center within Formula (XII) and Formula (XIII) is enriched in (S). In some embodiments, the stereochemistry of the chiral center within Formula (XII) and Formula (XIII) is substantially (R). In some embodiments, the stereochemistry of the chiral center within Formula (XII) and Formula (XIII) is substantially (S). In some embodiments, the stereochemistry of the chiral center within Formula (XII) and Formula (XIII) is (R). In some embodiments, the stereochemistry of the chiral center within Formula (XII) and Formula (XIII) is (S).

In some embodiments, n in the compounds of Formula (XII) and Formula (XIII) is in the range from about 5 to about 4600, or from about 10 to about 4000, or from about 20 to about 3000, or from about 100 to about 3000, or from about 100 to about 2900, or from about 150 to about 2900, or from about 125 to about 2900, or from about 100 to about 2500, or from about 100 to about 2000, or from about 100 to about 1900, or from about 100 to about 1850, or from about 100 to about 1750, or from about 100 to about 1650, or from about 100 to about 1500, or from about 100 to about 1400, or from about 100 to about 1300, or from about 100 to about 1250, or from about 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575. In some embodiments, n in the compounds of Formula (XII) and Formula (XIII) is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546.

In some embodiments, the position of the structure of Formula (XII), Formula (XIII), or a mixture of Formula (XII) and Formula (XIII) in the amino acid sequence of the IL-2 conjugate is selected from K34, F41, F43, K42, E61, P64, R37, T40, E67, Y44, V68, and L71, wherein the position of the structure of Formula (XII), Formula (XIII), or a mixture of Formula (XII) and Formula (XIII) in the amino acid sequence of the IL-2 conjugate is in reference to the positions in SEQ ID NO: 3. In some embodiments, the position of the structure of Formula (XII), Formula (XIII), or a mixture of Formula (XII) and Formula (XIII) in the amino acid sequence of the IL-2 conjugate of SEQ ID NO: 3 is selected from K34, F41, F43, K42, E61, P64, R37, T40, E67, Y44, V68, and L71. In some embodiments, the position of the structure of Formula (XII), Formula (XIII), or a mixture of Formula (XII) and Formula (XIII) in the amino acid sequence of the IL-2 conjugate of SEQ ID NO: 3 is at position K34. In some embodiments, the position of the structure of Formula (XII), Formula (XIII), or a mixture of Formula (XII) and Formula (XIII) in the amino acid sequence of the IL-2 conjugate of SEQ ID NO: 3 is at position F41. In some embodiments, the position of the structure of Formula (XII), Formula (XIII), or a mixture of Formula (XII) and Formula (XIII) in the amino acid sequence of the IL-2 conjugate of SEQ ID NO: 3 is at position F43. In some embodiments, the position of the structure of Formula (XII), Formula (XIII), or a mixture of Formula (XII) and Formula (XIII) in the amino acid sequence of the IL-2 conjugate of SEQ ID NO: 3 is at position K42. In some embodiments, the position of the structure of Formula (XII), Formula (XIII), or a mixture of Formula (XII) and Formula (XIII) in the amino acid sequence of the IL-2 conjugate of SEQ ID NO: 3 is at position E61. In some embodiments, the position of the structure of Formula (XII), Formula (XIII), or a mixture of Formula (XII) and Formula (XIII) in the amino acid sequence of the IL-2 conjugate of SEQ ID NO: 3 is at position P64. In some embodiments, the position of the structure of Formula (XII), Formula (XIII), or a mixture of Formula (XII) and Formula (XIII) in the amino acid sequence of the IL-2 conjugate of SEQ ID NO: 3 is at position R37. In some embodiments, the position of the structure of Formula (XII), Formula (XIII), or a mixture of Formula (XII) and Formula (XIII) in the amino acid sequence of the IL-2 conjugate of SEQ ID NO: 3 is at position T40. In some embodiments, the position of the structure of Formula (XII), Formula (XIII), or a mixture of Formula (XII) and Formula (XIII) in the amino acid sequence of the IL-2 conjugate of SEQ ID NO: 3 is at position E67. In some embodiments, the position of the structure of Formula (XII), Formula (XIII), or a mixture of Formula (XII) and Formula (XIII) in the amino acid sequence of the IL-2 conjugate of SEQ ID NO: 3 is at position Y44. In some embodiments, the position of the structure of Formula (XII), Formula (XIII), or a mixture of Formula (XII) and Formula (XIII) in the amino acid sequence of the IL-2 conjugate of SEQ ID NO: 3 is at position V68. In some embodiments, the position of the structure of Formula (XII), Formula (XIII), or a mixture of Formula (XII) and Formula (XIII) in the amino acid sequence of the IL-2 conjugate of SEQ ID NO: 3 is at position L71.

In some embodiments, the ratio of the amount of the structure of Formula (XII) to the amount of the structure of Formula (XIII) comprising the total amount of the IL-2 conjugate is about 1:1. In some embodiments, the ratio of the amount of the structure of Formula (XII) to the amount of the structure of Formula (XIII) comprising the total amount of the IL-2 conjugate is greater than 1:1. In some embodiments, the ratio of the amount of the structure of Formula (XII) to the amount of the structure of Formula (XIII) comprising the total amount of the IL-2 conjugate is less than 1:1.

In some embodiments, the IL-2 conjugate comprises the amino acid sequence of SEQ ID NO: 3 in which at least one amino acid residue in the IL-2 conjugate is replaced by the structure of Formula (XII), Formula (XIII), or a mixture of Formula (XII) and Formula (XIII), wherein the amino acid residue in SEQ ID NO: 3 that is replaced is selected from K34, F41, F43, K42, E61, P64, R37, T40, E67, Y44, V68, and L71, and wherein n is an integer from 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575. In some embodiments, n in the compounds of Formula (XII) and Formula (XIII) is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546.

In some embodiments, the IL-2 conjugate comprises the amino acid sequence of SEQ ID NO: 3 in which at least one amino acid residue in the IL-2 conjugate is replaced by the structure of Formula (XII), Formula (XIII), or a mixture of Formula (XII) and Formula (XIII), wherein the amino acid residue in in SEQ ID NO: 3 that is replaced is selected from F41, F43, K42, E61, and P64, and wherein n is an integer from about 450 to about 800, or from about 454 to about 796, or from about 454 to about 682, or from about 568 to about 909. In some embodiments, n in the compounds of Formula (XII) and Formula (XIII) is an integer selected from 454, 455, 568, 569, 680, 681, 682, 794, 795, 796, 908, 909, 910, 1021, 1022, 1023, 1135, 1136, 1137, and 1249.

In some embodiments, the IL-2 conjugate comprises the amino acid sequence of SEQ ID NO: 3 in which at least one amino acid residue in the IL-2 conjugate is replaced by the structure of Formula (XII), Formula (XIII), or a mixture of Formula (XII) and Formula (XIII), wherein the amino acid residue in SEQ ID NO: 3 that is replaced is selected from E61 and P64, and wherein n is an integer from about 450 to about 800, or from about 454 to about 796, or from about 454 to about 682, or from about 568 to about 909. In some embodiments, n in the compounds of Formula (XII) and Formula (XIII) is an integer selected from 454, 455, 568, 569, 680, 681, 682, 794, 795, 796, 908, 909, and 910.

In some embodiments, the IL-2 conjugate comprises the amino acid sequence of SEQ ID NO: 3 in which at least one amino acid residue in the IL-2 conjugate is replaced by the structure of Formula (XII), Formula (XIII), or a mixture of Formula (XII) and Formula (XIII), wherein the amino acid residue in SEQ ID NO: 3 that is replaced is E61, and wherein n is an integer from about 450 to about 800, or from about 454 to about 796, or from about 454 to about 682, or from about 568 to about 909. In some embodiments, n in the compounds of Formula (XII) and Formula (XIII) is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

In some embodiments, the IL-2 conjugate comprises the amino acid sequence of SEQ ID NO: 3 in which at least one amino acid residue in the IL-2 conjugate is replaced by the structure of Formula (XII), Formula (XIII), or a mixture of Formula (XII) and Formula (XIII), wherein the amino acid residue in in SEQ ID NO: 3 that is replaced is P64, and wherein n is an integer from about 450 to about 800, or from about 454 to about 796, or from about 454 to about 682, or from about 568 to about 909. In some embodiments, n in the compounds of Formula (XII) and Formula (XIII) is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

In some embodiments, n in the structure of Formula (XII) or Formula (XIII) is an integer such that the molecular weight of the PEG moiety is in the range from about 1,000 Daltons about 200,000 Daltons, or from about 2,000 Daltons to about 150,000 Daltons, or from about 3,000 Daltons to about 125,000 Daltons, or from about 4,000 Daltons to about 100,000 Daltons, or from about 5,000 Daltons to about 100,000 Daltons, or from about 6,000 Daltons to about 90,000 Daltons, or from about 7,000 Daltons to about 80,000 Daltons, or from about 8,000 Daltons to about 70,000 Daltons, or from about 5,000 Daltons to about 70,000 Daltons, or from about 5,000 Daltons to about 65,000 Daltons, or from about 5,000 Daltons to about 60,000 Daltons, or from about 5,000 Daltons to about 50,000 Daltons, or from about 6,000 Daltons to about 50,000 Daltons, or from about 7,000 Daltons to about 50,000 Daltons, or from about 7,000 Daltons to about 45,000 Daltons, or from about 7,000 Daltons to about 40,000 Daltons, or from about 8,000 Daltons to about 40,000 Daltons, or from about 8,500 Daltons to about 40,000 Daltons, or from about 8,500 Daltons to about 35,000 Daltons, or from about 9,000 Daltons to about 50,000 Daltons, or from about 9,000 Daltons to about 45,000 Daltons, or from about 9,000 Daltons to about 40,000 Daltons, or from about 9,000 Daltons to about 35,000 Daltons, or from about 9,000 Daltons to about 30,000 Daltons, or from about 9,500 Daltons to about 35,000 Daltons, or from about 9,500 Daltons to about 30,000 Daltons, or from about 10,000 Daltons to about 50,000 Daltons, or from about 10,000 Daltons to about 45,000 Daltons, or from about 10,000 Daltons to about 40,000 Daltons, or from about 10,000 Daltons to about 35,000 Daltons, or from about 10,000 Daltons to about 30,000 Daltons, or from about 15,000 Daltons to about 50,000 Daltons, or from about 15,000 Daltons to about 45,000 Daltons, or from about 15,000 Daltons to about 40,000 Daltons, or from about 15,000 Daltons to about 35,000 Daltons, or from about 15,000 Daltons to about 30,000 Daltons, or from about 20,000 Daltons to about 50,000 Daltons, or from about 20,000 Daltons to about 45,000 Daltons, or from about 20,000 Daltons to about 40,000 Daltons, or from about 20,000 Daltons to about 35,000 Daltons, or from about 20,000 Daltons to about 30,000 Daltons.

In some embodiments, the IL-2 conjugate comprises the amino acid sequence of SEQ ID NO: 3 in which at least one amino acid residue in the IL-2 conjugate is replaced by the structure of Formula (XII), Formula (XIII), or a mixture of Formula (XII) and Formula (XIII), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, about 50,000 Daltons, about 60,000 Daltons, about 70,000 Daltons, about 80,000 Daltons, about 90,000 Daltons, about 100,000 Daltons, about 125,000 Daltons, about 150,000 Daltons, about 175,000 Daltons or about 200,000 Daltons. In some embodiments, the IL-2 conjugate comprises the amino acid sequence of SEQ ID NO: 3 in which at least one amino acid residue in the IL-2 conjugate is replaced by the structure of Formula (XII), Formula (XIII), or a mixture of Formula (XII) and Formula (XIII), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, or about 50,000 Daltons.

In some embodiments, the IL-2 conjugate comprises the amino acid sequence of SEQ ID NO: 3 in which the amino acid residue at E61 or P64 in the IL-2 conjugate is replaced by the structure of Formula (VIII) or Formula (IX), or a mixture of Formula (VIII) and Formula (IX):

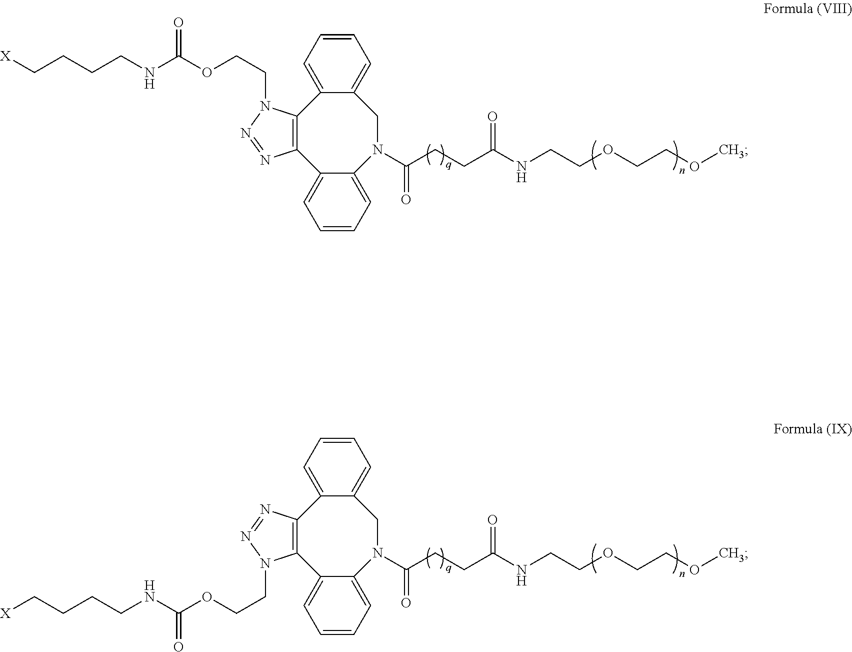

wherein:
n is an integer such that the molecular weight of the PEG group is from about 15,000 Daltons to about 60,000 Daltons;
q is 1, 2, or 3; and
X has the structure:

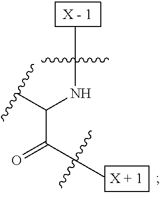

X−1 indicates the point of attachment to the preceding amino acid residue; and
X+1 indicates the point of attachment to the following amino acid residue.

In some embodiments of Formula (VIII) or Formula (IX), or a mixture of Formula (VIII) or Formula (IX), q is 1. In some embodiments of Formula (VIII) or Formula (IX), or a mixture of Formula (VIII) or Formula (IX), q is 2. In some embodiments of Formula (VIII) or Formula (IX), or a mixture of Formula (VIII) or Formula (IX), q is 3.

Here and throughout, the structure of Formula (VIII) encompasses pharmaceutically acceptable salts, solvates, or hydrates thereof. Here and throughout, the structure of Formula (IX) encompasses pharmaceutically acceptable salts, solvates, or hydrates thereof. In some embodiments, the IL-2 conjugate is a pharmaceutically acceptable salt, solvate, or hydrate.

In some embodiments, q is 1 and the structures of Formula (VIII) and Formula (IX) are the structures of Formula (VIIIa) and Formula (IXa):

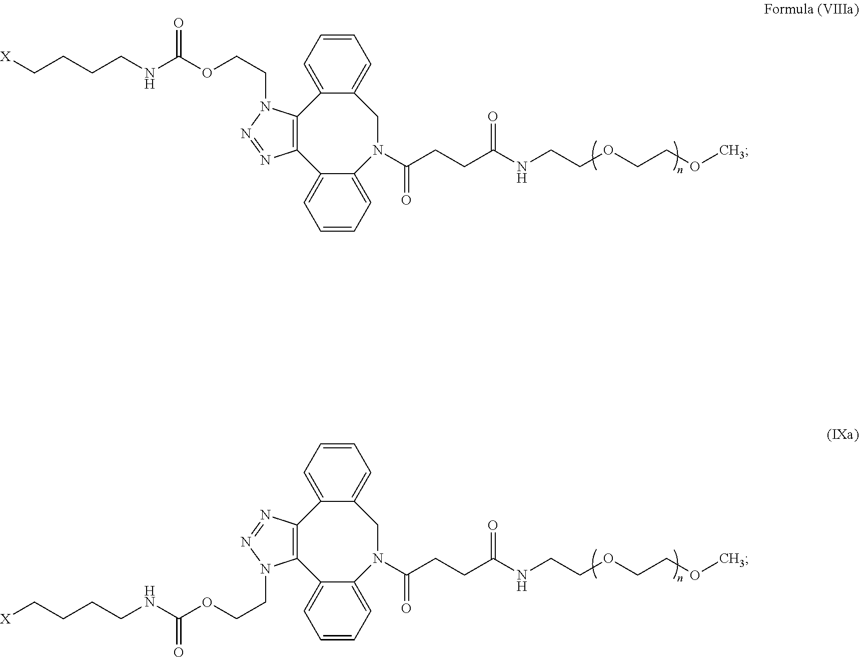

wherein:
n is an integer such that the molecular weight of the PEG group is from about 15,000 Daltons to about 60,000 Daltons; and
X has the structure:

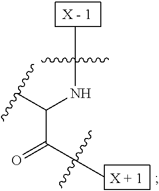

X−1 indicates the point of attachment to the preceding amino acid residue; and
X+1 indicates the point of attachment to the following amino acid residue.

In some embodiments, the amino acid residue at E61 in the IL-2 conjugate is replaced by the structure of Formula (VIII) or Formula (IX), or a mixture of Formula (VIII) and Formula (IX), and wherein n is an integer such that the molecular weight of the PEG group is from about 20,000 Daltons to about 40,000 Daltons. In some embodiments, n is an integer such that the molecular weight of the PEG group is from about 30,000 Daltons. In some embodiments, the amino acid residue at P64 in the IL-2 conjugate is replaced by the structure of Formula (VIII) or Formula (IX), or a mixture of Formula (VIII) and Formula (IX), and wherein n is an integer such that the molecular weight of the PEG group is from about 20,000 Daltons to about 40,000 Daltons. In some embodiments, n is an integer such that the molecular weight of the PEG group is from about 30,000 Daltons.

In some embodiments, the IL-2 conjugate comprises the amino acid sequence of SEQ ID NO: 3 in which the amino acid residue at E61 or P64 in the IL-2 conjugate is replaced by the structure of Formula (VIII) or (IX), or a mixture of (VIII) and (IX):

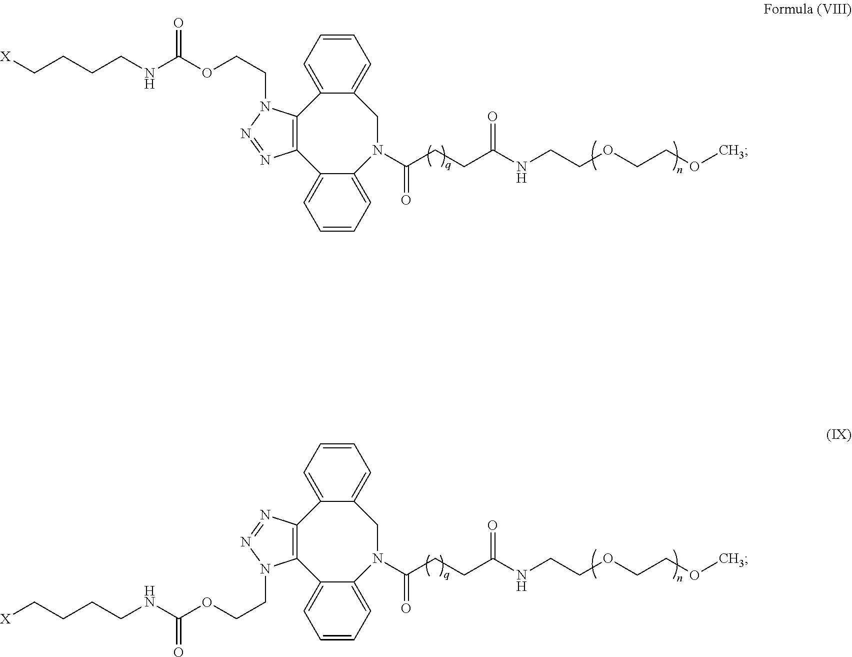

wherein:
n is an integer such that the molecular weight of the PEG group is from about 15,000 Daltons to about 60,000 Daltons;
q is 1, 2, or 3; and
X has the structure:

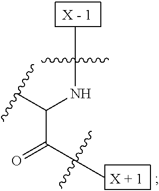

X−1 indicates the point of attachment to the preceding amino acid residue; and
X+1 indicates the point of attachment to the following amino acid residue.

In some embodiments, the amino acid residue at E61 in the IL-2 conjugate is replaced by the structure of Formula (VIII) or Formula (IX), or a mixture of Formula (VIII) and Formula (IX), and wherein n is an integer such that the molecular weight of the PEG group is from about 20,000 Daltons to about 40,000 Daltons. In some embodiments, n is an integer such that the molecular weight of the PEG group is from about 30,000 Daltons. In some embodiments, the amino acid residue at P64 in the IL-2 conjugate is replaced by the structure of Formula (VIII) or Formula (IX), or a mixture of Formula (VIII) and Formula (IX), and wherein n is an integer such that the molecular weight of the PEG group is from about 20,000 Daltons to about 40,000 Daltons. In some embodiments, n is an integer such that the molecular weight of the PEG group is from about 30,000 Daltons.

In some embodiments, the IL-2 conjugate comprises the amino acid sequence of SEQ ID NO: 1, SEQ ID NO: 3, or SEQ ID NO: 4 in which at least one amino acid residue in the IL-2 conjugate is replaced by a cysteine covalently bonded to a PEG group. In some embodiments, the PEG group has a molecular weight selected from 5 kDa, 10 kDa, 15 kDa, 20 kDa, 25 kDa, 30 kDa, 35 kDa, 40 kDa, 45 kDa, 50 kDa, and 60 kDa. In some embodiments, the PEG group has a molecular weight of 5 kDa. In some embodiments, the PEG group has a molecular weight of 10 kDa. In some embodiments, the PEG group has a molecular weight of 15 kDa. In some embodiments, the PEG group has a molecular weight of 20 kDa. In some embodiments, the PEG group has a molecular weight of 25 kDa. In some embodiments, the PEG group has a molecular weight of 30 kDa. In some embodiments, the PEG group has a molecular weight of 35 kDa. In some embodiments, the PEG group has a molecular weight of 40 kDa. In some embodiments, the PEG group has a molecular weight of 45 kDa. In some embodiments, the PEG group has a molecular weight of 50 kDa. In some embodiments, the PEG group has a molecular weight of 60 kDa. In some embodiments, the IL-2 conjugate comprises the amino acid sequence of SEQ ID NO: 3 and the at least one amino acid residue in the IL-2 conjugate that is replaced by a cysteine is selected from K34, T36, R37, T40, F41, K42, F43, Y44, E60, E61, E67, K63, P64, V68, L71, and Y106. In some embodiments, the IL-2 conjugate comprises the amino acid sequence of SEQ ID NO: 3 and the at least one amino acid residue in the IL-2 conjugate that is replaced by a cysteine is selected from K34, T40, F41, K42, Y44, E60, E61, E67, K63, P64, V68, and L71. In some embodiments, the IL-2 conjugate comprises the amino acid sequence of SEQ ID NO: 4 and the at least one amino acid residue in the IL-2 conjugate that is replaced by a cysteine is selected from K35, T37, R38, T41, F42, K43, F44, Y45, E61, E62, E68, K64, P65, V69, L72, and Y107

In some embodiments, the IL-2 conjugate comprises the amino acid sequence of SEQ ID NO: 3 in which at least one non-lysine residue is replaced by a lysine comprising a linker and a water-soluble polymer. In some embodiments, the water-soluble polymer is a PEG group.

In some embodiments, the IL-2 conjugate comprises a PEG group covalently bonded via a non-releasable linkage. In some embodiments, the IL-2 conjugate comprises a non-releasable, covalently bonded PEG group.

In some embodiments, the IL-2 conjugate comprises the amino acid sequence of SEQ ID NO: 3, wherein a non-lysine amino acid in the IL-2 conjugate is replaced by a lysine residue, and wherein the lysine residue comprises one or more water soluble polymers and a covalent linker. In some embodiments, the lysine residue is located in the region K34-Y106 of SEQ ID NO: 3. In some embodiments, the lysine residue is located at K34. In some embodiments, the lysine residue is located at F41. In some embodiments, the lysine residue is located at F43. In some embodiments, the lysine residue is located at K42. In some embodiments, the lysine residue is located at E61. In some embodiments, the lysine residue is located at P64. In some embodiments, the lysine residue is located at R37. In some embodiments, the lysine residue is located at T40. In some embodiments, the lysine residue is located at E67. In some embodiments, the lysine residue is located at Y44. In some embodiments, the lysine residue is located at V68. In some embodiments, the lysine residue is located at L71.

In some embodiments, the IL-2 conjugate comprises the amino acid sequence of SEQ ID NO: 3, wherein a non-lysine amino acid in the amino acid sequence of the IL-2 conjugate is replaced by an amino acid comprising: (a) a lysine; (b) a covalent linker; and (3) and one or more water-soluble polymers. In some embodiments, the one or more water-soluble polymers comprises a PEG group.

In some embodiments, the IL-2 conjugate comprises the amino acid sequence of SEQ ID NO: 3 in which at least one amino acid residue in the IL-2 conjugate is replaced by the structure of Formula (XIV) or Formula (XV), or a mixture of Formula (XIV) and Formula (XV):

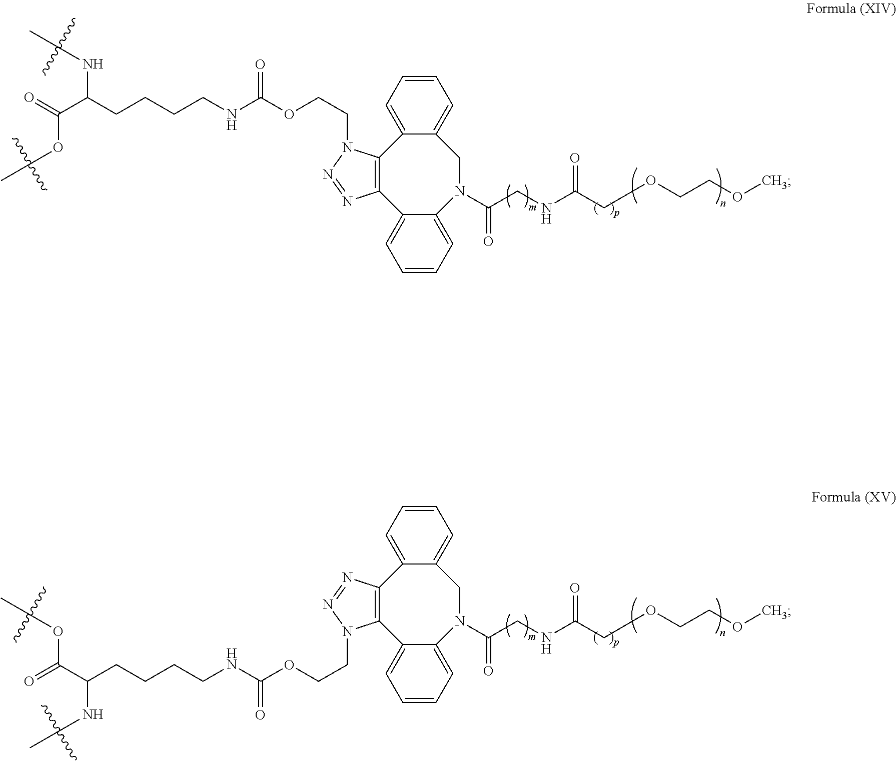

wherein:
m is an integer from 0 to 20;
p is an integer from 0 to 20;
n is an integer in the range from about 2 to about 5000; and
the wavy lines indicate covalent bonds to amino acid residues within SEQ ID NO: 3 that are not replaced.

Here and throughout, the structure of Formula (XIV) encompasses pharmaceutically acceptable salts, solvates, or hydrates thereof. Here and throughout, the structure of Formula (XV) encompasses pharmaceutically acceptable salts, solvates, or hydrates thereof. In some embodiments, the IL-2 conjugate is a pharmaceutically acceptable salt, solvate, or hydrate.

In some embodiments, the stereochemistry of the chiral center within Formula (XIV) and Formula (XV) is racemic, is enriched in (R), is enriched in (S), is substantially (R), is substantially (S), is (R) or is (S). In some embodiments, the stereochemistry of the chiral center within Formula (XIV) and Formula (XV) is racemic. In some embodiments, the stereochemistry of the chiral center within Formula (XIV) and Formula (XV) is enriched in (R). In some embodiments, the stereochemistry of the chiral center within Formula (XIV) and Formula (XV) is enriched in (S). In some embodiments, the stereochemistry of the chiral center within Formula (XIV) and Formula (XV) is substantially (R). In some embodiments, the stereochemistry of the chiral center within Formula (XIV) and Formula (XV) is substantially (S). In some embodiments, the stereochemistry of the chiral center within Formula (XIV) and Formula (XV) is (R). In some embodiments, the stereochemistry of the chiral center within Formula (XIV) and Formula (XV) is (S).

In some embodiments, the IL-2 conjugate comprises the amino acid sequence of SEQ ID NO: 3 in which m in the compounds of Formula (XIV) and Formula (XV) is from 0 to 20, or from 1 to 18, or from 1 to 16, or from 1 to 14, or from 1 to 12, or from 1 to 10, or from 1 to 9, or from 1 to 8, or from 1 to 7, or from 1 to 6, or from 1 to 5, or from 1 to 4, or from 1 to 3, or from 1 to 2. In some embodiments, m in the compounds of Formula (XIV) and Formula (XV) is 1. In some embodiments, m in the compounds of Formula (XIV) and Formula (XV) is 2. In some embodiments, m in the compounds of Formula (XIV) and Formula (XV) is 3. In some embodiments, m in the compounds of Formula (XIV) and Formula (XV) is 4. In some embodiments, m in the compounds of Formula (XIV) and Formula (XV) is 5. In some embodiments, m in the compounds of Formula (XIV) and Formula (XV) is 6. In some embodiments, m in the compounds of Formula (XIV) and Formula (XV) is 7. In some embodiments, m in the compounds of Formula (XIV) and Formula (XV) is 8. In some embodiments, m in the compounds of Formula (XIV) and Formula (XV) is 9. In some embodiments, m in the compounds of Formula (XIV) and Formula (XV) is 10. In some embodiments, m in the compounds of Formula (XIV) and Formula (XV) is 11. In some embodiments, m in the compounds of Formula (XIV) and Formula (XV) is 12. In some embodiments, m in the compounds of Formula (XIV) and Formula (XV) is 13. In some embodiments, m in the compounds of Formula (XIV) and Formula (XV) is 14. In some embodiments, m in the compounds of Formula (XIV) and Formula (XV) is 15. In some embodiments, m in the compounds of Formula (XIV) and Formula (XV) is 16. In some embodiments, m in the compounds of Formula (XIV) and Formula (XV) is 17. In some embodiments, m in the compounds of Formula (XIV) and Formula (XV) is 18. In some embodiments, m in the compounds of Formula (XIV) and Formula (XV) is 19. In some embodiments, m in the compounds of Formula (XIV) and Formula (XV) is 20.

In some embodiments, the IL-2 conjugate comprises the amino acid sequence of SEQ ID NO: 3 in which p in the compounds of Formula (XIV) and Formula (XV) is from 1 to 20, or from 1 to 18, or from 1 to 16, or from 1 to 14, or from 1 to 12, or from 1 to 10, or from 1 to 9, or from 1 to 8, or from 1 to 7, or from 1 to 6, or from 1 to 5, or from 1 to 4, or from 1 to 3, or from 1 to 2. In some embodiments, p in the compounds of Formula (XIV) and Formula (XV) is 1. In some embodiments, p in the compounds of Formula (XIV) and Formula (XV) is 2. In some embodiments, p in the compounds of Formula (XIV) and Formula (XV) is 3. In some embodiments, p in the compounds of Formula (XIV) and Formula (XV) is 4. In some embodiments, p in the compounds of Formula (XIV) and Formula (XV) is 5. In some embodiments, p in the compounds of Formula (XIV) and Formula (XV) is 6. In some embodiments, p in the compounds of Formula (XIV) and Formula (XV) is 7. In some embodiments, p in the compounds of Formula (XIV) and Formula (XV) is 8. In some embodiments, p in the compounds of Formula (XIV) and Formula (XV) is 9. In some embodiments, p in the compounds of Formula (XIV) and Formula (XV) is 10. In some embodiments, p in the compounds of Formula (XIV) and Formula (XV) is 11. In some embodiments, p in the compounds of Formula (XIV) and Formula (XV) is 12. In some embodiments, p in the compounds of Formula (XIV) and Formula (XV) is 13. In some embodiments, p in the compounds of Formula (XIV) and Formula (XV) is 14. In some embodiments, p in the compounds of Formula (XIV) and Formula (XV) is 15. In some embodiments, m in the compounds of Formula (XIV) and Formula (XV) is 16. In some embodiments, p in the compounds of Formula (XIV) and Formula (XV) is 17. In some embodiments, p in the compounds of Formula (XIV) and Formula (XV) is 18. In some embodiments, p in the compounds of Formula (XIV) and Formula (XV) is 19. In some embodiments, p in the compounds of Formula (XIV) and Formula (XV) is 20.

In some embodiments, the IL-2 conjugate comprises the amino acid sequence of SEQ ID NO: 3 in which n in the compounds of Formula (XIV) and Formula (XV) is in the range from about 5 to about 4600, or from about 10 to about 4000, or from about 20 to about 3000, or from about 100 to about 3000, or from about 100 to about 2900, or from about 150 to about 2900, or from about 125 to about 2900, or from about 100 to about 2500, or from about 100 to about 2000, or from about 100 to about 1900, or from about 100 to about 1850, or from about 100 to about 1750, or from about 100 to about 1650, or from about 100 to about 1500, or from about 100 to about 1400, or from about 100 to about 1300, or from about 100 to about 1250, or from about 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575.

In some embodiments, the IL-2 conjugate comprises the amino acid sequence of SEQ ID NO: 3 in which m in the compounds of Formula (XIV) and Formula (XV) is an integer from 1 to 6, p is an integer from 1 to 6, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XIV) and Formula (XV), m is an integer from 2 to 6, p is an integer from 2 to 6, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XIV) and Formula (XV), m is an integer from 2 to 4, p is an integer from 2 to 4, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XIV) and Formula (XV), m is 1, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XIV) and Formula (XV), m is 2, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XIV) and Formula (XV), m is 3, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XIV) and Formula (XV), m is 4, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XIV) and Formula (XV), m is 5, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XIV) and Formula (XV), m is 6, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XIV) and Formula (XV), m is 7, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XIV) and Formula (XV), m is 8, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XIV) and Formula (XV), m is 9, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XIV) and Formula (XV), m is 10, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XIV) and (XV), m is 11, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XIV) and Formula (XV), m is 11, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XIV) and Formula (XV), m is 2, p is 2, and n is an integer selected from 680, 681, 682, 794, 795, 796, 908, 909, 910, 1021, 1022, 1023, 1135, 1136, and 1137.

In some embodiments, the IL-2 conjugate comprises the amino acid sequence of SEQ ID NO: 3 in which n in the compounds of Formula (XIV) and Formula (XV) is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546. In some embodiments, the position of the structure of Formula (XIV), Formula (XV), or a mixture of Formula (XIV) and Formula (XV) in the amino acid sequence of the IL-2 conjugate is selected from K34, F41, F43, K42, E61, P64, R37, T40, E67, Y44, V68, and L71, wherein the position of the structure of Formula (XIV), Formula (XV), or a mixture of Formula (XIV) and Formula (XV) in the amino acid sequence of the IL-2 conjugate is in reference to the positions in SEQ ID NO: 3. In some embodiments, the position of the structure of Formula (XIV), Formula (XV), or a mixture of Formula (XIV) and Formula (XV) in the amino acid sequence of the IL-2 conjugate of SEQ ID NO: 3 is selected from K34, F41, F43, K42, E61, P64, R37, T40, E67, Y44, V68, and L71. In some embodiments, the position of the structure of Formula (XIV), Formula (XV), or a mixture of Formula (XIV) and Formula (XV) in the amino acid sequence of the IL-2 conjugate of SEQ ID NO: 3 is at position K34. In some embodiments, the position of the structure of Formula (XIV), Formula (XV), or a mixture of Formula (XIV) and Formula (XV) in the amino acid sequence of the IL-2 conjugate of SEQ ID NO: 3 is at position F41. In some embodiments, the position of the structure of Formula (XIV), Formula (XV), or a mixture of Formula (XIV) and Formula (XV) in the amino acid sequence of the IL-2 conjugate of SEQ ID NO: 3 is at position F43. In some embodiments, the position of the structure of Formula (XIV), Formula (XV), or a mixture of Formula (XIV) and Formula (XV) in the amino acid sequence of the IL-2 conjugate of SEQ ID NO: 3 is at position K42. In some embodiments, the position of the structure of Formula (XIV), Formula (XV), or a mixture of Formula (XIV) and Formula (XV) in the amino acid sequence of the IL-2 conjugate of SEQ ID NO: 3 is at position E61. In some embodiments, the position of the structure of Formula (XIV), Formula (XV), or a mixture of Formula (XIV) and Formula (XV) in the amino acid sequence of the IL-2 conjugate of SEQ ID NO: 3 is at position P64. In some embodiments, the position of the structure of Formula (XIV), Formula (XV), or a mixture of Formula (XIV) and Formula (XV) in the amino acid sequence of the IL-2 conjugate of SEQ ID NO: 3 is at position R37. In some embodiments, the position of the structure of Formula (XIV), Formula (XV), or a mixture of Formula (XIV) and Formula (XV) in the amino acid sequence of the IL-2 conjugate of SEQ ID NO: 3 is at position T40. In some embodiments, the position of the structure of Formula (XIV), Formula (XV), or a mixture of Formula (XIV) and Formula (XV) in the amino acid sequence of the IL-2 conjugate of SEQ ID NO: 3 is at position E67. In some embodiments, the position of the structure of Formula (XIV), Formula (XV), or a mixture of Formula (XIV) and Formula (XV) in the amino acid sequence of the IL-2 conjugate of SEQ ID NO: 3 is at position Y44. In some embodiments, the position of the structure of Formula (XIV), Formula (XV), or a mixture of Formula (XIV) and Formula (XV) in the amino acid sequence of the IL-2 conjugate of SEQ ID NO: 3 is at position V68. In some embodiments, the position of the structure of Formula (XIV), Formula (XV), or a mixture of Formula (XIV) and Formula (XV) in the amino acid sequence of the IL-2 conjugate of SEQ ID NO: 3 is at position L71. In some embodiments, the ratio of the amount of the structure of Formula (XIV) to the amount of the structure of Formula (XV) comprising the total amount of the IL-2 conjugate is about 1:1. In some embodiments, the ratio of the amount of the structure of Formula (XIV) to the amount of the structure of Formula (XV) comprising the total amount of the IL-2 conjugate is greater than 1:1. In some embodiments, the ratio of the amount of the structure of Formula (XIV) to the amount of the structure of Formula (XV) comprising the total amount of the IL-2 conjugate is less than 1:1.

In some embodiments, the IL-2 conjugate comprises the amino acid sequence of SEQ ID NO: 3 in which at least one amino acid residue in the IL-2 conjugate is replaced by the structure of Formula (XIV) or Formula (XV), or a mixture of Formula (XIV) and Formula (XV), wherein the amino acid residue in SEQ ID NO: 3 that is replaced is selected from K34, F41, F43, K42, E61, P64, R37, T40, E67, Y44, V68, and L71, and wherein n is an integer from 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575. In some embodiments, n in the compounds of Formula (XIV) and Formula (XV) is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546.

In some embodiments, the IL-2 conjugate comprises the amino acid sequence of SEQ ID NO: 3 in which at least one amino acid residue in the IL-2 conjugate is replaced by the structure of Formula (XIV) or Formula (XV), or a mixture of Formula (XIV) and Formula (XV), wherein the amino acid residue in in SEQ ID NO: 3 that is replaced is selected from F41, F43, K42, E61, and P64, and wherein n is an integer from about 450 to about 800, or from about 454 to about 796, or from about 454 to about 682, or from about 568 to about 909. In some embodiments, n in the compounds of Formula (XIV) and Formula (XV) is an integer selected from 454, 455, 568, 569, 680, 681, 682, 794, 795, 796, 908, 909, 910, 1021, 1022, 1023, 1135, 1136, 1137, and 1249.

In some embodiments, the IL-2 conjugate comprises the amino acid sequence of SEQ ID NO: 3 in which at least one amino acid residue in the IL-2 conjugate is replaced by the structure Formula (XIV) or Formula (XV), or a mixture of Formula (XIV) and Formula (XV), wherein the amino acid residue in SEQ ID NO: 3 that is replaced is selected from E61 and P64, and wherein n is an integer from about 450 to about 800, or from about 454 to about 796, or from about 454 to about 682, or from about 568 to about 909. In some embodiments, n in the compounds of Formula (XIV) and Formula (XV) is an integer selected from 454, 455, 568, 569, 680, 681, 682, 794, 795, 796, 908, 909, and 910.

In some embodiments, the IL-2 conjugate comprises the amino acid sequence of SEQ ID NO: 3 in which at least one amino acid residue in the IL-2 conjugate is replaced by the structure of Formula (XIV) or Formula (XV), or a mixture of Formula (XIV) and Formula (XV), wherein the amino acid residue in SEQ ID NO: 3 that is replaced is E61, and wherein n is an integer from about 450 to about 800, or from about 454 to about 796, or from about 454 to about 682, or from about 568 to about 909. In some embodiments, n in the compounds of Formula (XIV) and Formula (XV) is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

In some embodiments, the IL-2 conjugate comprises the amino acid sequence of SEQ ID NO: 3 in which at least one amino acid residue in the IL-2 conjugate is replaced by the structure of Formula (XIV) or Formula (XV), or a mixture of Formula (XIV) and Formula (XV), wherein the amino acid residue in in SEQ ID NO: 3 that is replaced is P64, and wherein n is an integer from about 450 to about 800, or from about 454 to about 796, or from about 454 to about 682, or from about 568 to about 909. In some embodiments, n in the compounds of Formula (XIV) and Formula (XV) is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

In some embodiments, the IL-2 conjugate comprises the amino acid sequence of SEQ ID NO: 3 in which at least one amino acid residue in the IL-2 conjugate is replaced by the structure of Formula (XIV) or Formula (XV), or a mixture of Formula (XIV) and Formula (XV), wherein n is an integer such that the molecular weight of the PEG moiety is in the range from about 1,000 Daltons about 200,000 Daltons, or from about 2,000 Daltons to about 150,000 Daltons, or from about 3,000 Daltons to about 125,000 Daltons, or from about 4,000 Daltons to about 100,000 Daltons, or from about 5,000 Daltons to about 100,000 Daltons, or from about 6,000 Daltons to about 90,000 Daltons, or from about 7,000 Daltons to about 80,000 Daltons, or from about 8,000 Daltons to about 70,000 Daltons, or from about 5,000 Daltons to about 70,000 Daltons, or from about 5,000 Daltons to about 65,000 Daltons, or from about 5,000 Daltons to about 60,000 Daltons, or from about 5,000 Daltons to about 50,000 Daltons, or from about 6,000 Daltons to about 50,000 Daltons, or from about 7,000 Daltons to about 50,000 Daltons, or from about 7,000 Daltons to about 45,000 Daltons, or from about 7,000 Daltons to about 40,000 Daltons, or from about 8,000 Daltons to about 40,000 Daltons, or from about 8,500 Daltons to about 40,000 Daltons, or from about 8,500 Daltons to about 35,000 Daltons, or from about 9,000 Daltons to about 50,000 Daltons, or from about 9,000 Daltons to about 45,000 Daltons, or from about 9,000 Daltons to about 40,000 Daltons, or from about 9,000 Daltons to about 35,000 Daltons, or from about 9,000 Daltons to about 30,000 Daltons, or from about 9,500 Daltons to about 35,000 Daltons, or from about 9,500 Daltons to about 30,000 Daltons, or from about 10,000 Daltons to about 50,000 Daltons, or from about 10,000 Daltons to about 45,000 Daltons, or from about 10,000 Daltons to about 40,000 Daltons, or from about 10,000 Daltons to about 35,000 Daltons, or from about 10,000 Daltons to about 30,000 Daltons, or from about 15,000 Daltons to about 50,000 Daltons, or from about 15,000 Daltons to about 45,000 Daltons, or from about 15,000 Daltons to about 40,000 Daltons, or from about 15,000 Daltons to about 35,000 Daltons, or from about 15,000 Daltons to about 30,000 Daltons, or from about 20,000 Daltons to about 50,000 Daltons, or from about 20,000 Daltons to about 45,000 Daltons, or from about 20,000 Daltons to about 40,000 Daltons, or from about 20,000 Daltons to about 35,000 Daltons, or from about 20,000 Daltons to about 30,000 Daltons. In some embodiments, the IL-2 conjugate comprises the amino acid sequence of SEQ ID NO: 3 in which at least one amino acid residue in the IL-2 conjugate is replaced by the structure of Formula (XIV) or Formula (XV), or a mixture of Formula (XIV) and Formula (XV), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, about 50,000 Daltons, about 60,000 Daltons, about 70,000 Daltons, about 80,000 Daltons, about 90,000 Daltons, about 100,000 Daltons, about 125,000 Daltons, about 150,000 Daltons, about 175,000 Daltons or about 200,000 Daltons. In some embodiments, the IL-2 conjugate comprises the amino acid sequence of SEQ ID NO: 3 in which at least one amino acid residue in the IL-2 conjugate is replaced by the structure of Formula (XIV) or Formula (XV), or a mixture of Formula (XIV) and Formula (XV), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, or about 50,000 Daltons.

In some embodiments, the IL-2 conjugate comprises the amino acid sequence of SEQ ID NO: 3 in which at least one amino acid residue in the IL-2 conjugate is replaced by the structure of Formula (XIV) or Formula (XV), or a mixture of Formula (XIV) and Formula (XV), wherein the amino acid residue in in SEQ ID NO: 3 that is replaced is selected from F41, F43, K42, E61, and P64, m is an integer from 1 to 6, p is an integer from 1 to 6, and n is an integer from about 450 to about 800, or from about 454 to about 796, or from about 454 to about 682, or from about 568 to about 909. In some embodiments of the compounds of Formula (XIV) and Formula (XV), m is 2, p is 2, and n is an integer selected from 454, 455, 568, 569, 680, 681, 682, 794, 795, 796, 908, 909, 910, 1021, 1022, 1023, 1135, 1136, 1137, and 1249.

In some embodiments, the IL-2 conjugate comprises the amino acid sequence of SEQ ID NO: 3 in which at least one amino acid residue in the IL-2 conjugate is replaced by the structure of Formula (XIV) or Formula (XV), or a mixture of Formula (XIV) and Formula (XV), wherein the amino acid residue in SEQ ID NO: 3 that is replaced is selected from E61 and P64, and wherein m is an integer from 1 to 6, p is an integer from 1 to 6, and n is an integer from about 450 to about 800, or from about 454 to about 796, or from about 454 to about 682, or from about 568 to about 909. In some embodiments of the compounds of Formula (XIV) and Formula (XV), m is 2, p is 2, and n is an integer selected from 454, 455, 568, 569, 680, 681, 682, 794, 795, 796, 908, 909, and 910.

In some embodiments, the IL-2 conjugate comprises the amino acid sequence of SEQ ID NO: 3 in which at least one amino acid residue in the IL-2 conjugate is replaced by the structure of Formula (XIV) or Formula (XV), or a mixture of Formula (XIV) and Formula (XV), wherein the amino acid residue in SEQ ID NO: 3 that is replaced is E61, and wherein m is an integer from 1 to 6, p is an integer from 1 to 6, and n is an integer from about 450 to about 800, or from about 454 to about 796, or from about 454 to about 682, or from about 568 to about 909. In some embodiments of the compounds of Formula (XIV) and Formula (XV), m is 2, p is 2, and n is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

In some embodiments, the IL-2 conjugate comprises the amino acid sequence of SEQ ID NO: 3 in which at least one amino acid residue in the IL-2 conjugate is replaced by the structure of Formula (XIV) or Formula (XV), or a mixture of Formula (XIV) and Formula (XV), wherein the amino acid residue in SEQ ID NO: 3 that is replaced is P64, and wherein m is an integer from 1 to 6, p is an integer from 1 to 6, and n is an integer from about 450 to about 800, or from about 454 to about 796, or from about 454 to about 682, or from about 568 to about 909. In some embodiments of the compounds of Formula (XIV) and Formula (XV), m is 2, p is 2, and n is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

In some embodiments, the IL-2 conjugate comprises the amino acid sequence of SEQ ID NO: 3 in which at least one amino acid residue in the IL-2 conjugate is replaced by the structure of Formula (XVI) or Formula (XVII), or a mixture of Formula (XVI) and Formula (XVII):

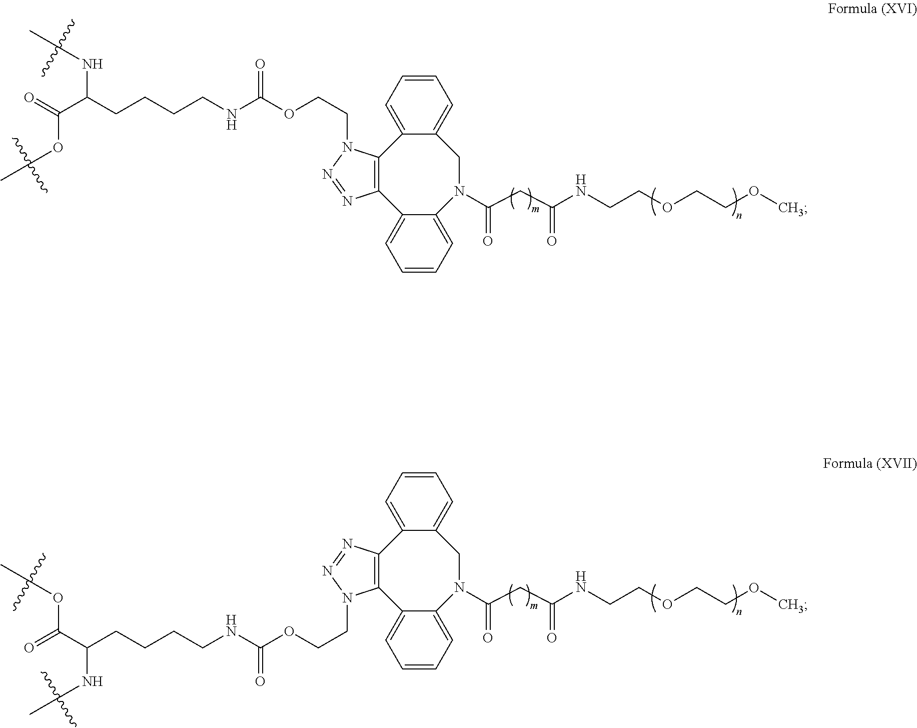

wherein:
m is an integer from 0 to 20;
n is an integer in the range from about 2 to about 5000; and
the wavy lines indicate covalent bonds to amino acid residues within SEQ ID NO: 3 that are not replaced.

Here and throughout, the structure of Formula (XVI) encompasses pharmaceutically acceptable salts, solvates, or hydrates thereof. Here and throughout, the structure of Formula (XVII) encompasses pharmaceutically acceptable salts, solvates, or hydrates thereof. In some embodiments, the IL-2 conjugate is a pharmaceutically acceptable salt, solvate, or hydrate.

In some embodiments, the stereochemistry of the chiral center within Formula (XVI) and Formula (XVII) is racemic, is enriched in (R), is enriched in (S), is substantially (R), is substantially (S), is (R) or is (S). In some embodiments, the stereochemistry of the chiral center within Formula (XVI) and Formula (XVII) is racemic. In some embodiments, the stereochemistry of the chiral center within Formula (XVI) and Formula (XVII) is enriched in (R). In some embodiments, the stereochemistry of the chiral center within Formula (XVI) and Formula (XVII) is enriched in (S). In some embodiments, the stereochemistry of the chiral center within Formula (XVI) and Formula (XVII) is substantially (R). In some embodiments, the stereochemistry of the chiral center within Formula (XVI) and Formula (XVII) is substantially (S). In some embodiments, the stereochemistry of the chiral center within Formula (XVI) and Formula (XVII) is (R). In some embodiments, the stereochemistry of the chiral center within Formula (XVI) and Formula (XVII) is (S).

In some embodiments, the IL-2 conjugate comprises the amino acid sequence of SEQ ID NO: 3 in which m in the compounds of Formula (XVI) and Formula (XVII) is from 1 to 20, or from 1 to 18, or from 1 to 16, or from 1 to 14, or from 1 to 12, or from 1 to 10, or from 1 to 9, or from 1 to 8, or from 1 to 7, or from 1 to 6, or from 1 to 5, or from 1 to 4, or from 1 to 3, or from 1 to 2. In some embodiments, m in the compounds of Formula (XVI) and Formula (XVII) is 1. In some embodiments, m in the compounds of Formula (XVI) and Formula (XVII) is 2. In some embodiments, m in the compounds of Formula (XVI) and Formula (XVII) is 3. In some embodiments, m in the compounds of Formula (XVI) and Formula (XVII) is 4. In some embodiments, m in the compounds of Formula (XVI) and Formula (XVII) is 5. In some embodiments, m in the compounds of Formula (XVI) and Formula (XVII) is 6. In some embodiments, m in the compounds of Formula (XVI) and Formula (XVII) is 7. In some embodiments, m in the compounds of Formula (XVI) and Formula (XVII) is 8. In some embodiments, m in the compounds of Formula (XVI) and Formula (XVII) is 9. In some embodiments, m in the compounds of Formula (XVI) and Formula (XVII) is 10. In some embodiments, m in the compounds of Formula (XVI) and Formula (XVII) is 11. In some embodiments, m in the compounds of Formula (XVI) and Formula (XVII) is 12. In some embodiments, m in the compounds of Formula (XVI) and Formula (XVII) is 13. In some embodiments, m in the compounds of Formula (XVI) and Formula (XVII) is 14. In some embodiments, m in the compounds of Formula (XVI) and Formula (XVII) is 15. In some embodiments, m in the compounds of Formula (XVI) and Formula (XVII) is 16. In some embodiments, m in the compounds of Formula (XVI) and Formula (XVII) is 17. In some embodiments, m in the compounds of Formula (XVI) and Formula (XVII) is 18. In some embodiments, m in the compounds of Formula (XVI) and Formula (XVII) is 19. In some embodiments, m in the compounds of Formula (XVI) and Formula (XVII) is 20.

In some embodiments, the IL-2 conjugate comprises the amino acid sequence of SEQ ID NO: 3 in which n in the compounds of Formula (XVI) and Formula (XVII) is in the range from about 5 to about 4600, or from about 10 to about 4000, or from about 20 to about 3000, or from about 100 to about 3000, or from about 100 to about 2900, or from about 150 to about 2900, or from about 125 to about 2900, or from about 100 to about 2500, or from about 100 to about 2000, or from about 100 to about 1900, or from about 100 to about 1850, or from about 100 to about 1750, or from about 100 to about 1650, or from about 100 to about 1500, or from about 100 to about 1400, or from about 100 to about 1300, or from about 100 to about 1250, or from about 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575.

In some embodiments, the IL-2 conjugate comprises the amino acid sequence of SEQ ID NO: 3 in which m in the compounds of Formula (XVI) and Formula (XVII) is an integer from 1 to 6, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XVI) and Formula (XVII), m is an integer from 2 to 6, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XVI) and Formula (XVII), m is an integer from 2 to 4, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XVI) and Formula (XVII), m is 1, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XVI) and Formula (XVII), m is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XVI) and Formula (XVII), m is 3, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XVI) and Formula (XVII), m is 4, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XVI) and Formula (XVII), m is 5, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XVI) and Formula (XVII), m is 6, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XVI) and Formula (XVII), m is 7, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XVI) and Formula (XVII), m is 8, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XVI) and Formula (XVII), m is 9, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XVI) and Formula (XVII), m is 10, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XVI) and Formula (XVII), m is 11, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XVI) and Formula (XVII), m is 12, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of the compounds of Formula (XVI) and Formula (XVII), m is 2, and n is an integer selected from 680, 681, 682, 794, 795, 796, 908, 909, 910, 1021, 1022, 1023, 1135, 1136, and 1137.

In some embodiments, the IL-2 conjugate comprises the amino acid sequence of SEQ ID NO: 3 in which n in the compounds of Formula (XVI) and Formula (XVII) is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546. In some embodiments, the position of the structure of Formula (XVI), Formula (XVII), or a mixture of Formula (XVI) and Formula (XVII) in the amino acid sequence of the IL-2 conjugate is selected from K34, F41, F43, K42, E61, P64, R37, T40, E67, Y44, V68, and L71, wherein the position of the structure of Formula (XVI), Formula (XVII), or a mixture of Formula (XVI) and Formula (XVII) in the amino acid sequence of the IL-2 conjugate is in reference to the positions in SEQ ID NO: 3. In some embodiments, the position of the structure of Formula (XVI), Formula (XVII), or a mixture of Formula (XVI) and Formula (XVII) in the amino acid sequence of the IL-2 conjugate of SEQ ID NO: 3 is selected from K34, F41, F43, K42, E61, P64, R37, T40, E67, Y44, V68, and L71. In some embodiments, the position of the structure of Formula (XVI), Formula (XVII), or a mixture of Formula (XVI) and Formula (XVII) in the amino acid sequence of the IL-2 conjugate of SEQ ID NO: 3 is at position K34. In some embodiments, the position of the structure of Formula (XVI), Formula (XVII), or a mixture of Formula (XVI) and Formula (XVII) in the amino acid sequence of the IL-2 conjugate of SEQ ID NO: 3 is at position F41. In some embodiments, the position of the structure of Formula (XVI), Formula (XVII), or a mixture of Formula (XVI) and Formula (XVII) in the amino acid sequence of the IL-2 conjugate of SEQ ID NO: 3 is at position F43. In some embodiments, the position of the structure of Formula (XVI), Formula (XVII), or a mixture of Formula (XVI) and Formula (XVII) in the amino acid sequence of the IL-2 conjugate of SEQ ID NO: 3 is at position K42. In some embodiments, the position of the structure of Formula (XVI), Formula (XVII), or a mixture of Formula (XVI) and Formula (XVII) in the amino acid sequence of the IL-2 conjugate of SEQ ID NO: 3 is at position E61. In some embodiments, the position of the structure of Formula (XVI), Formula (XVII), or a mixture of Formula (XVI) and Formula (XVII) in the amino acid sequence of the IL-2 conjugate of SEQ ID NO: 3 is at position P64. In some embodiments, the position of the structure of Formula (XVI), Formula (XVII), or a mixture of Formula (XVI) and Formula (XVII) in the amino acid sequence of the IL-2 conjugate of SEQ ID NO: 3 is at position R37. In some embodiments, the position of the structure of Formula (XVI), Formula (XVII), or a mixture of Formula (XVI) and Formula (XVII) in the amino acid sequence of the IL-2 conjugate of SEQ ID NO: 3 is at position T40. In some embodiments, the position of the structure of Formula (XVI), Formula (XVII), or a mixture of Formula (XVI) and Formula (XVII) in the amino acid sequence of the IL-2 conjugate of SEQ ID NO: 3 is at position E67. In some embodiments, the position of the structure of Formula (XVI), Formula (XVII), or a mixture of Formula (XVI) and Formula (XVII) in the amino acid sequence of the IL-2 conjugate of SEQ ID NO: 3 is at position Y44. In some embodiments, the position of the structure of Formula (XVI), Formula (XVII), or a mixture of Formula (XVI) and Formula (XVII) in the amino acid sequence of the IL-2 conjugate of SEQ ID NO: 3 is at position V68. In some embodiments, the position of the structure of Formula (XVI), Formula (XVII), or a mixture of Formula (XVI) and Formula (XVII) in the amino acid sequence of the IL-2 conjugate of SEQ ID NO: 3 is at position L71. In some embodiments, the ratio of the amount of the structure of Formula (XVI) to the amount of the structure of Formula (XVII) comprising the total amount of the IL-2 conjugate is about 1:1. In some embodiments, the ratio of the amount of the structure of Formula (XVI) to the amount of the structure of Formula (XVII) comprising the total amount of the IL-2 conjugate is greater than 1:1. In some embodiments, the ratio of the amount of the structure of Formula (XVI) to the amount of the structure of Formula (XVII) comprising the total amount of the IL-2 conjugate is less than 1:1.

In some embodiments, the IL-2 conjugate comprises the amino acid sequence of SEQ ID NO: 3 in which the at least one amino acid residue in the IL-2 conjugate is replaced by the structure of Formula (XVI), Formula (XVII), or a mixture of Formula (XVI) and Formula (XVII) is selected from K34, F41, F43, K42, E61, P64, R37, T40, E67, Y44, V68, and L71, and wherein n is an integer from 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575. In some embodiments, n in the compounds of Formula (XVI) and Formula (XVII) is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546.

In some embodiments, the IL-2 conjugate comprises the amino acid sequence of SEQ ID NO: 3 in which the at least one amino acid residue in the IL-2 conjugate replaced by the structure of Formula (XVI), Formula (XVII), or a mixture of Formula (XVI) and Formula (XVII), is selected from F41, F43, K42, E61, and P64, and wherein n is an integer from about 450 to about 800, or from about 454 to about 796, or from about 454 to about 682, or from about 568 to about 909. In some embodiments, n in the compounds of Formula (XVI) and Formula (XVII) is an integer selected from 454, 455, 568, 569, 680, 681, 682, 794, 795, 796, 908, 909, 910, 1021, 1022, 1023, 1135, 1136, 1137, and 1249.

In some embodiments, the IL-2 conjugate comprises the amino acid sequence of SEQ ID NO: 3 in which the at least one amino acid residue in the IL-2 conjugate replaced by the structure of Formula (XVI), Formula (XVII), or a mixture of Formula (XVI) and Formula (XVII), is selected from E61 and P64, and wherein n is an integer from about 450 to about 800, or from about 454 to about 796, or from about 454 to about 682, or from about 568 to about 909. In some embodiments, n in the compounds of Formula (XVI) and Formula (XVII) is an integer selected from 454, 455, 568, 569, 680, 681, 682, 794, 795, 796, 908, 909, and 910.

In some embodiments, the IL-2 conjugate comprises the amino acid sequence of SEQ ID NO: 3 in which the at least one amino acid residue in the IL-2 conjugate replaced by the structure of Formula (XVI), Formula (XVII), or a mixture of Formula (XVI) and Formula (XVII), that is replaced is E61, and wherein n is an integer from about 450 to about 800, or from about 454 to about 796, or from about 454 to about 682, or from about 568 to about 909. In some embodiments, n in the compounds of Formula (XVI) and Formula (XVII) is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

In some embodiments, the IL-2 conjugate comprises the amino acid sequence of SEQ ID NO: 3 in which the at least one amino acid residue in the IL-2 conjugate is replaced by the structure of Formula (XVI), Formula (XVII), or a mixture of Formula (XVI) and Formula (XVII), that is replaced is P64, and wherein n is an integer from about 450 to about 800, or from about 454 to about 796, or from about 454 to about 682, or from about 568 to about 909. In some embodiments, n in the compounds of Formula (XVI) and Formula (XVII) is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

In some embodiments, the IL-2 conjugate comprises the amino acid sequence of SEQ ID NO: 3 in which the at least one amino acid residue in the IL-2 conjugate replaced by the structure of Formula (XVI), Formula (XVII), or a mixture of Formula (XVI) and Formula (XVII), wherein n is an integer such that the molecular weight of the PEG moiety is in the range from about 1,000 Daltons about 200,000 Daltons, or from about 2,000 Daltons to about 150,000 Daltons, or from about 3,000 Daltons to about 125,000 Daltons, or from about 4,000 Daltons to about 100,000 Daltons, or from about 5,000 Daltons to about 100,000 Daltons, or from about 6,000 Daltons to about 90,000 Daltons, or from about 7,000 Daltons to about 80,000 Daltons, or from about 8,000 Daltons to about 70,000 Daltons, or from about 5,000 Daltons to about 70,000 Daltons, or from about 5,000 Daltons to about 65,000 Daltons, or from about 5,000 Daltons to about 60,000 Daltons, or from about 5,000 Daltons to about 50,000 Daltons, or from about 6,000 Daltons to about 50,000 Daltons, or from about 7,000 Daltons to about 50,000 Daltons, or from about 7,000 Daltons to about 45,000 Daltons, or from about 7,000 Daltons to about 40,000 Daltons, or from about 8,000 Daltons to about 40,000 Daltons, or from about 8,500 Daltons to about 40,000 Daltons, or from about 8,500 Daltons to about 35,000 Daltons, or from about 9,000 Daltons to about 50,000 Daltons, or from about 9,000 Daltons to about 45,000 Daltons, or from about 9,000 Daltons to about 40,000 Daltons, or from about 9,000 Daltons to about 35,000 Daltons, or from about 9,000 Daltons to about 30,000 Daltons, or from about 9,500 Daltons to about 35,000 Daltons, or from about 9,500 Daltons to about 30,000 Daltons, or from about 10,000 Daltons to about 50,000 Daltons, or from about 10,000 Daltons to about 45,000 Daltons, or from about 10,000 Daltons to about 40,000 Daltons, or from about 10,000 Daltons to about 35,000 Daltons, or from about 10,000 Daltons to about 30,000 Daltons, or from about 15,000 Daltons to about 50,000 Daltons, or from about 15,000 Daltons to about 45,000 Daltons, or from about 15,000 Daltons to about 40,000 Daltons, or from about 15,000 Daltons to about 35,000 Daltons, or from about 15,000 Daltons to about 30,000 Daltons, or from about 20,000 Daltons to about 50,000 Daltons, or from about 20,000 Daltons to about 45,000 Daltons, or from about 20,000 Daltons to about 40,000 Daltons, or from about 20,000 Daltons to about 35,000 Daltons, or from about 20,000 Daltons to about 30,000 Daltons. In some embodiments, the IL-2 conjugate comprises the amino acid sequence of SEQ ID NO: 3 in which at least one amino acid residue in the IL-2 conjugate is replaced by the structure of Formula (XVI), Formula (XVII), or a mixture of Formula (XVI) and Formula (XVII), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, about 50,000 Daltons, about 60,000 Daltons, about 70,000 Daltons, about 80,000 Daltons, about 90,000 Daltons, about 100,000 Daltons, about 125,000 Daltons, about 150,000 Daltons, about 175,000 Daltons or about 200,000 Daltons. In some embodiments, the IL-2 conjugate comprises the amino acid sequence of SEQ ID NO: 3 in which at least one amino acid residue in the IL-2 conjugate is replaced by the structure of Formula (XVI), Formula (XVII), or a mixture of Formula (XVI) and Formula (XVII), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, or about 50,000 Daltons.

In some embodiments, the IL-2 conjugate comprises the amino acid sequence of SEQ ID NO: 3 in which the at least one amino acid residue in the IL-2 conjugate is replaced by the structure of Formula (XVI), Formula (XVII), or a mixture of Formula (XVI) and Formula (XVII), is selected from F41, F43, K42, E61, and P64, m is an integer from 1 to 6, and n is an integer from about 450 to about 800, or from about 454 to about 796, or from about 454 to about 682, or from about 568 to about 909. In some embodiments of the compounds of Formula (XVI) and Formula (XVII), m is 2, and n is an integer selected from 454, 455, 568, 569, 680, 681, 682, 794, 795, 796, 908, 909, 910, 1021, 1022, 1023, 1135, 1136, 1137, and 1249.

In some embodiments, the IL-2 conjugate comprises the amino acid sequence of SEQ ID NO: 3 in which the at least one amino acid residue in the IL-2 conjugate replaced by the structure of Formula (XVI), Formula (XVII), or a mixture of Formula (XVI) and Formula (XVII), is selected from E61 and P64, and wherein m is an integer from 1 to 6, and n is an integer from about 450 to about 800, or from about 454 to about 796, or from about 454 to about 682, or from about 568 to about 909. In some embodiments of the compounds of Formula (XVI) and Formula (XVII), m is 2, and n is an integer selected from 454, 455, 568, 569, 680, 681, 682, 794, 795, 796, 908, 909, and 910.

In some embodiments, the IL-2 conjugate comprises the amino acid sequence of SEQ ID NO: 3 in which the at least one amino acid residue in the IL-2 conjugate replaced by the structure of Formula (XVI), Formula (XVII), or a mixture of Formula (XVI) and Formula (XVII), that is replaced is E61, and wherein m is an integer from 1 to 6, and n is an integer from about 450 to about 800, or from about 454 to about 796, or from about 454 to about 682, or from about 568 to about 909. In some embodiments of the compounds of Formula (XVI) and Formula (XVII), m is 2, and n is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

In some embodiments, the IL-2 conjugate comprises the amino acid sequence of SEQ ID NO: 3 in which the at least one amino acid residue in the IL-2 conjugate replaced by the structure of Formula (XVI), Formula (XVII), or a mixture of Formula (XVI) and Formula (XVII), is P64, and wherein m is an integer from 1 to 6, and n is an integer from about 450 to about 800, or from about 454 to about 796, or from about 454 to about 682, or from about 568 to about 909. In some embodiments of the compounds of Formula (XVI) and Formula (XVII), m is 2, and n is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

In some embodiments, the IL-2 conjugate comprises SEQ ID NOs.: 1-98. In some embodiments, the IL-2 conjugate comprises SEQ ID NOs.: 15-29. In some embodiments, the IL-2 conjugate comprises SEQ ID NOs.: 40-54. In some embodiments, the IL-2 conjugate comprises SEQ ID NOs.: 55-69. In some embodiments, the IL-2 conjugate comprises SEQ ID NOs.: 70-84. In some embodiments, the IL-2 conjugate comprises a structure of Formula (I). In some embodiments, the IL-2 conjugate comprises a structure of Formula (II). In some embodiments, the IL-2 conjugate comprises a structure of Formula (III). In some embodiments, the IL-2 conjugate comprises a structure of Formula (IV). In some embodiments, the IL-2 conjugate comprises a structure of Formula (V). In some embodiments, the IL-2 conjugate comprises SEQ ID NO: 1. In some embodiments, the IL-2 conjugate comprises SEQ ID NO: 2. In some embodiments, the IL-2 conjugate comprises SEQ ID NO: 3. In some embodiments, the IL-2 conjugate comprises SEQ ID NO: 4. In some embodiments, the IL-2 conjugate comprises SEQ ID NO: 5. In some embodiments, the IL-2 conjugate comprises SEQ ID NO: 6. In some embodiments, the IL-2 conjugate comprises SEQ ID NO: 7. In some embodiments, the IL-2 conjugate comprises SEQ ID NO: 8. In some embodiments, the IL-2 conjugate comprises SEQ ID NO: 9. In some embodiments, the IL-2 conjugate comprises SEQ ID NO: 10. In some embodiments, the IL-2 conjugate comprises SEQ ID NO: 11. In some embodiments, the IL-2 conjugate comprises SEQ ID NO: 12. In some embodiments, the IL-2 conjugate comprises SEQ ID NO: 13. In some embodiments, the IL-2 conjugate comprises SEQ ID NO: 14. In some embodiments, the IL-2 conjugate comprises SEQ ID NO: 15. In some embodiments, the IL-2 conjugate comprises SEQ ID NO: 16. In some embodiments, the IL-2 conjugate comprises SEQ ID NO: 17. In some embodiments, the IL-2 conjugate comprises SEQ ID NO: 18. In some embodiments, the IL-2 conjugate comprises SEQ ID NO: 19. In some embodiments, the IL-2 conjugate comprises SEQ ID NO: 20. In some embodiments, the IL-2 conjugate comprises SEQ ID NO: 21. In some embodiments, the IL-2 conjugate comprises SEQ ID NO: 22. In some embodiments, the IL-2 conjugate comprises SEQ ID NO: 23. In some embodiments, the IL-2 conjugate comprises SEQ ID NO: 24. In some embodiments, the IL-2 conjugate comprises SEQ ID NO: 25. In some embodiments, the IL-2 conjugate comprises SEQ ID NO: 26. In some embodiments, the IL-2 conjugate comprises SEQ ID NO: 27. In some embodiments, the IL-2 conjugate comprises SEQ ID NO: 28. In some embodiments, the IL-2 conjugate comprises SEQ ID NO: 24. In some embodiments, the IL-2 conjugate comprises SEQ ID NO: 25. In some embodiments, the IL-2 conjugate comprises SEQ ID NO: 26. In some embodiments, the IL-2 conjugate comprises SEQ ID NO: 27. In some embodiments, the IL-2 conjugate comprises SEQ ID NO: 28. In some embodiments, the IL-2 conjugate comprises SEQ ID NO: 29. In some embodiments, the IL-2 conjugate comprises SEQ ID NO: 30. In some embodiments, the IL-2 conjugate comprises SEQ ID NO: 31. In some embodiments, the IL-2 conjugate comprises SEQ ID NO: 32. In some embodiments, the IL-2 conjugate comprises SEQ ID NO: 33. In some embodiments, the IL-2 conjugate comprises SEQ ID NO: 34. In some embodiments, the IL-2 conjugate comprises SEQ ID NO: 35. In some embodiments, the IL-2 conjugate comprises SEQ ID NO: 36. In some embodiments, the IL-2 conjugate comprises SEQ ID NO: 37. In some embodiments, the IL-2 conjugate comprises SEQ ID NO: 38. In some embodiments, the IL-2 conjugate comprises SEQ ID NO: 39. In some embodiments, the IL-2 conjugate comprises SEQ ID NO: 40. In some embodiments, the IL-2 conjugate comprises SEQ ID NO: 41. In some embodiments, the IL-2 conjugate comprises SEQ ID NO: 42. In some embodiments, the IL-2 conjugate comprises SEQ ID NO: 43. In some embodiments, the IL-2 conjugate comprises SEQ ID NO: 44. In some embodiments, the IL-2 conjugate comprises SEQ ID NO: 45. In some embodiments, the IL-2 conjugate comprises SEQ ID NO: 46. In some embodiments, the IL-2 conjugate comprises SEQ ID NO: 47. In some embodiments, the IL-2 conjugate comprises SEQ ID NO: 48. In some embodiments, the IL-2 conjugate comprises SEQ ID NO: 49. In some embodiments, the IL-2 conjugate comprises SEQ ID NO: 50. In some embodiments, the IL-2 conjugate comprises SEQ ID NO: 51. In some embodiments, the IL-2 conjugate comprises SEQ ID NO: 52. In some embodiments, the IL-2 conjugate comprises SEQ ID NO: 53. In some embodiments, the IL-2 conjugate comprises SEQ ID NO: 54. In some embodiments, the IL-2 conjugate comprises SEQ ID NO: 55. In some embodiments, the IL-2 conjugate comprises SEQ ID NO: 56. In some embodiments, the IL-2 conjugate comprises SEQ ID NO: 57. In some embodiments, the IL-2 conjugate comprises SEQ ID NO: 58. In some embodiments, the IL-2 conjugate comprises SEQ ID NO: 59. In some embodiments, the IL-2 conjugate comprises SEQ ID NO: 60. In some embodiments, the IL-2 conjugate comprises SEQ ID NO: 61. In some embodiments, the IL-2 conjugate comprises SEQ ID NO: 62. In some embodiments, the IL-2 conjugate comprises SEQ ID NO: 63. In some embodiments, the IL-2 conjugate comprises SEQ ID NO: 64. In some embodiments, the IL-2 conjugate comprises SEQ ID NO: 65. In some embodiments, the IL-2 conjugate comprises SEQ ID NO: 66. In some embodiments, the IL-2 conjugate comprises SEQ ID NO: 67. In some embodiments, the IL-2 conjugate comprises SEQ ID NO: 68. In some embodiments, the IL-2 conjugate comprises SEQ ID NO: 69. In some embodiments, the IL-2 conjugate comprises SEQ ID NO: 70. In some embodiments, the IL-2 conjugate comprises SEQ ID NO: 71. In some embodiments, the IL-2 conjugate comprises SEQ ID NO: 72. In some embodiments, the IL-2 conjugate comprises SEQ ID NO: 73. In some embodiments, the IL-2 conjugate comprises SEQ ID NO: 74. In some embodiments, the IL-2 conjugate comprises SEQ ID NO: 75. In some embodiments, the IL-2 conjugate comprises SEQ ID NO: 76. In some embodiments, the IL-2 conjugate comprises SEQ ID NO: 77. In some embodiments, the IL-2 conjugate comprises SEQ ID NO: 78. In some embodiments, the IL-2 conjugate comprises SEQ ID NO: 79. In some embodiments, the IL-2 conjugate comprises SEQ ID NO: 80. In some embodiments, the IL-2 conjugate comprises SEQ ID NO: 81. In some embodiments, the IL-2 conjugate comprises SEQ ID NO: 82. In some embodiments, the IL-2 conjugate comprises SEQ ID NO: 83. In some embodiments, the IL-2 conjugate comprises SEQ ID NO: 84. In some embodiments, the IL-2 conjugate comprises SEQ ID NO: 85. In some embodiments, the IL-2 conjugate comprises SEQ ID NO: 86. In some embodiments, the IL-2 conjugate comprises SEQ ID NO: 87. In some embodiments, the IL-2 conjugate comprises SEQ ID NO: 88. In some embodiments, the IL-2 conjugate comprises SEQ ID NO: 89. In some embodiments, the IL-2 conjugate comprises SEQ ID NO: 90. In some embodiments, the IL-2 conjugate comprises SEQ ID NO: 91. In some embodiments, the IL-2 conjugate comprises SEQ ID NO: 92. In some embodiments, the IL-2 conjugate comprises SEQ ID NO: 93. In some embodiments, the IL-2 conjugate comprises SEQ ID NO: 94. In some embodiments, the IL-2 conjugate comprises SEQ ID NO: 95. In some embodiments, the IL-2 conjugate comprises SEQ ID NO: 96. In some embodiments, the IL-2 conjugate comprises SEQ ID NO: 97. In some embodiments, the IL-2 conjugate comprises SEQ ID NO: 98.

In some embodiments, the IL-2 conjugate comprises the amino acid sequence of any one of SEQ ID NOS: 86, 88, 90, 92, 94, 96, and 98. In any of these embodiments, the structure of Formula (I), or any variation thereof, such as Formula (II)-Formula (XV) or any variation thereof, is incorporated into the site comprising the unnatural amino acid.

In some embodiments, the IL-2 conjugate is modified at an amino acid position. In some instances, the modification is to a natural amino acid. In some instances, the modification is to an unnatural amino acid. In some instances, described herein is an isolated and modified IL-2 polypeptide that comprises at least one unnatural amino acid. In some cases, the IL-2 polypeptide comprises about 80%, 85%, 90%, 95%, 96%, 97%, 98%, or 99% sequence identity to any one of SEQ ID NOS: 3 to 84.

In some embodiments, the IL-2 conjugate further comprises an additional mutation. In some cases, the additional mutation is at an amino acid position selected from K35, T37, R38, T41, F42, K43, F44, Y45, E61, E62, E68, K64, P65, V69, L72, and Y107. In such cases, the amino acid is conjugated to an additional conjugating moiety for increase in serum half-life, stability, or a combination thereof. Alternatively, the amino acid is first mutated to a natural amino acid such as lysine, cysteine, histidine, arginine, aspartic acid, glutamic acid, serine, threonine, or tyrosine; or to an unnatural amino acid prior to binding to the additional conjugating moiety.

In some cases, the PEG group is not limited to a particular structure. In some cases, the PEG is linear (e.g., an end capped, e.g., alkoxy PEG or a bifunctional PEG), branched or multi-armed (e.g., forked PEG or PEG attached to a polyol core), a dendritic (or star) architecture, each with or without one or more degradable linkages. Moreover, the internal structure of the water-soluble polymer can be organized in any number of different repeat patterns and can be selected from the group consisting of homopolymer, alternating copolymer, random copolymer, block copolymer, alternating tripolymer, random tripolymer, and block tripolymer.

PEGs will typically comprise a number of (OCH₂CH₂) monomers [or (CH₂CH₂O) monomers, depending on how the PEG is defined]. As used herein, the number of repeating units is identified by the subscript “n” in “(OCH₂CH₂)ₙ.” Thus, the value of (n) typically falls within one or more of the following ranges: from 2 to about 3400, from about 100 to about 2300, from about 100 to about 2270, from about 136 to about 2050, from about 225 to about 1930, from about 450 to about 1930, from about 1200 to about 1930, from about 568 to about 2727, from about 660 to about 2730, from about 795 to about 2730, from about 795 to about 2730, from about 909 to about 2730, and from about 1,200 to about 1,900. For any given polymer in which the molecular weight is known, it is possible to determine the number of repeating units (i.e., “n”) by dividing the total weight-average molecular weight of the polymer by the molecular weight of the repeating monomer.

In some instances, the PEG is an end-capped polymer, that is, a polymer having at least one terminus capped with a relatively inert group, such as a lower C₁₋₆ alkoxy group, or a hydroxyl group. When the polymer is PEG, for example, a methoxy-PEG (commonly referred to as mPEG) may be used, which is a linear form of PEG wherein one terminus of the polymer is a methoxy (—OCH₃) group, while the other terminus is a hydroxyl or other functional group that can be optionally chemically modified.

In some embodiments, the PEG group comprising the IL-2 conjugates disclosed herein is a linear or branched PEG group. In some embodiments, the PEG group is a linear PEG group. In some embodiments, the PEG group is a branched PEG group. In some embodiments, the PEG group is a methoxy PEG group. In some embodiments, the PEG group is a linear or branched methoxy PEG group. In some embodiments, the PEG group is a linear methoxy PEG group. In some embodiments, the PEG group is a branched methoxy PEG group. In some embodiments, the PEG group is a linear or branched PEG group having an average molecular weight of from about 100 Daltons to about 150,000 Daltons. Exemplary ranges include, for example, weight-average molecular weights in the range of greater than 5,000 Daltons to about 100,000 Daltons, in the range of from about 6,000 Daltons to about 90,000 Daltons, in the range of from about 10,000 Daltons to about 85,000 Daltons, in the range of greater than 10,000 Daltons to about 85,000 Daltons, in the range of from about 20,000 Daltons to about 85,000 Daltons, in the range of from about 53,000 Daltons to about 85,000 Daltons, in the range of from about 25,000 Daltons to about 120,000 Daltons, in the range of from about 29,000 Daltons to about 120,000 Daltons, in the range of from about 35,000 Daltons to about 120,000 Daltons, and in the range of from about 40,000 Daltons to about 120,000 Daltons. Exemplary weight-average molecular weights for the PEG group include about 100 Daltons, about 200 Daltons, about 300 Daltons, about 400 Daltons, about 500 Daltons, about 600 Daltons, about 700 Daltons, about 750 Daltons, about 800 Daltons, about 900 Daltons, about 1,000 Daltons, about 1,500 Daltons, about 2,000 Daltons, about 2,200 Daltons, about 2,500 Daltons, about 3,000 Daltons, about 4,000 Daltons, about 4,400 Daltons, about 4,500 Daltons, about 5,000 Daltons, about 5,500 Daltons, about 6,000 Daltons, about 7,000 Daltons, about 7,500 Daltons, about 8,000 Daltons, about 9,000 Daltons, about 10,000 Daltons, about 11,000 Daltons, about 12,000 Daltons, about 13,000 Daltons, about 14,000 Daltons, about 15,000 Daltons, about 20,000 Daltons, about 22,500 Daltons, about 25,000 Daltons, about 30,000 Daltons, about 35,000 Daltons, about 40,000 Daltons, about 45,000 Daltons, about 50,000 Daltons, about 55,000 Daltons, about 60,000 Daltons, about 65,000 Daltons, about 70,000 Daltons, about 75,000 Daltons, about 80,000 Daltons, about 90,000 Daltons, about 95,000 Daltons, and about 100,000 Daltons. In some embodiments, the PEG group is a linear PEG group having an average molecular weight as disclosed above. In some embodiments, the PEG group is a branched PEG group having an average molecular weight as disclosed above. In some embodiments, the PEG group comprising the IL-2 conjugates disclosed herein is a linear or branched PEG group having a defined molecular weight±10%, or 15% or 20% or 25%. For example, included within the scope of the present disclosure are IL-2 conjugates comprising a PEG group having a molecular weight of 30,000 Da±3000 Da, or 30,000 Da±4,500 Da, or 30,000 Da±6,000 Da.

In some embodiments, the PEG group comprising the IL-2 conjugates disclosed herein is a linear or branched PEG group having an average molecular weight of from about 5,000 Daltons to about 60,000 Daltons. In some embodiments, the PEG group is a linear or branched PEG group having an average molecular weight of about 5,000 Daltons, about 5,500 Daltons, about 6,000 Daltons, about 7,000 Daltons, about 7,500 Daltons, about 8,000 Daltons, about 9,000 Daltons, about 10,000 Daltons, about 11,000 Daltons, about 12,000 Daltons, about 13,000 Daltons, about 14,000 Daltons, about 15,000 Daltons, about 20,000 Daltons, about 22,500 Daltons, about 25,000 Daltons, about 30,000 Daltons, about 35,000 Daltons, about 40,000 Daltons, about 45,000 Daltons, about 50,000 Daltons, about 55,000 Daltons, about 60,000 Daltons, about 65,000 Daltons, about 70,000 Daltons, about 75,000 Daltons, about 80,000 Daltons, about 90,000 Daltons, about 95,000 Daltons, and about 100,000 Daltons. In some embodiments, the PEG group is a linear or branched PEG group having an average molecular weight of about 5,000 Daltons, about 10,000 Daltons, about 20,000 Daltons, about 30,000 Daltons, about 50,000 Daltons, or about 60,000 Daltons. In some embodiments, the PEG group is a linear or branched PEG group having an average molecular weight of about 5,000 Daltons, about 30,000 Daltons, about 50,000 Daltons, or about 60,000 Daltons. In some embodiments, the PEG group is a linear PEG group having an average molecular of about 5,000 Daltons, about 10,000 Daltons, about 20,000 Daltons, about 30,000 Daltons, about 50,000 Daltons, or about 60,000 Daltons. In some embodiments, the PEG group is a branched PEG group having an average molecular weight of about 5,000 Daltons, about 10,000 Daltons, about 20,000 Daltons, about 30,000 Daltons, about 50,000 Daltons, or about 60,000 Daltons.

In some embodiments, the PEG group comprising the IL-2 conjugates disclosed herein is a linear methoxy PEG group having an average molecular weight of from about 5,000 Daltons to about 60,000 Daltons. In some embodiments, the PEG group is a linear methoxy PEG group having an average molecular weight of about 5,000 Daltons, about 5,500 Daltons, about 6,000 Daltons, about 7,000 Daltons, about 7,500 Daltons, about 8,000 Daltons, about 9,000 Daltons, about 10,000 Daltons, about 11,000 Daltons, about 12,000 Daltons, about 13,000 Daltons, about 14,000 Daltons, about 15,000 Daltons, about 20,000 Daltons, about 22,500 Daltons, about 25,000 Daltons, about 30,000 Daltons, about 35,000 Daltons, about 40,000 Daltons, about 45,000 Daltons, about 50,000 Daltons, about 55,000 Daltons, about 60,000 Daltons, about 65,000 Daltons, about 70,000 Daltons, about 75,000 Daltons, about 80,000 Daltons, about 90,000 Daltons, about 95,000 Daltons, and about 100,000 Daltons. In some embodiments, the PEG group is a linear methoxy PEG group having an average molecular weight of about 5,000 Daltons, about 10,000 Daltons, about 20,000 Daltons, about 30,000 Daltons, about 50,000 Daltons, or about 60,000 Daltons. In some embodiments, the PEG group is a linear methoxy PEG group having an average molecular weight of about 5,000 Daltons, about 30,000 Daltons, about 50,000 Daltons, or about 60,000 Daltons. In some embodiments, the PEG group is a linear methoxy PEG group having an average molecular of about 5,000 Daltons, about 10,000 Daltons, about 20,000 Daltons, about 30,000 Daltons, about 50,000 Daltons, or about 60,000 Daltons. In some embodiments, the PEG group is a linear methoxy PEG group having an average molecular of about 5,000 Daltons. In some embodiments, the PEG group is a linear methoxy PEG group having an average molecular of about 10,000 Daltons. In some embodiments, the PEG group is a linear methoxy PEG group having an average molecular of about 20,000 Daltons. In some embodiments, the PEG group is a linear methoxy PEG group having an average molecular of about 30,000 Daltons. In some embodiments, the PEG group is a linear methoxy PEG group having an average molecular of about 50,000 Daltons. In some embodiments, the PEG group is a linear methoxy PEG group having an average molecular of about 60,000 Daltons. In some embodiments, the PEG group comprising the IL-2 conjugates disclosed herein is a linear methoxy PEG group having a defined molecular weight±10%, or 15% or 20% or 25%. For example, included within the scope of the present disclosure are IL-2 conjugates comprising a linear methoxy PEG group having a molecular weight of 30,000 Da±3000 Da, or 30,000 Da±4,500 Da, or 30,000 Da±6,000 Da.

In some embodiments, the PEG group comprising the IL-2 conjugates disclosed herein is a branched methoxy PEG group having an average molecular weight of from about 5,000 Daltons to about 60,000 Daltons. In some embodiments, the PEG group is a branched methoxy PEG group having an average molecular weight of about 5,000 Daltons, about 5,500 Daltons, about 6,000 Daltons, about 7,000 Daltons, about 7,500 Daltons, about 8,000 Daltons, about 9,000 Daltons, about 10,000 Daltons, about 11,000 Daltons, about 12,000 Daltons, about 13,000 Daltons, about 14,000 Daltons, about 15,000 Daltons, about 20,000 Daltons, about 22,500 Daltons, about 25,000 Daltons, about 30,000 Daltons, about 35,000 Daltons, about 40,000 Daltons, about 45,000 Daltons, about 50,000 Daltons, about 55,000 Daltons, about 60,000 Daltons, about 65,000 Daltons, about 70,000 Daltons, about 75,000 Daltons, about 80,000 Daltons, about 90,000 Daltons, about 95,000 Daltons, and about 100,000 Daltons. In some embodiments, the PEG group is a branched methoxy PEG group having an average molecular weight of about 5,000 Daltons, about 10,000 Daltons, about 20,000 Daltons, about 30,000 Daltons, about 50,000 Daltons, or about 60,000 Daltons. In some embodiments, the PEG group is a branched methoxy PEG group having an average molecular weight of about 5,000 Daltons, about 30,000 Daltons, about 50,000 Daltons, or about 60,000 Daltons. In some embodiments, the PEG group is a branched methoxy PEG group having an average molecular of about 5,000 Daltons, about 10,000 Daltons, about 20,000 Daltons, about 30,000 Daltons, about 50,000 Daltons, or about 60,000 Daltons. In some embodiments, the PEG group is a branched methoxy PEG group having an average molecular of about 5,000 Daltons, about 10,000 Daltons, about 20,000 Daltons, about 30,000 Daltons, about 50,000 Daltons, or about 60,000 Daltons. In some embodiments, the PEG group is a branched methoxy PEG group having an average molecular of about 5,000 Daltons. In some embodiments, the PEG group is a branched methoxy PEG group having an average molecular of about 10,000 Daltons. In some embodiments, the PEG group is a branched methoxy PEG group having an average molecular of about 20,000 Daltons. In some embodiments, the PEG group is a branched methoxy PEG group having an average molecular of about 30,000 Daltons. In some embodiments, the PEG group is a branched methoxy PEG group having an average molecular of about 50,000 Daltons. In some embodiments, the PEG group is a branched methoxy PEG group having an average molecular of about 60,000 Daltons. In some embodiments, the PEG group comprising the IL-2 conjugates disclosed herein is a branched methoxy PEG group having a defined molecular weight±10%, or 15% or 20% or 25%. For example, included within the scope of the present disclosure are IL-2 conjugates comprising a branched methoxy PEG group having a molecular weight of 30,000 Da #3000 Da, or 30,000 Da±4,500 Da, or 30,000 Da±6,000 Da.

In some embodiments, exemplary PEG groups include, but are not limited to, linear or branched discrete PEG (dPEG) from Quanta Biodesign, Ltd; linear, branched, or forked PEGs from Nektar Therapeutics; and Y-shaped PEG derivatives from JenKem Technology.

In any of the embodiments or variations of Formula (I) described herein and pharmaceutical compositions comprising the same, average molecular weight encompasses both weight average molecular weight and number average molecular weight; in other words, for example, both a 30 kDa number average molecular weight and a 30 kDa weight average molecular weight qualify as a 30 kDa molecular weight. In some embodiments, the average molecular weight is weight average molecular weight. In other embodiments, the average molecular weight is number average molecular weight. It is understood that in the methods provided herein, administering an IL-2 conjugate as described herein to a subject comprises administering more than a single molecule of IL-2 conjugate; as such, use of the term “average” to describe the molecular weight of the PEG group refers to the average molecular weight of the PEG groups of the IL-2 conjugate molecules in a dose administered to the subject.

### Conjugation Chemistry

Various conjugation reactions are used to conjugate linkers, conjugation moieties, and unnatural amino acids incorporated into the IL-2 polypeptides described herein. Such conjugation reactions are often compatible with aqueous conditions, such as “bioorthogonal” reactions. In some embodiments, conjugation reactions are mediated by chemical reagents such as catalysts, light, or reactive chemical groups found on linkers, conjugation moieties, or unnatural amino acids. In some embodiments, conjugation reactions are mediated by enzymes. In some embodiments, a conjugation reaction used herein is described in Gong, Y., Pan, L. Tett. Lett. 2015, 56, 2123, the disclosure of which is herein incorporated by reference. In some embodiments, a conjugation reaction used herein is described in Chen, X.; Wu. Y-W. Org. Biomol. Chem. 2016, 14, 5417, the disclosure of which is herein incorporated by reference.

In some variation, the IL-2 conjugates described herein can be prepared by a conjugation reaction comprising a 1,3-dipolar cycloaddition reaction. In some embodiments, the 1,3-dipolar cycloaddition reaction comprises reaction of an azide and a phosphine (“Click” reaction). In some embodiments, the conjugation reaction is catalyzed by copper. In some embodiments, a conjugation reaction described herein results in cytokine peptide comprising a linker or conjugation moiety attached via a triazole. In some embodiments, a conjugation reaction described herein comprises reaction of an azide with a strained olefin. In some embodiments, a conjugation reaction described herein comprises reaction of an azide with a strained alkyne. In some embodiments, a conjugation reaction described herein comprises reaction of an azide with a cycloalkyne, for example DBCO.

In some embodiments described herein, a conjugation reaction described herein comprises the reaction outlined in Scheme 1, wherein X is the position in the IL-2 conjugate comprising an unnatural amino acid, such as in any one of SEQ ID NOS: 5, 6, 7, 8, 9, 30, 31, 32, 33, and 34.

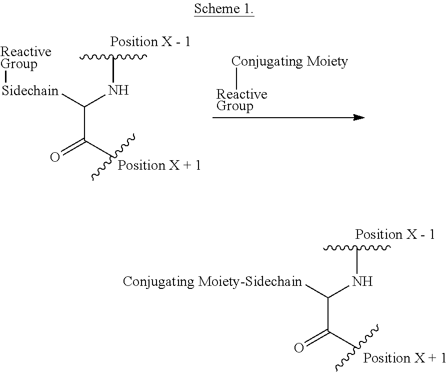

In some embodiments, the conjugating moiety comprises a water soluble polymer. In some embodiments, a reactive group comprises an alkyne or azide.

In some embodiments, a conjugation reaction described herein comprises the reaction outlined in Scheme 2, wherein X is the position in the IL-2 conjugate comprising an unnatural amino acid, such as in any one of SEQ ID NOS: 5, 6, 7, 8, 9, 30, 31, 32, 33, and 34.

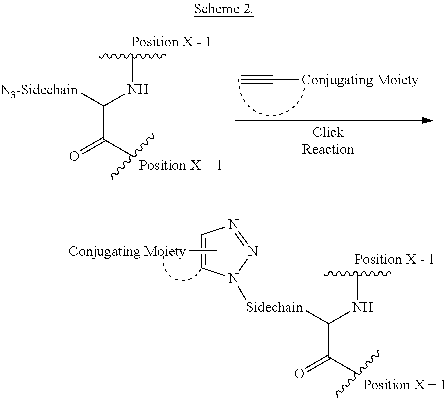

In some embodiments, a conjugation reaction described herein comprises the reaction outlined in Scheme 3, wherein X is the position in the IL-2 conjugate comprising an unnatural amino acid, such as in any one of SEQ ID NOS: 5, 6, 7, 8, 9, 30, 31, 32, 33, and 34.

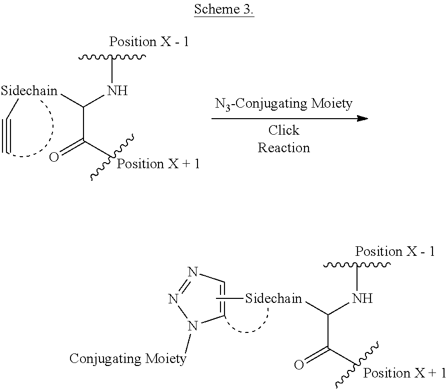

In some embodiments described herein, a conjugation reaction described herein comprises the reaction outlined in Scheme 4, wherein X is the position in the IL-2 conjugate comprising an unnatural amino acid, such as in any one of SEQ ID NOS: 5, 6, 7, 8, 9, 30, 31, 32, 33, and 34.

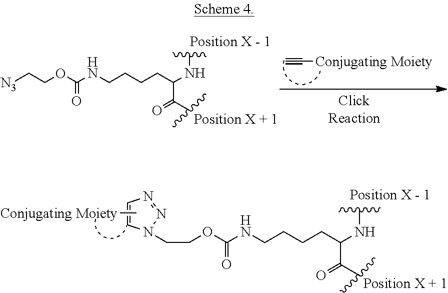

In some embodiments, a conjugation reaction described herein comprises a cycloaddition reaction between an azide moiety, such as that contained in a protein containing an amino acid residue derived from N6-((2-azidoethoxy)-carbonyl)-L-lysine (AzK), and a strained cycloalkyne, such as that derived from DBCO, which is a chemical moiety comprising a dibenzocyclooctyne group. PEG groups comprising a DBCO moiety are commercially available or may be prepared by methods know to those of ordinary skill in the art. An exemplary reaction is shown in Schemes 5 and 6.

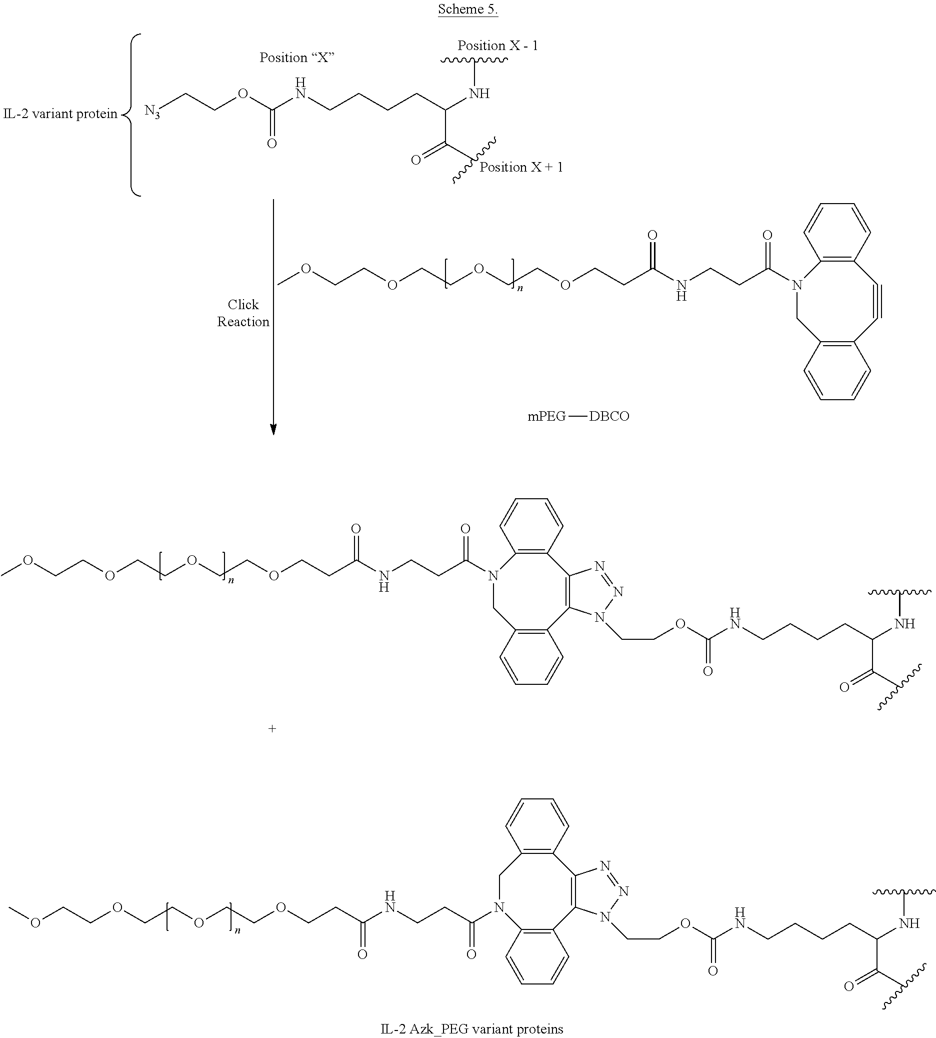

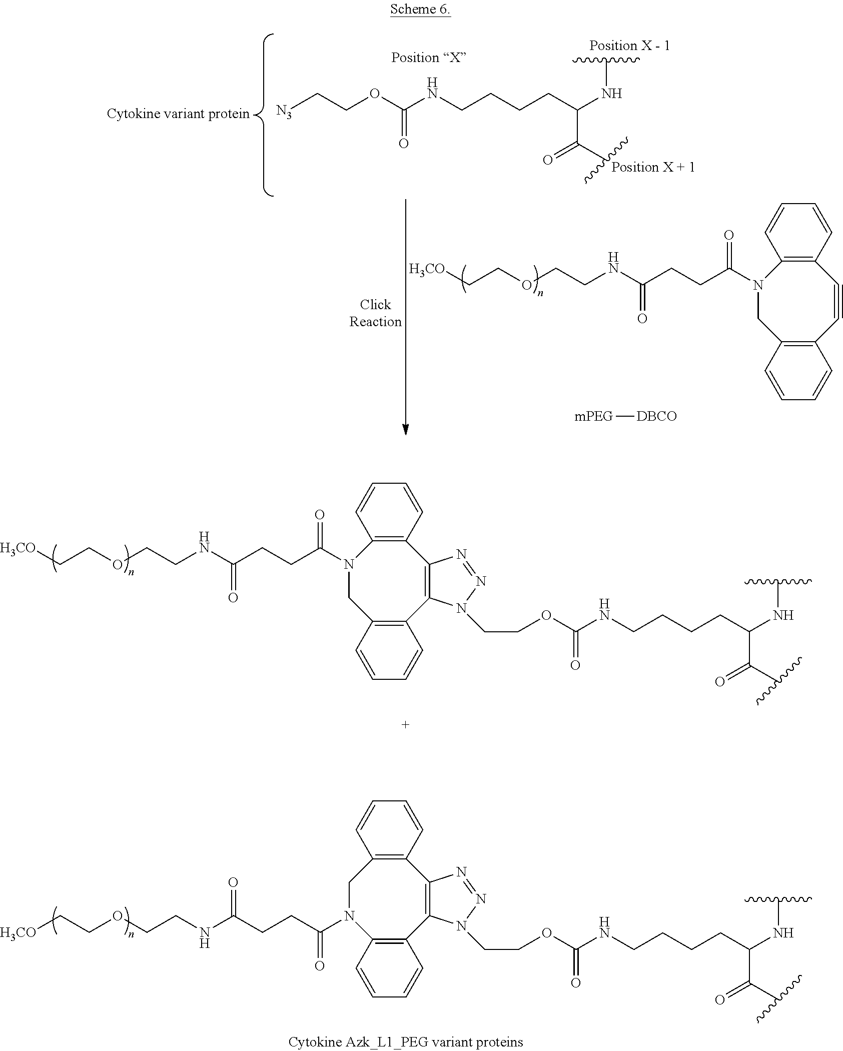

Conjugation reactions such as a click reaction described herein may generate a single regioisomer, or a mixture of regioisomers. In some instances, the ratio of regioisomers is about 1:1. In some instances, the ratio of regioisomers is about 2:1. In some instances, the ratio of regioisomers is about 1.5:1. In some instances, the ratio of regioisomers is about 1.2:1. In some instances, the ratio of regioisomers is about 1.1:1. In some instances the ratio of regioisomers is greater than 1:1.

### IL-2 Polypeptide Production

In some instances, the IL-2 conjugates described herein, either containing a natural amino acid mutation or an unnatural amino acid mutation, are generated recombinantly or are synthesized chemically. In some instances, IL-2 conjugates described herein are generated recombinantly, for example, either by a host cell system, or in a cell-free system. In any of the embodiments or variations described herein, the amino acid may be an L-amino acid or a D-amino acid. In some embodiments, the amino acid is an L-amino acid. In other embodiments, the amino acid is a D-amino acid.

In some instances, IL-2 conjugates are generated recombinantly through a host cell system. In some cases, the host cell is a eukaryotic cell (e.g., mammalian cell, insect cells, yeast cells or plant cell) or a prokaryotic cell (e.g., gram-positive bacterium or a gram-negative bacterium). In some cases, a eukaryotic host cell is a mammalian host cell. In some cases, a mammalian host cell is a stable cell line, or a cell line that has incorporated a genetic material of interest into its own genome and has the capability to express the product of the genetic material after many generations of cell division. In other cases, a mammalian host cell is a transient cell line, or a cell line that has not incorporated a genetic material of interest into its own genome and does not have the capability to express the product of the genetic material after many generations of cell division.

Exemplary mammalian host cells include 293T cell line, 293A cell line, 293 FT cell line, 293F cells, 293 H cells, A549 cells, MDCK cells, CHO DG44 cells, CHO-S cells, CHO-K1 cells, Expi293F™ cells, Flp-In™ T-REX™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REX™ Jurkat cell line, Per.C6 cells, T-REX™-293 cell line, T-REX™-CHO cell line, and T-REX™-HeLa cell line.

In some embodiments, a eukaryotic host cell is an insect host cell. Exemplary insect host cell include Drosophila S2 cells, Sf9 cells, Sf21 cells, High Five™ cells, and expresSF+® cells.

In some embodiments, a eukaryotic host cell is a yeast host cell. Exemplary yeast host cells include Pichia pastoris (K. phaffii) yeast strains such as GS115, KM71H, SMD1168, SMD1168H, and X−33, and Saccharomyces cerevisiae yeast strain such as INVScl.

In some embodiments, a eukaryotic host cell is a plant host cell. In some instances, the plant cells comprise a cell from algae. Exemplary plant cell lines include strains from Chlamydomonas reinhardtii 137c, or Synechococcus elongatus PPC 7942.

In some embodiments, a host cell is a prokaryotic host cell. Exemplary prokaryotic host cells include BL21, Mach 1™, DH10B™, TOP10, DH5a, DH10Bac™, OmniMax™, MegaX™, DH12S™, INV110, TOP10F′, INVαF, TOP10/P3, ccdB Survival, PIR1, PIR2, Stb12™, Stb13™, or Stb14™.

In some instances, suitable polynucleic acid molecules or vectors for the production of an IL-2 polypeptide described herein include any suitable vectors derived from either a eukaryotic or prokaryotic source. Exemplary polynucleic acid molecules or vectors include vectors from bacteria (e.g., E. coli), insects, yeast (e.g., Pichia pastoris, K. phaffii), algae, or mammalian source. Bacterial vectors include, for example, pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Insect vectors include, for example, pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT2, or MAT vectors such as pPolh-MAT1, or pPolh-MAT2.

Yeast vectors include, for example, Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichi pastoris (K. phaffii) vector, pGAPZA, B, & C Pichia pastoris (K. phaffii) vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Algae vectors include, for example, pChlamy-4 vector or MCS vector.

Mammalian vectors include, for example, transient expression vectors or stable expression vectors. Exemplary mammalian transient expression vectors include p3×FLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3×FLAG-CMV 7.1, pFLAG-CMV 20, p3×FLAG-Myc-CMV 24, pCMV-FLAG-MATI, pCMV-FLAG-MAT2, pBICEP-CMV 3, or pBICEP-CMV 4. Exemplary mammalian stable expression vectors include pFLAG-CMV 3, p3×FLAG-CMV 9, p3×FLAG-CMV 13, pFLAG-Myc-CMV 21, p3×FLAG-Myc-CMV 25, pFLAG-CMV 4, p3×FLAG-CMV 10, p3×FLAG-CMV 14, pFLAG-Myc-CMV 22, p3×FLAG-Myc-CMV 26, pBICEP-CMV 1, or pBICEP-CMV 2.

In some instances, a cell-free system is used for the production of an IL-2 polypeptide described herein. In some cases, a cell-free system comprises a mixture of cytoplasmic and/or nuclear components from a cell and is suitable for in vitro nucleic acid synthesis. In some instances, a cell-free system utilizes prokaryotic cell components. In other instances, a cell-free system utilizes eukaryotic cell components. Nucleic acid synthesis is obtained in a cell-free system based on, for example, Drosophila cell, Xenopus egg, Archaea, or HeLa cells. Exemplary cell-free systems include E. coli S30 Extract system, E. coli T7 S30 system, or PURExpress®, XpressCF, and XpressCF+.

Cell-free translation systems variously comprise components such as plasmids, mRNA, DNA, tRNAs, synthetases, release factors, ribosomes, chaperone proteins, translation initiation and elongation factors, natural and/or unnatural amino acids, and/or other components used for protein expression. Such components are optionally modified to improve yields, increase synthesis rate, increase protein product fidelity, or incorporate unnatural amino acids. In some embodiments, cytokines described herein are synthesized using cell-free translation systems described in U.S. Pat. No. 8,778,631; US 2017/0283469; US 2018/0051065; US 2014/0315245; or U.S. Pat. No. 8,778,631. In some embodiments, cell-free translation systems comprise modified release factors, or even removal of one or more release factors from the system. In some embodiments, cell-free translation systems comprise a reduced protease concentration. In some embodiments, cell-free translation systems comprise modified tRNAs with re-assigned codons used to code for unnatural amino acids. In some embodiments, the synthetases described herein for the incorporation of unnatural amino acids are used in cell-free translation systems. In some embodiments, tRNAs are pre-loaded with unnatural amino acids using enzymatic or chemical methods before being added to a cell-free translation system. In some embodiments, components for a cell-free translation system are obtained from modified organisms, such as modified bacteria, yeast, or other organism.

In some embodiments, an IL-2 polypeptide is generated as a circularly permuted form, either via an expression host system or through a cell-free system.

### Production of IL-2 Polypeptide Comprising an Unnatural Amino Acid

An orthogonal or expanded genetic code can be used in the present disclosure, in which one or more specific codons present in the nucleic acid sequence of an IL-2 polypeptide are allocated to encode the unnatural amino acid so that it can be genetically incorporated into the IL-2 by using an orthogonal tRNA synthetase/tRNA pair. The orthogonal tRNA synthetase/tRNA pair is capable of charging a tRNA with an unnatural amino acid and is capable of incorporating that unnatural amino acid into the polypeptide chain in response to the codon.

In some instances, the codon is the codon amber, ochre, opal or a quadruplet codon. In some cases, the codon corresponds to the orthogonal tRNA which will be used to carry the unnatural amino acid. In some cases, the codon is amber. In other cases, the codon is an orthogonal codon.

In some instances, the codon is a quadruplet codon, which can be decoded by an orthogonal ribosome ribo-Q1. In some cases, the quadruplet codon is as illustrated in Neumann, et al., “Encoding multiple unnatural amino acids via evolution of a quadruplet-decoding ribosome,” Nature, 464 (7287): 441-444 (2010), the disclosure of which is herein incorporated by reference.

In some instances, a codon used in the present disclosure is a recoded codon, e.g., a synonymous codon or a rare codon that is replaced with alternative codon. In some cases, the recoded codon is as described in Napolitano, et al., “Emergent rules for codon choice elucidated by editing rare arginine codons in Escherichia coli,” PNAS, 113 (38): E5588-5597 (2016), the disclosure of which is herein incorporated by reference. In some cases, the recoded codon is as described in Ostrov et al., “Design, synthesis, and testing toward a 57-codon genome,” Science 353 (6301): 819-822 (2016), the disclosure of which is herein incorporated by reference.

In some instances, unnatural nucleic acids are utilized leading to incorporation of one or more unnatural amino acids into the IL-2. Exemplary unnatural nucleic acids include, but are not limited to, uracil-5-yl, hypoxanthin-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifiuoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain unnatural nucleic acids, such as 5-substituted pyrimidines, 6-azapyrimidines and N-2 substituted purines, N-6 substituted purines, O-6 substituted purines, 2-aminopropyladenine, 5-propynyluracil, 5-propynylcytosine, 5-methylcytosine, those that increase the stability of duplex formation, universal nucleic acids, hydrophobic nucleic acids, promiscuous nucleic acids, size-expanded nucleic acids, fluorinated nucleic acids, 5-substituted pyrimidines, 6-azapyrimidines and N-2, N-6 and 0-6 substituted purines, including 2-aminopropyladenine, 5-propynyluracil and 5-propynylcytosine. 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—C≡C-CH₃) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyladenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine ([5,4-b][1,4]benzoxazin-2 (3H)-one), phenothiazine cytidine (1H-pyrimido[5,4-b][1,4]benzothiazin-2 (3H)-one), G-clamps, phenoxazine cytidine (e.g. 9-(2-aminoethoxy)-H-pyrimido[5,4-b][1,4]benzoxazin-2 (3H)-one), carbazole cytidine (2H-pyrimido[4,5-b]indol-2-one), pyridoindole cytidine (H-pyrido[3′,2′: 4,5]pyrrolo[2,3-d]pyrimidin-2-one), those in which the purine or pyrimidine base is replaced with other heterocycles, 7-deaza-adenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 5-propynyl-uracil, 4-thio-uracil, N4-ethylcytosine, 7-deazaguanine, 7-deaza-8-azaguanine, 5-hydroxycytosine, 2′-deoxyuridine, 2-amino-2′-deoxyadenosine, and those described in U.S. Pat. Nos. 3,687,808; 4,845,205; 4,910,300; 4,948,882; 5,093,232; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096; WO 99/62923; Kandimalla et al., (2001) Bioorg. Med. Chem. 9:807-813; The Concise Encyclopedia of Polymer Science and Engineering, Kroschwitz, J. I., Ed., John Wiley & Sons, 1990, 858-859; Englisch et al., Angewandte Chemie, International Edition, 1991, 30, 613; and Sanghvi, Chapter 15, Antisense Research and Applications, Crooke and Lebleu Eds., CRC Press, 1993, 273-288. Additional base modifications can be found, for example, in U.S. Pat. No. 3,687,808; Englisch et al., Angewandte Chemie, International Edition, 1991, 30, 613; and Sanghvi, Chapter 15, Antisense Research and Applications, pages 289-302, Crooke and Lebleu ed., CRC Press, 1993; the disclosure of each of which is herein incorporated by reference.

Unnatural nucleic acids comprising various heterocyclic bases and various sugar moieties (and sugar analogs) are available in the art, and the nucleic acids in some cases include one or several heterocyclic bases other than the principal five base components of naturally-occurring nucleic acids. For example, the heterocyclic base includes, in some cases, uracil-5-yl, cytosin-5-yl, adenin-7-yl, adenin-8-yl, guanin-7-yl, guanin-8-yl, 4-aminopyrrolo[2,3-d]pyrimidin-5-yl, 2-amino-4-oxopyrolo[2, 3-d]pyrimidin-5-yl, 2-amino-4-oxopyrrolo[2,3-d]pyrimidin-3-yl groups, where the purines are attached to the sugar moiety of the nucleic acid via the 9-position, the pyrimidines via the 1-position, the pyrrolopyrimidines via the 7-position and the pyrazolopyrimidines via the 1-position.

In some embodiments, nucleotide analogs are also modified at the phosphate moiety. Modified phosphate moieties include, but are not limited to, those with modification at the linkage between two nucleotides and contains, for example, a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides are through a 3′-5′ linkage or a 2′-5′ linkage, and the linkage contains inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050; the disclosure of each of which is herein incorporated by reference.

In some embodiments, unnatural nucleic acids include 2′,3′-dideoxy-2′,3′-didehydro-nucleosides (PCT/US2002/006460), 5′-substituted DNA and RNA derivatives (PCT/US2011/033961; Saha et al., J. Org Chem., 1995, 60, 788-789; Wang et al., Bioorganic & Medicinal Chemistry Letters, 1999, 9, 885-890; Mikhailov et al., Nucleosides & Nucleotides, 1991, 10(1-3), 339-343; Leonid et al., 1995, 14 (3-5), 901-905; Eppacher et al., Helvetica Chimica Acta, 2004, 87, 3004-3020; PCT/JP2000/004720; PCT/JP2003/002342; PCT/JP2004/013216; PCT/JP2005/020435; PCT/JP2006/315479; PCT/JP2006/324484; PCT/JP2009/056718; PCT/JP2010/067560), or 5′-substituted monomers made as the monophosphate with modified bases (Wang et al., Nucleosides Nucleotides & Nucleic Acids, 2004, 23 (1 & 2), 317-337); the disclosure of each of which is herein incorporated by reference.

In some embodiments, unnatural nucleic acids include modifications at the 5′-position and the 2′-position of the sugar ring (PCT/US94/02993), such as 5′-CH₂-substituted 2′-O-protected nucleosides (Wu et al., Helvetica Chimica Acta, 2000, 83, 1127-1143 and Wu et al., Bioconjugate Chem. 1999, 10, 921-924, the disclosure of which is herein incorporated by reference). In some cases, unnatural nucleic acids include amide linked nucleoside dimers have been prepared for incorporation into oligonucleotides wherein the 3′ linked nucleoside in the dimer (5′ to 3′) comprises a 2′-OCH₃ and a 5′-(S)—CH₃ (Mesmaeker et al., Synlett, 1997, 1287-1290). Unnatural nucleic acids can include 2′-substituted 5′-CH₂ (or O) modified nucleosides (PCT/US92/01020). Unnatural nucleic acids can include 5′-methylenephosphonate DNA and RNA monomers, and dimers (Bohringer et al., Tet. Lett., 1993, 34, 2723-2726; Collingwood et al., Synlett, 1995, 7, 703-705; and Hutter et al., Helvetica Chimica Acta, 2002, 85, 2777-2806). Unnatural nucleic acids can include 5′-phosphonate monomers having a 2′-substitution (US2006/0074035) and other modified 5′-phosphonate monomers (WO1997/35869). Unnatural nucleic acids can include 5′-modified methylenephosphonate monomers (EP614907 and EP629633). Unnatural nucleic acids can include analogs of 5′ or 6′-phosphonate ribonucleosides comprising a hydroxyl group at the 5′ and/or 6′-position (Chen et al., Phosphorus, Sulfur and Silicon, 2002, 777, 1783-1786; Jung et al., Bioorg. Med. Chem., 2000, 8, 2501-2509; Gallier et al., Eur. J. Org. Chem., 2007, 925-933; and Hampton et al., J. Med. Chem., 1976, 19 (8), 1029-1033). Unnatural nucleic acids can include 5′-phosphonate deoxyribonucleoside monomers and dimers having a 5′-phosphate group (Nawrot et al., Oligonucleotides, 2006, 16 (1), 68-82). Unnatural nucleic acids can include nucleosides having a 6′-phosphonate group wherein the 5′ or/and 6′-position is unsubstituted or substituted with a thio-tert-butyl group (SC(CH₃)₃) (and analogs thereof); a methyleneamino group (CH₂NH₂) (and analogs thereof) or a cyano group (CN) (and analogs thereof) (Fairhurst et al., Synlett, 2001, 4, 467-472; Kappler et al., J. Med. Chem., 1986, 29, 1030-1038; Kappler et al., J. Med. Chem., 1982, 25, 1179-1184; Vrudhula et al., J. Med. Chem., 1987, 30, 888-894; Hampton et al., J. Med. Chem., 1976, 19, 1371-1377; Geze et al., J. Am. Chem. Soc, 1983, 105 (26), 7638-7640; and Hampton et al., J. Am. Chem. Soc, 1973, 95 (13), 4404-4414). The disclosure of each reference listed in this paragraph is herein incorporated by reference.

In some embodiments, unnatural nucleic acids also include modifications of the sugar moiety. In some cases, nucleic acids contain one or more nucleosides wherein the sugar group has been modified. Such sugar modified nucleosides may impart enhanced nuclease stability, increased binding affinity, or some other beneficial biological property. In certain embodiments, nucleic acids comprise a chemically modified ribofuranose ring moiety. Examples of chemically modified ribofuranose rings include, without limitation, addition of substituent groups (including 5′ and/or 2′ substituent groups; bridging of two ring atoms to form bicyclic nucleic acids (BNA); replacement of the ribosyl ring oxygen atom with S, N(R), or C(R₁)(R₂) (R═H, C₁-C₁₂ alkyl or a protecting group); and combinations thereof. Examples of chemically modified sugars can be found in WO2008/101157, US2005/0130923, and WO2007/134181, the disclosure of each of which is herein incorporated by reference.

In some instances, a modified nucleic acid comprises modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety may be the furanoside of ribose, deoxyribose, arabinose or 2′-O-alkylribose, and the sugar can be attached to the respective heterocyclic bases either in [alpha] or [beta] anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-uridine or 2′-O-methyl-cytidine. Sugar modifications include 2′-O-alkyl-substituted deoxyribonucleosides and 2′-O-ethyleneglycol like ribonucleosides. The preparation of these sugars or sugar analogs and the respective “nucleosides” wherein such sugars or analogs are attached to a heterocyclic base (nucleic acid base) is known. Sugar modifications may also be made and combined with other modifications.

Modifications to the sugar moiety include natural modifications of the ribose and deoxy ribose as well as unnatural modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀, alkyl or C₂ to C₁₀ alkenyl and alkynyl. 2′ sugar modifications also include but are not limited to —O[(CH₂)ₙO]ₘCH₃, —O(CH₂)ₙOCH₃, —O(CH₂)ₙNH₂, —O(CH₂)ₙCH₃, —O(CH₂)ₙONH₂, and —O(CH₂)ₙON[(CH₂)nCH₃)]₂, where n and m are from 1 to about 10.

Other modifications at the 2′ position include but are not limited to: C₁ to C₁₀ lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of the 5′ terminal nucleotide. Modified sugars also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures and which detail and describe a range of base modifications, such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,700,920, the disclosure of each of which is herein incorporated by reference in its entirety.

Examples of nucleic acids having modified sugar moieties include, without limitation, nucleic acids comprising 5′-vinyl, 5′-methyl (R or S), 4′-S, 2′-F, 2′-OCH₃, and 2′-O(CH₂) 2OCH₃ substituent groups. The substituent at the 2′ position can also be selected from allyl, amino, azido, thio, O-allyl, O—(C₁-C₁₀ alkyl), OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl.

In certain embodiments, nucleic acids described herein include one or more bicyclic nucleic acids. In certain such embodiments, the bicyclic nucleic acid comprises a bridge between the 4′ and the 2′ ribosyl ring atoms. In certain embodiments, nucleic acids provided herein include one or more bicyclic nucleic acids wherein the bridge comprises a 4′ to 2′ bicyclic nucleic acid. Examples of such 4′ to 2′ bicyclic nucleic acids include, but are not limited to, one of the Formulae: 4′-(CH₂)—O-2′ (LNA); 4′-(CH₂)—S-2′; 4′-(CH₂) 2-O-2′ (ENA); 4′-CH(CH₃)—O-2′ and 4′-CH(CH₂OCH₃)—O-2′, and analogs thereof (see, U.S. Pat. No. 7,399,845); 4′-C(CH₃)(CH₃)—O-2′ and analogs thereof, (see WO2009/006478, WO2008/150729, US2004/0171570, U.S. Pat. No. 7,427,672, Chattopadhyaya et al., J. Org. Chem., 209, 74, 118-134, and WO2008/154401). Also see, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129 (26) 8362-8379; Elayadi et al., Curr. Opinion Invens. Drugs, 2001, 2, 558-561; Braasch et al., Chem. Biol, 2001, 8, 1-7; Oram et al., Curr. Opinion Mol. Ther., 2001, 3, 239-243; U.S. Pat. Nos. 4,849,513; 5,015,733; 5,118,800; 5,118,802; 7,053,207; 6,268,490; 6,770,748; 6,794,499; 7,034,133; 6,525,191; 6,670,461; and 7,399,845; International Publication Nos. WO2004/106356, WO1994/14226, WO2005/021570, WO2007/090071, and WO2007/134181; U.S. Patent Publication Nos. US2004/0171570, US2007/0287831, and US2008/0039618; U.S. Provisional Application Nos. 60/989,574, 61/026,995, 61/026,998, 61/056,564, 61/086,231, 61/097,787, and 61/099,844; and International Applications Nos. PCT/US2008/064591, PCT US2008/066154, PCT US2008/068922, and PCT/DK98/00393. The disclosure of each reference listed in this paragraph is herein incorporated by reference.

In certain embodiments, nucleic acids comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P═S). Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N*-dimethylhydrazine (—CH₂—N(CH₃)—N(CH₃)). In certain embodiments, inter nucleic acids linkages having a chiral atom can be prepared as a racemic mixture, as separate enantiomers, e.g., alkylphosphonates and phosphorothioates. Unnatural nucleic acids can contain a single modification. Unnatural nucleic acids can contain multiple modifications within one of the moieties or between different moieties.

Backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and may be used in any combination. Other non-phosphate linkages may also be used.

In some embodiments, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

In some instances, a phosphorous derivative (or modified phosphate group) is attached to the sugar or sugar analog moiety in and can be a monophosphate, diphosphate, triphosphate, alkylphosphonate, phosphorothioate, phosphorodithioate, phosphoramidate or the like. Exemplary polynucleotides containing modified phosphate linkages or non-phosphate linkages can be found in Peyrottes et al., 1996, Nucleic Acids Res. 24:1841-1848; Chaturvedi et al., 1996, Nucleic Acids Res. 24:2318-2323; Schultz et al., (1996) Nucleic Acids Res. 24:2966-2973; Matteucci, 1997, “Oligonucleotide Analogs: an Overview” in Oligonucleotides as Therapeutic Agents, (Chadwick and Cardew, ed.) John Wiley and Sons, New York, NY; Zon, 1993, “Oligonucleoside Phosphorothioates” in Protocols for Oligonucleotides and Analogs, Synthesis and Properties, Humana Press, pp. 165-190; Miller et al., 1971, JACS 93:6657-6665; Jager et al., 1988, Biochem. 27:7247-7246; Nelson et al., 1997, JOC 62:7278-7287; U.S. Pat. No. 5,453,496; and Micklefield, 2001, Curr. Med. Chem. 8:1157-1179; the disclosure of each of which is herein incorporated by reference.

In some cases, backbone modification comprises replacing the phosphodiester linkage with an alternative moiety such as an anionic, neutral or cationic group. Examples of such modifications include: anionic internucleoside linkage; N3′ to P5′ phosphoramidate modification; boranophosphate DNA; prooligonucleotides; neutral internucleoside linkages such as methylphosphonates; amide linked DNA; methylene(methylimino) linkages; formacetal and thioformacetal linkages; backbones containing sulfonyl groups; morpholino oligos; peptide nucleic acids (PNA); and positively charged deoxyribonucleic guanidine (DNG) oligos (Micklefield, 2001, Current Medicinal Chemistry 8:1157-1179). A modified nucleic acid may comprise a chimeric or mixed backbone comprising one or more modifications, e.g. a combination of phosphate linkages such as a combination of phosphodiester and phosphorothioate linkages.

Substitutes for the phosphate include, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439. It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. See also Nielsen et al., Science, 1991, 254, 1497-1500. It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. KY. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EM5OJ, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1-di-O-hexadecyl-rac-glycero-S-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochem. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937). Numerous United States patents teach the preparation of such conjugates and include, but are not limited to U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214, 136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514, 785; 5,565,552; 5,567,810; 5,574,142; 5,585,481; 5,587,371; 5,595,726; 5,597,696; 5,599,923; 5,599,928 and 5,688,941. The disclosure of each reference listed in this paragraph is herein incorporated by reference.

In some cases, the unnatural nucleic acids further form unnatural base pairs. Exemplary unnatural nucleotides capable of forming an unnatural DNA or RNA base pair (UBP) under conditions in vivo includes, but is not limited to, TAT1, dTAT1, 5FM, d5FM, TPT3, dTPT3, 5SICS, d5SICS, NaM, dNaM, CNMO, dCNMO, and combinations thereof. In some embodiments, unnatural nucleotides include:

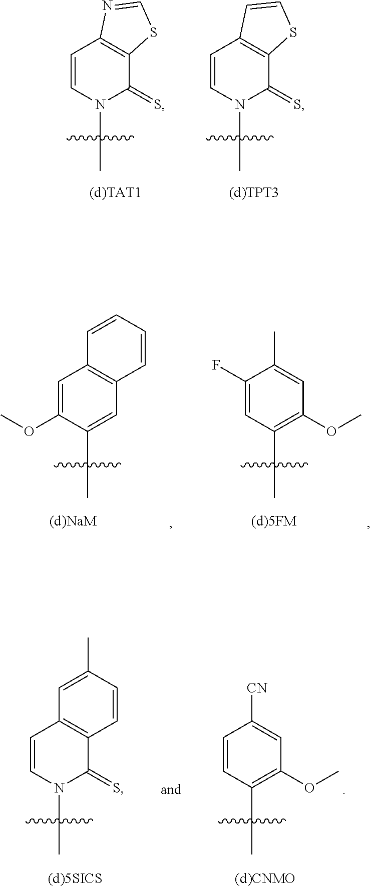 [SMILES: *n1ccc2ncsc2c1=S ; *n1ccc2ccsc2c1=S ; *c1cc2ccccc2cc1OC ; *c1cc(F)c(C)cc1OC ; *n1ccc2cc(C)ccc2c1=S ; *c1ccc(C#N)cc1OC]

Exemplary unnatural base pairs include: (d)TPT3-(d)NaM; (d)5SICS-(d)NaM; (d)CNMO-(d)TAT1; (d)NaM-(d)TAT1; (d)CNMO-(d)TPT3; and (d)5FM-(d)TAT1.

Other examples of unnatural nucleotides capable of forming unnatural UBPs that may be used to prepare the IL-2 conjugates disclosed herein may be found in Dien et al., J Am Chem Soc., 2018, 140:16115-16123; Feldman et al., J Am Chem Soc, 2017, 139:11427-11433; Ledbetter et al., J Am Chem Soc., 2018, 140:758-765; Dhami et al., Nucleic Acids Res. 2014, 42:10235-10244; Malyshev et al., Nature, 2014, 509:385-388; Betz et al., J Am Chem Soc., 2013, 135:18637-18643; Lavergne et al., J Am Chem Soc. 2013, 135:5408-5419; and Malyshev et al. Proc Natl Acad Sci USA, 2012, 109:12005-12010; the disclosure of each of which is herein incorporated by reference. In some embodiments, unnatural nucleotides include:

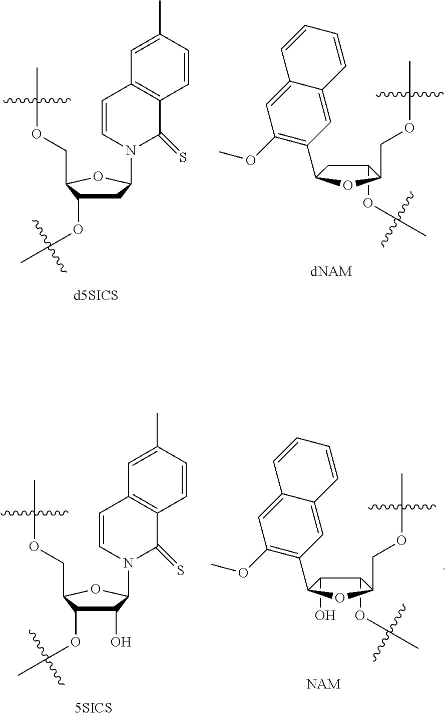

In some embodiments, the unnatural nucleotides that may be used to prepare the IL-2 conjugates disclosed herein may be derived from a compound of the Formula

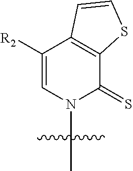

wherein R₂ is selected from hydrogen, alkyl, alkenyl, alkynyl, methoxy, methanethiol, methaneseleno, halogen, cyano, and azido; and
the wavy line indicates a bond to a ribosyl or 2′-deoxyribosyl, wherein the 5′-hydroxy group of the ribosyl or 2′-deoxyribosyl moiety is in free form, is connected to a monophosphate, diphosphate, triphosphate, a-thiotriphosphate, B-thiotriphosphate, or y-thiotriphosphate group, or is included in an RNA or a DNA or in an RNA analog or a DNA analog.

In some embodiments, the unnatural nucleotides that may be used to prepare the IL-2 conjugates disclosed herein may be derived from a compound of the Formula

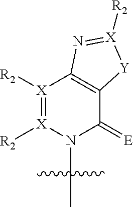

wherein:
each X is independently carbon or nitrogen;
R₂ is absent when X is nitrogen, and is present when X is carbon and is independently hydrogen, alkyl, alkenyl, alkynyl, methoxy, methanethiol, methaneseleno, halogen, cyano, or azide;
Y is sulfur, oxygen, selenium, or secondary amine;
E is oxygen, sulfur, or selenium; and
the wavy line indicates a point of bonding to a ribosyl, deoxyribosyl, or dideoxyribosyl moiety or an analog thereof, wherein the ribosyl, deoxyribosyl, or dideoxyribosyl moiety or analog thereof is in free form, is connected to a mono-phosphate, diphosphate, triphosphate, α-thiotriphosphate, β-thiotriphosphate, or γ-thiotriphosphate group, or is included in an RNA or a DNA or in an RNA analog or a DNA analog.

In some embodiments, each X is carbon. In some embodiments, at least one X is carbon. In some embodiments, one X is carbon. In some embodiments, at least two X are carbon. In some embodiments, two X are carbon. In some embodiments, at least one X is nitrogen. In some embodiments, one X is nitrogen. In some embodiments, at least two X are nitrogen. In some embodiments, two X are nitrogen.

In some embodiments, Y is sulfur. In some embodiments, Y is oxygen. In some embodiments, Y is selenium. In some embodiments, Y is a secondary amine.

In some embodiments, E is sulfur. In some embodiments, E is oxygen. In some embodiments, E is selenium.

In some embodiments, R₂ is present when X is carbon. In some embodiments, R² is absent when X is nitrogen. In some embodiments, each R₂, where present, is hydrogen. In some embodiments, R₂ is alkyl, such as methyl, ethyl, or propyl. In some embodiments, R₂ is alkenyl, such as —CH₂═CH₂. In some embodiments, R₂ is alkynyl, such as ethynyl. In some embodiments, R₂ is methoxy. In some embodiments, R₂ is methanethiol. In some embodiments, R₂ is methaneseleno. In some embodiments, R₂ is halogen, such as chloro, bromo, or fluoro. In some embodiments, R₂ is cyano. In some embodiments, R₂ is azide.

In some embodiments, E is sulfur, Y is sulfur, and each X is independently carbon or nitrogen. In some embodiments, E is sulfur, Y is sulfur, and each X is carbon.

In some embodiments, the unnatural nucleotides that may be used to prepare the IL-2 conjugates disclosed herein may be derived from

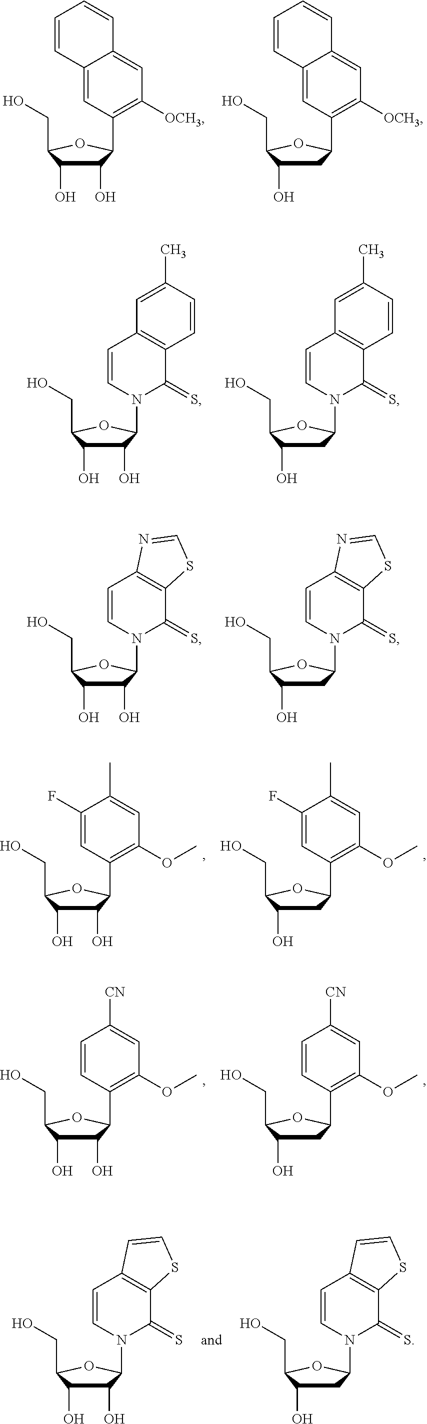

In some embodiments, the unnatural nucleotides that may be used to prepare the IL-2 conjugates disclosed herein include

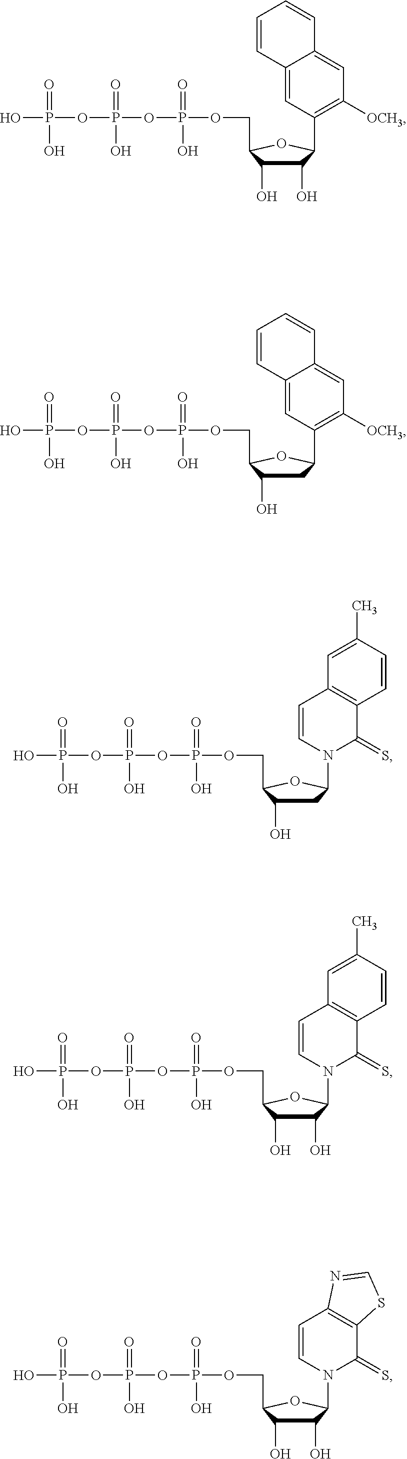

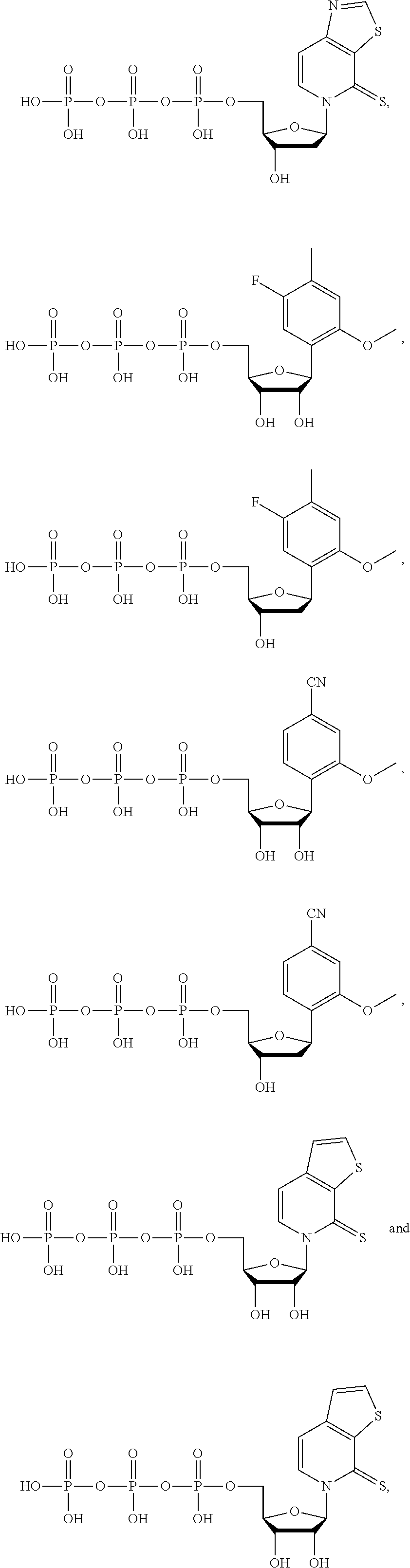

or salts thereof.

In some embodiments, the unnatural nucleotides that may be used to prepare the IL-2 conjugates disclosed herein may be derived from

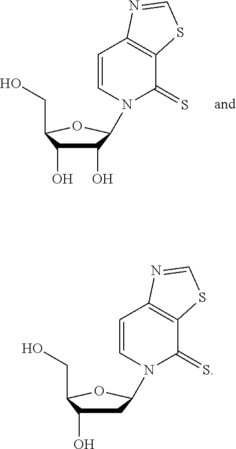

In some embodiments, the unnatural nucleotides that may be used to prepare the IL-2 conjugates disclosed herein include

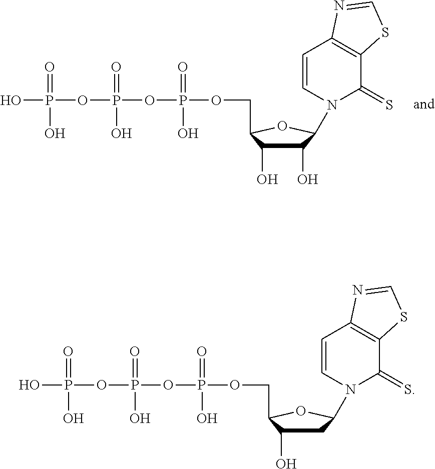

In some embodiments, an unnatural base pair generates an unnatural amino acid as described in Dumas et al., “Designing logical codon reassignment—Expanding the chemistry in biology,” Chemical Science, 6:50-69 (2015).

In some embodiments, the unnatural amino acid is incorporated into the IL-2 polypeptide by a synthetic codon comprising an unnatural nucleic acid. In some instances, the unnatural amino acid is incorporated into the IL-2 by an orthogonal, modified synthetase/tRNA pair. Such orthogonal pairs comprise an unnatural synthetase that is capable of charging the unnatural tRNA with the unnatural amino acid, while minimizing charging of a) other endogenous amino acids onto the unnatural tRNA and b) unnatural amino acids onto other endogenous tRNAs. Such orthogonal pairs comprise tRNAs that are capable of being charged by the unnatural synthetase, while avoiding being charged with a) other endogenous amino acids by endogenous synthetases. In some embodiments, such pairs are identified from various organisms, such as bacteria, yeast, Archaea, or human sources. In some embodiments, an orthogonal synthetase/tRNA pair comprises components from a single organism. In some embodiments, an orthogonal synthetase/tRNA pair comprises components from two different organisms. In some embodiments, an orthogonal synthetase/tRNA pair comprising components that prior to modification, promote translation of two different amino acids. In some embodiments, an orthogonal synthetase is a modified alanine synthetase. In some embodiments, an orthogonal synthetase is a modified arginine synthetase. In some embodiments, an orthogonal synthetase is a modified asparagine synthetase. In some embodiments, an orthogonal synthetase is a modified aspartic acid synthetase. In some embodiments, an orthogonal synthetase is a modified cysteine synthetase. In some embodiments, an orthogonal synthetase is a modified glutamine synthetase. In some embodiments, an orthogonal synthetase is a modified glutamic acid synthetase. In some embodiments, an orthogonal synthetase is a modified alanine glycine. In some embodiments, an orthogonal synthetase is a modified histidine synthetase. In some embodiments, an orthogonal synthetase is a modified leucine synthetase. In some embodiments, an orthogonal synthetase is a modified isoleucine synthetase. In some embodiments, an orthogonal synthetase is a modified lysine synthetase. In some embodiments, an orthogonal synthetase is a modified methionine synthetase. In some embodiments, an orthogonal synthetase is a modified phenylalanine synthetase. In some embodiments, an orthogonal synthetase is a modified proline synthetase. In some embodiments, an orthogonal synthetase is a modified serine synthetase. In some embodiments, an orthogonal synthetase is a modified threonine synthetase. In some embodiments, an orthogonal synthetase is a modified tryptophan synthetase. In some embodiments, an orthogonal synthetase is a modified tyrosine synthetase. In some embodiments, an orthogonal synthetase is a modified valine synthetase. In some embodiments, an orthogonal synthetase is a modified phosphoserine synthetase. In some embodiments, an orthogonal tRNA is a modified alanine tRNA. In some embodiments, an orthogonal tRNA is a modified arginine tRNA. In some embodiments, an orthogonal tRNA is a modified asparagine tRNA. In some embodiments, an orthogonal tRNA is a modified aspartic acid tRNA. In some embodiments, an orthogonal tRNA is a modified cysteine tRNA. In some embodiments, an orthogonal tRNA is a modified glutamine tRNA. In some embodiments, an orthogonal tRNA is a modified glutamic acid tRNA. In some embodiments, an orthogonal tRNA is a modified alanine glycine. In some embodiments, an orthogonal tRNA is a modified histidine tRNA. In some embodiments, an orthogonal tRNA is a modified leucine tRNA. In some embodiments, an orthogonal tRNA is a modified isoleucine tRNA. In some embodiments, an orthogonal tRNA is a modified lysine tRNA. In some embodiments, an orthogonal tRNA is a modified methionine tRNA. In some embodiments, an orthogonal tRNA is a modified phenylalanine tRNA. In some embodiments, an orthogonal tRNA is a modified proline tRNA. In some embodiments, an orthogonal tRNA is a modified serine tRNA. In some embodiments, an orthogonal tRNA is a modified threonine tRNA. In some embodiments, an orthogonal tRNA is a modified tryptophan tRNA. In some embodiments, an orthogonal tRNA is a modified tyrosine tRNA. In some embodiments, an orthogonal tRNA is a modified valine tRNA. In some embodiments, an orthogonal tRNA is a modified phosphoserine tRNA.

In some embodiments, the unnatural amino acid is incorporated into the IL-2 polypeptide by an aminoacyl (aaRS or RS)-tRNA synthetase-tRNA pair. Exemplary aaRS-tRNA pairs include, but are not limited to, Methanococcus jannaschii (Mj-Tyr) aaRS/tRNA pairs, E. coli TyrRS (Ec-Tyr)/B. stearothermophilus tRNA_{CUA} pairs, E. coli LeuRS (Ec-Leu)/B. stearothermophilus tRNA_{CUA} pairs, and pyrrolysyl-tRNA pairs. In some instances, the unnatural amino acid is incorporated into the IL-2 polypeptide by a Mj-TyrRS/tRNA pair. Exemplary UAAs that can be incorporated by a Mj-TyrRS/tRNA pair include, but are not limited to, para-substituted phenylalanine derivatives such as p-aminophenylalanine and p-methoyphenylalanine; meta-substituted tyrosine derivatives such as 3-aminotyrosine, 3-nitrotyrosine, 3,4-dihydroxyphenylalanine, and 3-iodotyrosine; phenylselenocysteine; p-boronophenylalanine; and o-nitrobenzyltyrosine.

In some instances, the unnatural amino acid is incorporated into the IL-2 polypeptide by a Ec-Tyr/tRNA_{CUA} or a Ec-Leu/tRNA_{CUA} pair. Exemplary UAAs that can be incorporated by a Ec-Tyr/tRNA_{CUA} or a Ec-Leu/tRNA_{CUA} pair include, but are not limited to, phenylalanine derivatives containing benzophenone, ketone, iodide, or azide substituents; O-propargyltyrosine; α-aminocaprylic acid, O-methyl tyrosine, O-nitrobenzyl cysteine; and 3-(naphthalene-2-ylamino)-2-amino-propanoic acid.

In some instances, the unnatural amino acid is incorporated into the IL-2 polypeptide by a pyrrolysyl-tRNA pair. In some cases, the PylRS is obtained from an archaebacterial, e.g., from a methanogenic archaebacterial. In some cases, the PylRS is obtained from Methanosarcina barkeri, Methanosarcina mazei, or Methanosarcina acetivorans. Exemplary UAAs that can be incorporated by a pyrrolysyl-tRNA pair include, but are not limited to, amide and carbamate substituted lysines such as 2-amino-6-((R)-tetrahydrofuran-2-carboxamido) hexanoic acid, N-ε-D-prolyl-L-lysine, and N-ε-cyclopentyloxycarbonyl-L-lysine; N-ε-Acryloyl-L-lysine; N-ε-[(1-(6-nitrobenzo[d][1,3]dioxol-5-yl) ethoxy) carbonyl]-L-lysine; and N-ε-(1-methylcyclopro-2-enecarboxamido) lysine. In some embodiments, the IL-2 conjugates disclosed herein may be prepared by use of M. mazei tRNA which is selectively charged with a non-natural amino acid such as N6-((2-azidoethoxy)-carbonyl)-L-lysine (AzK) by the M. barkeri pyrrolysyl-tRNA synthetase (Mb PyIRS). Other methods are known to those of ordinary skill in the art, such as those disclosed in Zhang et al., Nature 2017, 551 (7682): 644-647, the disclosure of which is herein incorporated by reference.

In some instances, an unnatural amino acid is incorporated into the IL-2 polypeptide by a synthetase disclosed in U.S. Pat. Nos. 9,988,619 and 9,938,516, the disclosure of each of which is herein incorporated by reference.

The host cell into which the constructs or vectors disclosed herein are introduced is cultured or maintained in a suitable medium such that the tRNA, the tRNA synthetase and the protein of interest are produced. The medium also comprises the unnatural amino acid(s) such that the protein of interest incorporates the unnatural amino acid(s). In some embodiments, a nucleoside triphosphate transporter (NTT) from bacteria, plant, or algae is also present in the host cell. In some embodiments, the IL-2 conjugates disclosed herein are prepared by use of a host cell that expresses a NTT. In some embodiments, the nucleotide nucleoside triphosphate transporter used in the host cell may be selected from TpNTT1, TpNTT2, TpNTT3, TpNTT4, TpNTT5, TpNTT6, TpNTT7, TpNTT8 (T. pseudonana), PtNTT1, PtNTT2, PtNTT3, PtNTT4, PtNTT5, PtNTT6 (P. tricornutum), GsNTT (Galdieria sulphuraria), AtNTT1, AtNTT2 (Arabidopsis thaliana), CtNTT1, CtNTT2 (Chlamydia trachomatis), PamNTT1, PamNTT2 (Protochlamydia amoebophila), CcNTT (Caedibacter caryophilus), RpNTT1 (Rickettsia prowazekii). In some embodiments, the NTT is selected from PtNTT1, PtNTT2, PtNTT3, PtNTT4, PtNTT5, and PtNTT6. In some embodiments, the NTT is PtNTT1. In some embodiments, the NTT is PtNTT2. In some embodiments, the NTT is PtNTT3. In some embodiments, the NTT is PtNTT4. In some embodiments, the NTT is PtNTT5. In some embodiments, the NTT is PtNTT6. Other NTTs that may be used are disclosed in Zhang et al., Nature 2017, 551 (7682): 644-647; Malyshev et al. Nature 2014 (509 (7500), 385-388; and Zhang et al. Proc Natl Acad Sci USA, 2017, 114:1317-1322; the disclosure of each of which is herein incorporated by reference.

The orthogonal tRNA synthetase/tRNA pair charges a tRNA with an unnatural amino acid and incorporates the unnatural amino acid into the polypeptide chain in response to the codon. Exemplary aaRS-tRNA pairs include, but are not limited to, Methanococcus jannaschii (Mj-Tyr) aaRS/tRNA pairs, E. coli TyrRS (Ec-Tyr)/B. stearothermophilus tRNA_{CUA} pairs, E. coli LeuRS (Ec-Leu)/B. stearothermophilus tRNA_{CUA} pairs, and pyrrolysyl-tRNA pairs. Other aaRS-tRNA pairs that may be used according to the present disclosure include those derived from M. mazei those described in Feldman et al., J Am Chem Soc., 2018 140:1447-1454; and Zhang et al. Proc Natl Acad Sci USA, 2017, 114:1317-1322; the disclosure of each of which is herein incorporated by reference.

In some embodiments are provided methods of preparing the IL-2 conjugates disclosed herein in a cellular system that expresses a NTT and a tRNA synthetase. In some embodiments described herein, the NTT is selected from PtNTT1, PtNTT2, PtNTT3, PtNTT4, PtNTT5, and PtNTT6, and the tRNA synthetase is selected from Methanococcus jannaschii, E. coli TyrRS (Ec-Tyr)/B. stearothermophilus, and M. mazei. In some embodiments, the NTT is PtNTT1 and the tRNA synthetase is derived from Methanococcus jannaschii, E. coli TyrRS (Ec-Tyr)/B. stearothermophilus, or M. mazei. In some embodiments, the NTT is PtNTT2 and the tRNA synthetase is derived from Methanococcus jannaschii, E. coli TyrRS (Ec-Tyr)/B. stearothermophilus, or M. mazei. In some embodiments, the NTT is PtNTT3 and the tRNA synthetase is derived from Methanococcus jannaschii, E. coli TyrRS (Ec-Tyr)/B. stearothermophilus, or M. mazei. In some embodiments, the NTT is PtNTT3 and the tRNA synthetase is derived from Methanococcus jannaschii, E. coli TyrRS (Ec-Tyr)/B. stearothermophilus, or M. mazei. In some embodiments, the NTT is PtNTT4 and the tRNA synthetase is derived from Methanococcus jannaschii, E. coli TyrRS (Ec-Tyr)/B. stearothermophilus, or M. mazei. In some embodiments, the NTT is PtNTT5 and the tRNA synthetase is derived from Methanococcus jannaschii, E. coli TyrRS (Ec-Tyr)/B. stearothermophilus, or M. mazei. In some embodiments, the NTT is PtNTT6 and the tRNA synthetase is derived from Methanococcus jannaschii, E. coli TyrRS (Ec-Tyr)/B. stearothermophilus, or M. mazei.

In some embodiments, the IL-2 conjugates disclosed herein may be prepared in a cell, such as E. coli, comprising (a) nucleotide triphosphate transporter PtNTT2 (including a truncated variant in which the first 65 amino acid residues of the full-length protein are deleted), (b) a plasmid comprising a double-stranded oligonucleotide that encodes an IL-2 variant having a desired amino acid sequence and that contains a unnatural base pair comprising a first unnatural nucleotide and a second unnatural nucleotide to provide a codon at the desired position at which an unnatural amino acid, such as N6-((2-azidoethoxy)-carbonyl)-L-lysine (AzK), will be incorporated, (c) a plasmid encoding a tRNA derived from M. mazei and which comprises an unnatural nucleotide to provide a recognized anticodon (to the codon of the IL-2 variant) in place of its native sequence, and (d) a plasmid encoding a M. barkeri derived pyrrolysyl-tRNA synthetase (Mb PyIRS), which may be the same plasmid that encodes the tRNA or a different plasmid. In some embodiments, the cell is further supplemented with deoxyribo triphosphates comprising one or more unnatural bases. In some embodiments, the cell is further supplemented with ribo triphosphates comprising one or more unnatural bases. In some embodiments, the cells is further supplemented with one or more unnatural amino acids, such as N6-((2-azidoethoxy)-carbonyl)-L-lysine (AzK). In some embodiments, the double-stranded oligonucleotide that encodes the amino acid sequence of the desired IL-2 variant contains a codon AXC at, for example, position 34, 37, 40, 41, 42, 43, 44, 61, 64, 68, or 71 of the sequence that encodes the protein having SEQ ID NO: 3, or at position 35, 38, 41, 42, 43, 45, 62, 65, 69, or 72 of the sequence that encodes the protein having SEQ ID NO: 4, wherein X is an unnatural nucleotide. In some embodiments, the cell further comprises a plasmid, which may be the protein expression plasmid or another plasmid, that encodes an orthogonal tRNA gene from M. mazei that comprises an AXC-matching anticodon GYT in place of its native sequence, wherein Y is an unnatural nucleotide that is complementary and may be the same or different as the unnatural nucleotide in the codon. In some embodiments, the unnatural nucleotide in the codon is different than and complimentary to the unnatural nucleotide in the anti-codon. In some embodiments, the unnatural nucleotide in the codon is the same as the unnatural nucleotide in the anti-codon. In some embodiments, the first and second unnatural nucleotides comprising the unnatural base pair in the double-stranded oligonucleotide may be derived from

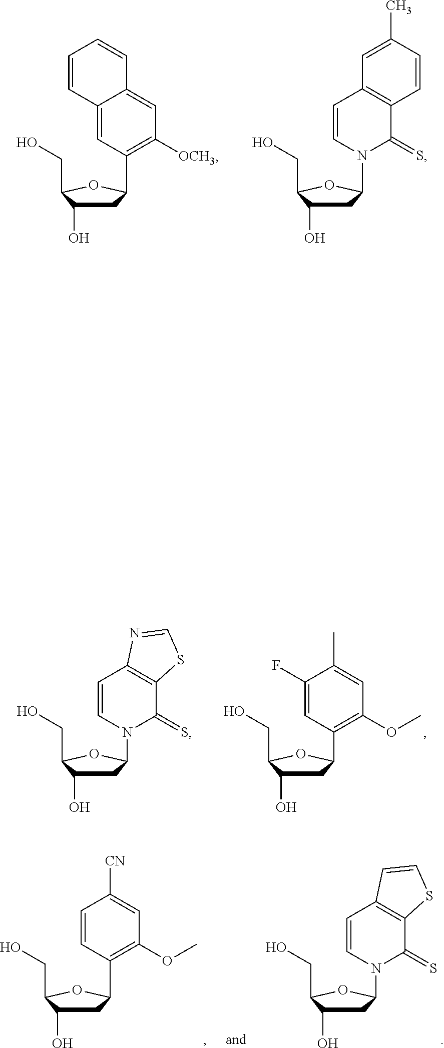

In some embodiments, the first and second unnatural nucleotides comprising the unnatural base pair in the double-stranded oligonucleotide may be derived from

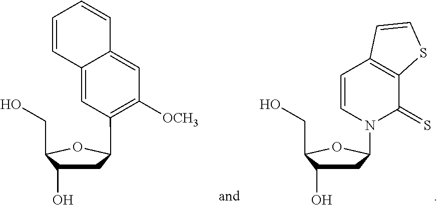

In some embodiments, the triphosphates of the first and second unnatural nucleotides include,

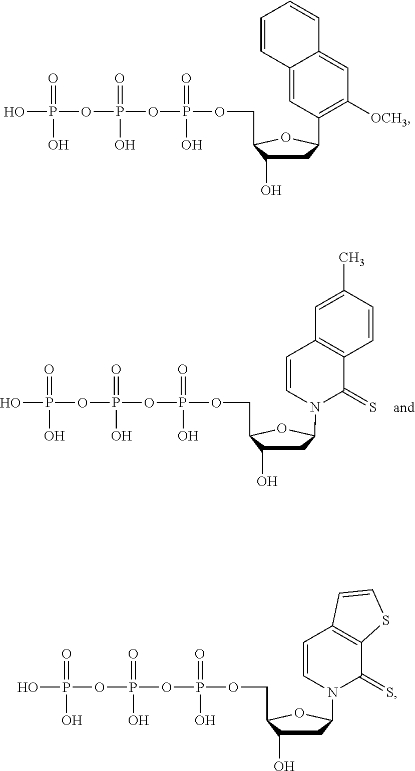

or salts thereof. In some embodiments, the triphosphates of the first and second unnatural nucleotides include,

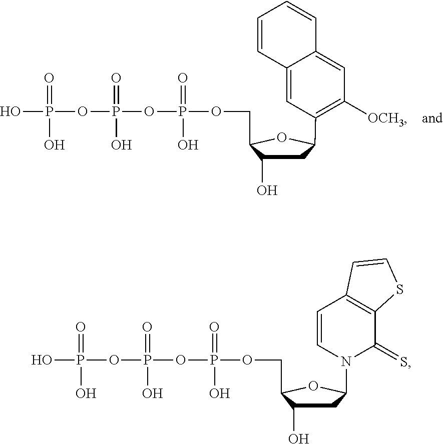

or salts thereof. In some embodiments, the mRNA derived the double-stranded oligonucleotide comprising a first unnatural nucleotide and a second unnatural nucleotide may comprise a codon comprising an unnatural nucleotide derived from

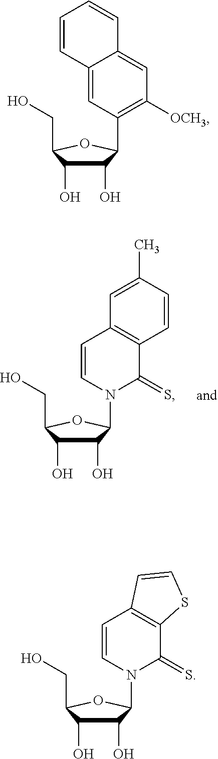

In some embodiments, the M. mazei tRNA may comprise an anti-codon comprising an unnatural nucleotide that recognizes the codon comprising the unnatural nucleotide of the mRNA. The anti-codon in the M. mazei tRNA may comprise an unnatural nucleotide derived from

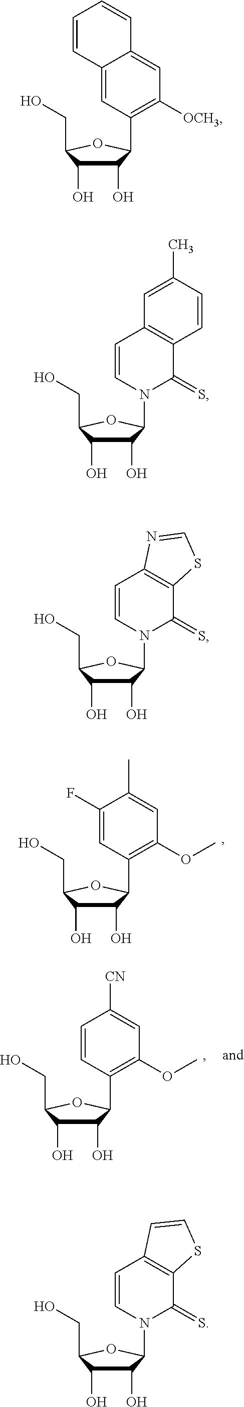

In some embodiments, the mRNA comprises an unnatural nucleotide derived from

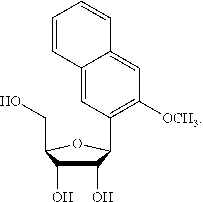

In some embodiments, the mRNA comprises an unnatural nucleotide derived from

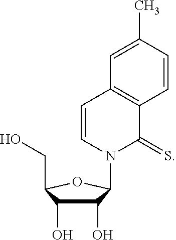

In some embodiments, the mRNA comprises an unnatural nucleotide derived from

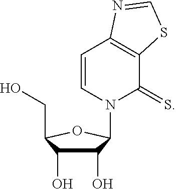

In some embodiments, the mRNA comprises an unnatural nucleotide derived from

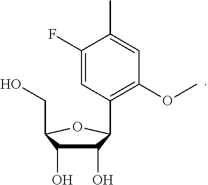

In some embodiments, the mRNA comprises an unnatural nucleotide derived from

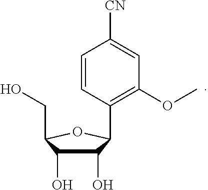

In some embodiments, the mRNA comprises an unnatural nucleotide derived from

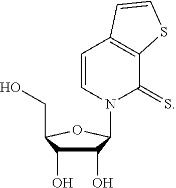

In some embodiments, the tRNA comprises an unnatural nucleotide derived from

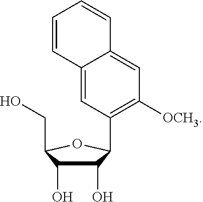

In some embodiments, the tRNA comprises an unnatural nucleotide derived from

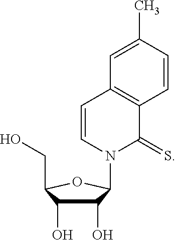

In some embodiments, the tRNA comprises an unnatural nucleotide derived from

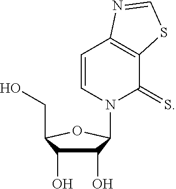

In some embodiments, the tRNA comprises an unnatural nucleotide derived from

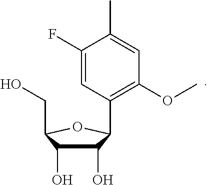

In some embodiments, the tRNA comprises an unnatural nucleotide derived from

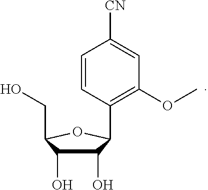

In some embodiments, the tRNA comprises an unnatural nucleotide derived from

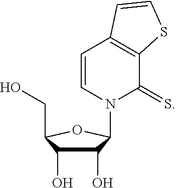

In some embodiments, the mRNA comprises an unnatural nucleotide derived from

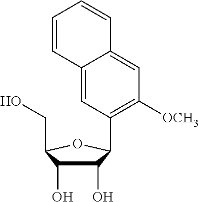

and the tRNA comprises an unnatural nucleotide derived from

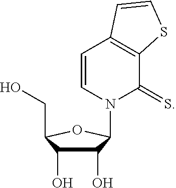

In some embodiments, the mRNA comprises an unnatural nucleotide derived from

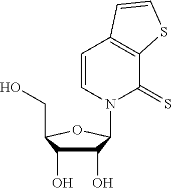

and the tRNA comprises an unnatural nucleotide derived from

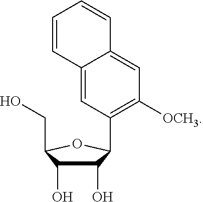

In some embodiments, the mRNA comprises an unnatural nucleotide derived from

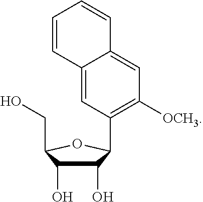

and the tRNA comprises an unnatural nucleotide derived from

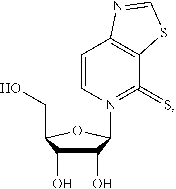

In some embodiments, the mRNA comprises an unnatural nucleotide derived from

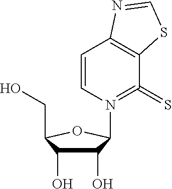

and the tRNA comprises an unnatural nucleotide derived from

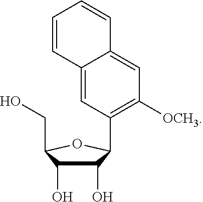

The host cell is cultured in a medium containing appropriate nutrients, and is supplemented with (a) the triphosphates of the deoxyribo nucleosides comprising one or more unnatural bases that are necessary for replication of the plasmid(s) encoding the cytokine gene harboring the codon, (b) the triphosphates of the ribo nucleosides comprising one or more unnatural bases necessary for transcription of (i) the mRNA corresponding to the coding sequence of the cytokine and containing the codon comprising one or more unnatural bases, and (ii) the tRNA containing the anticodon comprising one or more unnatural bases, and (c) the unnatural amino acid(s) to be incorporated in to the polypeptide sequence of the cytokine of interest. The host cells are then maintained under conditions which permit expression of the protein of interest.

The resulting AzK-containing protein that is expressed may be purified by methods known to those of ordinary skill in the art and may then be allowed to react with an alkyne, such as DBCO comprising a PEG chain having a desired average molecular weight as disclosed herein, under conditions known to those of ordinary skill in the art, to afford the IL-2 conjugates disclosed herein. Other methods are known to those of ordinary skill in the art, such as those disclosed in Zhang et al., Nature 2017, 551 (7682): 644-647; WO 2015157555; WO 2015021432; WO 2016115168; WO 2017106767; WO 2017223528; WO 2019014262; WO 2019014267; WO 2019028419; and WO2019/028425; the disclosure of each of which is herein incorporated by reference.

The resulting protein comprising the one or more unnatural amino acids, Azk for example, that is expressed may be purified by methods known to those of ordinary skill in the art and may then be allowed to react with an alkyne, such as DBCO comprising a PEG chain having a desired average molecular weight as disclosed herein, under conditions known to those of ordinary skill in the art, to afford the IL-2 conjugates disclosed herein. Other methods are known to those of ordinary skill in the art, such as those disclosed in Zhang et al., Nature 2017, 551 (7682): 644-647; WO 2015157555; WO 2015021432; WO 2016115168; WO 2017106767; WO 2017223528; WO 2019014262; WO 2019014267; WO 2019028419; and WO2019/028425; the disclosure of which is herein incorporated by reference.

Alternatively, IL-2 polypeptides comprising an unnatural amino acid(s) are prepared by introducing the nucleic acid constructs described herein comprising the tRNA and aminoacyl tRNA synthetase and comprising a nucleic acid sequence of interest with one or more in-frame orthogonal (stop) codons into a host cell. The host cell is cultured in a medium containing appropriate nutrients, is supplemented with (a) the triphosphates of the deoxyribo nucleosides comprising one or more unnatural bases required for replication of the plasmid(s) encoding the cytokine gene harboring the new codon and anticodon, (b) the triphosphates of the ribo nucleosides required for transcription of the mRNA corresponding to (i) the cytokine sequence containing the codon, and (ii) the orthogonal tRNA containing the anticodon, and (c) the unnatural amino acid(s). The host cells are then maintained under conditions which permit expression of the protein of interest. The unnatural amino acid(s) is incorporated into the polypeptide chain in response to the unnatural codon. For example, one or more unnatural amino acids are incorporated into the IL-2 polypeptide. Alternatively, two or more unnatural amino acids may be incorporated into the IL-2 polypeptide at two or more sites in the protein.

Once the IL-2 polypeptide incorporating the unnatural amino acid(s) has been produced in the host cell it can be extracted therefrom by a variety of techniques known in the art, including enzymatic, chemical and/or osmotic lysis and physical disruption. The IL-2 polypeptide can be purified by standard techniques known in the art such as preparative ion exchange chromatography, hydrophobic chromatography, affinity chromatography, or any other suitable technique known to those of ordinary skill in the art.

Suitable host cells may include bacterial cells (e.g., E. coli, BL21 (DE3)), but most suitably host cells are eukaryotic cells, for example insect cells (e.g. Drosophila such as Drosophila melanogaster), yeast cells, nematodes (e.g. C. elegans), mice (e.g. Mus musculus), or mammalian cells (such as Chinese hamster ovary cells (CHO) or COS cells, human 293T cells, HeLa cells, NIH 3T3 cells, and mouse erythroleukemia (MEL) cells) or human cells or other eukaryotic cells. Other suitable host cells are known to those skilled in the art. Suitably, the host cell is a mammalian cell-such as a human cell or an insect cell. In some embodiments, the suitable host cells comprise E. coli.

Other suitable host cells which may be used generally in the embodiments of the invention are those mentioned in the examples section. Vector DNA can be introduced into host cells via conventional transformation or transfection techniques. As used herein, the terms “transformation” and “transfection” are intended to refer to a variety of well-recognized techniques for introducing a foreign nucleic acid molecule (e.g., DNA) into a host cell, including calcium phosphate or calcium chloride co-precipitation, DEAE-dextran-mediated transfection, lipofection, or electroporation. Suitable methods for transforming or transfecting host cells are well known in the art.

When creating cell lines, it is generally preferred that stable cell lines are prepared. For stable transfection of mammalian cells for example, it is known that, depending upon the expression vector and transfection technique used, only a small fraction of cells may integrate the foreign DNA into their genome. In order to identify and select these integrants, a gene that encodes a selectable marker (for example, for resistance to antibiotics) is generally introduced into the host cells along with the gene of interest. Preferred selectable markers include those that confer resistance to drugs, such as G418, hygromycin, or methotrexate. Nucleic acid molecules encoding a selectable marker can be introduced into a host cell on the same vector or can be introduced on a separate vector. Cells stably transfected with the introduced nucleic acid molecule can be identified by drug selection (for example, cells that have incorporated the selectable marker gene will survive, while the other cells die).

In one embodiment, the constructs described herein are integrated into the genome of the host cell. An advantage of stable integration is that the uniformity between individual cells or clones is achieved. Another advantage is that selection of the best producers may be carried out. Accordingly, it is desirable to create stable cell lines. In another embodiment, the constructs described herein are transfected into a host cell. An advantage of transfecting the constructs into the host cell is that protein yields may be maximized. In one aspect, there is described a cell comprising the nucleic acid construct or the vector described herein.

### Anti-EGFR Antibodies

The methods of treating cancer described herein include administration of an anti-EGFR antibody in combination with the IL-2 conjugates described herein.

In some embodiments, the anti-EGFR antibody is an inhibitory antibody. In some embodiments, the anti-EGFR inhibitor antibody is selected from cetuximab (Erbitux), panitumumab (Vectibix), necitumumab (Portrazza), JNJ-61186372 (Amivantamab), IMC-C225, ABX-EGF, ICR62, and EMD 55900. In some embodiments, the anti-EGFR inhibitor antibody is cetuximab (Erbitux). In some embodiments, the anti-EGFR inhibitor antibody is panitumumab (Vectibix). In some embodiments, the anti-EGFR inhibitor antibody is necitumumab (Portrazza). In some embodiments, the anti-EGFR inhibitor antibody is JNJ-61186372 (Amivantamab). In some embodiments, the anti-EGFR inhibitor antibody is IMC-C225. In some embodiments, the anti-EGFR inhibitor antibodies is ABX-EGF. In some embodiments, the anti-EGFR inhibitor antibody is ICR62. In some embodiments, the anti-EGFR inhibitor antibody is EMD 55900.x

### Methods of Treatment

In one aspect, provided herein are methods of treating cancer in a subject in need thereof, comprising administering to the subject an effective amount of: (a) an IL-2 conjugate as described herein, and (b) an anti-EGFR antibody.

Also provided is an IL-2 conjugate as described herein for use in a method disclosed herein of treating cancer in a subject in need thereof.

In a further aspect, provided is use of an IL-2 conjugate as described herein for the manufacture of a medicament for a method disclosed herein of treating cancer in a subject in need thereof.

### Cancer Types

In some embodiments, the cancer is selected from renal cell carcinoma (RCC), non-small cell lung cancer (NSCLC), head and neck squamous cell cancer (HNSCC), classical Hodgkin lymphoma (cHL), primary mediastinal large B-cell lymphoma (PMBCL), urothelial carcinoma, microsatellite unstable cancer, microsatellite stable cancer, gastric cancer, colon cancer, colorectal cancer (CRC), cervical cancer, hepatocellular carcinoma (HCC), Merkel cell carcinoma (MCC), melanoma, small cell lung cancer (SCLC), esophageal, esophageal squamous cell carcinoma (ESCC), glioblastoma, mesothelioma, breast cancer, triple-negative breast cancer, prostate cancer, castrate-resistant prostate cancer, metastatic castrate-resistant prostate cancer, or metastatic castrate-resistant prostate cancer having DNA damage response (DDR) defects, bladder cancer, ovarian cancer, tumors of moderate to low mutational burden, cutaneous squamous cell carcinoma (CSCC), squamous cell skin cancer (SCSC), tumors of low-to non-expressing PD-L1, tumors disseminated systemically to the liver and CNS beyond their primary anatomic originating site, and diffuse large B-cell lymphoma.

In some embodiments, the cancer in the subject is selected from renal cell carcinoma (RCC), non-small cell lung cancer (NSCLC), urothelial carcinoma, melanoma, Merkel cell carcinoma (MCC), and head and neck squamous cell cancer (HNSCC). In some embodiments, the cancer is renal cell carcinoma (RCC). In some embodiments, the cancer is non-small cell lung cancer (NSCLC). In some embodiments, the cancer is urothelial carcinoma. In some embodiments, the cancer is melanoma. In some embodiments, the cancer is Merkel cell carcinoma (MCC). In some embodiments, the cancer is head and neck squamous cell cancer (HNSCC).

In some embodiments, the cancer is in the form of a solid tumor. In some embodiments, the cancer is an advanced or metastatic solid tumor. In some embodiments, the cancer is in the form of a liquid tumor.

### Administration

In some embodiments, the response is a complete response, a partial response or stable disease. In some embodiments, the IL-2 conjugate is administered to the subject by intravenous, subcutaneous, intramuscular, intracerebral, intranasal, intra-arterial, intra-articular, intradermal, intravitreal, intraosseous infusion, intraperitoneal, or intrathecal administration. In some embodiments, the IL-2 conjugate is administered to the subject by intravenous, subcutaneous, or intramuscular administration. In some embodiments, the IL-2 conjugate is administered to the subject by intravenous administration. In some embodiments, the IL-2 conjugate is administered to the subject by subcutaneous administration. In some embodiments, the IL-2 conjugate is administered to the subject by intramuscular administration.

In some embodiments, the duration of the treatment is up to 24 months, such as 1 month, 2 months, 3 months, 6 months, 9 months, 12 months, 15 months, 18 months, 21 months or 24 months. In some embodiments, the duration of treatment is further extended by up to another 24 months.

In some embodiments, the IL-2 conjugate is administered to the subject prior to the administration to the subject of the anti-EGFR antibody. In some embodiments, the anti-EGFR antibody is administered to the subject prior to the administration to the subject of the IL-2 conjugate. In some embodiments, the IL-2 conjugate and the anti-EGFR antibody are simultaneously administered to the subject. In some embodiments, the IL-2 conjugate is administered to the subject separately from the administration of the anti-EGFR antibody. In some embodiments, the IL-2 conjugate and the anti-EGFR antibody are administered sequentially to the subject. In some embodiments, the IL-2 conjugate and the anti-EGFR antibody are administered to the subject on the same day. In some embodiments, the IL-2 conjugate and the anti-EGFR antibody are administered to the subject on different days.

In some embodiments, an effective amount of the IL-2 conjugate is administered to a subject in need thereof once per week, once every two weeks, once every three weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks, once every 17 weeks, once every 18 weeks, once every 19 weeks, once every 20 weeks, once every 21 weeks, once every 22 weeks, once every 23 weeks, once every 24 weeks, once every 25 weeks, once every 26 weeks, once every 27 weeks, or once every 28 weeks. In some embodiments, an effective amount of the IL-2 conjugate is administered to a subject in need thereof once per week. In some embodiments, an effective amount of the IL-2 conjugate is administered to a subject in need thereof once every two weeks. In some embodiments, an effective amount of the IL-2 conjugate is administered to a subject in need thereof once every three weeks. In some embodiments, an effective amount of the IL-2 conjugate is administered to a subject in need thereof once every 4 weeks. In some embodiments, an effective amount of the IL-2 conjugate is administered to a subject in need thereof once every 5 weeks. In some embodiments, an effective amount of the IL-2 conjugate is administered to a subject in need thereof once every 6 weeks. In some embodiments, an effective amount of the IL-2 conjugate is administered to a subject in need thereof once every 7 weeks. In some embodiments, an effective amount of the IL-2 conjugate is administered to a subject in need thereof once every 8 weeks. In some embodiments, an effective amount of the IL-2 conjugate is administered to a subject in need thereof once every 9 weeks. In some embodiments, an effective amount of the IL-2 conjugate is administered to a subject in need thereof once every 10 weeks. In some embodiments, an effective amount of the IL-2 conjugate is administered to a subject in need thereof once every 11 weeks. In some embodiments, an effective amount of the IL-2 conjugate is administered to a subject in need thereof once every 12 weeks. In some embodiments, an effective amount of the IL-2 conjugate is administered to a subject in need thereof once every 13 weeks. In some embodiments, an effective amount of the IL-2 conjugate is administered to a subject in need thereof once every 14 weeks. In some embodiments, an effective amount of the IL-2 conjugate is administered to a subject in need thereof once every 15 weeks. In some embodiments, an effective amount of the IL-2 conjugate is administered to a subject in need thereof once every 16 weeks. In some embodiments, an effective amount of the IL-2 conjugate is administered to a subject in need thereof once every 17 weeks. In some embodiments, an effective amount of the IL-2 conjugate is administered to a subject in need thereof once every 18 weeks. In some embodiments, an effective amount of the IL-2 conjugate is administered to a subject in need thereof once every 19 weeks. In some embodiments, an effective amount of the IL-2 conjugate is administered to a subject in need thereof once every 20 weeks. In some embodiments, an effective amount of the IL-2 conjugate is administered to a subject in need thereof once every 21 weeks. In some embodiments, an effective amount of the IL-2 conjugate is administered to a subject in need thereof once every 22 weeks. In some embodiments, an effective amount of the IL-2 conjugate is administered to a subject in need thereof once every 23 weeks. In some embodiments, an effective amount of the IL-2 conjugate is administered to a subject in need thereof once every 24 weeks. In some embodiments, an effective amount of the IL-2 conjugate is administered about once every 7, 8, 9, 10, 11, 12, 13, 14, 15, 16, 17, 18, 19, 20, or 21 days. In some embodiments, an effective amount of the IL-2 conjugate is administered about once every 14, 15, 16, 17, 18, 19, 20, or 21 days.

In some embodiments, the amount of a given agent that corresponds to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

In some embodiments, the IL-2 conjugate is administered at a dose from about 8 μg/kg to 24 μg/kg. In some embodiments, the IL-2 conjugate is administered at a dose of about 8 μg/kg. In some embodiments, the IL-2 conjugate is administered at a dose of about 16 μg/kg. In some embodiments, the IL-2 conjugate is administered at a dose of about 24 μg/kg. In any of these embodiments, the IL-2 conjugate is administered at a dose as described herein every 3 weeks.

In some embodiments, an anti-EGFR antibody may be administered at a dose and using a dosing regimen that has been determined to be safe and efficacious for that antibody alone or in combination with an IL-2 conjugate. In some embodiments, an anti-EGFR antibody is administered by intravenous infusion. In some embodiments, cetuximab (or another anti-EGFR antibody) is administered to a subject in need thereof once per week, once every two weeks, once every three weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks, once every 17 weeks, once every 18 weeks, once every 19 weeks, once every 20 weeks, once every 21 weeks, once every 22 weeks, once every 23 weeks, once every 24 weeks, once every 25 weeks, once every 26 weeks, once every 27 weeks, or once every 28 weeks. In some embodiments, cetuximab (or another anti-EGFR antibody) is administered to a subject in need thereof once per week. In some embodiments, cetuximab (or another anti-EGFR antibody) is administered to a subject in need thereof once every two weeks. In some embodiments, cetuximab (or another anti-EGFR antibody) is administered to a subject in need thereof once every three weeks. In some embodiments, cetuximab (or another anti-EGFR antibody) is administered to a subject in need thereof once every 4 weeks. In some embodiments, cetuximab (or another anti-EGFR antibody) is administered to a subject in need thereof once every 5 weeks. In some embodiments, cetuximab (or another anti-EGFR antibody) is administered to a subject in need thereof once every 6 weeks. In some embodiments, cetuximab (or another anti-EGFR antibody) is administered to a subject in need thereof once every 7 weeks. In some embodiments, cetuximab (or another anti-EGFR antibody) is administered to a subject in need thereof once every 8 weeks. In some embodiments, cetuximab (or another anti-EGFR antibody) is administered to a subject in need thereof once every 9 weeks. In some embodiments, cetuximab (or another anti-EGFR antibody) is administered to a subject in need thereof once every 10 weeks. In some embodiments, cetuximab (or another anti-EGFR antibody) is administered to a subject in need thereof once every 11 weeks. In some embodiments, cetuximab (or another anti-EGFR antibody) is administered to a subject in need thereof once every 12 weeks. In some embodiments, cetuximab (or another anti-EGFR antibody) is administered to a subject in need thereof once every 13 weeks. In some embodiments, cetuximab (or another anti-EGFR antibody) is administered to a subject in need thereof once every 14 weeks. In some embodiments, cetuximab (or another anti-EGFR antibody) is administered to a subject in need thereof once every 15 weeks. In some embodiments, cetuximab (or another anti-EGFR antibody) is administered to a subject in need thereof once every 16 weeks. In some embodiments, cetuximab (or another anti-EGFR antibody) is administered to a subject in need thereof once every 17 weeks. In some embodiments, cetuximab (or another anti-EGFR antibody) is administered to a subject in need thereof once every 18 weeks. In some embodiments, cetuximab (or another anti-EGFR antibody) is administered to a subject in need thereof once every 19 weeks. In some embodiments, cetuximab (or another anti-EGFR antibody) is administered to a subject in need thereof once every 20 weeks. In some embodiments, cetuximab (or another anti-EGFR antibody) is administered to a subject in need thereof once every 21 weeks. In some embodiments, cetuximab (or another anti-EGFR antibody) is administered to a subject in need thereof once every 22 weeks. In some embodiments, cetuximab (or another anti-EGFR antibody) is administered to a subject in need thereof once every 23 weeks. In some embodiments, cetuximab (or another anti-EGFR antibody) is administered to a subject in need thereof once every 24 weeks. In some embodiments, cetuximab (or another anti-EGFR antibody) is administered about once every 7, 8, 9, 10, 11, 12, 13, 14, 15, 16, 17, 18, 19, 20, or 21 days.

In some embodiments, the anti-EGFR antibody is cetuximab. In some embodiments, cetuximab is administered at a loading dose from about 100 mg/m² to about 500 mg/m² by intravenous infusion. In any of the embodiments described herein, the loading dose of cetuximab is mg/m² of the subject's body surface area. In some embodiments, cetuximab is administered at a loading dose of about 100 mg/m² by intravenous infusion. In some embodiments, cetuximab is administered at a loading dose of about 150 mg/m² by intravenous infusion. In some embodiments, cetuximab is administered at a loading dose of about 200 mg/m² by intravenous infusion. In some embodiments, cetuximab is administered at a loading dose of about 250 mg/m² by intravenous infusion. In some embodiments, cetuximab is administered at a loading dose of about 300 mg/m² by intravenous infusion. In some embodiments, cetuximab is administered at a loading dose of about 350 mg/m² by intravenous infusion. In some embodiments, cetuximab is administered at a loading dose of about 400 mg/m² by intravenous infusion. In some embodiments, cetuximab is administered at a loading dose of about 450 mg/m² by intravenous infusion. In some embodiments, cetuximab is administered at a loading dose of about 500 mg/m² by intravenous infusion. In some embodiments, the initial dose of cetuximab is administered at a loading dose of about 400 mg/m² by intravenous infusion, and all subsequent doses of cetuximab are administered at a loading dose of about 250 mg/m² by intravenous infusion. In any of these embodiments, cetuximab is infused over about 30-240 minutes. In some embodiments, cetuximab is infused over about 30 minutes. In some embodiments, cetuximab is infused over about 60 minutes. In some embodiments, cetuximab is infused over about 90 minutes. In some embodiments, cetuximab is infused over about 120 minutes. In some embodiments, cetuximab is infused over about 150 minutes. In some embodiments, cetuximab is infused over about 180 minutes. In some embodiments, cetuximab is infused over about 210 minutes. In some embodiments, cetuximab is infused over about 240 minutes. In any of these embodiments, cetixumab is administered at an infusion rate of about 1 mg/min to about 10 mg/min, such as 1 mg/min, 2 mg/min, 3 mg/min, 4 mg/min, 5 mg/min, 6 mg/min, 7 mg/min, 8 mg/min, 9 mg/min, or 10 mg/min. In some embodiments, the first dose of cetuximab is administered at a higher loading dose than the dose of subsequent doses of cetuximab. In some embodiments, the infusion time of the first dose of cetuximab is longer than the infusion time of subsequent doses of cetuximab. In some embodiments, cetuximab is administered at a dose as described herein every 3 weeks. In some embodiments, cetuximab is administered at a dose as described herein every 2 weeks. In some embodiments, cetuximab is administered at a dose as described herein every week.

### Additional Agents Premedication

In some embodiments, any of the methods described herein further comprises administering an antihistamine. In some embodiments, the antihistamine is cetirizine. In some embodiments, the antihistamine is promethazine. In some embodiments, the antihistamine is dexchlorpheniramine. In some embodiments, the antihistamine is diphenhydramine. In some embodiments, diphenhydramine is administered intravenously at a dose from about 25 to 50 mg.

In some embodiments, any of the methods described herein further comprises administering an analgesic, such as acetaminophen. In some embodiments, acetaminophen is administered orally at a dose from about 650 to 1000 mg.

In some embodiments, any of the methods described herein further comprises administering a serotonin 5-HT₃ receptor antagonist. In some embodiments, the serotonin 5-HT₃ receptor antagonist is granisetron. In some embodiments, the serotonin 5-HT₃ receptor antagonist is dolasetron. In some embodiments, the serotonin 5-HT₃ receptor antagonist is tropisetron. In some embodiments, the serotonin 5-HT₃ receptor antagonist is palonosetron. In some embodiments, the serotonin 5-HT₃ receptor antagonist is ondansetron. In some embodiments, ondansetron is administered intravenously at a dose from about 8 mg to 0.15 mg/kg.

In some embodiments, any of the methods described herein further comprises administering an antihistamine (such as cetirizine, promethazine, dexchlorpheniramine, or diphenhydramine), an analgesic (such as acetaminophen), and/or a serotonin 5-HT₃ receptor antagonist (such as granisetron, dolasetron, tropisetron, palonosetron, or ondansetron). In some embodiments, the method further comprises administering an antihistamine (such as cetirizine, promethazine, dexchlorpheniramine, or diphenhydramine) and an analgesic (such as acetaminophen). In some embodiments, the method further comprising administering an antihistamine (such as cetirizine, promethazine, dexchlorpheniramine, or diphenhydramine) and a serotonin 5-HT₃ receptor antagonist (such as granisetron, dolasetron, tropisetron, palonosetron, or ondansetron). In some embodiments, the method further comprising administering an analgesic (such as acetaminophen) and a serotonin 5-HT₃ receptor antagonist (such as granisetron, dolasetron, tropisetron, palonosetron, or ondansetron). In some embodiments, any of the methods described herein further comprises administering an antihistamine (such as cetirizine, promethazine, dexchlorpheniramine, or diphenhydramine), an analgesic (such as acetaminophen), and a serotonin 5-HT₃ receptor antagonist (such as granisetron, dolasetron, tropisetron, palonosetron, or ondansetron).

In some embodiments, any of the methods described herein further comprises administering a premedication, for example to prevent or reduce the acute effect of infusion-associated reactions (IAR) or flu-like symptoms. In some embodiments, the premedication is administered prior to administering the IL-2 conjugate and/or the anti-EGFR antibody (such as cetuximab). In some embodiments, the premedication is administered prior to administering the IL-2 conjugate. In some embodiments, the premedication is administered prior to administering the anti-EGFR antibody (such as cetuximab). In some embodiments, the premedication is administered prior to administering the IL-2 conjugate and the anti-EGFR antibody (such as cetuximab).

In some embodiments, the premedication for the IL-2 conjugate is different from the premedication for the anti-EGFR antibody (such as cetuximab). In some embodiments, the premedication for the IL-2 conjugate is the same as the premedication for the anti-EGFR antibody (such as cetuximab). In some instances where the premedication for the IL-2 conjugate and the anti-EGFR antibody (such as cetuximab) is the same, only a single dose of premedication is administered. In other instances where the premedication for the IL-2 conjugate and the anti-EGFR antibody (such as cetuximab) is the same, multiple doses of premedication are administered. In some embodiments, the premedication is administered for all doses administered of the IL-2 conjugate. In some embodiments, the premedication is administered for the first 1, 2, 3, 4, 5, 6, 7, 8, 9, or 10 doses of the IL-2 conjugate and not for any subsequent doses of the IL-2 conjugate. In some embodiments, the premedication is administered for the first 4 doses of the IL-2 conjugate and not for any subsequent doses of the IL-2 conjugate. In some embodiments, the premedication is administered for all doses administered of the anti-EGFR antibody (such as cetuximab). In some embodiments, the premedication is administered for the first 1, 2, 3, 4, 5, 6, 7, 8, 9, or 10 doses of the anti-EGFR antibody (such as cetuximab) and not for any subsequent doses of the anti-EGFR antibody. In some embodiments, the premedication is administered for the first dose of the anti-EGFR antibody (such as cetuximab) and not for any subsequent doses of the anti-EGFR antibody.

In some embodiments, any of the methods described herein further comprises administering premedication prior to administering the IL-2 conjugate. In some embodiments, the IL-2 conjugate premedication is an antihistamine, such as cetirizine, promethazine, dexchlorpheniramine, or diphenhydramine. In some embodiments, the antihistamine is diphenhydramine. In some embodiments, diphenhydramine is administered intravenously at a dose from about 25 to 50 mg. In some embodiments, the IL-2 conjugate premedication is a serotonin 5-HT₃ receptor antagonist (such as granisetron, dolasetron, tropisetron, palonosetron, or ondansetron). In some embodiments, the serotonin 5-HT₃ receptor antagonist is ondansetron. In some embodiments, ondansetron is administered intravenously at a dose from about 8 mg to 0.15 mg/kg. In some embodiments, the IL-2 conjugate premedication is an analgesic (such as acetaminophen). In some embodiments, acetaminophen is administered orally at a dose from about 650 to 1000 mg.

In some embodiments, any of the methods described herein further comprises administering premedication prior to administering the anti-EGFR antibody (such as cetuximab). In some embodiments, the anti-EGFR antibody premedication is an antihistamine, such as cetirizine, promethazine, dexchlorpheniramine, or diphenhydramine. In some embodiments, the antihistamine is diphenhydramine. In some embodiments, diphenhydramine is administered intravenously at a dose from about 25 to 50 mg. In some embodiments, the cetuximab premedication is a serotonin 5-HT₃ receptor antagonist (such as granisetron, dolasetron, tropisetron, palonosetron, or ondansetron). In some embodiments, the serotonin 5-HT₃ receptor antagonist is ondansetron. In some embodiments, ondansetron is administered intravenously at a dose from about 8 mg to 0.15 mg/kg. In some embodiments, the anti-EGFR antibody premedication is an analgesic (such as acetaminophen). In some embodiments, acetaminophen is administered orally at a dose from about 650 to 1000 mg.

In some embodiments, any of the methods described herein further comprises administering a first dose of premedication prior to administering the IL-2 conjugate and a second dose of premedication prior to administering the anti-EGFR antibody (such as cetuximab). In some embodiments, the premedication for the IL-2 conjugate is the same as the premedication for the anti-EGFR antibody (such as cetuximab). In some embodiments, the premedication for the IL-2 conjugate is different from the premedication for the anti-EGFR antibody (such as cetuximab). In some embodiments, the premedication is an antihistamine, such as cetirizine, promethazine, dexchlorpheniramine, or diphenhydramine. In some embodiments, the antihistamine is diphenhydramine. In some embodiments, diphenhydramine is administered intravenously at a dose from about 25 to 50 mg. In some embodiments, the premedication is a serotonin 5-HT₃ receptor antagonist (such as granisetron, dolasetron, tropisetron, palonosetron, or ondansetron). In some embodiments, the serotonin 5-HT₃ receptor antagonist is ondansetron. In some embodiments, ondansetron is administered intravenously at a dose from about 8 mg to 0.15 mg/kg. In some embodiments, the premedication is an analgesic (such as acetaminophen). In some embodiments, acetaminophen is administered orally at a dose from about 650 to 1000 mg. In some embodiments, the premedication comprises an antihistamine and a serotonin 5-HT₃ receptor antagonist. In some embodiments, the premedication comprises an antihistamine and an analgesic. In some embodiments, the premedication comprises a serotonin 5-HT₃ receptor antagonist and an analgesic. In some embodiments, the premedication comprises an antihistamine, a serotonin 5-HT₃ receptor antagonist, and an analgesic. In some instances where the premedication for the IL-2 conjugate and the anti-EGFR antibody (such as cetuximab) is the same (such as diphenhydramine), only a single dose of premedication is administered. In other instances where the premedication for the IL-2 conjugate and the anti-EGFR antibody (such as cetuximab) is the same, multiple doses of premedication are administered.

In some embodiments of the methods described herein, the dosing sequence is as follows: (i) premedication for the anti-EGFR antibody (such as cetuximab); (ii) the anti-EGFR antibody (such as cetuximab); (iii) premedication for the IL-2 conjugate; and (iv) the IL-2 conjugate. In some variations where the premedication for the anti-EGFR antibody (such as cetuximab) is the same as the premedication for the IL-2 conjugate (such as diphenhydramine), administering the premedication for the IL-2 conjugate may be omitted. In some embodiments, the dosing sequence is as follows: (i) premedication for the IL-2 conjugate; (ii) the IL-2 conjugate; (iii) premedication for the anti-EGFR antibody (such as cetuximab); and (iv) the anti-EGFR antibody (such as cetuximab). In some variations where the premedication for the anti-EGFR antibody (such as cetuximab) is the same as the premedication for the IL-2 conjugate (such as diphenhydramine), administering the premedication for the anti-EGFR antibody may be omitted.

### Subject

In some embodiments, administration of the effective amount of the IL-2 conjugate and an anti-EGFR antibody is to an adult. In some embodiments, the adult is a male. In other embodiments, the adult is a female. In some embodiments, the adult is at least age 20, 25, 30, 35, 40, 45, 50, 55, 60, 65, 70, 75, 80, 85, 90, or 95 years of age. In some embodiments, administration of the effective amount of the IL-2 conjugate and an anti-EGFR antibody is to an infant, child, or adolescent. In some embodiments, the subject is at least 1 month, 2 months, 3 months, 6 months, 9 months or 12 months of age. In some embodiments, the subject is at least 1, 2, 3, 4, 5, 6, 7, 8, 9, 10, 11, 12, 13, 14, 15, 16, 17, 18, or 19 years of age.

In some embodiments, the subject has measurable disease (i.e., cancer) as determined by RECIST v1.1. In some embodiments, the subject has been determined to have Eastern Cooperative Oncology Group (ECOG) performance status of 0 or 1. In some embodiments, the subject has adequate cardiovascular, hematological, liver, and renal function, as determined by a physician. In some embodiments, the subject has been determined (e.g., by a physician) to have a life expectancy greater than or equal to 12 weeks.

In some embodiments, the subject has adequate cardiovascular, hematological, liver, and renal function.

In some embodiments, the subject has histologically or cytologically confirmed diagnosis of advanced and/or metastatic solid tumors with at least one tumor lesion with location accessible to safely biopsy per clinical judgment (i.e., as determined by a physician). In some embodiments, the subject has had prior anti-cancer therapy before administration of the first treatment dose. In some embodiments, treatment related toxicity of the prior anti-cancer therapy has been resolved to an appropriate level.

In some embodiments, the subject is a female of childbearing potential and is using a medically-accepted method of birth control during the treatment and for at least 3 months after the last treatment dose is administered. In some embodiments, the subject is a pre-menopausal female who has tested negative for pregnancy (by a serum pregnancy test) within 7 days prior to administration of the first treatment dose. In some embodiments, the subject is a female less than 12 months after menopause who has tested negative for pregnancy (by a serum pregnancy test) within 7 days prior to administration of the first treatment dose.

In some embodiments, the subject is a male who is not surgically sterile and who is using a medically-accepted method of birth control during the treatment and for at least 3 months after the last dose is administered. In some embodiments, the male is not donating or banking sperm during the treatment period and for at least 3 months after administration of the last treatment dose.

In some embodiments, the subject has not received radiotherapy within 14 days of administration of the first treatment dose. In some embodiments, the subject has not received palliative radiation or stereotactic radiosurgery within 7 days of administration of the first treatment dose.

In some embodiments, the subject has not been treated with systemic anti-cancer therapy or an investigational anti-cancer agent within 2 weeks of administration of the first treatment dose. In some embodiments, the subject has not been treated with immunotherapy or tyrosine kinase inhibitor therapy within 4 weeks of administration of the first treatment dose.

In some embodiments, the subject has not had major surgery within 30 days of administration of the first treatment dose. In some embodiments, the subject has had major surgery more than 30 days prior to administration of the first treatment dose and has recovered to at least Grade 1 from any adverse effects associated with the procedure. In some embodiments, the subject does not anticipate the need for major surgery during the course of treatment.

In some embodiments, the subject has not had active autoimmune disease requiring systemic treatment within 3 months prior to administration of the first treatment dose. In some embodiments, the subject has not had a documented history of clinically severe autoimmune disease that requires systemic steroids or immunosuppressive agents prior to administration of the first treatment dose.

In some embodiments, the subject does not have primary central nervous system (CNS) disease or leptomeningeal disease. In some embodiments, the subject has known CNS metastases but has received appropriate treatment and is asymptomatic, without evidence of radiological progression for at least 8 weeks prior to administration of the first treatment dose, and has had no requirement for steroids or enzyme inducing anticonvulsants within 14 days prior to administration of the first treatment dose.

In some embodiments, the subject has not had abnormal pulmonary function, including pneumonitis, active pneumonitis, interstitial lung disease requiring the use of steroids, idiopathic pulmonary fibrosis, confirmed pleural effusion, and severe dyspnea at rest or requiring supplementary oxygen therapy, within 6 months of administration of the first treatment dose.

In some embodiments, the subject has not taken parenteral antibiotics within 14 days of administration of the first treatment dose.

In some embodiments, the subject does not have a history of allogenic or solid organ transplant. In some embodiments, the subject does not have human immunodeficiency virus (HIV) infection or active infection with hepatitis C. In some embodiments, the subject does not have uncontrolled hepatitis B virus (HBV) infection.

In some embodiments, the subject has had no clinically significant bleeding (e.g., gastrointestinal bleeding, intracranial hemorrhage) within 2 weeks prior to administration of the first treatment dose. In some embodiments, the subject has not had a prior diagnosis of deep vein thrombosis or pulmonary embolism within 3 months of administration of the first treatment dose.

In some embodiments, the subject has not had a severe or unstable cardiac condition (such as congestive heart failure (New York Heart Association Class III or IV), cardiac bypass surgery or coronary artery stent placement, angioplasty, cardiac ejection fraction below the lower limit of normal, unstable angina, medically uncontrolled hypertension (e.g. ≥160 mm Hg systolic or ≥100 mm Hg diastolic), uncontrolled cardiac arrhythmia requiring medication (≥grade 2, according to NCI CTCAE v5.0), or myocardial infarction) within 6 months prior to administration of the first treatment dose.

In some embodiments, the subject has no history of non-pharmacologically induced prolonged corrected QT interval determined using Fridericia's formula (QTcF)>450 milliseconds (msec) in males or >470 msec in females.

In some embodiments, the subject has no known hypersensitivity or contraindications to any of the IL-2 conjugates disclosed herein, PEG, pegylated drugs, or anti-EGFR antibody, such as, for example, cetuximab.

In some embodiments, the subject does not have an active second malignancy. In some embodiments, the subject does not have a history of a previous malignancy. In some embodiments, the subject has had a non-melanomatous skin cancer or cervical cancer that has been curatively surgically resected prior to administration of the first treatment dose.

In some embodiments, the subject does not have any serious medical condition (including pre-existing autoimmune disease or inflammatory disorder), laboratory abnormality, psychiatric condition, or any other significant or unstable concurrent medical illness that would preclude treatment or would make treatment inappropriate.

In some embodiments, the subject is not pregnant or breastfeeding. In some embodiments, the subject is not expecting to conceive or father children during the course of the treatment and following up to 3 months after administration of the final treatment dose.

In some embodiments, the subject is not receiving a concurrent therapy with any investigational agent, vaccine, or device during the course of treatment. In some embodiments, the subject is receiving concurrent therapy with an investigational agent, vaccine, or device during the course of treatment after physician approval.

### Effects of Administration

In some embodiments, following administration of the IL-2 conjugate and an anti-EGFR antibody, the subject experiences a response as measured by the Immune-related Response Evaluation Criteria in Solid Tumors (iRECIST). In some embodiments, following administration of the IL-2 conjugate and an anti-EGFR antibody, the subject experiences an Objective Response Rate (ORR) according to RECIST version 1.1. In some embodiments, following administration of the IL-2 conjugate and an anti-EGFR antibody, the subject experiences Duration of Response (DOR) according to RECIST versions 1.1. In some embodiments, following administration of the IL-2 conjugate and an anti-EGFR antibody, the subject experiences Progression-Free Survival (PFS) according to RECIST version 1.1. In some embodiments, following administration of the IL-2 conjugate and an anti-EGFR antibody, the subject experiences Overall Survival according to RECIST version 1.1. In some embodiments, following administration of the IL-2 conjugate and an anti-EGFR antibody, the subject experiences Time to Response (TTR) according to RECIST version 1.1. In some embodiments, following administration of the IL-2 conjugate and an anti-EGFR antibody, the subject experiences Disease Control Rate (DCR) according to RECIST version 1.1. In any of these embodiments, the subject's experience is based on a physician's review of a radiographic image taken of the subject.

In some embodiments, treatment is discontinued based on a physician's review of a radiographic image taken of the subject.

In some embodiments, treatment is discontinued based on a physician's review of immunophenotyping of peripheral blood at various timepoints. In some embodiments, treatment is discontinued based on a physician's review of immunophenotyping of tumor samples at various timepoints. In some embodiments, treatment is discontinued based on a physician's review of the presence of antibodies to any of the IL-2 conjugates disclosed herein at various timepoints. In some embodiments, treatment is discontinued based on a physician's review of the plasma concentration of any of the IL-2 conjugates disclosed herein at various timepoints. In any of these embodiments, the physican's review is based on an appropriate assay of the relevant parameters.

In some embodiments, administration of the effective amount of the IL-2 conjugate and an anti-EGFR antibody to the subject does not cause vascular leak syndrome in the subject. In some embodiments, administration of the effective amount of the IL-2 conjugate and an anti-EGFR antibody to the subject does not cause Grade 2, Grade 3, or Grade 4 vascular leak syndrome in the subject. In some embodiments, administration of the effective amount of the IL-2 conjugate and an anti-EGFR antibody to the subject does not cause Grade 2 vascular leak syndrome in the subject. In some embodiments, administration of the effective amount of the IL-2 conjugate and an anti-EGFR antibody to the subject does not cause Grade 3 vascular leak syndrome in the subject. In some embodiments, administration of the effective amount of the IL-2 conjugate and an anti-EGFR antibody to the subject does not cause Grade 4 vascular leak syndrome in the subject.

In some embodiments, administration of the effective amount of the IL-2 conjugate and an anti-EGFR antibody to the subject does not cause loss of vascular tone in the subject.

In some embodiments, administration of the effective amount of the IL-2 conjugate and an anti-EGFR antibody to the subject does not cause extravasation of plasma proteins and fluid into the extravascular space in the subject.

In some embodiments, administration of the effective amount of the IL-2 conjugate and an anti-EGFR antibody to the subject does not cause hypotension and reduced organ perfusion in the subject.

In some embodiments, administration of the effective amount of the IL-2 conjugate and an anti-EGFR antibody to the subject does not cause impaired neutrophil function in the subject. In some embodiments, administration of the effective amount of the IL-2 conjugate and an anti-EGFR antibody to the subject does not cause reduced chemotaxis in the subject.

In some embodiments, administration of the effective amount of the IL-2 conjugate and an anti-EGFR antibody to the subject is not associated with an increased risk of disseminated infection in the subject. In some embodiments, the disseminated infection is sepsis or bacterial endocarditis. In some embodiments, the disseminated infection is sepsis. In some embodiments, the disseminated infection is bacterial endocarditis. In some embodiments, the subject is treated for any preexisting bacterial infections prior to administration of the IL-2 conjugate and an anti-EGFR antibody. In some embodiments, the subject is treated with an antibacterial agent selected from oxacillin, nafcillin, ciprofloxacin, and vancomycin prior to administration of the IL-2 conjugate and an anti-EGFR antibody.

In some embodiments, administration of the effective amount of the IL-2 conjugate and an anti-EGFR antibody to the subject does not exacerbate a pre-existing or initial presentation of an autoimmune disease or an inflammatory disorder in the subject. In some embodiments, the administration of the effective amount of the IL-2 conjugate and an anti-EGFR antibody to the subject does not exacerbate a pre-existing or initial presentation of an autoimmune disease in the subject. In some embodiments, the administration of the effective amount of the IL-2 conjugate and an anti-EGFR antibody to the subject does not exacerbate a pre-existing or initial presentation of an inflammatory disorder in the subject. In some embodiments, the autoimmune disease or inflammatory disorder in the subject is selected from Crohn's disease, scleroderma, thyroiditis, inflammatory arthritis, diabetes mellitus, oculo-bulbar myasthenia gravis, crescentic IgA glomerulonephritis, cholecystitis, cerebral vasculitis, Stevens-Johnson syndrome and bullous pemphigoid. In some embodiments, the autoimmune disease or inflammatory disorder in the subject is Crohn's disease. In some embodiments, the autoimmune disease or inflammatory disorder in the subject is scleroderma. In some embodiments, the autoimmune disease or inflammatory disorder in the subject is thyroiditis. In some embodiments, the autoimmune disease or inflammatory disorder in the subject is inflammatory arthritis. In some embodiments, the autoimmune disease or inflammatory disorder in the subject is diabetes mellitus. In some embodiments, the autoimmune disease or inflammatory disorder in the subject is oculo-bulbar myasthenia gravis. In some embodiments, the autoimmune disease or inflammatory disorder in the subject is crescentic IgA glomerulonephritis. In some embodiments, the autoimmune disease or inflammatory disorder in the subject is cholecystitis. In some embodiments, the autoimmune disease or inflammatory disorder in the subject is cerebral vasculitis. In some embodiments, the autoimmune disease or inflammatory disorder in the subject is Stevens-Johnson syndrome. In some embodiments, the autoimmune disease or inflammatory disorder in the subject is bullous pemphigoid.

In some embodiments, administration of the effective amount of the IL-2 conjugate and an anti-EGFR antibody to the subject does not cause changes in mental status, speech difficulties, cortical blindness, limb or gait ataxia, hallucinations, agitation, obtundation, or coma in the subject. In some embodiments, administration of the effective amount of the IL-2 conjugate and an anti-EGFR antibody to the subject does not cause seizures in the subject. In some embodiments, administration of the effective amount of the IL-2 conjugate and an anti-EGFR antibody to the subject is not contraindicated in subjects having a known seizure disorder.

In some embodiments, administration of the effective amount of the IL-2 conjugate and an anti-EGFR antibody to the subject does not cause capillary leak syndrome in the subject. In some embodiments, administration of the effective amount of the IL-2 conjugate and an anti-EGFR antibody to the subject does not cause Grade 2, Grade 3, or Grade 4 capillary leak syndrome in the subject. In some embodiments, administration of the effective amount of the IL-2 conjugate and an anti-EGFR antibody to the subject does not cause Grade 2 capillary leak syndrome in the subject. In some embodiments, administration of the effective amount of the IL-2 conjugate and an anti-EGFR antibody to the subject does not cause Grade 3 capillary leak syndrome in the subject. In some embodiments, administration of the effective amount of the IL-2 conjugate and an anti-EGFR antibody to the subject does not cause Grade 4 capillary leak syndrome in the subject.

In some embodiments, administration of the effective amount of the IL-2 conjugate and an anti-EGFR antibody to the subject does not cause a drop in mean arterial blood pressure in the subject following administration of the IL-2 conjugate to the subject. In some embodiments, administration of the effective amount of the IL-2 conjugate and an anti-EGFR antibody to the subject does cause hypotension in the subject. In some embodiments, administration of the effective amount of the IL-2 conjugate and an anti-EGFR antibody to the subject does not cause the subject to experience a systolic blood pressure below 90 mm Hg or a 20 mm Hg drop from baseline systolic pressure.

In some embodiments, administration of the effective amount of the IL-2 conjugate and an anti-EGFR antibody to the subject does not cause edema or impairment of kidney or liver function in the subject.

In some embodiments, administration of the effective amount of the IL-2 conjugate and an anti-EGFR antibody to the subject does not cause eosinophilia in the subject. In some embodiments, administration of the effective amount of the IL-2 conjugate and an anti-EGFR antibody to the subject does not cause the eosinophil count in the peripheral blood of the subject to exceed 500 per μL. In some embodiments, administration of the effective amount of the IL-2 conjugate and an anti-EGFR antibody to the subject does not cause the eosinophil count in the peripheral blood of the subject to exceed 500 μL to 1500 per μL. In some embodiments, administration of the effective amount of the IL-2 conjugate and an anti-EGFR antibody to the subject does not cause the eosinophil count in the peripheral blood of the subject to exceed 1500 per μL to 5000 per μL. In some embodiments, administration of the effective amount of the IL-2 conjugate and an anti-EGFR antibody to the subject does not cause the eosinophil count in the peripheral blood of the subject to exceed 5000 per μL. In some embodiments, administration of the effective amount of the IL-2 conjugate and an anti-EGFR antibody to the subject is not contraindicated in subjects on an existing regimen of psychotropic drugs.

In some embodiments, administration of the effective amount of the IL-2 conjugate and an anti-EGFR antibody to the subject is not contraindicated in subjects on an existing regimen of nephrotoxic, myelotoxic, cardiotoxic, or hepatotoxic drugs. In some embodiments, administration of the effective amount of the IL-2 conjugate and an anti-EGFR antibody the subject is not contraindicated in subjects on an existing regimen of aminoglycosides, cytotoxic chemotherapy, doxorubicin, methotrexate, or asparaginase. In some embodiments, administration of the effective amount of the IL-2 conjugate and an anti-EGFR antibody to the subject is not contraindicated in subjects receiving combination regimens containing antineoplastic agents. In some embodiments, the antineoplastic agent is selected from dacarbazine, cis-platinum, tamoxifen and interferon-alpha.

In some embodiments, administration of the effective amount of the IL-2 conjugate and an anti-EGFR antibody to the subject does not cause one or more Grade 4 adverse events in the subject following administration of the IL-2 conjugate to the subject. In some embodiments, Grade 4 adverse events are selected from hypothermia; shock; bradycardia; ventricular extrasystoles; myocardial ischemia; syncope; hemorrhage; atrial arrhythmia; phlebitis; AV block second degree; endocarditis; pericardial effusion; peripheral gangrene; thrombosis; coronary artery disorder; stomatitis; nausea and vomiting; liver function tests abnormal; gastrointestinal hemorrhage; hematemesis; bloody diarrhea; gastrointestinal disorder; intestinal perforation; pancreatitis; anemia; leukopenia; leukocytosis; hypocalcemia; alkaline phosphatase increase; blood urea nitrogen (BUN) increase; hyperuricemia; non-protein nitrogen (NPN) increase; respiratory acidosis; somnolence; agitation; neuropathy; paranoid reaction; convulsion; grand mal convulsion; delirium; asthma, lung edema; hyperventilation; hypoxia; hemoptysis; hypoventilation; pneumothorax; mydriasis; pupillary disorder; kidney function abnormal; kidney failure; and acute tubular necrosis. In some embodiments, administration of the effective amount of the IL-2 conjugate and an anti-EGFR antibody to a group of subjects does not cause one or more Grade 4 adverse events in greater than 1% of the subjects following administration of the IL-2 conjugate to the subjects. In some embodiments, Grade 4 adverse events are selected from hypothermia; shock; bradycardia; ventricular extrasystoles; myocardial ischemia; syncope; hemorrhage; atrial arrhythmia; phlebitis; AV block second degree; endocarditis; pericardial effusion; peripheral gangrene; thrombosis; coronary artery disorder; stomatitis; nausea and vomiting; liver function tests abnormal; gastrointestinal hemorrhage; hematemesis; bloody diarrhea; gastrointestinal disorder; intestinal perforation; pancreatitis; anemia; leukopenia; leukocytosis; hypocalcemia; alkaline phosphatase increase; blood urea nitrogen (BUN) increase; hyperuricemia; non-protein nitrogen (NPN) increase; respiratory acidosis; somnolence; agitation; neuropathy; paranoid reaction; convulsion; grand mal convulsion; delirium; asthma, lung edema; hyperventilation; hypoxia; hemoptysis; hypoventilation; pneumothorax; mydriasis; pupillary disorder; kidney function abnormal; kidney failure; and acute tubular necrosis.

In some embodiments, administration of the effective amount of the IL-2 conjugate and an anti-EGFR antibody to a group of subjects does not cause one or more adverse events in greater than 1% of the subjects following administration of the IL-2 conjugate to the subjects, wherein the one or more adverse events is selected from duodenal ulceration; bowel necrosis; myocarditis; supraventricular tachycardia; permanent or transient blindness secondary to optic neuritis; transient ischemic attacks; meningitis; cerebral edema; pericarditis; allergic interstitial nephritis; and tracheo-esophageal fistula.

In some embodiments, administration of the effective amount of the IL-2 conjugate and an anti-EGFR antibody to a group of subjects does not cause one or more adverse events in greater than 1% of the subjects following administration, wherein the one or more adverse events is selected from malignant hyperthermia; cardiac arrest; myocardial infarction; pulmonary emboli; stroke; intestinal perforation; liver or renal failure; severe depression leading to suicide; pulmonary edema; respiratory arrest; respiratory failure.

In some embodiments, administration of the IL-2 conjugate and an anti-EGFR antibody to the subject increases the number of peripheral CD8+T and NK cells in the subject without increasing the number of peripheral CD4+ regulatory T cells in the subject. In some embodiments, administration of the IL-2 conjugate and an anti-EGFR antibody to the subject increases the number of peripheral CD8+T and NK cells in the subject without increasing the number of peripheral eosinophils in the subject. In some embodiments, administration of the IL-2 conjugate and an anti-EGFR antibody to the subject increases the number of peripheral CD8+T and NK cells in the subject without increasing the number of intratumoral CD8+T and NK cells in the subject without increasing the number of intratumoral CD4+ regulatory T cells in the subject.

In some embodiments, administration of the effective amount of the IL-2 conjugate and an anti-EGFR antibody to the subject does not require the availability of an intensive care facility or skilled specialists in cardiopulmonary or intensive care medicine. In some embodiments, administration of the effective amount of the IL-2 conjugate to the subject does not require the availability of an intensive care facility or skilled specialists in cardiopulmonary or intensive care medicine. In some embodiments, administration of the effective amount of the IL-2 conjugate to the subject does not require the availability of an intensive care facility. In some embodiments, administration of the effective amount of the IL-2 conjugate to the subject does not require the availability of skilled specialists in cardiopulmonary or intensive care medicine.

In some embodiments, administration of the IL-2 conjugate and the anti-EGFR antibody combination therapy improves an ADCC response to a cancer, for example, by improving the ADCC function of the anti-EGFR antibody. In some embodiments, administration of the IL-2 conjugate and the anti-EGFR antibody combination therapy expands innate and adaptive immune cells. In some embodiments, administration of the IL-2 conjugate and the anti-EGFR antibody combination therapy promotes immune activation within the tumor microenvironment. In some embodiments, administration of the IL-2 conjugate and the anti-EGFR antibody results in a synergistic improvement in the anti-cancer activity of the combination of the two agents when compared to the anti-cancer activity of either agent alone. In some embodiments, administration of the IL-2 conjugate increases the number and amount of activation of NK cells, which potentiates the ADCC triggered by the anti-EGFR antibody.

### Additional Agents

In some embodiments, the methods further comprise administering to the subject a therapeutically effective amount of one or more chemotherapeutic agents, in addition to an anti-EGFR antibody. In some embodiments, the one or more chemotherapeutic agents comprises one or more platinum-based chemotherapeutic agents. In some embodiments, the one or more chemotherapeutic agents comprises carboplatin and pemetrexed. In some embodiments, the one or more chemotherapeutic agents comprises carboplatin and nab-paclitaxel. In some embodiments, the one or more chemotherapeutic agents comprises carboplatin and docetaxel. In some embodiments, the cancer in the subject is non-small cell lung cancer (NSCLC).

### Kits/Article of Manufacture

Disclosed herein, in certain embodiments, are kits and articles of manufacture for use with one or more methods and compositions described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic. The kit comprises an IL-2 conjugate and an anti-EGFR antibody.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself, a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for drugs, or the approved product insert. In one embodiment, compositions containing a compound provided herein formulated in a compatible pharmaceutical carrier are also prepared, placed in an appropriate container, and labeled for treatment of an indicated condition.

### Exemplary Embodiments

The present disclosure is further described by the following embodiments. The features of each of the embodiments are combinable with any of the other embodiments where appropriate and practical.

Embodiment P1. A method of treating a cancer in a subject in need thereof, comprising administering to the subject a therapeutically effective amount of (a) an IL-2 conjugate, and (b) an anti-EGFR antibody, wherein the IL-2 conjugate comprises the amino acid sequence of SEQ ID NO: 3 in which at least one amino acid residue in the IL-2 conjugate is replaced by the structure of Formula (I):

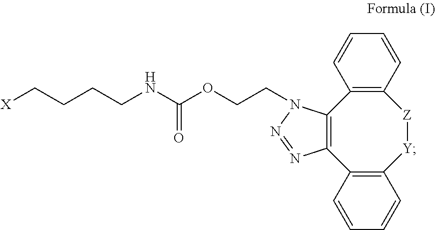

wherein:
Z is CH₂ and Y is

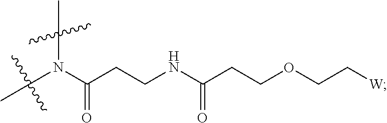

Y is CH₂ and Z is

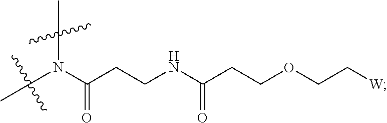

Z is CH₂ and Y is

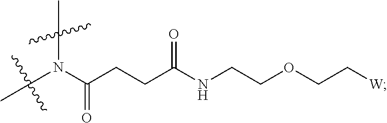

or
Y is CH₂ and Z is

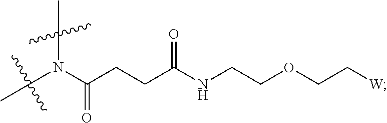

W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa; and
X has the structure:

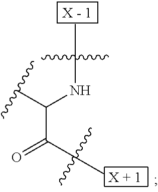

X−1 indicates the point of attachment to the preceding amino acid residue; and
X+1 indicates the point of attachment to the following amino acid residue;
wherein the position of the structure of Formula (I) in the amino acid sequence of the IL-2 conjugate is selected from K34, F41, F43, K42, E61, P64, R₃₇, T40, E67, Y44, V68, and L71, or a pharmaceutically acceptable salt, solvate, or hydrate thereof.

Embodiment P2. The method of embodiment P1, wherein in the IL-2 conjugate Z is CH₂ and Y is

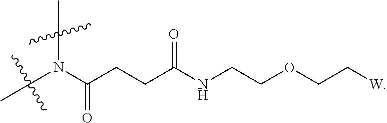

Embodiment P3. The method of embodiment P1, wherein in the IL-2 conjugate Y is CH₂ and Z is

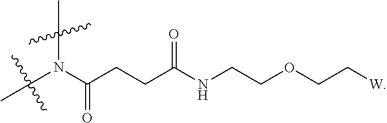

Embodiment P4. The method of any one of embodiments P1-3, wherein in the IL-2 conjugate the PEG group has an average molecular weight of 25 kDa, 30 kDa, or 35 kDa.

Embodiment P5. The method of embodiment 4, wherein in the IL-2 conjugate the PEG group has an average molecular weight of 30 kDa.

Embodiment P6. The method of any one of embodiments P1-5, wherein the position of the structure of Formula (I) in the amino acid sequence of the IL-2 conjugate is P64.

Embodiment P7. The method of embodiment P1, wherein the structure of Formula (I) has the structure of Formula (XII) or Formula (XIII), or is a mixture of the structures of Formula (XII) and Formula (XIII):

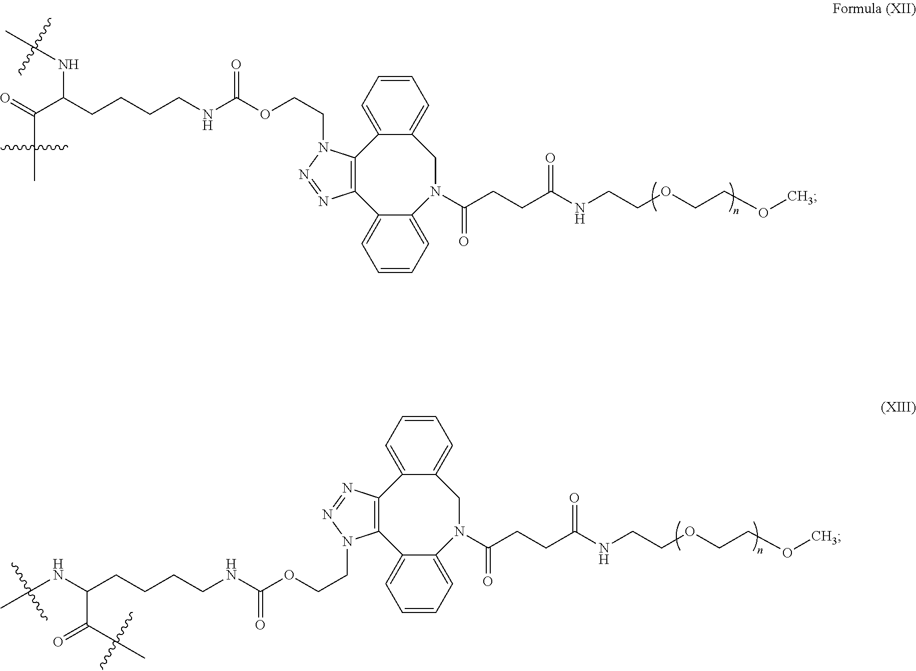

wherein:
n is an integer in the range from about 2 to about 5000; and
the wavy lines indicate covalent bonds to amino acid residues within SEQ ID NO: 3 that are not replaced,
or a pharmaceutically acceptable salt, solvate, or hydrate thereof.

Embodiment P8. The method of embodiment P7, wherein in the IL-2 conjugate n is an integer such that —(OCH₂CH₂)ₙ—OCH₃ has a molecular weight of about 25 kDa, 30 kDa, or 35 kDa.

Embodiment P9. The method of embodiment P8, wherein in the IL-2 conjugate n is an integer such that —(OCH₂CH₂)ₙ—OCH₃ has a molecular weight of about 30 kDa.

Embodiment P10. The method of any one of embodiments P7-9, wherein the position of the structure of Formula (XII) or Formula (XIII) in the amino acid sequence of the IL-2 conjugate is P64.

Embodiment P11. A method of treating a cancer in a subject in need thereof, comprising administering to the subject a therapeutically effective amount of (a) an IL-2 conjugate, and (b) an anti-EGFR antibody, wherein the IL-2 conjugate comprises the amino acid sequence of SEQ ID NO: 50, wherein [AzK_L1_PEG30 kD] has the structure of Formula (IV) or Formula (V), or is a mixture of the structures of Formula (IV) and Formula (V):

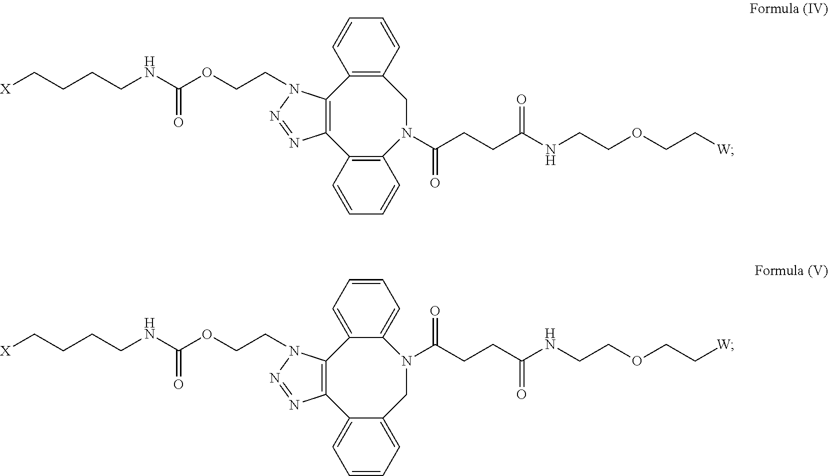

wherein:
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa;
X has the structure:

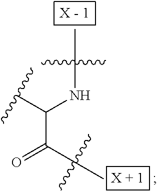 [SMILES: *NC(*)C(*)=O]

X−1 indicates the point of attachment to the preceding amino acid residue; and
X+1 indicates the point of attachment to the following amino acid residue;
or a pharmaceutically acceptable salt, solvate, or hydrate thereof.

Embodiment P12. The method according to embodiment P11, wherein W is a PEG group having an average molecular weight selected from 25 kDa, 30 kDa, or 35 kDa.

Embodiment P13. The method according to embodiment P12, wherein W is a PEG group having an average molecular weight of 30 kDa.

Embodiment P14. The method according to any one of embodiments P1-13, wherein the anti-EGFR antibody is cetuximab.

Embodiment P15. A method of treating a cancer in a subject in need thereof, comprising administering to the subject a therapeutically effective amount of (a) an IL-2 conjugate, and (b) cetuximab, wherein the IL-2 conjugate comprises the amino acid sequence of SEQ ID NO: 50, wherein [AzK_L1_PEG30 kD] has the structure of Formula (XII) or Formula (XIII), or is a mixture of the structures of Formula (XII) and Formula (XIII):

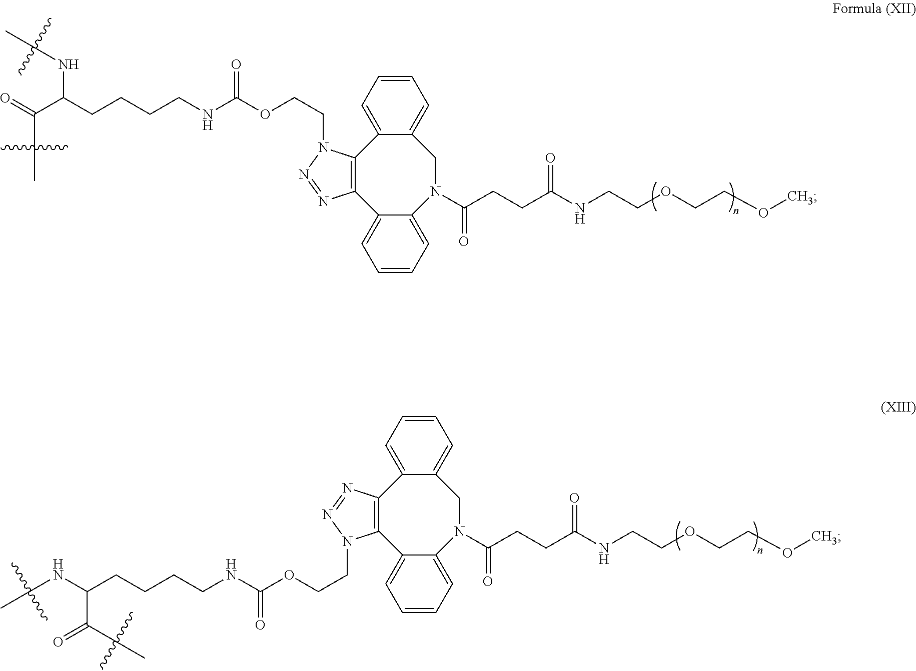

wherein:
n is an integer such that —(OCH₂CH₂)ₙ—OCH₃ has a molecular weight of about 30 kDa; and
the wavy lines indicate covalent bonds to amino acid residues within SEQ ID NO: 50 that are not replaced,
or a pharmaceutically acceptable salt, solvate, or hydrate thereof.

Embodiment P16. The method according to any one of embodiments P1-15, wherein the cancer is selected from renal cell carcinoma (RCC), non-small cell lung cancer (NSCLC), head and neck squamous cell cancer (HNSCC), classical Hodgkin lymphoma (cHL), primary mediastinal large B-cell lymphoma (PMBCL), urothelial carcinoma, microsatellite unstable cancer, microsatellite stable cancer, gastric cancer, colon cancer, colorectal cancer (CRC), cervical cancer, hepatocellular carcinoma (HCC), Merkel cell carcinoma (MCC), melanoma, small cell lung cancer (SCLC), esophageal, esophageal squamous cell carcinoma (ESCC), glioblastoma, mesothelioma, breast cancer, triple-negative breast cancer, prostate cancer, castrate-resistant prostate cancer, metastatic castrate-resistant prostate cancer, or metastatic castrate-resistant prostate cancer having DNA damage response (DDR) defects, bladder cancer, ovarian cancer, tumors of moderate to low mutational burden, cutaneous squamous cell carcinoma (CSCC), squamous cell skin cancer (SCSC), tumors of low-to non-expressing PD-L1, tumors disseminated systemically to the liver and CNS beyond their primary anatomic originating site, and diffuse large B-cell lymphoma.

Embodiment P17. The method according to any one of embodiments P1-16, wherein the IL-2 conjugate is administered to the subject once per week, once every two weeks, once every three weeks, once every 4 weeks, once every 5 weeks, once every 6 weeks, once every 7 weeks, or once every 8 weeks.

Embodiment P18. The method according to any one of embodiments P1-17, wherein the IL-2 conjugate is administered to a subject by intravenous administration.

### EXAMPLES

These examples are provided for illustrative purposes only and not to limit the scope of the claims provided herein.

### Example 1. Preparation of the IL-2 Conjugate IL-2_P65_[AzK_L1_PEG30 kD]-1

IL-2 employed for bioconjugation was expressed as inclusion bodies in E. coli using methods disclosed herein, using: (a) an expression plasmid encoding (i) the protein with the desired amino acid sequence, which gene contains a first unnatural base pair to provide a codon at the desired position at which the unnatural amino acid N6-((2-azidoethoxy)-carbonyl)-L-lysine (AzK) was incorporated and (ii) a tRNA derived from M. mazei Pyl, which gene comprises a second unnatural nucleotide to provide a matching anticodon in place of its native sequence; (b) a plasmid encoding a M. barkeri derived pyrrolysyl-tRNA synthetase (Mb PylRS), (c) AzK; and (d) a truncated variant of nucleotide triphosphate transporter PtNTT2 in which the first 65 amino acid residues of the full-length protein were deleted. The double-stranded oligonucleotide that encodes the amino acid sequence of the IL-2 variant contained a codon AXC as codon 64 of the sequence that encodes the protein having SEQ ID NO: 3 in which P64 is replaced with an unnatural amino acid described herein. The plasmid encoding an orthogonal tRNA gene from M. mazei comprised an AXC-matching anticodon GYT in place of its native sequence, wherein Y is an unnatural nucleotide as disclosed herein. X and Y were selected from unnatural nucleotides dTPT3 and dNaM as disclosed herein. The expressed protein was extracted from inclusion bodies and re-folded using standard procedures before site-specifically pegylating the AzK-containing IL-2 product using DBCO-mediated copper-free click chemistry to attach stable, covalent mPEG moieties (methoxy, linear PEG group having an average molecular weight of 30 kDa) to the AzK (as outlined in Scheme 6 above).

The IL-2 conjugate “IL-2_P65_[AzK_L1_PEG30 kD]-1” comprises SEQ ID NO: 50 in which the proline at position 64 is replaced by AzK_L1_PEG30 kD, wherein AzK L1 PEG30 kD is defined as a structure of Formula (IV) or Formula (V), or a mixture of Formula (IV) and Formula (V), and a 30 kDa, linear mPEG chain. The IL-2 conjugate “IL-2_P65_[AzK_L1_PEG30 kD]-1” is also defined as the compound comprising SEQ ID NO: 3 in which the proline residue at position 64 (P64) is replaced by the structure of Formula (VIII) or Formula (IX), or a mixture of Formula (VIII) and Formula (IX), wherein n is an integer such that the molecular weight of the PEG group is about 30 kDa. The IL-2 conjugate “IL-2_P65 [AzK_L1_PEG30 kD]-1” is also defined as the compound comprising SEQ ID NO: 3 in which the proline residue at position 64 (P64) is replaced by the structure of Formula (XII) or Formula (XIII), or a mixture of Formula (XII) and Formula (XIII), wherein n is an integer such that the molecular weight of the PEG group is about 30 kDa.

### Example 2. Antibody Dependent Cellular Cytotoxicity (ADCC) Assays Using IL-2_P65_[AzK_L1_PEG30 kD]-1 and Cetuximab

The effect of IL-2_P65 [AzK_L1_PEG30 kD]-1 on ADCC function of cetuximab is examined using a calcein-acetyoxymethyl (Calcein-AM; Invitrogen) release assay.

### Materials.

The following cell lines are used: A431 (EGFR high expression cell line), human PBMCs are obtained from healthy donors, and enriched using EasySep Human NK Cell Enrichment Kit (Stemcell).

The following reagents are used: calcein-acetyoxymethyl (Calcein-AM; Invitrogen), Probenecid (Invitrogen), ultra low IgG fetal bovine serum (Thermofisher), and human isotype IgG1 antibody (Biolegend).

### Procedure.

Human primary NK cells are negatively selected from PBMC using a RoboSep™ instrument according to manufacturer recommended protocols. Purified NK cells are cultured with IL-2_P65_[AzK_L1_PEG30 kD]-1 at varying concentrations (0.1 μg/mL, 0.01 μg/mL, 0.001 μg/mL, and 0 μg/mL) in RPMI 1640 media supplemented with 1% low IgG FBS for 18 hours at 37° C. in a humidified incubator with 5% CO₂. These cultured cells are used as effector cells. Human EGFR positive cancer cell line (A431) is labeled with calcein-AM for 30 min (50 ug diluted in 25 μL DMSO to prepare a stock solution, then 10 μL of calcein stock solution is added to 4 mL RPMI 1640+1% low IgG FBS+1% probenecid for the staining of 4×10⁶ cells), then washed and plated onto 96-well round bottom plates at a density of 5×10³ cells/well. Cetuximab and isotype human IgG1 antibody are added at various concentrations (from 10 μg/mL to 1 μg/mL) for 30 min to allow opsonization before adding NK cells. The NK cells activated with IL-2_P65_[AzK_L1_PEG30 kD]-1 are collected and added as effector cells at an E:T ratio of 3:1 (6×10+NK cells for 2×10⁴ target cells). The plates are then incubated for 1 hour at 37° C. in a humidified incubator with 5% CO₂, and 90 μL of supernatants are harvested and transferred into opaque 96-well microplates for analysis using fluorometry on an Envision 2104 plate reader (excitation: 492 nm; emission: 515 nm).

For maximal release, the cells are lysed with 2% Triton X−100. The fluorescence value of the culture medium background is subtracted from that of the experimental release (A), the target cell spontaneous release (B), and the target cell maximal release (C).

The cytotoxicity and ADCC percentages for each plate (in duplicate) are calculated using the following formulas:

$$Cytotoxicity ( \% ) = ( A - B ) / ( C - B ) \times 100$$

$$ADCC ( \% ) = Cytotoxicity ( \% , with antibody ) - Cytotoxicity ( \% , without antibody )$$

For each experiment, measurements are conducted in triplicate using three replicate wells. Each experiment is repeated at least 3 times. The half-maximal effective concentration (EC50) values are calculated by fitting the data points to a 4-parameter equation using GraphPad Prism 5 (GraphPad Software, Inc., San Diego, CA)

### Results.

Without activation by IL-2_P65_[AzK_L1_PEG30 kD]-1, cetuximab-treated human NK cells exhibit cytotoxicity against EGFR expressed cancer cell lines (A431 and A549), but not against EGFR null expression cells (NCI-H69). After activation by IL-2_P65_[AzK_L1_PEG30 kD]-1, cetuximab-treated human NK cells exhibit an enhanced cytotoxicity against EGFR expressed cancer cell lines (A431 and A549).

### Example 3. In Vitro Study of IL-2 Conjugate and Cetuximab (PBMC ADCC Assay)

A study was performed to investigate the effects of antibody dependent cellular cytotoxicity (ADCC) by the IL-2 conjugate of Example 1 in combination with cetuximab using a co-culture of human PBMCs with calcein-labeled cancer cell lines (CAL27 and A431).

### CAL27 Cells.

### Reagents.

Bioassay buffer: 1% ultra low IgG FBS added to phenol-red-free RPMI. Complete assay buffer: 450 μL probenecid added to 45 mL bioassay buffer with final probenecid concentration of 77 g/mL. Calcein-acetoxymethyl ester (Calcein-AM): 50 μg in 25 μL DMSO. Calcein-AM staining buffer: 10 μL Calcein-AM added to 4 mL complete assay buffer (final Calcein-AM concentration of 5 μg/mL). Triton-X−100 lysis buffer: 20 μL Triton-X−100 added to 4 mL complete assay buffer (final concentration of 0.5%).

### Procedure.

On Day 1, a 6-point, 1 in 5 dilution series (in PBS) of the IL-2 conjugate was prepared. The IL-2 conjugate concentrations were 2, 0.4, 0.08, 0.016, 0.0032, and 0 μg/mL. PBMCs were collected by centrifugation at 200×g for 5 minutes and resuspended in phenol red-free RPMI+10% ultra-low IgG at 20 million cells/mL. Appropriate volumes of these PBMCs were transferred to 6 sections of a multi-well reservoir to which a range of the IL-2 conjugate dilutions was added. PBMCs were mixed well with the IL-2 conjugate by pipetting up and down and 50 μL were transferred into round-bottomed 96 well plates using a multi-channel pipette (final PBMC number per well was 1 million). Six empty wells were reserved for controls to be added the following day. The plates were incubated overnight in a humidified incubator at 37° C. in the presence of 5% carbon dioxide.

On Day 2, CAL27 cells (EGFR-expressing oral epithelial squamous cell carcinoma cell line) were harvested using TrypLE express dissociation buffer and collected by centrifugation at 200×g for 5 minutes. Cells were counted and 5 million cells were resuspended in 4 mL calcein-AM staining buffer and incubated for 30 minutes at 37° C. in the presence of 5% carbon dioxide. Cells were then collected and washed twice in complete assay buffer by centrifugation at 200×g for 5 minutes. Cells were counted and resuspended at 0.4 million cells/mL for a final target cell number of 20,000/well.

Cetuximab antibody (Eli Lilly & Co.) was diluted to a working concentration of 3× (3, 0.3, 0.03, 0.003 μg/mL) for final assay concentrations of 1, 0.1, 0.001, 0.0001 μg/mL. The isotype control (hIgG1, Biolegend) was diluted to 3 μg/mL for a final concentration of 1 μg/mL in complete assay buffer. Equal volumes of stained CAL27 cells at 0.4 million cells/mL were mixed with antibody dilutions or isotype control and incubated for 30 minutes at 4° C. to allow antibody to bind. Following incubation, 100 μL of antibody-CAL27 cell mixture were added to the 96 well plates containing 50 μL of the IL-2 conjugate treated PBMCs from Day 1.

Control wells without PBMCs but with 50 μL calcein-AM stained CAL27 cells treated with complete assay buffer (background signal) or stained CAL27 with 50 μL Triton-X−100 treatment (for maximum signal following cell lysis), both made up to 150 μL final volume with complete assay buffer were prepared in triplicate. The plates were centrifuged for 1 minute at 200×g, and then incubated for 60 minutes at 37° C. in the presence of 5% carbon dioxide. After incubation, the plates were again briefly centrifuged before transferring 90 μL of supernatant into fresh black, clear-bottomed plates, and the fluorescence signal was read on an Envision 2104 plate reader (excitation: 492 nm; emission: 515 nm).

The cytotoxicity was calculated using the following formula:

$$Cytotoxicity ( \% ) = ( A - B ) / ( C - B ) \times 100$$

where A is the fluorescence value for treated cells; B is the background from target cells alone; and C is the maximum release valued obtained from Triton-X-100 treatment.

The data represent the % cytotoxicity of the IL-2 conjugate treated human PBMCs on target cancer cells in the presence of cetuximab. The mean percentage from the technical replicates was converted to a proportion. The analysis was conducted using a two-way generalized linear mixed model (GLMM), with factors for the IL-2 conjugate, cetuximab and their interaction, with random donor effects, treating proportion as a pseudo-binomial variable. It was followed by a post-hoc test (with Dunnett-Hsu adjustment) to compare the IL-2 conjugate treated groups to the control group. Statistical analyses were performed using SAS (1) version 9.4 software. A probability less than 5% (p<0.05) was considered as significant.

### Results.

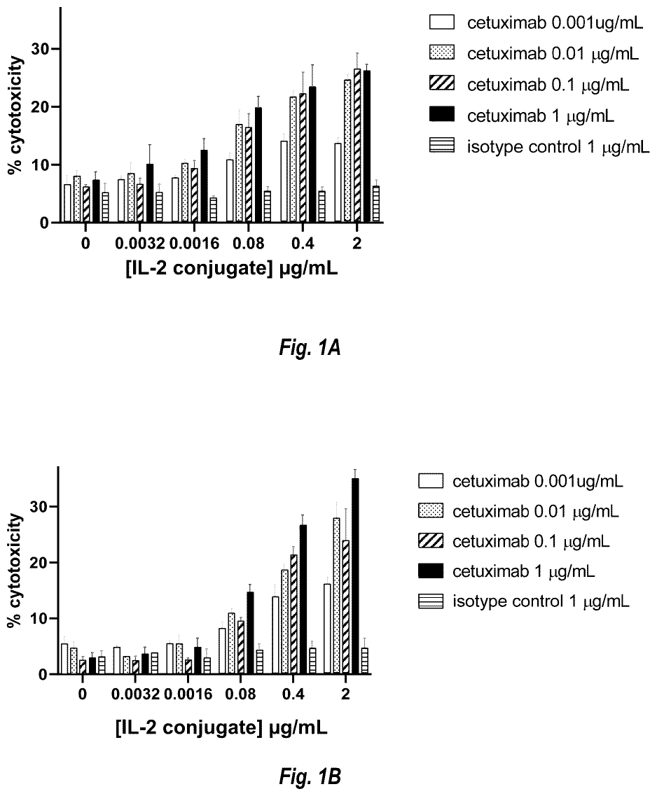

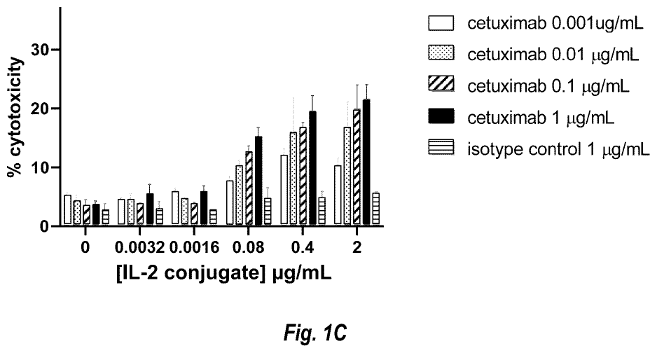

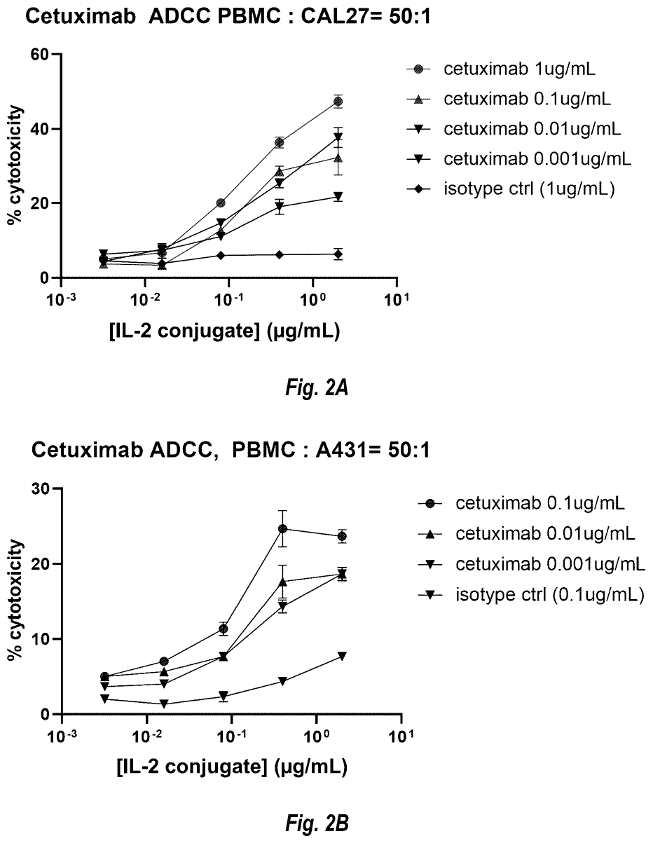

At cetuximab dose levels of 1, 0.1, and 0.01 mg/mL, the IL-2 conjugate enhanced ADCC function of cetuximab against EGFR expressing CAL27 cells (p<0.05) at concentrations of 0.08, 0.4 and 2 mg/mL (FIGS. 1A-C). At a cetuximab dose level of 0.001 mg/mL, the IL-2 conjugate enhanced ADCC function of cetuximab against EGFR expressing CAL27 cells (p<0.05) at concentrations of 0.4 and 2 mg/mL. FIG. 2A further shows the enhanced ADCC function of cetuximab against EGFR expressing CAL27 cells (PBMC to CAL27 ratio 50:1).

The tests of fixed effects from the GLMM model indicate that the factors IL-2 conjugate, cetuximab and their interaction have a significant effect on the cytotoxicity, i.e., the differences between IL-2 conjugate groups vary significantly for the different cetuximab concentrations. The pairwise comparisons indicated a significant difference between the IL-2 conjugate 2 mg/mL group versus the control group (p=0.0001) and between the IL-2 conjugate 0.4 mg/mL group versus the control group (p=0.0001) at a cetuximab concentration of 0.001 mg/mL. The pairwise comparisons also indicated a significant difference between the IL-2 conjugate 2 mg/mL group versus the control group (p<0.0001), between the IL-2 conjugate 0.4 mg/mL group versus the control group (p<0.0001), and between the IL-2 conjugate 0.08 mg/mL group versus the control group (p=0.0003) at a cetuximab concentration of 0.01 mg/mL. In addition, the pairwise comparisons indicated a significant difference between the IL-2 conjugate 2 mg/mL group versus the control group (p<0.0001), between the IL-2 conjugate 0.4 mg/mL group versus the control group (p<0.0001), and between the IL-2 conjugate 0.08 mg/mL group versus the control group (p<0.0001) at a cetuximab concentration of 0.1 mg/mL. Lastly, the pairwise comparisons indicated a significant difference between the IL-2 conjugate 2 mg/mL group versus the control group (p<0.0001), between the IL-2 conjugate 0.4 mg/mL group versus the control group (p<0.0001), and between the IL-2 conjugate 0.08 mg/mL group versus the control group (p<0.0001) at a cetuximab concentration of 1 mg/mL.

The data demonstrate that the IL-2 conjugate enhanced ADCC function of cetuximab against EGFR expressing CAL27 cancer cells. No significant differences were observed using the IL-2 conjugate in combination with the isotype control.

### A431 Cells.

Studies were performed using EGFR expressing A431 cells (epidermoid carcinoma) following the procedure outlined above for CAL27 cells. FIG. 2B shows the enhanced ADCC function of cetuximab against EGFR expressing A431 cells (PBMC to A431 ratio 50:1). The data demonstrate that the IL-2 conjugate enhanced ADCC function of cetuximab against EGFR expressing A431 cancer cells.

### Example 4. ADCC Assay Using an Engineered Cell Line NK-92.CD16 V as Effector Cells

The effect of IL-2_P65_[AzK_L1_PEG30 kD]-1 on ADCC function of cetuximab was examined using a calcein-acetyoxymethyl (Calcein-AM; Invitrogen) release assay.

### Materials.

NK-92.CD16 V (high affinity variant) (Conkwest Inc., San Diego, CA) was used as the effector cell line. The following cell lines were used as target cells: CAL27, A431, DLD-1, and FaDu.

The following reagents were used: cetuximab antibody (Eli Lilly & Co.); human isotype IgG1 antibody (Biolegend); calcein-acetyoxymethyl (Calcein-AM; Invitrogen C3100MP), and probenecid (Invitrogen; P36400). The bioassay medium was phenol red-free RPMI with 1% ultra low IgG fetal bovine serum, supplemented with 1% probenecid for complete assay medium. MyeloCult H5100 (Stemcell Cat #05150) supplemented with IL-2 (100 U/mL) and hydrocortisone (Sigma H6909; 10 mL at 50 μM) was used for the NK-92.CD16 V cell culture.

### Procedure.

IL-2 supplement was withdrawn from the NK-92.CD16 V cell culture, which was then incubated overnight prior to starting the assay. The next day, cells were plated in 96-well round-bottom plates (60,000 cells were plated for a 3:1 ratio of effector to target cells) in the presence of IL-2_P65_[AzK_L1_PEG30 kD]-1 at varying concentrations (0.1 μg/mL, 0.01 μg/mL, 0.001 μg/mL, and 0 μg/mL) in phenol red-free RPMI 1640 media supplemented with 1% low IgG FBS for 18 hours at 37° C. in a humidified incubator with 5% CO₂. These cells are used as the effector cells. The following day, human EGFR positive cancer cell lines (A431, DLD-1, FaDu, or CAL27) were labeled with calcein-AM for 30 min (50 ug diluted in 25 μL DMSO to prepare a stock solution, then 10 μL of calcein stock solution was added to 4 mL RPMI 1640 containing 1% low IgG FBS and 1% probenecid for the staining of 5×10⁶ cells) and then washed. Cells were divided into several labeled tubes for incubation with varying concentrations of cetuximab or isotype control. Cetuximab and isotype human IgG1 antibody were added at 3× concentrations (for final assay concentrations from 10 μg/mL to 1 μg/mL), and the labeled target cells and antibody were mixed and allowed to sit for 30 min to allow opsonization. After this incubation, target cells (20,000) and antibody were added on top of NK-92.CD16 V cells in 100 μL. The plate was centrifuged briefly for 1 minute at 1100 rpm before incubating at 37° C. and 5% CO₂ for 1 hour. Following incubation, the plates were again briefly centrifuged as before, and 90 μL of supernatant was transferred from each well to black plates with clear bottom without disturbing the cells. The fluorescence signal was read using Envision 2104 (excitation: 492 nm; emission: 515 nm).

For maximal release, the cells were lysed with 2% Triton X−100. The fluorescence value of the culture medium background was subtracted from that of the experimental release (A), the target cell spontaneous release (B), and the target cell maximal release (C).

The cytotoxicity and ADCC percentages for each plate (in duplicate) were calculated using the following formulas:

$$Cytotoxicity ( \% ) = ( A - B ) / ( C - B ) \times 100$$

$$ADCC ( \% ) = Cytotoxicity ( \% , with antibody ) - Cytotoxicity ( \% , without antibody )$$

For each experiment, measurements were conducted in triplicate using three replicate wells. Each experiment is repeated at least 3 times. The half-maximal effective concentration (EC50) values are calculated by fitting the data points to a 4-parameter equation using GraphPad Prism 5 (GraphPad Software, Inc., San Diego, CA).

### Results.

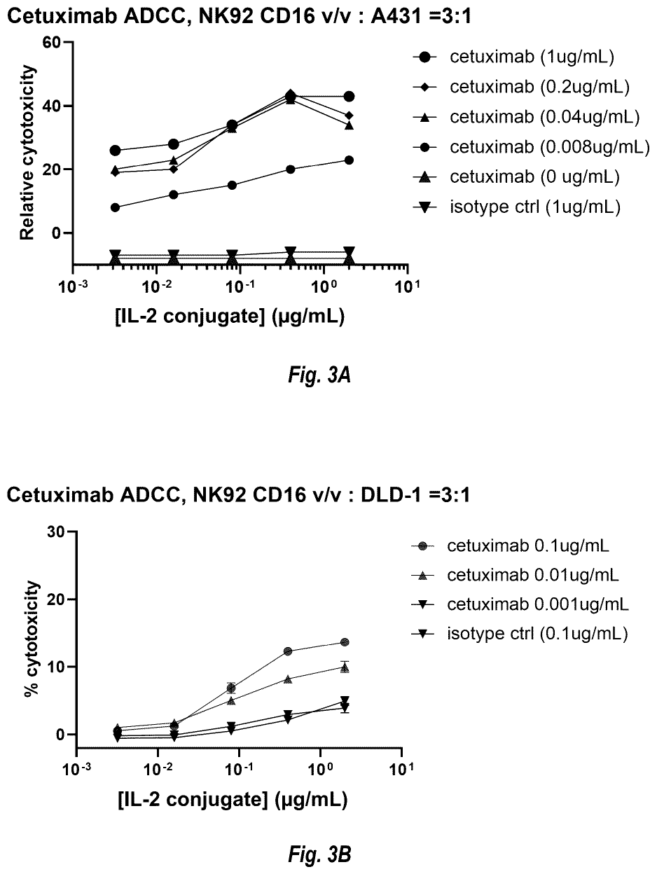

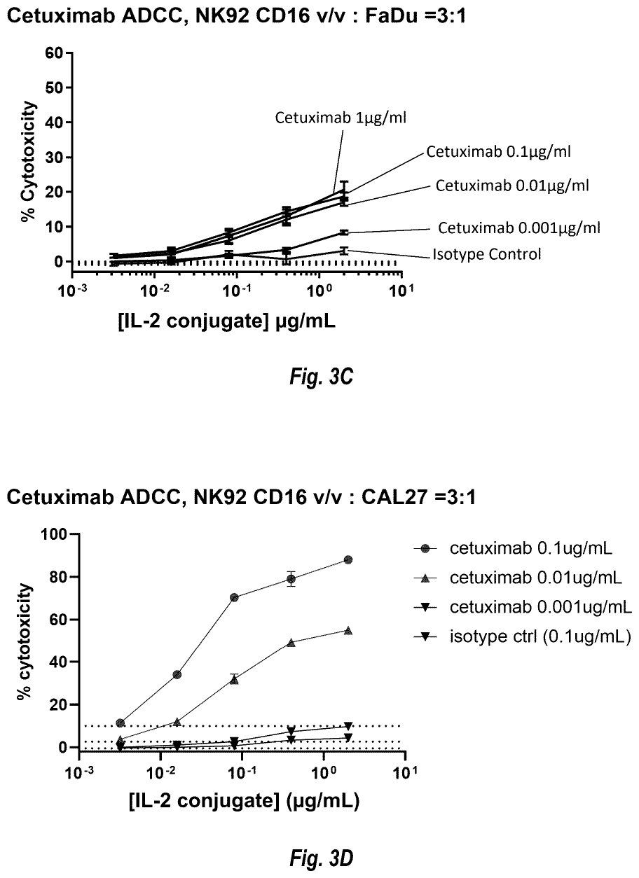

Cytotoxicity data using the NK92 cell line ADCC assay is shown in FIGS. 3A-D for EGFR expressing A431 (epidermoid carcinoma) (NK92 to A431 ratio 3:1), DLD-1 (adenocarcinoma, colorectal) (NK92 to DLD-1 ratio 3:1), FaDu (epithelial squamous cell carcinoma) (NK92 to FaDu ratio 3:1), and CAL27 (epithelial squamous cell carcinoma) (NK92 to CAL27 ratio 3:1) cells, respectively. The data demonstrate that the IL-2 conjugate enhanced ADCC function of cetuximab against EGFR expressing cancer cells.

### Example 5. Clinical Study of Combination Therapy Using an IL-2 Conjugate and Cetuximab

Overview. Monotherapy using the IL-2 conjugate of Example 1 has been demonstrated to promote a peripheral increase in the number of NK cells, which are important effector cells mediating antibody-dependent cellular cytotoxicity (ADCC) for IgG1 antibodies such as cetuximab.

A Phase 1/2, open-label, multi-center study assessing the clinical benefit of the IL-2 conjugate described in Example 1 in combination with cetuximab for the treatment of participants with advanced or metastatic solid tumors was undertaken.

Participants received the IL-2 conjugate (16 or 24 μg/kg dose) by IV infusion once every 3 weeks. Here and throughout discussion of this cohort, drug mass per kg subject (e.g., 16 μg/kg) refers to IL-2 mass exclusive of PEG and linker mass. Cetuximab was given on Cycle 1 Day 1 as an initial loading dose of 400 mg/m² infused over 120 minutes (maximum infusion rate 10 mg/min), followed by 250 mg/m² infused over 60 minutes (maximum infusion rate 10 mg/min) for all subsequent doses starting with the Cycle 1 Day 8 administration. Cetuximab was given on days 1, 8, and 15 of each 21 day cycle. The infusion time of the IL-2 conjugate was about 30 minutes each. For each cycle of treatment, prior to administering the IL-2 conjugate, all participants received IL-2 conjugate premedication to prevent or reduce the acute effect of infusion-associated reactions (IAR) or flu-like symptoms, 30 to 60 minutes prior to infusion of the IL-2 conjugate. The IL-2 conjugate premedication was as follows: anti-pyretic, orally, and anti-histamine (H1 blocker). Antiemetics were provided at the discretion of the supervising physician. Prior to administration of the first dose of cetuximab, all participants were pre-medicated with diphenhydramine (about 25 to 50 mg, intravenous). Premedication for subsequent doses of cetuximab was optional based on the supervising physician's assessment. When the IL-2 conjugate and cetuximab were given on the same day, participants who received diphenhydramine as cetuximab premedication may have skipped the diphenhydramine as the IL-2 conjugate premedication. The dosing sequence was as follows: (i) premedication for cetuximab (30-60 min. prior to the start of cetuximab infusion); (ii) cetuximab; (iii) premedication for the IL-2 conjugate (administered 30-60 min. prior to the start of the IL-2 conjugate infusion); and (iv) IL-2 conjugate. Treatment was repeated for up to a total of 35 cycles or for a duration up to 735 days.

The following biomarkers serve as surrogate predictors of safety and/or efficacy:
Eosinophilia (elevated peripheral eosinophil count): Cell surrogate marker for IL-2-induced proliferation of cells (eosinophils) linked to vascular leak syndrome (VLS);
Interleukin 5 (IL-5): Cytokine surrogate marker for IL-2 induced activation of type 2 innate lymphoid cells and release of this chemoattractant that leads to eosinophilia and potentially VLS;
Interleukin 6 (IL-6): Cytokine surrogate marker for IL-2 induced cytokine release syndrome (CRS); and
Interferon γ (IFN-γ): Cytokine surrogate marker for IL-2 induced activation of CD8+ cytotoxic T lymphocytes.

The following biomarkers serve as surrogate predictors of anti-tumor immune activity:
Peripheral CD8+Effector Cells: Marker for IL-2-induced proliferation of these target cells in the periphery that upon infiltration become a surrogate marker of inducing a potentially latent therapeutic response;
Peripheral CD8+Memory Cells: Marker for IL-2-induced proliferation of these target cells in the periphery that upon infiltration become a surrogate marker of inducing a potentially durable latent therapeutic and maintenance of the memory population;
Peripheral NK Cells: Marker for IL-2-induced proliferation of these target cells in the periphery that upon infiltration become a surrogate marker of inducing a potentially rapid therapeutic response; and
Peripheral CD4+Regulatory Cells: Marker for IL-2-induced proliferation of these target cells in the periphery that upon infiltration become a surrogate marker of inducing an immunosuppressive TME and offsetting of an effector-based therapeutic effect.
   First Cohort Using 16 μg/kg Dose of IL-2 Conjugate

Results. The 5 subjects included two human males and 3 females with a median age of 69 years (ranging from 65-72 years). All subjects had an Eastern Cooperative Oncology Group (ECOG) performance status of 0 or 1, and had received 1 to 4 prior lines of systemic therapies. The cancers were anal cancer (1 subject), colon adenocarcinoma (1 subject), adrenocortical cancer (1 subject), squamous cell carcinoma of the lung (1 subject), and small intestinal cancer (1 subject). All five subjects had metastatic disease.

The subjects received the IL-2 conjugate (16 ug/kg) and cetuximab combination treatment for 2-7 cycles (2-7 doses of the IL-2 conjugate). Two subjects, one with anal cancer and one with metastatic adrenocortical carcinoma, showed progressive disease (PD) following 2 cycles of combination treatment, leading to discontinuation of the IL-2 conjugate and cetuximab combination treatment. One subject with colon cancer showed disease progression after 5 cycles. Two subjects are ongoing: one at 3 cycles with squamous cell carcinoma of the lung and one at 7 cycles with small intestinal carcinoma.

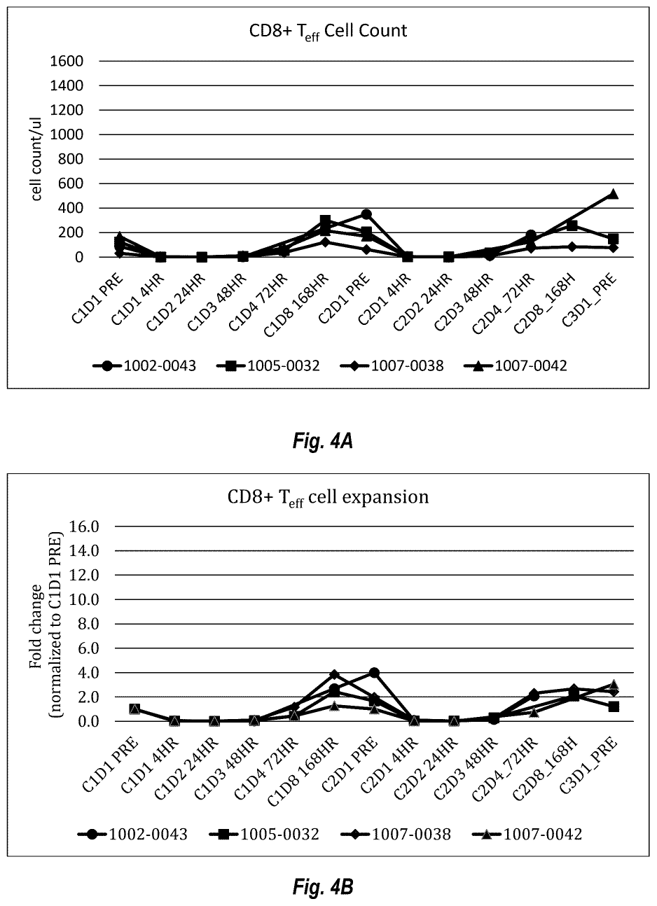

Peripheral CD8+T_{eff}cell counts were measured (FIGS. 4A-B). Prolonged CD8+ expansion over baseline (e.g., greater than or equal to 2-fold change) was observed at 3 weeks after the previous dose in some subjects.

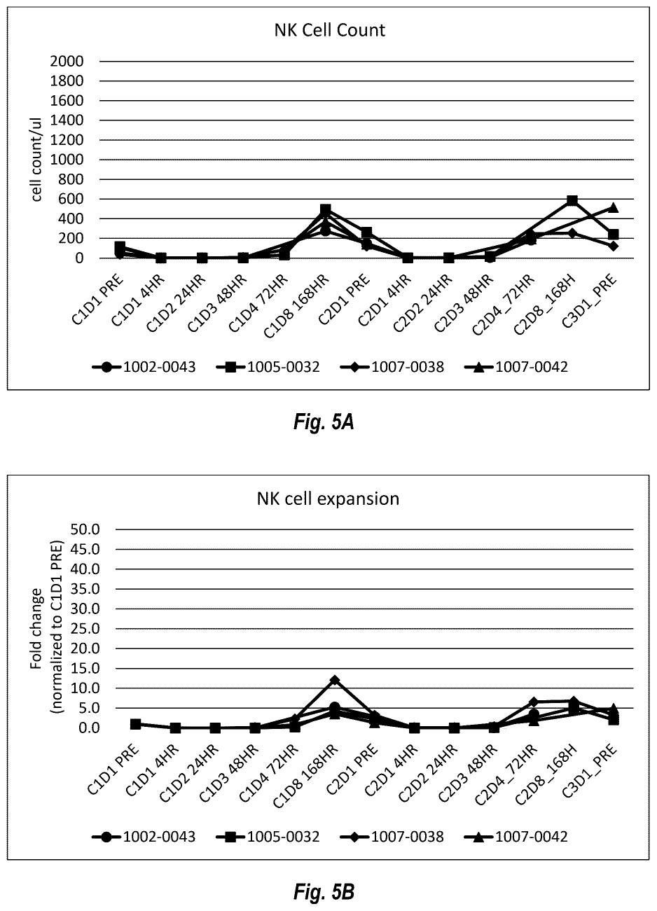

Peripheral NK cell counts are shown in FIGS. 5A-B. An increase in NK cell count was observed in each subject. Subjects generally showed elevated NK cell counts over baseline at 8 days and 3 weeks after the previous dose.

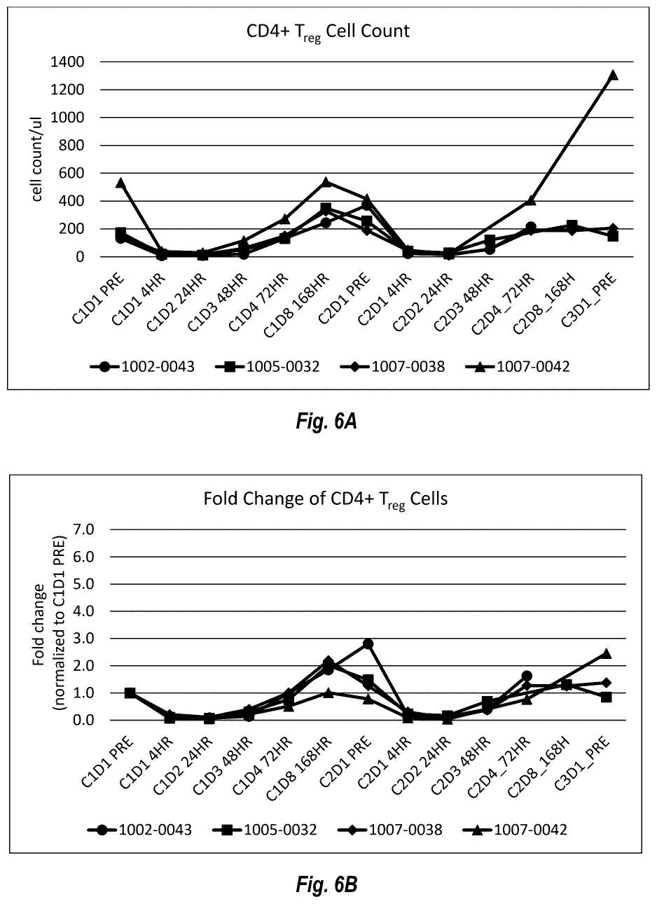

Peripheral CD4+T_{reg} counts are shown in FIGS. 6A-B.

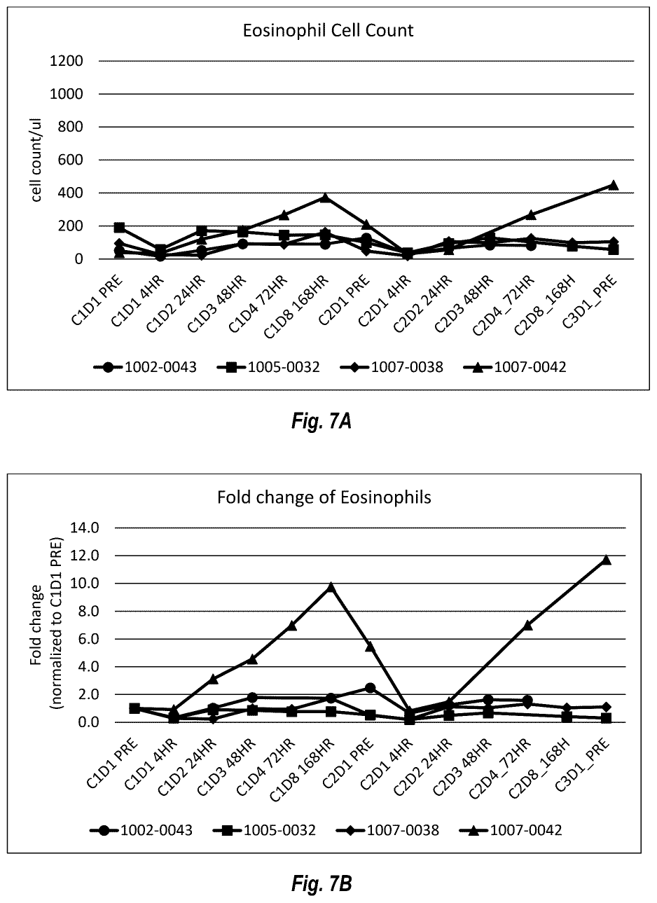

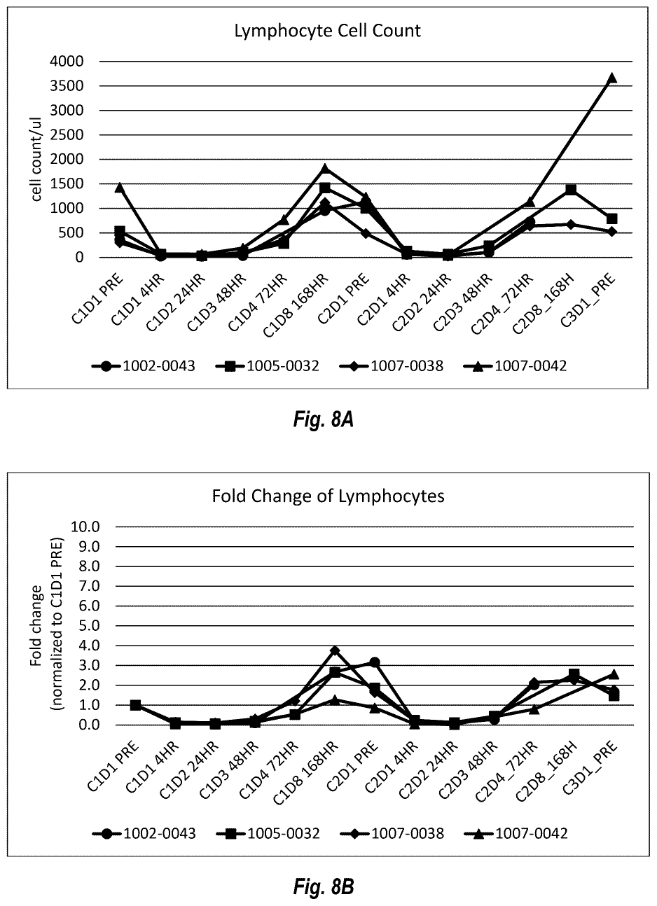

Eosinophil counts were measured (FIGS. 7A-B). The measured values did not exceed a four-fold increase and were consistently below the range of 2328-15958 eosinophils/μL in patients with IL-2 induced eosinophilia as reported in Pisani et al., Blood 1991 Sep. 15; 78 (6): 1538-44. Lymphocyte counts were also measured (FIGS. 8A-B).

Summary of Results and Discussion. All subjects tested had post-dose peripheral expansion of CD8+T effector (Teff) cells, NK cells, and CD4+T_{reg} cells.

An adverse event (AE) was any untoward medical occurrence in a clinical investigation subject administered a pharmaceutical product, regardless of causal attribution. Dose-limiting toxicities were defined as an AE occurring within Day 1 through Day 29 (inclusive)±1 day of a treatment cycle that was not clearly or incontrovertibly solely related to an extraneous cause and that met at least one of the following criteria:
Grade 3 neutropenia (absolute neutrophil count <1000/mm³ >500/mm³) lasting ≥7 days, or Grade 4 neutropenia of any duration
Grade 3+ febrile neutropenia
Grade 4+ thrombocytopenia (platelet count <25,000/mm³)·
Grade 3+ thrombocytopenia (platelet count <50,000-25,000/mm³) lasting ≥5 days, or associated with clinically significant bleeding or requiring platelet transfusion
Failure to meet recovery criteria of an absolute neutrophil count of at least 1,000 cells/mm³ and a platelet count of at least 75,000 cells/mm³ within 10 days
Any other grade 4+ hematologic toxicity lasting ≥5 days
Grade 3+ ALT or AST in combination with a bilirubin >2 times ULN with no evidence of cholestasis or another cause such as viral infection or other drugs (i.e. Hy's law)
Grade 3 infusion-related reaction that occurs with premedication; Grade 4 infusion-related reaction
Grade 3 Vascular Leak Syndrome defined as hypotension associated with fluid retention and pulmonary edema
Grade 3+ anaphylaxis
Grade 3+ hypotension
Grade 3+ AE that does not resolve to grade <2 within 7 days of starting accepted standard of care medical management
Grade 3+ cytokine release syndrome

The following exceptions applied to non-hematologic AEs:
Grade 3 fatigue, nausea, vomiting, or diarrhea that resolves to grade≤2 with optimal medical management in ≤3 days
Grade 3 fever (as defined by >40° C. for ≤24 hours)
Grade 3 infusion-related reaction that occurs without premedication; subsequent doses should use premedication and if reaction recurs then it will be a DLT
Grade 3 arthralgia or rash that resolves to grade≤2 within 7 days of starting accepted standard of care medical management (e.g., systemic corticosteroid therapy)
   If a subject had grade 1 or 2 ALT or AST elevation at baseline considered secondhand to liver metastases, a grade 3 elevation must also be ≥3 times baseline and last >7 days.

Serious AEs were defined as any AE that results in any of the following outcomes: death; life-threatening AE; inpatient hospitalization or prolongation of an existing hospitalization; a persistent or significant incapacity or substantial disruption of the ability to conduct normal life functions; or a congenital anomaly/birth defect. Important medical events that may not result in death, be life-threatening, or require hospitalization may be considered serious when, based upon appropriate medical judgment, they may jeopardize the subject and may require medical or surgical intervention to prevent one of the outcomes listed above. Examples of such medical events include allergic bronchospasm requiring intensive treatment in an emergency room or at home, blood dyscrasias or convulsions that do not result in inpatient hospitalization, or the development of drug dependency or drug abuse.

There were no meaningful elevations in IL-5. There was no cumulative toxicity. There was no end organ toxicity. There was no QTc prolongation or other cardiac toxicity. Overall, the IL-2 conjugate was considered well-tolerated.

Four of the 5 subjects had at least one treatment-emergent AE (TEAE). Most of the TEAEs were Grade 1-2, one subject had at least one Grade 3, and one subject at least one Grade 4 TEAE. Four subjects had treatment related AEs. These included: one Grade 1 infusion reaction; one Grade 1 nausea; one Grade 1 fatigue; one Grade 2 diarrhea; and one Grade 4 lymphocyte count decrease. Two subjects had 3 unrelated SAEs: one dysphagia and spinal cord compression; and one pleural effusion. The TEAEs did not result in any drug discontinuations, no dose reductions, no DLTs, and no anaphylaxis or CRS. The treatment-related AEs resolved with accepted standard of care. TEAEs are detailed in Table 2, and treatment-related adverse events are summarized in Table 3.

TABLE 2
Treatment Emergent Adverse Events: n = 5.

| System Organ Class | Grade 1 | Grade 2 | Grade 3 | Grade 4 | Grade 5 |
|---|---|---|---|---|---|
| General disorders and | 2/5 (40%) | 0/5 (0%) | 0/5 (0%) | 0/5 (0%) | 0/5 (0%) |
| administration site conditions | | | | | |
| Gastrointestinal disorders | 2/5 (40%) | 0/5 (0%) | 1/5 (20%) | 0/5 (0%) | 0/5 (0%) |
| Investigations | 0/5 (0%) | 0/5 (0%) | 0/5 (0%) | 1/5(20%) | 0/5 (0%) |
| Infections and infestations | 0/5 (0%) | 1/5 (20%) | 0/5 (0%) | 0/5 (0%) | 0/5 (0%) |
| Injury, Procedural | 2/5 (40%) | 0/5 (0%) | 0/5 (0%) | 0/5 (0%) | 0/5 (0%) |
| Complications | | | | | |
| Nervous system disorders | 0/5 (0%) | 0/5 (0%) | 2/5(40%) | 0/5 (0%) | 0/5 (0%) |
| Skin and subcutaneous tissue | 3/5 (60%) | 0/5 (0%) | 0/5 (0%) | 0/5 (0%) | 0/5 (0%) |
| disorders | | | | | |
| Blood and lymphatic system | 1/5 (20%) | 0/5 (0%) | 0/5 (0%) | 0/5 (0%) | 0/5 (0%) |
| disorders | | | | | |
| Eye Disorders | 1/5 (20%) | 0/5 (0%) | 0/5 (0%) | 0/5 (0%) | 0/5 (0%) |
| Metabolism and nutrition | 1/5 (20%) | 0/5 (0%) | 0/5 (0%) | 0/5 (0%) | 0/5 (0%) |
| disorders | | | | | |
| Respiratory, thoracic and | 0/5 (0%) | 1/5 (20%) | 0/5 (0%) | 0/5 (0%) | 0/5 (0%) |
| mediastinal disorders | | | | | |
| Endocrine Disorders | 0/5 (0%) | 1/5 (20%) | 0/5 (0%) | 0/5 (0%) | 0/5 (0%) |
| Musculoskeletal and | 1/5 (20%) | 1/5 (20%) | 0/5 (0%) | 0/5 (0%) | 0/5 (0%) |
| connective tissue disorders | | | | | |
| Psychiatric disorders | 1/5 (20%) | 0/5 (0%) | 0/5 (0%) | 0/5 (0%) | 0/5 (0%) |
| Reproductive and Breast | 1/5 (20%) | 0/5 (0%) | 0/5 (0%) | 0/5 (0%) | 0/5 (0%) |
| Disorders | | | | | |
| Renal and Urinary Disorders | 1/5 (20%) | 0/5 (0%) | 0/5 (0%) | 0/5 (0%) | 0/5 (0%) |

TABLE 3
Treatment Related Adverse Events: n = 5.

| System Organ Class | Grade 1 | Grade 2 | Grade 3 | Grade 4 | Grade 5 |
|---|---|---|---|---|---|
| Endocrine Disorders | 0/5 (0%) | 1/5 (20%) | 0/5 (0%) | 0/5 (0%) | 0/5 (0%) |
| General disorders and | 2/5 (40%) | 0/5 (0%) | 0/5 (0%) | 0/5 (0%) | 0/5 (0%) |
| administration site conditions | | | | | |
| Gastrointestinal disorders | 1/5 (20%) | 1/5 (205%) | 0/5 (0%) | 0/5 (0%) | 0/5 (0%) |
| Investigations | 0/5 (0%) | 0/5 (0%) | 0/5 (0%) | 1/5 (20%) | 0/5 (0%) |
| Injury, Procedural | 1/5 (20%) | 0/5 (0%) | 0/5 (0%) | 0/5 (0%) | 0/5 (0%) |
| Complications | | | | | |
| Nervous system disorders | 0/5 (0%) | 0/5 (0%) | 1/5(20%) | 0/5 (0%) | 0/5 (0%) |

Second Cohort Using 24 μg/kg Dose of IL-2 Conjugate

Results. The 3 subjects were human males with a median age of 71 years (ranging from 65-75 years). All subjects had an Eastern Cooperative Oncology Group (ECOG) performance status of 1. One subject had received 1 prior line of therapy, and a second subject had received 4 prior lines of therapy. The cancers were gastric cancer (1 subject), head and neck squamous cell carcinoma (HNSCC) (1 subject), and colon cancer (1 subject). All of the subjects had metastatic disease. The subjects received the IL-2 conjugate (24 ug/kg) and cetuximab combination treatment for 1 cycle (1 dose of the IL-2 conjugate). One subject showed progressive disease (PD) following the first cycle of combination treatment, preventing administration of a further treatment dose of the IL-2 conjugate and cetuximab combination treatment.

Two of the subjects experienced at least one TEAE. One of the subjects experienced at least one Grade 3 treatment-related AE, which was Grade 3 chills. None of the subjects had related SAEs. No DLTs were observed and no drug discontinuations resulted from the TEAEs. TEAEs are detailed in Table 4, and treatment-related adverse events are summarized in Table 5.

TABLE 4
Treatment Emergent Adverse Events: n = 3.

| System Organ Class | Grade 1 | Grade 2 | Grade 3 | Grade 4 | Grade 5 |
|---|---|---|---|---|---|
| Gastrointestinal disorders | 0/3 (0%) | 1/3 (33.3%) | 0/3 (0%) | 0/3 (0%) | 0/3 (0%) |
| General disorders and | 1/3 (33.3%) | 0/3 (0%) | 1/3 (33.3%) | 0/3 (0%) | 0/3 (0%) |
| administration conditions | | | | | |
| Infections and infestations | 0/3 (0%) | 2/3 (66.7%) | 0/3 (0%) | 0/3 (0%) | 0/3 (0%) |
| Metabolism and nutrition | 0/3 (0%) | 2/3 (66.7%) | 0/3 (0%) | 0/3 (0%) | 0/3 (0%) |
| disorders | | | | | |
| Musculoskeletal and | 1/3 (33.3%) | 0/3 (0%) | 0/3 (0%) | 0/3 (0%) | 0/3 (0%) |
| connective tissue disorders | | | | | |
| Nervous system disorders | 0/3 (0%) | 1/3 (33.3%) | 0/3 (0%) | 0/3 (0%) | 0/3 (0%) |
| Renal and urinary disorders | 1/3 (33.3%) | 0/3 (0%) | 0/3 (0%) | 0/3 (0%) | 0/3 (0%) |
| Respiratory and mediastinal | 0/3 (0%) | 1/3 (33.3%) | 0/3 (0%) | 0/3 (0%) | 0/3 (0%) |
| disorders | | | | | |
| Skin and subcutaneous | 1/3 (33.3%) | 1/3 (33.3%) | 0/3 (0%) | 0/3 (0%) | 0/3 (0%) |
| tissue disorders | | | | | |

TABLE 5
Treatment Related Adverse Events: n = 3.

| System Organ Class | Grade 1 | Grade 2 | Grade 3 | Grade 4 | Grade 5 |
|---|---|---|---|---|---|
| Gastrointestinal disorders | 0/3 (0%) | 1/3 (33.3%) | 0/3 (0%) | 0/3 (0%) | 0/3 (0%) |
| General disorders and | 1/3 (33.3%) | 0/3 (0%) | 1/3 (33.3%) | 0/3 (0%) | 0/3 (0%) |
| administration conditions | | | | | |
| Infections and infestations | 0/3 (0%) | 2/3 (66.7%) | 0/3 (0%) | 0/3 (0%) | 0/3 (0%) |
| Metabolism and nutrition | 0/3 (0%) | 2/3 (66.7%) | 0/3 (0%) | 0/3 (0%) | 0/3 (0%) |
| disorders | | | | | |
| Nervous system disorders | 0/3 (0%) | 1/3 (33.3%) | 0/3 (0%) | 0/3 (0%) | 0/3 (0%) |
| Respiratory and mediastinal | 0/3 (0%) | 1/3 (33.3%) | 0/3 (0%) | 0/3 (0%) | 0/3 (0%) |
| disorders | | | | | |
| Skin and subcutaneous | 0/3 (0%) | 1/3 (33.3%) | 0/3 (0%) | 0/3 (0%) | 0/3 (0%) |
| tissue disorders | | | | | |

Although the foregoing invention has been described in some detail by way of illustration and example for purposes of clarity of understanding, the descriptions and examples should not be construed as limiting the scope of the invention. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby. The disclosures of all patent and scientific literature cited herein are expressly incorporated herein in their entirety by reference. To the extent any material incorporated herein by reference is inconsistent with the express content of this disclosure, the express content controls.

1. A method of treating a cancer in a subject in need thereof, comprising administering to the subject a therapeutically effective amount of (a) an IL-2 conjugate, and (b) an anti-EGFR antibody, wherein the IL-2 conjugate comprises the amino acid sequence of SEQ ID NO: 3 in which at least one amino acid residue in the IL-2 conjugate is replaced by the structure of Formula (I): 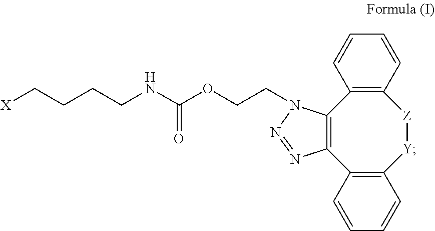
wherein:
Z is CH₂ and Y is 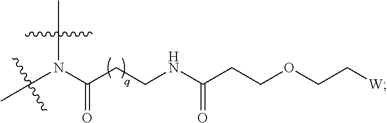
Y is CH₂ and Z is 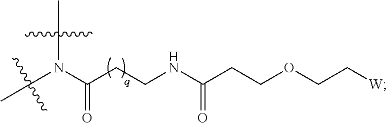
Z is CH₂ and Y is 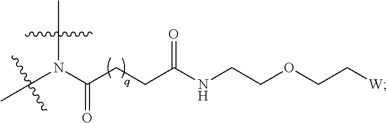
or
Y is CH₂ and Z is 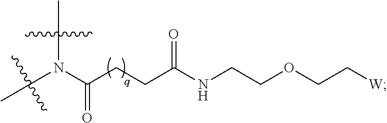
W is a PEG group having a molecular weight of about 5 kDa, about 10 kDa, about 15 kDa, about 20 kDa, about 25 kDa, about 30 kDa, about 35 kDa, about 40 kDa, about 45 kDa, about 50 kDa, or about 60 kDa; and
X has the structure: 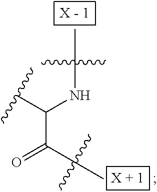 [SMILES: *NC(*)C(*)=O]
X−1 indicates the point of attachment to the preceding amino acid residue; and
X+1 indicates the point of attachment to the following amino acid residue;
wherein the position of the structure of Formula (I) in the amino acid sequence of the IL-2 conjugate is selected from K34, F41, F43, K42, E61, P64, R₃₇, T40, E67, Y44, V68, and L71.

2. (canceled)

3. (canceled)

4. The method of claim 1, wherein in the IL-2 conjugate Z is CH₂ and Y is

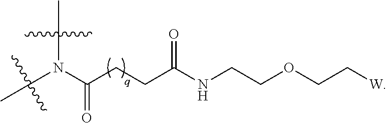

5. The method of claim 1, wherein in the IL-2 conjugate Y is CH₂ and Z is

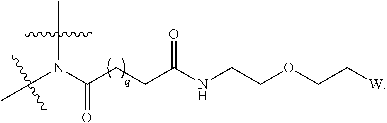

6. The method of claim 1, wherein in the IL-2 conjugate the PEG group has an average molecular weight of about 25 kDa, about 30 kDa, or about 35 kDa.

7. The method of claim 6, wherein in the IL-2 conjugate the PEG group has an average molecular weight of about 30 kDa.

8. The method of claim 1, wherein the position of the structure of Formula (I) in the amino acid sequence of the IL-2 conjugate is P64.

9. The method of claim 1, wherein the structure of Formula (I) has the structure of Formula (IV) or Formula (V), or the IL-2 conjugate is provided as is a mixture comprising an IL-2 conjugate in which the structure of Formula (I) has the structure of Formula (IV) and an IL-2 conjugate in which the structure of Formula (I) has the structure of Formula (V): 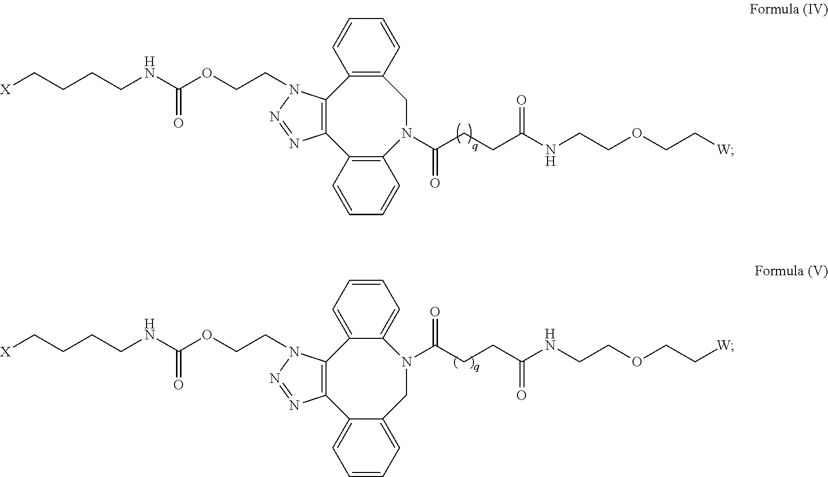
wherein:
W is a PEG group having a molecular weight of about 25 kDa, about 30 kDa, or about 35 kDa;
q is 1, 2, or 3;
X has the structure: 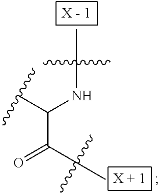 [SMILES: *NC(*)C(*)=O]
X−1 indicates the point of attachment to the preceding amino acid residue; and
X+1 indicates the point of attachment to the following amino acid residue.

10.-12. (canceled)

13. The method of claim 1, wherein q is 1.

14.-18. (canceled)

19. The method of claim 1, wherein the IL-2 conjugate is administered to the subject about once every two weeks, about once every three weeks, or about once every 4 weeks.

20. (canceled)

21. The method of claim 1, wherein the IL-2 conjugate is administered to a subject by intravenous administration.

22. The method of claim 1, wherein the IL-2 conjugate and the anti-EGFR antibody are administered separately, optionally wherein the IL-2 conjugate and the anti-EGFR antibody are administered sequentially.

23. (canceled)

24. The method of claim 1, wherein the cancer is selected from renal cell carcinoma (RCC), non-small cell lung cancer (NSCLC), head and neck squamous cell cancer (HNSCC), classical Hodgkin lymphoma (cHL), primary mediastinal large B-cell lymphoma (PMBCL), urothelial carcinoma, microsatellite unstable cancer, microsatellite stable cancer, gastric cancer, colon cancer, colorectal cancer (CRC), cervical cancer, hepatocellular carcinoma (HCC), Merkel cell carcinoma (MCC), melanoma, small cell lung cancer (SCLC), esophageal, esophageal squamous cell carcinoma (ESCC), glioblastoma, mesothelioma, breast cancer, triple-negative breast cancer, prostate cancer, castrate-resistant prostate cancer, metastatic castrate-resistant prostate cancer, or metastatic castrate-resistant prostate cancer having DNA damage response (DDR) defects, bladder cancer, ovarian cancer, tumors of moderate to low mutational burden, cutaneous squamous cell carcinoma (CSCC), squamous cell skin cancer (SCSC), tumors of low-to non-expressing PD-L1, tumors disseminated systemically to the liver and CNS beyond their primary anatomic originating site, and diffuse large B-cell lymphoma.

25. (canceled)

26. (canceled)

27. The method of claim 1, wherein the anti-EGFR antibody is selected from cetuximab, panitumumab (Vectibix), necitumumab (Portrazza), JNJ-61186372 (Amivantamab), IMC-C₂₂₅, ABX-EGF, ICR62, and EMD 55900.

28. (canceled)

29. (canceled)

30. A method of improving the antibody-dependent cellular cytotoxicity (ADCC) of an anti-EGFR antibody, comprising administering to the subject a therapeutically effective amount of (a) an IL-2 conjugate, and (b) an anti-EGFR antibody, wherein the IL-2 conjugate comprises the amino acid sequence of SEQ ID NO: 3 in which at least one amino acid residue in the IL-2 conjugate is replaced by the structure of Formula (I): 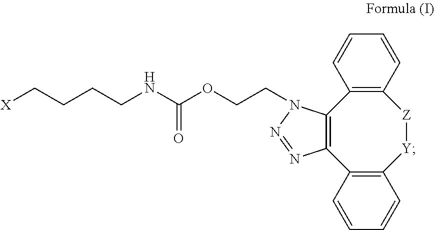
wherein:
Z is CH₂ and Y is 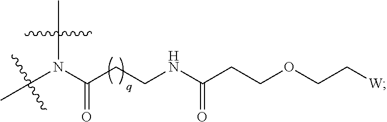
Y is CH₂ and Z is 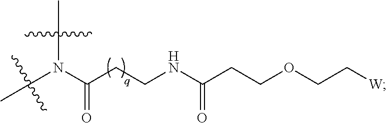
Z is CH₂ and Y is 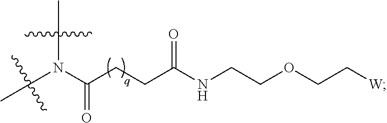
or
Y is CH₂ and Z is 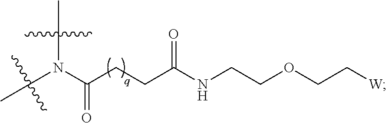
W is a PEG group having a molecular weight of about 5 kDa, about 10 kDa, about 15 kDa, about 20 kDa, about 25 kDa, about 30 kDa, about 35 kDa, about 40 kDa, about 45 kDa, about 50 kDa, or about 60 kDa; and
X has the structure: 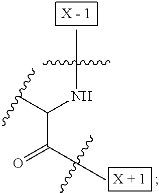 [SMILES: *NC(*)C(*)=O]
X−1 indicates the point of attachment to the preceding amino acid residue; and
X+1 indicates the point of attachment to the following amino acid residue;
wherein the position of the structure of Formula (I) in the amino acid sequence of the IL-2 conjugate is selected from R₃₇, T40, F41, K42, F43, Y44, E61, P64, E67, V68, and L71 in reference to the amino acid positions within SEQ ID NO: 3.

31. The method of claim 30, wherein in the IL-2 conjugate the PEG group has an average molecular weight of about 25 kDa, about 30 kDa, or about 35 kDa.

32. The method of claim 30, wherein the position of the structure of Formula (I) in the amino acid sequence of the IL-2 conjugate is F41, E61, or P64.

33. The method of claim 30, wherein the structure of Formula (I) has the structure of Formula (IV) or Formula (V), or the IL-2 conjugate is provided as a mixture comprising an IL-2 conjugate in which the structure of Formula (I) has the structure of Formula (IV) and an IL-2 conjugate in which the structure of Formula (I) has the structure of Formula (V): 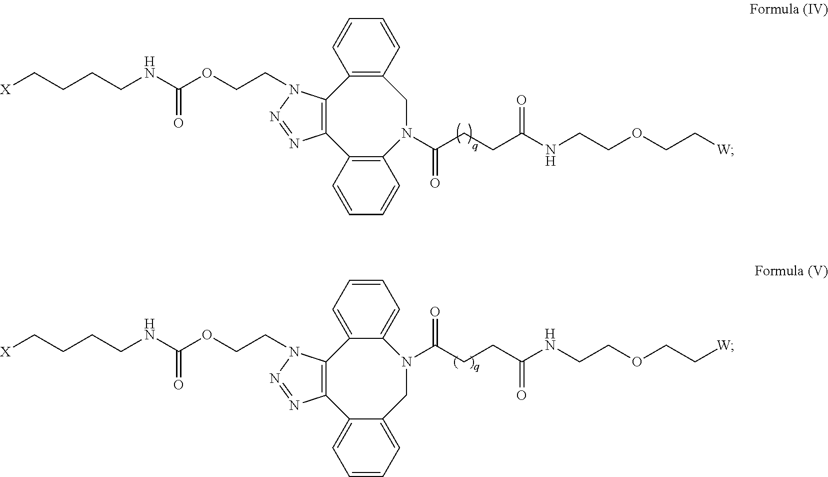
wherein:
W is a PEG group having a molecular weight of about 25 kDa, about 30 kDa, or about 35 kDa;
q is 1, 2, or 3;
X has the structure: 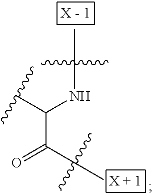 [SMILES: *NC(*)C(*)=O]
X−1 indicates the point of attachment to the preceding amino acid residue; and
X+1 indicates the point of attachment to the following amino acid residue.

34. A method of stimulating CD8+ T cells and/or NK cells, comprising administering to the subject a therapeutically effective amount of (a) an IL-2 conjugate, and (b) an anti-EGFR antibody, wherein the IL-2 conjugate comprises the amino acid sequence of SEQ ID NO: 3 in which at least one amino acid residue in the IL-2 conjugate is replaced by the structure of Formula (I): 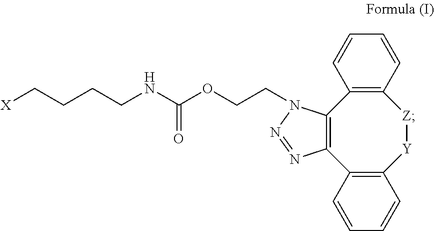
wherein:
Z is CH₂ and Y is 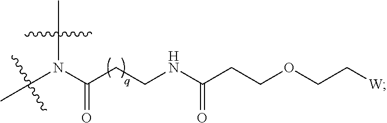
Y is CH₂ and Z is 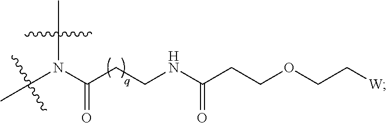
Z is CH₂ and Y is 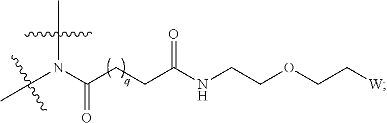
or
Y is CH₂ and Z is 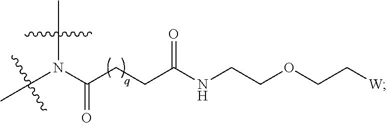
W is a PEG group having a molecular weight of about 5 kDa, about 10 kDa, about 15 kDa, about 20 kDa, 2 about 5 kDa, about 30 kDa, about 35 kDa, about 40 kDa, about 45 kDa, about 50 kDa, or about 60 kDa; and
X has the structure: 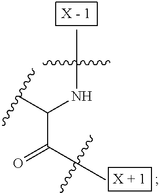 [SMILES: *NC(*)C(*)=O]
X−1 indicates the point of attachment to the preceding amino acid residue; and
X+1 indicates the point of attachment to the following amino acid residue;
wherein the position of the structure of Formula (I) in the amino acid sequence of the IL-2 conjugate is selected from R₃₇, T40, F41, K42, F43, Y44, E61, P64, E67, V68, and L71 in reference to the amino acid positions within SEQ ID NO: 3.

35. The method of claim 34, wherein in the IL-2 conjugate the PEG group has an average molecular weight of about 25 kDa, about 30 kDa, or about 35 kDa.

36. The method of claim 34, wherein the position of the structure of Formula (I) in the amino acid sequence of the IL-2 conjugate is F41, E61, or P64.

37. The method of claim 34, wherein the structure of Formula (I) has the structure of Formula (IV) or Formula (V), or the IL-2 conjugate is provided as a mixture comprising an IL-2 conjugate in which the structure of Formula (I) has the structure of Formula (IV) and an IL-2 conjugate in which the structure of Formula (I) has the structure of Formula (V): 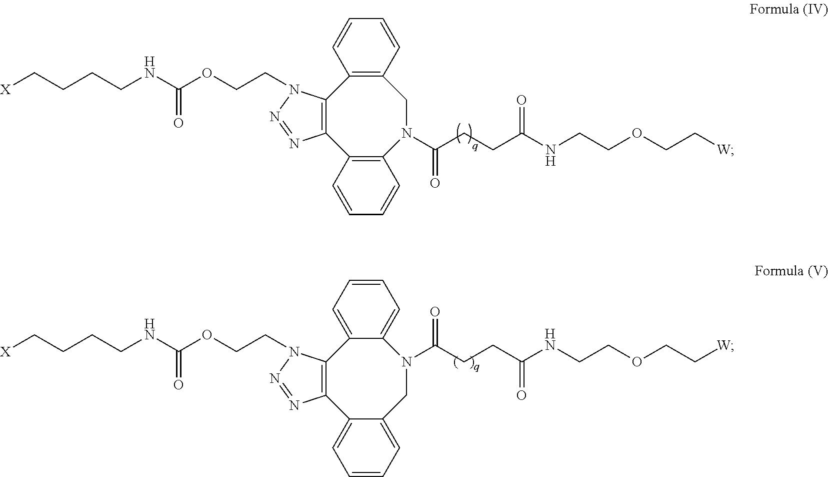
wherein:
W is a PEG group having a molecular weight of about 25 kDa, about 30 kDa, or about 35 kDa;
q is 1, 2, or 3;
X has the structure: 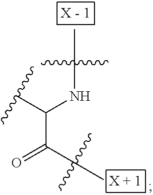 [SMILES: *NC(*)C(*)=O]
X−1 indicates the point of attachment to the preceding amino acid residue; and
X+1 indicates the point of attachment to the following amino acid residue.
